(19) United States
(12) Patent Application Publication
(10) Pub. No.: US 2015/0266857 A1
(43) Pub. Date: Sep. 24, 2015

(54) NOVEL 3-INDOL SUBSTITUTED DERIVATIVES, PHARMACEUTICAL COMPOSITIONS AND METHODS FOR USE

(71) Applicant: iTeos Therapeutics, Charleroi (BE)

(72) Inventors: Stefano Crosignani, Nivelles (BE); Sandra Cauwenberghs, Halle (BE); Gregory Driessens, Watermael-Boitsfort (BE); Frederik Deroose, Destelbergen (BE)

(21) Appl. No.: 14/660,082

(22) Filed: Mar. 17, 2015

Related U.S. Application Data
(60) Provisional application No. 61/996,975, filed on Mar. 18, 2014.

(51) Int. Cl. C07D 403/04; C07D 413/04; C07D 487/10; C07D 409/04; C07D 405/04; C07D 417/04; C07D 401/14; C07D 413/14

(52) U.S. Cl. CPC C07D 403/04; C07D 401/14; C07D 413/04; C07D 413/14; C07D 409/04; C07D 405/04; C07D 417/04; C07D 487/10

(57) ABSTRACT

The present invention relates to compound of Formula I

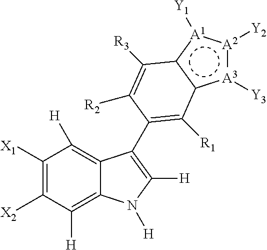

or pharmaceutically acceptable enantiomers, salts or solvates thereof. The invention further relates to the use of the compounds of Formula I as TDO2 inhibitors. The invention also relates to the use of the compounds of Formula I for the treatment and/or prevention of cancer, neurodegenerative disorders such as Parkinson's disease, Alzheimer's disease and Huntington's disease, chronic viral infections such as HCV and HIV, depression, and obesity. The invention also relates to a process for manufacturing compounds of Formula I.

### FIELD OF INVENTION

The present invention relates to novel 3-(indol-3-yl)-pyridine derivatives, including pharmaceutically acceptable enantiomers, salts and solvates thereof. Compounds of the invention are inhibitors of TDO2 (tryptophan 2,3-dioxygenase) and are useful as therapeutic compounds, particularly in the treatment and/or prevention of cancers.

### BACKGROUND OF INVENTION

Two decades after the importance of tryptophan catabolism for maintaining the immune privilege of the placenta was discovered (Munn, D. H. et al., Science, 1998, 281, 1191-1193), increasing evidence is extending its biological relevance beyond immune tolerance to non-self. According to the generally accepted concept, tryptophan, an essential amino acid, is catabolized in the local microenvironment of tumors, immune-privileged sites, or sites of inflammation (Mellor A L and Munn D H., Nat Rev Immunol, 2008, 8, 74-80). In these tissues, cancer cells, immune cells, or specialized epithelial cells (e.g., syncytiotrophoblasts in the placenta) create an immunosuppressive environment in tumors that shuts down antitumor immune responses in tumors and in tumor-draining lymph nodes by inducing T-cell anergy and apoptosis through depletion of tryptophan and accumulation of immunosuppressive tryptophan catabolites (Munn D H et al., J Exp Med., 1999, 189, 1363-1372; Fallarino F et al., Cell Death Differ., 2002, 9, 1069-1077).

It has recently been discovered that a key enzyme in tryptophan catabolism, tryptophan 2,3-dioxygenase (TDO2), which is considered responsible for regulating systemic tryptophan levels in the liver, is constitutively expressed in a wide variety of cancers, such as for example in bladder carcinoma, hepatocarcinoma, melanoma, mesothelioma, neuroblastoma, sarcoma, breast carcinoma, leukemia, renal cell carcinoma, colorectal carcinoma, head and neck carcinoma, lung carcinoma, brain tumor, glioblastoma, astrocytoma, myeloma, and pancreatic carcinoma (Pilotte L et al., Proc Natl Acad Sci USA, 2012, 109(7), 2497-502). TDO2 expression in tumor cells prevents tumor surveillance by the immune system and thus prevents tumor rejection by locally degrading tryptophan (Opitz C A et al., Nature, 2011, 478(7368), 197-203). The first evidence for this was provided through inhibition of TDO2 by a small molecule which inhibited tumor growth in a P815 mastocytoma tumor model with a prophylactic vaccination approach (Pilotte L et al., Proc Natl Acad Sci USA, 2012, 109(7), 2497-502). P815mTDO2 expressing tumors were rejected less in comparison to P815 tumors transfected with an empty vector, clearly demonstrating a growth benefit for TDO2 expressing tumors. Inhibition with a TDO2 inhibitor strongly decreased tumor growth in P815mTDO2 implanted tumors. Anti-tumor activity with the TDO2 inhibitor was equally observed in the P815 control implanted tumors negative for TDO2, thus providing evidence for an effect of TDO2 expressed in the immune system of the animal. These experiments for the first time provided clear evidence for a role of TDO2 in regulating tumor growth through expression in the cancer cell as well as immune compartment.

In line with its expression profile in liver, TDO2 was found predominantly in hepatocellular carcinoma (HCC) (Pilotte L et al., Proc Natl Acad Sci USA, 2012, 109(7), 2497-502). Inhibition of tryptophan catabolism and thus restoration of tryptophan concentration and decreased production of downstream metabolites could prove beneficial in the context of liver disease progressing to the stage of liver carcinoma. More particularly: (i) several reports have shown evidence that increased availability of tryptophan through supplementation is beneficial for example, cirrhotic livers, allowing the direct use of tryptophan for protein synthesis (Ohta et al., Amino Acids, 1996, 10(4), 369-78); (ii) there is a correlation between increased downstream serum tryptophan metabolites, such as quinolinic acid, and hepatic dysfunction in patients with liver cirrhosis (Lahdou et al., Hum Immunol, 2013, 74(1), 60-6) and (iii) increased secretion of another tryptophan metabolite, indole-3-lactic acid, has been associated with alcohol-induced liver disease in mice (Manna et al., J Proteome Res, 2011, 10(9), 4120-33). In the context of liver carcinoma itself, very high RNA expression is a good indication for therapeutic evaluation of TDO2 inhibitors (Pilotte L et al., Proc Natl Acad Sci USA, 2012, 109(7), 2497-502). The above thus provides a clear rationale for TDO2 activity modulation in the control of liver tumor development.

In addition to expression in liver, TDO2 is expressed in neurons, microglia and astrocytes and the potential benefit of TDO2 inhibition in the context of glioma was shown in another animal model. Platten and collaborators demonstrated that the tryptophan catabolite kynurenine produced by TDO expressed in the tumor cells suppresses antitumour immune responses and promotes tumor-cell survival and motility through the AHR in an autocrine/paracrine fashion (Opitz C A et al., Nature, 2011, 478(7368), 197-203). The TDO-AHR pathway is active in human brain tumors and is associated with malignant progression and poor survival. Further evidence came from the accumulation of a downstream metabolite, quinolinic acid which accumulates in human gliomas and was associated with a malignant phenotype (Sahm et al., Cancer Res, 2013, 73(11), 3225-34). Here tryptophan catabolism was shown to occur in microglia cells as well. The above data thus provides evidence for TDO2 targeting in glioma with brain-penetrant small molecules.

Other tumor types in which TDO2 mRNA was found are breast carcinoma, bladder, renal cell, pancreatic, colorectal, head & neck carcinoma and lung carcinoma as well as melanoma thus broadening the scope of TDO2 targeting beyond HCC and glioma (Pilotte L et al., Proc Natl Acad Sci USA, 2012, 109(7), 2497-502).

The enhanced Tryptophan degradation observed in patients with gynecological cancers (ovarian carcinoma, cervical cancer, endometrial cancer) provides additional rationale for TDO2 targeting in those cancers (Sperner-Unterweger B et al, Immunology, 2011, 216 (3); 296-301).

The tryptophan catabolism in some cancers might be also increased by the expression of indoleamine 2,3-dioxygenase (IDO1) by tumor cells (Uyttenhove, C. et al., Nat. Med., 2003, 9, 1269-1274).

Because tryptophan catabolism is induced by inflammatory mediators, notably IFN-gamma, it is thought to represent an endogenous mechanism that restricts excessive immune responses, thereby preventing immunopathology. However in the context of cancer, there is strong evidence that suppression of antitumor immune responses in precancerous lesions and established cancers by tryptophan catabolism promotes tumor growth, which would make such catabolism an attractive target for therapeutic intervention (Dolu\check{s}ić E and Frédérick R., Expert Opin Ther Pat., 2013, 23(10), 1367-81). Hence, a considerable effort is being made to identify selective and efficient inhibitors of tryptophan catabolism to enhance the efficacy of conventional chemotherapy, immune checkpoints (Holmgaard R B et al., J Exp Med., 2013, 210(7), 1389-402) or therapeutic vaccines.

In the context of neurological brain disorders, TDO2 expression has been demonstrated in neurons, brain vasculature and additionally in the case of schizophrenia in astroglial cells (Miller C et al., 2004, Neurobiology Dis, 15(3):618-29). The kynurenine pathway is now considered as a therapeutic target in cognitive diseases like bipolar disorder or Tourette syndrome and neurodegenerative disorders like Alzheimer, motor neuron disease like Amyotrophic lateral sclerosis, Multiple sclerosis, Huntington or Parkinson's disease (Stone T W, 2013, Br J of Pharmacol, 169(6): 1211-27; Wu et al, 2013, Plos One, 8(4):e59749; Füvesi et al, 2012, J Neural Transm, 119(2):225-34; Widner et al, 2002, J Neural Transm, 109(2):181-9; Comings et al, 1996, Pharmacogenetics, 6(4):307-18; Forrest 2010, J Neurochem, 112(1):112-22).

Cognitive changes related to Tryptophan catabolism have also been shown in patients infected with human immunodeficiency virus type-1 (HIV), called HIV-associated neurocognitive disorder (HAND) (Davies et al, 2010, Int J of Tryptophan Res, 3:121-40). In addition, T cell hyporesponsiveness has been recently associated with the Tryptophan catabolic pathway in HIV-infected patients with possibly extension to other chronic infectious diseases like e.g. Hepatitis C.

Some TDO2 inhibitors were proposed in WO2010/008427 and by Dolusic, E. et al. (Dolusic et al., J. Med. Chem., 2011, 54, 5320-5334), however either their affinity for the target is limited, or their pharmacokinetic properties are not suitable for development as a drug for human use.

Therefore, there is a need for new TDO2 inhibitors with improved efficacy for cancer treatment and/or prevention.

### SUMMARY OF THE INVENTION

The present invention provides new TDO2 inhibitors which may be administered to a mammalian subject having a condition or disease where it is desirable to modulate, and in particular decrease, activity of TDO2, including, without limitation, patients diagnosed with cancer, or any subject being at risk of developing a cancer. Also provided are compositions containing these compounds and uses thereof.

In one aspect, a compound of Formula I is provided or a pharmaceutically acceptable salt, solvent or solvate thereof, where A¹, A², A³, Y¹, Y², Y³, R¹, R², R³, X¹ and X² are as defined herein.
In one embodiment, a first one of A¹, A² and A³ is N, a second one of A¹, A² and A³ is C and the third one of A¹, A² and A³ is N or O. In another embodiment, A² is N and one of A¹ and A³ is N or S and the other is C. In yet another embodiment, A¹ or A³ is S, A¹-Y¹ or A³-Y³ is SO₂. In still a further embodiment, A² is C and one of A¹ and A³ is N and the other is N or O. In yet a further embodiment, A or A³ is O, and C is substituted with CR⁶R⁷R⁸ as defined herein and one of R⁶, R⁷, R⁸ is a 1. wherein A or A³ is O, and C is substituted with CR⁶R⁷R⁸ and R⁶, R⁷ or R⁸ is heterocyclyl or C1-heterocycyl, the heterocyclyl is piperidine, pyrrolidine, piperazine, morpholine, or 2,6-diazaspiro[3.3]heptane, any of which may be optionally substituted with one or more of C1-C3 alkyl, amino, hydroxyl, halogen, COCH₃, COOH, or SO₂CH₃. In yet a further embodiment, one of A¹, A² or A³ is S or A¹-Y¹, A²-Y², or A³-Y³ is SO₂; one of A¹, A² or A³ is N or C and one of A¹, A² and A³ is C. In a further embodiment, X¹ and X² are independently H or F. In still another embodiment, R¹, R² and R³ represent each independently H, halogen or methyl, or may each be H.
In a further aspect, a pharmaceutical composition is provided which comprises a compound according to Formula I is provided, or a pharmaceutically acceptable enantiomer, salt or solvate thereof, and at least one pharmaceutically acceptable carrier, diluent, excipient and/or adjuvant.
In yet another aspect, a medicament is provided which comprises a compound according to Formula I, or a pharmaceutically acceptable enantiomer, salt or solvate thereof.
In yet a further aspect, a compound of Formula I, or a pharmaceutically acceptable enantiomer, salt or solvate thereof is provided, for use in the treatment and/or prevention of cancer, neurodegenerative disorders such as Parkinson's disease, Alzheimer's disease and Huntington's disease, chronic viral infections such as HCV and HIV, depression, and obesity, or for use as TDO2 inhibitor.
In still another aspect, a method of treating and/or preventing of cancer, neurodegenerative disorders such as Parkinson's disease, Alzheimer's disease and Huntington's disease, chronic viral infections such as HCV and HIV, depression, and obesity, or inhibiting TD02 is provide. The method comprises administering a compound of Formula I, or a pharmaceutically acceptable salt thereof.
In a further aspect, a process for manufacturing a compound of Formula I or a pharmaceutically acceptable enantiomer, salt or solvate thereof is provide. The process comprises:
   (a1) reacting a compound of Formula (i)

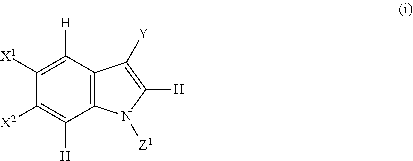

wherein
X¹ and X² represent each independently H, halogen, alkyl, haloalkyl;
Z¹ represents H or an amino protecting group;
Y represents an halogen, an alkylsulfonyloxy having 1-6 carbon atoms or arylsulfonyloxy having 6-10 carbon atoms;
with a compound of Formula (ii)

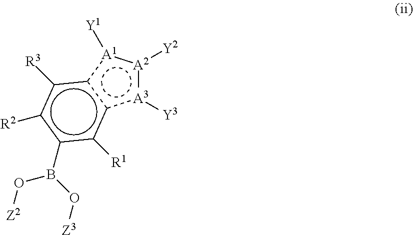

wherein
R¹, R², R³, A¹, A², A³, Y¹, Y² and Y³ are as defined herein; and Z² and Z³ represent H or alkyl groups, with the possibility for Z² and Z³ to form a ring;
so as to obtain a compound of Formula (iii),

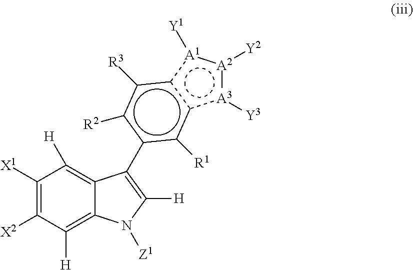

wherein Z¹, X¹, X², R¹, R², R³, A¹, A², A³, Y¹, Y² and Y³ are defined herein; and
(b1) in the case wherein Z¹ is not H, deprotecting the indole amine of compound of Formula (iii), to afford compound of Formula I.
Still other aspects and advantages of the invention will be apparent from the following detailed description of the invention.

### DETAILED DESCRIPTION

### Compounds

This invention relates to compounds of Formula I

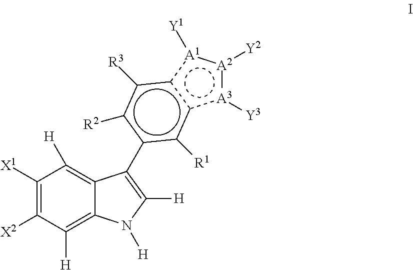

or a pharmaceutically acceptable enantiomer, salt or solvate thereof, wherein:
X¹ and X² represent each independently H, halogen, alkyl, or haloalkyl;
R¹, R² and R³ represent each independently H, halogen, C1-C6 alkyl, alkoxy, or haloalkyl, optionally substituted by one or more substituents selected from halogen, hydroxyl, OR⁴, COOR⁴, CONR⁴R⁵, NR⁴COR⁵, NR⁴R⁵, SO₂R⁴, SO₂NR⁴R⁵, NR⁴SO₂R⁵, SO₂R⁴, aryl, CO-alkyl, or C1-C6 alkyl which is optionally substituted by one or more groups selected from halogen, hydroxyl, amino or COOH; wherein R⁴ and R⁵ represent each independently a hydrogen atom or a group, optionally substituted, selected from C1-C6 alkyl, aryl, arylalkyl, alkylaryl, heteroaryl, heteroarylalkyl, alkylheteroaryl, or amino;
A¹, A² and A³ represent each independently C, N, S or O, wherein when A¹, A² or A³ is S, A¹-Y¹, A²-Y² or A³-Y³ is optionally SO₂;
each of Y¹, Y² and Y³ is either absent or represent independently
   a) a hydrogen atom;
   b) oxo;
   c) SH
   d) CR⁶R⁷R⁸, NR⁶R⁷ and OR⁶ wherein R⁶, R⁷ and R⁸ represent each independently:
      i) a hydrogen atom;
      ii) halogen;
      iii) hydroxyl;
      iv) OR⁹ or NR⁹R¹⁰ wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino, CO-alkyl, or SO₂R¹¹, wherein R¹¹ represents a hydrogen atom or a group, optionally substituted, selected from C1-C6 alkyl, aryl, arylalkyl, alkylaryl, heteroaryl, heteroarylalkyl, alkylheteroaryl, hydroxyl, or amino;
      v) C1-C10 alkyl, linear or branched; optionally substituted with up to three substituents selected from halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, or CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, or amino;
      vi) heterocyclyl or C1-C2 alkyl-heterocyclyl; wherein the heterocyclyl is optionally substituted with up to three substituents halogen, hydroxyl, oxo, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, or alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino;
      vii) —CO—R¹¹ or —SO₂R¹¹ wherein R¹¹ represents a group selected from hydroxy, amine, alkyl, heterocyclyl; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, or C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino;
      viii) optionally when Y1, Y2 or Y3 is CR⁶R⁷R⁸, R⁶, R⁷ and the carbon atom to which they are attached form together a ring selected from:
         cycloalkyl, optionally substituted with up to three substituents selected from halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, or C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino; or
         heterocyclyl, optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, or a C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino; or
      ix) optionally when Y1, Y2 or Y3 is NR⁶R⁷, R⁶, R⁷ and the nitrogen atom to which they are attached form together a ring; optionally substituted with up to three substituents selected from halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹¹, SO₂R⁹, aryl, CO-alkyl, or C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino; and wherein R¹¹ represents a hydrogen atom or an optionally substituted group selected from aryl, arylalkyl, alkylaryl, heteroaryl, heteroarylalkyl, alkylheteroaryl, or amino; or R¹ represents an alkyl group optionally substituted with up to three substituents selected from halogen, hydroxyl, OR¹², COOR¹², CONR¹²R¹³, NR¹²COR¹³, NR¹²R¹³, SO₂R¹², SO₂NR¹²R¹³, NR¹²SO₂R¹³, SO₂R¹², or aryl; wherein R¹² and R¹³ represent each independently a hydrogen atom or a group, optionally substituted, selected from C1-C6 alkyl, aryl, arylalkyl, alkylaryl, heteroaryl, heteroarylalkyl, alkylheteroaryl, or amino;
         dotted lines stand for single or double bonds;
         provided that A¹, A² and A³ are not all C;
         provided that when one of A¹ or A³ is N the two others are not both C; and provided that when one of A¹, A² and A³ is S, only one S is present. at least one of the other two is C, and the other is C or N;
         provided that compound of Formula I is not 3-(benzofuran-5-yl)-6-chloro-1H-indole or 3-(benzo[d][1,3]dioxol-5-yl)-1H-indole.

In another embodiment, in Formula I:
X¹ and X² represent each independently H, halogen, alkyl, haloalkyl, preferably H or F;
R¹, R² and R³ represent each independently H, halogen, C1-C6 alkyl, alkoxy, haloalkyl, optionally substituted by one or more substituents selected from the group comprising halogen, hydroxyl, OR⁴, COOR⁴, CONR⁴R⁵, NR⁴COR⁵, NR⁴R⁵, SO₂R⁴, SO₂NR⁴R⁵, NR⁴SO₂R⁵, SO₂R⁴, aryl, CO-alkyl, alkyl, the alkyl group being

optionally substituted by one or more groups selected from halogen, hydroxyl, amino or COOH; wherein R⁴ and R⁵ represent each independently a hydrogen atom or a group, optionally substituted, selected from C1-C6 alkyl, aryl, arylalkyl, alkylaryl, heteroaryl, heteroarylalkyl, alkylheteroaryl, amino;
preferably R¹, R² and R³ represent each independently H, halogen or alkyl,
preferably R¹, R² and R³ represent each independently H, halogen or methyl,
preferably R¹, R² and R³ represent each H;
A¹, A² and A³ represent each independently C, N or O;
each of Y¹, Y² and Y³ is either absent or represent independently
   a hydrogen atom;
   oxo;
   CR⁶R⁷R⁸, NR⁶R⁷ and OR⁶ wherein R⁶, R⁷ and R⁸ represent each independently:
      a hydrogen atom;
      halogen, preferably F, Cl or I, more preferably F;
      hydroxyl;
      OR⁹ or NR⁹R¹⁰ wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino, CO-alkyl, SO₂R¹¹, wherein R¹¹ represents a hydrogen atom or a group, optionally substituted, selected from C1-C6 alkyl, aryl, arylalkyl, alkylaryl, heteroaryl, heteroarylalkyl, alkylheteroaryl, amino;
      C1-C10 alkyl, linear or branched, preferably methyl, ethyl or propyl; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, amino;
      heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
      —CO—R¹¹ or —SO₂R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
      in CR⁶R⁷R⁸, R⁶, R⁷ and the carbon atom to which they are attached form together a ring, said ring being preferably selected from:
         cycloalkyl, optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
         heterocyclyl, preferably selected from morpholine, piperazine or piperidine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
      in NR⁶R⁷, R⁶, R⁷ and the nitrogen atom to which they are attached form together a ring, said ring being preferably an heterocyclyl, preferably selected from morpholine, piperazine or piperidine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹¹, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino; and wherein R¹¹ represents a hydrogen atom or an optionally substituted group selected from aryl, arylalkyl, alkylaryl, heteroaryl, heteroarylalkyl, alkylheteroaryl, amino; or R¹¹ represents an alkyl group optionally substituted with up to three substituents selected from the group comprising halogen, hydroxyl, OR¹², COOR¹², CONR¹²R¹³, NR¹²COR¹³, NR¹²R¹³, SO₂R¹², SO₂NR¹²R¹³, NR¹²SO₂R¹³, SO₂R¹², aryl; wherein R¹² and R¹³ represent each independently a hydrogen atom or a group, optionally substituted, selected from C1-C6 alkyl, aryl, arylalkyl, alkylaryl, heteroaryl, heteroarylalkyl, alkylheteroaryl, amino;
         dotted lines stand for single or double bonds;
         provided that A¹, A² and A³ are not all C;
         provided that when one of A¹, A² and A³ is N, the two others are not both C;
         provided that compound of Formula I is not 3-(benzofuran-5-yl)-6-chloro-1H-indole or 3-(benzo[d][1,3]dioxol-5-yl)-1H-indole.

According to one embodiment, the fused heterocycle which substitutes the indole ring in Formula I is aromatic. In another embodiment, the fused heterocycle which substituted the indole ring in Formula I is partially aromatic. In a further embodiment, the heterocycle which substituted the indole ring in Formula I is non-aromatic.

According to a preferred embodiment, in Formula I, X¹ and X² represent each independently H or halogen, preferably H or F. According to a specific embodiment, X¹ represents H and X² represents F. According to a preferred embodiment, in Formula I, R¹, R² and R³ represent each H. According to a specific embodiment, in Formula I, X¹ and X² represent each independently H or halogen and R¹, R² and R³ represent each H; preferably X¹ represents H and X² represents F and R¹, R² and R³ represent each H

According to one embodiment, in Formula I, a first one of A¹, A² and A³ is N, a second one of A, A² and A³ is C and the third one of A¹, A² and A³ is N or O.

According to one embodiment, in Formula I, A² is N and one of A and A³ is N or S r and the other is C.

According to one embodiment, in Formula I, A² is C and one of A¹ and A³ is N and the other is N or O. In one embodiment, wherein A¹ or A³ is O, and C is substituted with CR⁶R⁷R⁸ and R⁶, R⁷ or R⁸ is heterocyclyl or C1-heterocycyl, the heterocyclyl is piperidine, pyrrolidine, piperazine, morpholine, or 2,6-diazaspiro[3.3]heptane, any of which may be optionally substituted with one or more of C1-C3 alkyl, amino, hydroxyl, halogen, COCH₃, COOH, or SO₂CH₃.

According to another embodiment, in Formula I, A¹ or A³ is S, A¹-Y or A³-Y³ is SO₂. Optionally, wherein one of A¹, A² or A³ is S or A-Y, A²-Y², or A³-Y³ is SO₂; one of A¹, A² or A³ is N or C and one of A¹, A² and A³ is C.

In one embodiment, preferred compounds of Formula I are those of Formula II-1

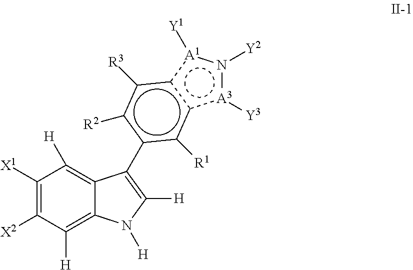

and pharmaceutically acceptable enantiomers, salts and solvates thereof, wherein X¹, X², R¹, R², R³, A¹, A³, Y¹, Y² and Y³ are as defined in Formula I.

According to one embodiment, in Formula II-1, A and A³ are not both C. According to another embodiment, one of A and A¹ is S and the other is C, A¹-Y¹ or A³-Y³ is optionally SO₂, and the C is optionally substituted with oxo.

In one embodiment, preferred compounds of Formula I are those of Formula II-2

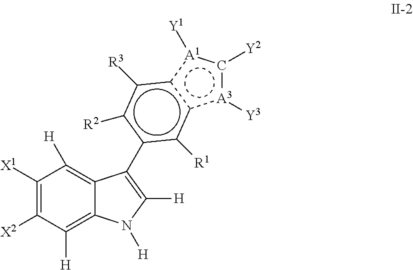

and pharmaceutically acceptable enantiomers, salts and solvates thereof, wherein X¹, X², R¹, R², R³, A¹, A³, Y¹, Y² and Y³ are as defined in Formula I.

According to one embodiment, in Formula II-1, {A¹, A³} are not {C, N}, {N, C} or {C, C}.

In one embodiment, preferred compounds of Formula I are those of Formula II-1a

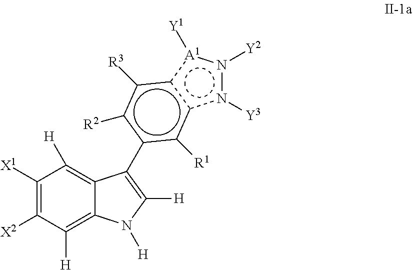

and pharmaceutically acceptable enantiomers, salts and solvates thereof, wherein X¹, X², R¹, R², R³, A¹, Y¹, Y² and Y³ are as defined in Formula I.

In one embodiment, preferred compounds of Formula I are those of Formula II-1b

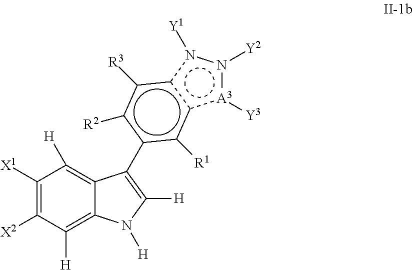

and pharmaceutically acceptable enantiomers, salts and solvates thereof, wherein X¹, X², R¹, R², R³, A³, Y¹, Y² and Y³ are as defined in Formula I.

In one embodiment, preferred compounds of Formula I are those of Formula II-2a

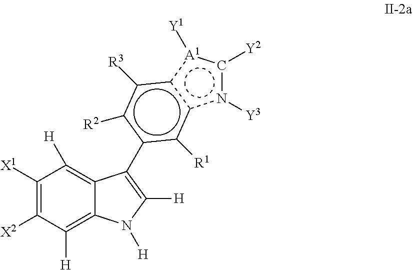

and pharmaceutically acceptable enantiomers, salts and solvates thereof, wherein X¹, X², R¹, R², R³, A¹, Y¹, Y² and Y³ are as defined in Formula I.

According to one embodiment, in Formula II-2a, A is not C.

In one embodiment, preferred compounds of Formula I are those of Formula II-2b

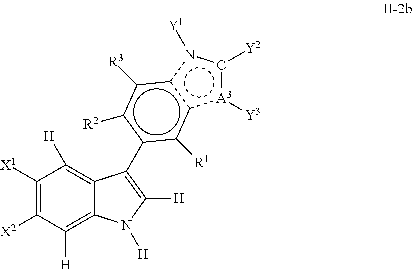

and pharmaceutically acceptable enantiomers, salts and solvates thereof, wherein X¹, X², R¹, R², R³, A³, Y¹, Y² and Y³ are as defined in Formula I.

According to one embodiment, in Formula II-2b, A³ is not C. In one embodiment, preferred compounds of Formula I are those of Formula II-1a1

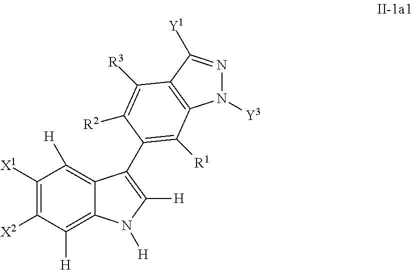

and pharmaceutically acceptable enantiomers, salts and solvates thereof, wherein X¹, X², R¹, R², R³, Y¹ and Y³ are as defined in Formula I.

According to a preferred embodiment, in Formula II-1a1, Y¹ represents H and Y³ is as defined in Formula I, preferably, Y³ represents:
a hydrogen atom;
CR⁶R⁷R⁸ wherein R⁶, R⁷ and R⁸ represent each independently:
   a hydrogen atom;
   C1-C10 alkyl, linear or branched, preferably methyl, ethyl or propyl; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SOR⁹, aryl, CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, amino;
   heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   —CO—R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   R⁶, R⁷ and the carbon atom to which they are attached form together a ring, said ring being preferably an heterocyclyl, preferably selected from morpholine, piperazine or piperidine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.

In one embodiment, preferred compounds of Formula I are those of Formula II-1a2

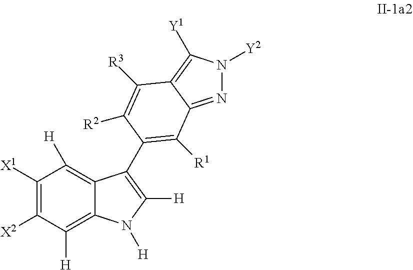

and pharmaceutically acceptable enantiomers, salts and solvates thereof, wherein X¹, X², R¹, R², R³, Y¹ and Y² are as defined in Formula I.

According to a preferred embodiment, in Formula II-1a2, Y¹ represents H and Y² is as defined in Formula I, preferably, Y² represents:
a hydrogen atom;
CR⁶R⁷R⁸ wherein R⁶, R⁷ and R⁸ represent each independently:
   a hydrogen atom;
   C1-C10 alkyl, linear or branched, preferably methyl, ethyl or propyl; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SOR⁹, aryl, CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, amino;
   heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   —CO—R¹¹ or —SO₂R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   R⁶, R⁷ and the carbon atom to which they are attached form together a ring, said ring being preferably an heterocyclyl, preferably selected from morpholine, piperazine or piperidine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.

In one embodiment, preferred compounds of Formula I are those of Formula II-1b1

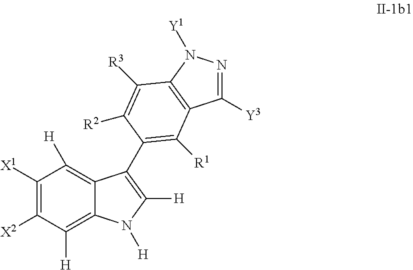

and pharmaceutically acceptable enantiomers, salts and solvates thereof, wherein X¹, X², R¹, R², R³, Y¹ and Y³ are as defined in Formula I.

According to a preferred embodiment, in Formula II-1 b1, Y³ represents H and Y is as defined in Formula I, preferably, Y¹ represents:
a hydrogen atom;
CR⁶R⁷R⁸ wherein R⁶, R⁷ and R⁸ represent each independently:
   a hydrogen atom;
   C1-C10 alkyl, linear or branched, preferably methyl, ethyl or propyl; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SOR⁹, aryl, CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, amino;
   heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   —CO—R¹¹ or —SO₂R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   R⁶, R⁷ and the carbon atom to which they are attached form together a ring, said ring being preferably an heterocyclyl, preferably selected from morpholine, piperazine or piperidine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.

In one embodiment, preferred compounds of Formula I are those of Formula II-1b2

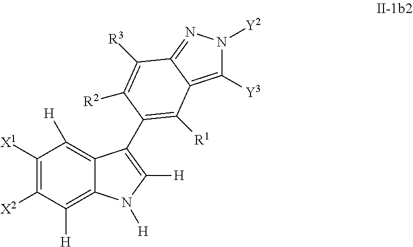

and pharmaceutically acceptable enantiomers, salts and solvates thereof, wherein X¹, X², R¹, R², R³, Y² and Y³ are as defined in Formula I.

According to a preferred embodiment, in Formula II-1b2, Y³ represents H and Y² is as defined in Formula I, preferably, Y² represents:
a hydrogen atom;
CR⁶R⁷R⁸ wherein R⁶, R⁷ and R⁸ represent each independently:
   a hydrogen atom;
   C1-C10 alkyl, linear or branched, preferably methyl, ethyl or propyl; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SOR⁹, aryl, CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, amino;
   heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   —CO—R¹¹ or —SO₂R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   R⁶, R⁷ and the carbon atom to which they are attached form together a ring, said ring being preferably an heterocyclyl, preferably selected from morpholine, piperazine or piperidine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.

In one embodiment, preferred compounds of Formula I are those of Formula II-2a1

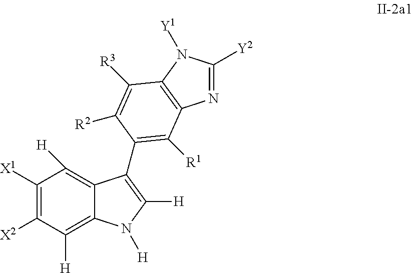

and pharmaceutically acceptable enantiomers, salts and solvates thereof, wherein X¹, X², R¹, R², R³, Y¹ and Y² are as defined in Formula I.

According to a preferred embodiment, in Formula II-2a1, Y¹ represents H and Y² is as defined in Formula I, preferably, Y² represents:
a hydrogen atom;
CR⁶R⁷R⁸, NR⁶R⁷ and OR⁶ wherein R⁶, R⁷ and R⁸ represent each independently:
   a hydrogen atom;
   OR⁹ or NR⁹R¹⁰ wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino, CO-alkyl, SO₂R¹¹, wherein R¹¹ represents a hydrogen atom or a group, optionally substituted, selected from C1-C6 alkyl, aryl, arylalkyl, alkylaryl, heteroaryl, heteroarylalkyl, alkylheteroaryl, amino;
   C1-C10 alkyl, linear or branched, preferably methyl, ethyl or propyl; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, amino;
   heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   —CO—R¹¹ or —SO₂R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   R⁶, R⁷ and the carbon atom to which they are attached form together a ring, said ring being preferably an heterocyclyl, preferably selected from morpholine, piperazine or piperidine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.

In one embodiment, preferred compounds of Formula I are those of Formula II-2a2

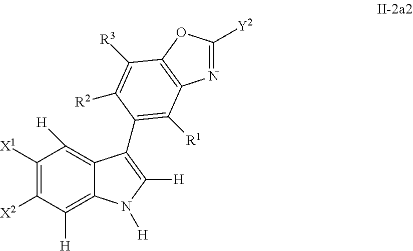

and pharmaceutically acceptable enantiomers, salts and solvates thereof, wherein X¹, X², R¹, R², R³ and Y² are as defined in Formula I.

According to a preferred embodiment, in Formula II-2a2, Y² represents:
a hydrogen atom;
CR⁶R⁷R⁸, NR⁶R⁷ and OR⁶ wherein R⁶, R⁷ and R⁸ represent each independently:
   a hydrogen atom;
   heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   —CO—R¹¹ or —SO₂R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.

In one embodiment, preferred compounds of Formula I are those of Formula II-2b1

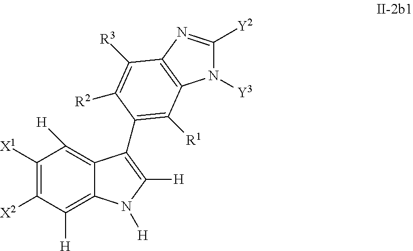

and pharmaceutically acceptable enantiomers, salts and solvates thereof, wherein X¹, X², R¹, R², R³, Y² and Y³ are as defined in Formula I.

According to a preferred embodiment, in Formula II-2b1, Y³ represents H and Y² is as defined in Formula I, preferably, Y² represents:
a hydrogen atom;
CR⁶R⁷R⁸ wherein R⁶, R⁷ and R⁸ represent each independently:
   a hydrogen atom;
   OR⁹ or NR⁹R¹⁰ wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino, CO-alkyl, SO₂R¹¹, wherein R¹¹ represents a hydrogen atom or a group, optionally substituted, selected from C1-C6 alkyl, aryl, arylalkyl, alkylaryl, heteroaryl, heteroarylalkyl, alkylheteroaryl, amino;
   C1-C10 alkyl, linear or branched, preferably methyl, ethyl or propyl; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SOR⁹, aryl, CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, amino;
   heterocyclyl, preferably selected from piperidine, pyrrolidine, piperazine, morpholine; optionally substituted with up to three substituents selected from the group comprising halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.

In one embodiment, preferred compounds of Formula I are those of Formula II-2b2

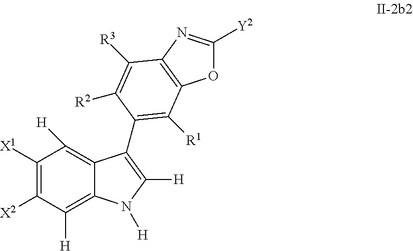

and pharmaceutically acceptable enantiomers, salts and solvates thereof, wherein X¹, X², R¹, R², R³ and Y² are as defined in Formula I, wherein the dotted line is an absent or a bond.

According to a preferred embodiment, in Formula II-2b2, when the dotted line is absent and there is a single bond between the N and C, Y2 is oxo; and when the dotted line is present and there is a double bond between the N and C Y² represents:
a hydrogen atom;
CR⁶R⁷R⁸, NR⁶R⁷ and OR⁶ wherein R⁶, R⁷ and R⁸ represent each independently:
   a hydrogen atom;
   heterocyclyl or C1-heterocycl, preferably selected from piperidine, pyrrolidine, piperazine, morpholine or 2,6-diazaspiro[3.3]heptane; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino;
   —CO—R¹¹ or —SO₂R¹¹ wherein R¹¹ represents a group selected from amine, alkyl, heterocyclyl (preferably piperidine, pyrrolidine, piperazine or tetrahydrothiopyrandioxide); optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino.
In one embodiment, one ore more of the heterocycles is substituted with at least one of H or a group selected from SO₂CH₃, C1-C3 lower akyl, preferably CH₃, or COOH.

In one embodiment, preferred compounds of Formula I are those of Formula II-2b3

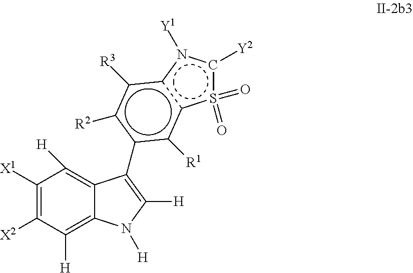

and pharmaceutically acceptable enantiomers, salts and solvates thereof, wherein X¹, X², R¹, R², R³, Y¹, and Y² and Y³ are as defined in Formula I.

In one embodiment of Formula II-2b3, R¹, R² or R³ are each independently H, halogen, or C1-C6 alkyl, preferably, C1 alkyl. In a further embodiment of Formula II-2b3, X¹ and X² are independently H or halogen.

Particularly preferred compounds of Formula I of the invention are those listed in Table 1 hereafter.

TABLE 1

| Cpd n° | Structure | Chemical name |
|---|---|---|
| 1 | 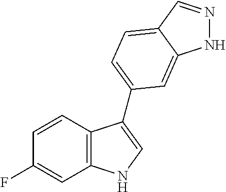 [SMILES: Fc1ccc2c(-c3ccc4cn[nH]c4c3)c[nH]c2c1] | 6-(6-fluoro-1H-indol-3-yl)-1H- indazole |
| 2 | 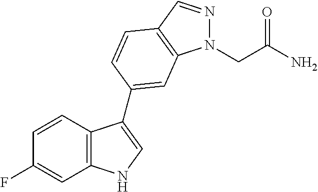 [SMILES: NC(=O)Cn1ncc2ccc(-c3c[nH]c4cc(F)ccc34)cc21] | 2-(6-(6-fluoro-1H-indol-3-yl)- 1H-indazol-1-yl)acetamide |
| 3 | 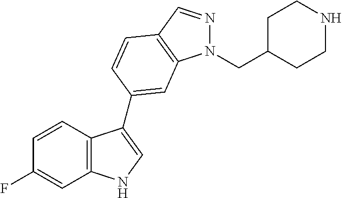 [SMILES: Fc1ccc2c(-c3ccc4cnn(CC5CCNCC5)c4c3)c[nH]c2c1] | 6-(6-fluoro-1H-indol-3-yl)-1- (piperidin-4-ylmethyl)-1H- indazole |
| 4 | 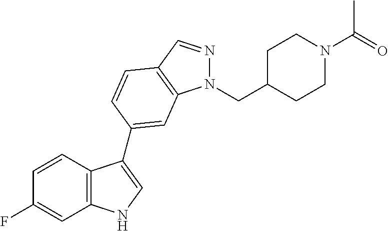 [SMILES: CC(=O)N1CCC(Cn2ncc3ccc(-c4c[nH]c5cc(F)ccc45)cc32)CC1] | 1-(4-((6-(6-fluoro-1H-indol-3- yl)-1H-indazol-1- yl)methyl)piperidin-1- yl)ethanone |
| 5 | 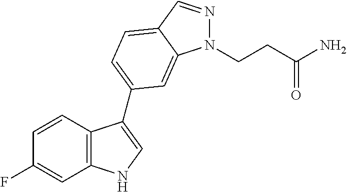 [SMILES: NC(=O)CCn1ncc2ccc(-c3c[nH]c4cc(F)ccc34)cc21] | 3-(6-(6-fluoro-1H-indol-3-yl)- 1H-indazol-1-yl)propanamide |
| 6 | 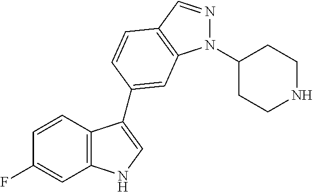 [SMILES: Fc1ccc2c(-c3ccc4cnn(C5CCNCC5)c4c3)c[nH]c2c1] | 6-(6-fluoro-1H-indol-3-yl)-1- (piperidin-4-yl)-1H-indazole |
| 7 | 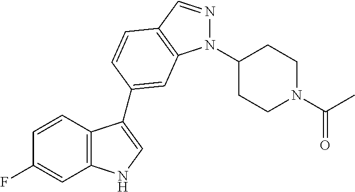 [SMILES: CC(=O)N1CCC(n2ncc3ccc(-c4c[nH]c5cc(F)ccc45)cc32)CC1] | 1-(4-(6-(6-fluoro-1H-indol-3- yl)-1H-indazol-1-yl)piperidin-1- yl)ethanone |
| 8 | 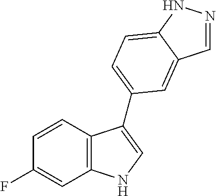 [SMILES: Fc1ccc2c(-c3ccc4[nH]ncc4c3)c[nH]c2c1] | 5-(6-fluoro-1H-indol-3-yl)-1H- indazole |
| 9 | 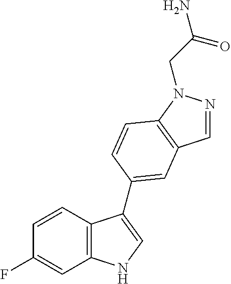 [SMILES: NC(=O)Cn1ncc2cc(-c3c[nH]c4cc(F)ccc34)ccc21] | 2-(5-(6-fluoro-1H-indol-3-yl)- 1H-indazol-1-yl)acetamide |
| 10 | 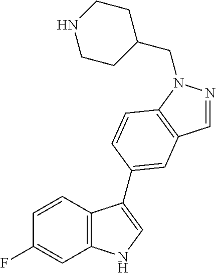 [SMILES: Fc1ccc2c(-c3ccc4c(cnn4CC4CCNCC4)c3)c[nH]c2c1] | 5-(6-fluoro-1H-indol-3-yl)-1- (piperidin-4-ylmethyl)-1H- indazole |
| 11 | 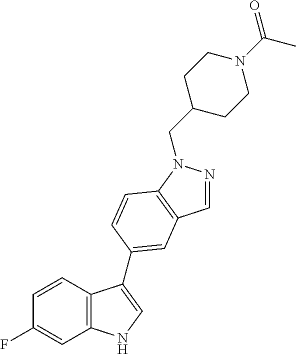 [SMILES: CC(=O)N1CCC(Cn2ncc3cc(-c4c[nH]c5cc(F)ccc45)ccc32)CC1] | 1-(4-((5-(6-fluoro-1H-indol-3- yl)-1H-indazol-1- yl)methyl)piperidin-1- yl)ethanone |
| 12 | 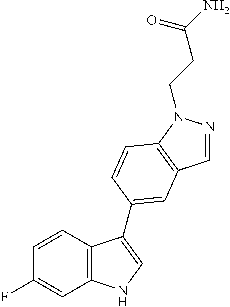 [SMILES: NC(=O)CCn1ncc2cc(-c3c[nH]c4cc(F)ccc34)ccc21] | 3-(5-(6-fluoro-1H-indol-3-yl)- 1H-indazol-1-yl)propanamide |
| 13 | 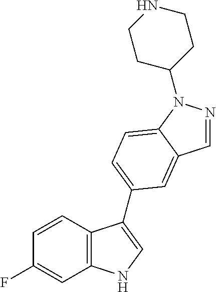 [SMILES: Fc1ccc2c(-c3ccc4c(cnn4C4CCNCC4)c3)c[nH]c2c1] | 5-(6-fluoro-1H-indol-3-yl)-1- (piperidin-4-yl)-1H-indazole |
| 14 | 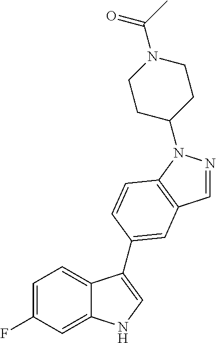 [SMILES: CC(=O)N1CCC(n2ncc3cc(-c4c[nH]c5cc(F)ccc45)ccc32)CC1] | 1-(4-(5-(6-fluoro-1H-indol-3- yl)-1H-indazol-1-yl)piperidin-1- yl)ethanone |
| 15 | 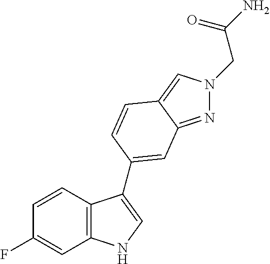 [SMILES: NC(=O)Cn1cc2ccc(-c3c[nH]c4cc(F)ccc34)cc2n1] | 2-(6-(6-fluoro-1H-indol-3-yl)- 2H-indazol-2-yl)acetamide |
| 16 | 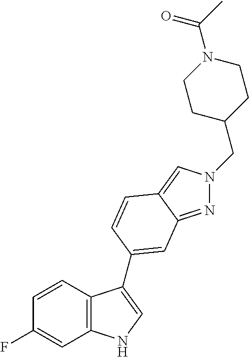 [SMILES: CC(=O)N1CCC(Cn2cc3ccc(-c4c[nH]c5cc(F)ccc45)cc3n2)CC1] | 1-(4-((6-(6-fluoro-1H-indol-3- yl)-2H-indazol-2- yl)methyl)piperidin-1- yl)ethanone |
| 17 | 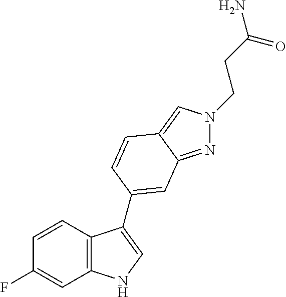 [SMILES: NC(=O)CCn1cc2ccc(-c3c[nH]c4cc(F)ccc34)cc2n1] | 3-(6-(6-fluoro-1H-indol-3-yl)- 2H-indazol-2-yl)propanamide |
| 18 | 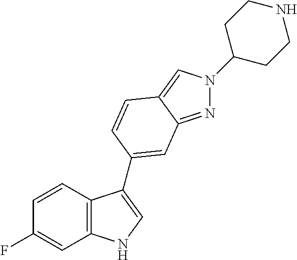 [SMILES: Fc1ccc2c(-c3ccc4cn(C5CCNCC5)nc4c3)c[nH]c2c1] | 6-(6-fluoro-1H-indol-3-yl)-2- (piperidin-4-yl)-2H-indazole |
| 19 | 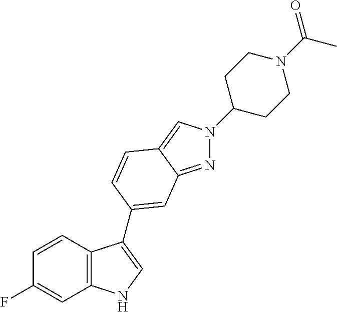 [SMILES: CC(=O)N1CCC(n2cc3ccc(-c4c[nH]c5cc(F)ccc45)cc3n2)CC1] | 1-(4-(6-(6-fluoro-1H-indol-3- yl)-2H-indazol-2-yl)piperidin-1- yl)ethanone |
| 20 | 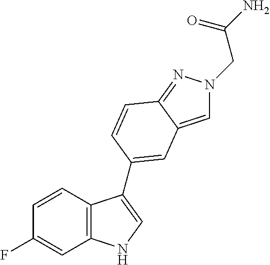 [SMILES: NC(=O)Cn1cc2cc(-c3c[nH]c4cc(F)ccc34)ccc2n1] | 2-(5-(6-fluoro-1H-indol-3-yl)- 2H-indazol-2-yl)acetamide |
| 21 | 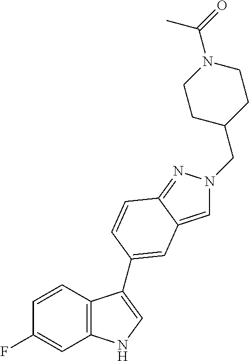 [SMILES: CC(=O)N1CCC(Cn2cc3cc(-c4c[nH]c5cc(F)ccc45)ccc3n2)CC1] | 1-(4-((5-(6-fluoro-1H-indol-3- yl)-2H-indazol-2- yl)methyl)piperidin-1- yl)ethanone |
| 22 | 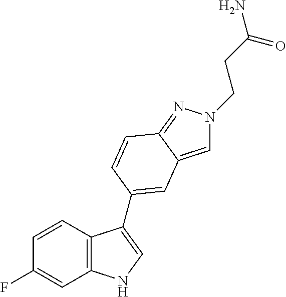 [SMILES: NC(=O)CCn1cc2cc(-c3c[nH]c4cc(F)ccc34)ccc2n1] | 3-(5-(6-fluoro-1H-indol-3-yl)- 2H-indazol-2-yl)propanamide |
| 23 | 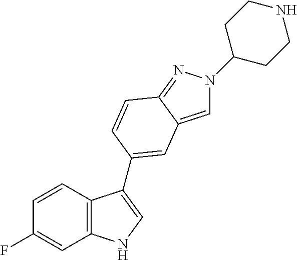 [SMILES: Fc1ccc2c(-c3ccc4nn(C5CCNCC5)cc4c3)c[nH]c2c1] | 5-(6-fluoro-1H-indol-3-yl)-2- (piperidin-4-yl)-2H-indazole |
| 24 | 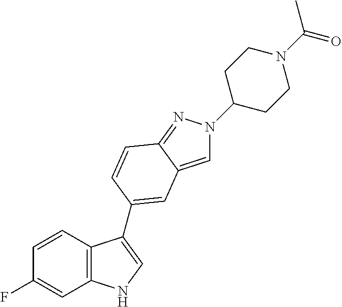 [SMILES: CC(=O)N1CCC(n2cc3cc(-c4c[nH]c5cc(F)ccc45)ccc3n2)CC1] | 1-(4-(5-(6-fluoro-1H-indol-3- yl)-2H-indazol-2-yl)piperidin-1- yl)ethanone |
| 25 | 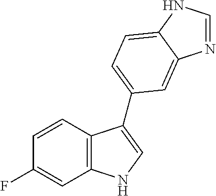 [SMILES: Fc1ccc2c(-c3ccc4[nH]cnc4c3)c[nH]c2c1] | 5-(6-fluoro-1H-indol-3-yl)-1H- benzo[d]imidazole |
| 26 | 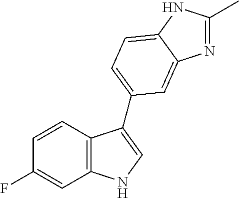 [SMILES: Cc1nc2cc(-c3c[nH]c4cc(F)ccc34)ccc2[nH]1] | 5-(6-fluoro-1H-indol-3-yl)-2- methyl-1H-benzo[d]imidazole |
| 27 | 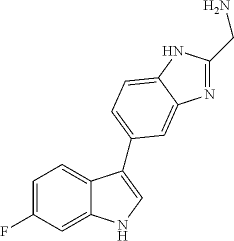 [SMILES: NCc1nc2cc(-c3c[nH]c4cc(F)ccc34)ccc2[nH]1] | (5-(6-fluoro-1H-indol-3-yl)-1H- benzo[d]imidazol-2- yl)methanamine |
| 28 | 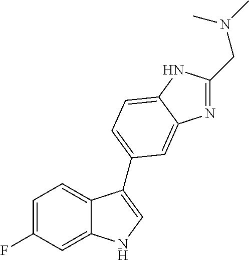 [SMILES: CN(C)Cc1nc2cc(-c3c[nH]c4cc(F)ccc34)ccc2[nH]1] | 1-(5-(6-fluoro-1H-indol-3-yl)- 1H-benzo[d]imidazol-2-yl)- N,N-dimethylmethanamine |
| 29 | 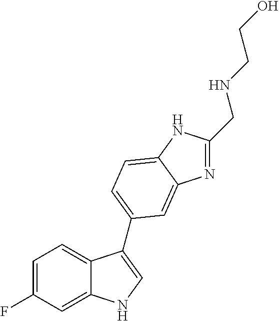 [SMILES: OCCNCc1nc2cc(-c3c[nH]c4cc(F)ccc34)ccc2[nH]1] | 2-(((5-(6-fluoro-1H-indol-3-yl)- 1H-benzo[d]imidazol-2- yl)methyl)amino)ethanol |
| 30 | 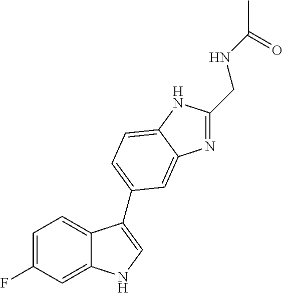 [SMILES: CC(=O)NCc1nc2cc(-c3c[nH]c4cc(F)ccc34)ccc2[nH]1] | N-((5-(6-fluoro-1H-indol-3-yl)- 1H-benzo[d]imidazol-2- yl)methyl)acetamide |
| 31 | 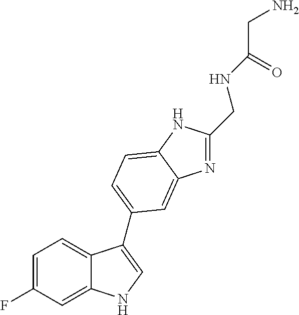 [SMILES: NCC(=O)NCc1nc2cc(-c3c[nH]c4cc(F)ccc34)ccc2[nH]1] | 2-amino-N-((5-(6-fluoro-1H- indol-3-yl)-1H- benzo[d]imidazol-2- yl)methyl)acetamide |
| 32 | 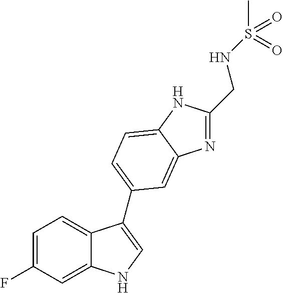 [SMILES: CS(=O)(=O)NCc1nc2cc(-c3c[nH]c4cc(F)ccc34)ccc2[nH]1] | N-((5-(6-fluoro-1H-indol-3-yl)- 1H-benzo[d]imidazol-2- yl)methyl)methanesulfonamide |
| 33 | 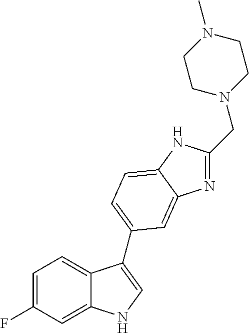 [SMILES: CN1CCN(Cc2nc3cc(-c4c[nH]c5cc(F)ccc45)ccc3[nH]2)CC1] | 5-(6-fluoro-1H-indol-3-yl)-2- ((4-methylpiperazin-1- yl)methyl)-1H- benzo[d]imidazole |
| 34 | 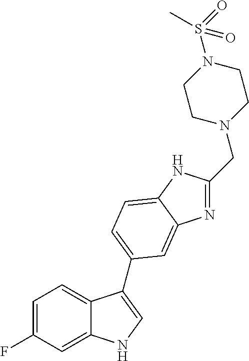 [SMILES: CS(=O)(=O)N1CCN(Cc2nc3cc(-c4c[nH]c5cc(F)ccc45)ccc3[nH]2)CC1] | 5-(6-fluoro-1H-indol-3-yl)-2- ((4-(methylsulfonyl)piperazin- 1-yl)methyl)-1H- benzo[d]imidazole |
| 35 | 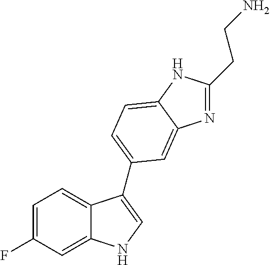 [SMILES: NCCc1nc2cc(-c3c[nH]c4cc(F)ccc34)ccc2[nH]1] | 2-(5-(6-fluoro-1H-indol-3-yl)- 1H-benzo[d]imidazol-2- yl)ethanamine |
| 36 | 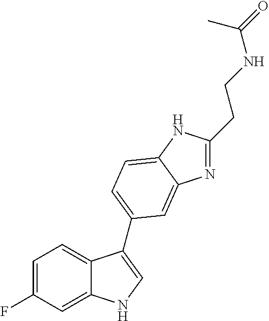 [SMILES: CC(=O)NCCc1nc2cc(-c3c[nH]c4cc(F)ccc34)ccc2[nH]1] | N-(2-(5-(6-fluoro-1H-indol-3- yl)-1H-benzo[d]imidazol-2- yl)ethyl)acetamide |
| 37 | 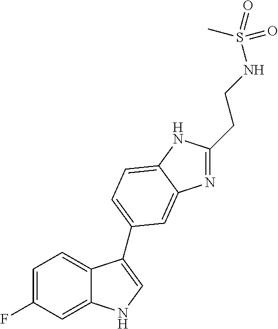 [SMILES: CS(=O)(=O)NCCc1nc2cc(-c3c[nH]c4cc(F)ccc34)ccc2[nH]1] | N-(2-(5-(6-fluoro-1H-indol-3- yl)-1H-benzo[d]imidazol-2- yl)ethyl)methanesulfonamide |
| 38 | 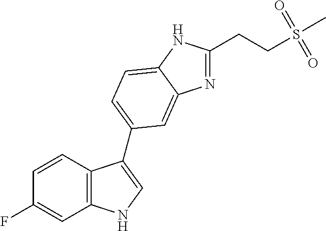 [SMILES: CS(=O)(=O)CCc1nc2cc(-c3c[nH]c4cc(F)ccc34)ccc2[nH]1] | 5-(6-fluoro-1H-indol-3-yl)-2-(2- (methylsulfonyl)ethyl)-1H- benzo[d]imidazole |
| 39 | 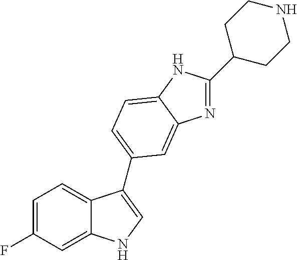 [SMILES: Fc1ccc2c(-c3ccc4[nH]c(C5CCNCC5)nc4c3)c[nH]c2c1] | 5-(6-fluoro-1H-indol-3-yl)-2- (piperidin-4-yl)-1H- benzo[d]imidazole |
| 40 | 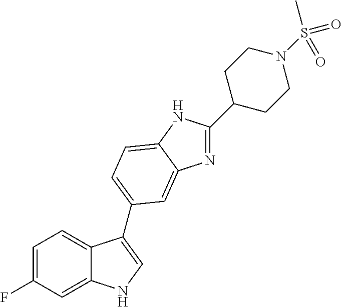 [SMILES: CS(=O)(=O)N1CCC(c2nc3cc(-c4c[nH]c5cc(F)ccc45)ccc3[nH]2)CC1] | 5-(6-fluoro-1H-indol-3-yl)-2-(1- (methylsulfonyl)piperidin-4-yl)- 1H-benzo[d]imidazole |
| 41 | 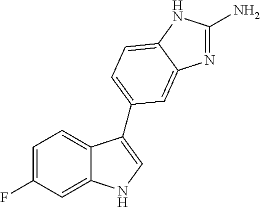 [SMILES: Nc1nc2cc(-c3c[nH]c4cc(F)ccc34)ccc2[nH]1] | 5-(6-fluoro-1H-indol-3-yl)-1H- benzo[d]imidazol-2-amine |
| 42 | 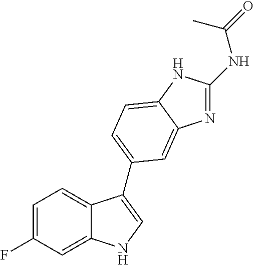 [SMILES: CC(=O)Nc1nc2cc(-c3c[nH]c4cc(F)ccc34)ccc2[nH]1] | N-(5-(6-fluoro-1H-indol-3-yl)- 1H-benzo[d]imidazol-2- yl)acetamide |
| 43 | 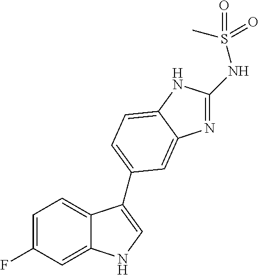 [SMILES: CS(=O)(=O)Nc1nc2cc(-c3c[nH]c4cc(F)ccc34)ccc2[nH]1] | N-(5-(6-fluoro-1H-indol-3-yl)- 1H-benzo[d]imidazol-2- yl)methanesulfonamide |
| 44 | 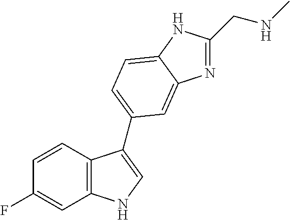 [SMILES: CNCc1nc2cc(-c3c[nH]c4cc(F)ccc34)ccc2[nH]1] | 1-(5-(6-fluoro-1H-indol-3-yl)- 1H-benzo[d]imidazol-2-yl)-N- methylmethanamine |
| 45 | 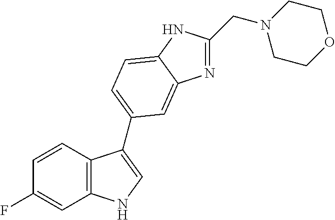 [SMILES: Fc1ccc2c(-c3ccc4[nH]c(CN5CCOCC5)nc4c3)c[nH]c2c1] | 4-((5-(6-fluoro-1H-indol-3-yl)- 1H-benzo[d]imidazol-2- yl)methyl)morpholine |
| 46 | 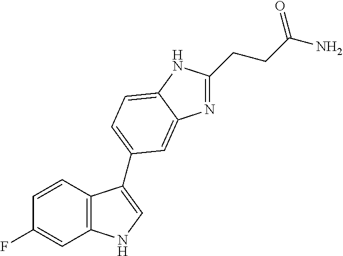 [SMILES: NC(=O)CCc1nc2cc(-c3c[nH]c4cc(F)ccc34)ccc2[nH]1] | 3-(5-(6-fluoro-1H-indol-3-yl)- 1H-benzo[d]imidazol-2- yl)propanamide |
| 47 | 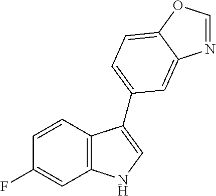 [SMILES: Fc1ccc2c(-c3ccc4ocnc4c3)c[nH]c2c1] | 5-(6-fluoro-1H-indol-3- yl)benzo[d]oxazole |
| 48 | 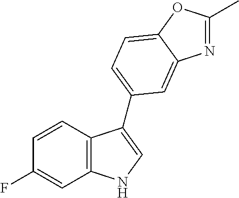 [SMILES: Cc1nc2cc(-c3c[nH]c4cc(F)ccc34)ccc2o1] | 5-(6-fluoro-1H-indol-3-yl)-2- methylbenzo[d]oxazole |
| 49 | 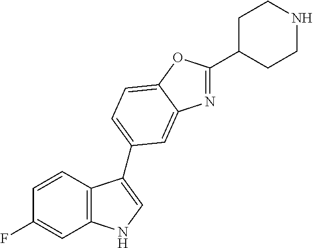 [SMILES: Fc1ccc2c(-c3ccc4oc(C5CCNCC5)nc4c3)c[nH]c2c1] | 5-(6-fluoro-1H-indol-3-yl)-2- (piperidin-4-yl)benzo[d]oxazole |
| 50 | 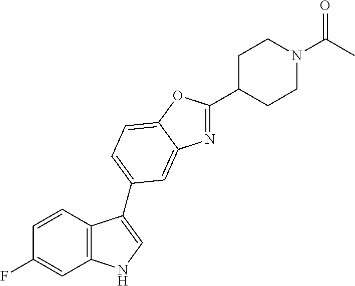 [SMILES: CC(=O)N1CCC(c2nc3cc(-c4c[nH]c5cc(F)ccc45)ccc3o2)CC1] | 1-(4-(5-(6-fluoro-1H-indol-3- yl)benzo[d]oxazol-2- yl)piperidin-1-yl)ethanone |
| 51 | 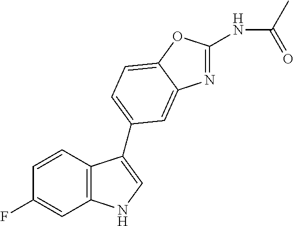 [SMILES: CC(=O)Nc1nc2cc(-c3c[nH]c4cc(F)ccc34)ccc2o1] | N-(5-(6-fluoro-1H-indol-3- yl)benzo[d]oxazol-2- yl)acetamide |
| 52 | 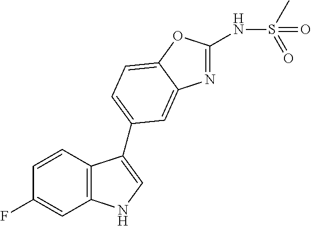 [SMILES: CS(=O)(=O)Nc1nc2cc(-c3c[nH]c4cc(F)ccc34)ccc2o1] | N-(5-(6-fluoro-1H-indol-3- yl)benzo[d]oxazol-2- yl)methanesulfonamide |
| 53 | 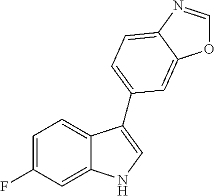 [SMILES: Fc1ccc2c(-c3ccc4ncoc4c3)c[nH]c2c1] | 6-(6-fluoro-1H-indol-3- yl)benzo[d]oxazole |
| 54 | 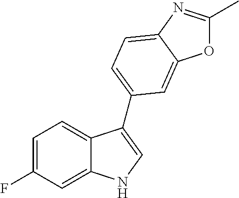 [SMILES: Cc1nc2ccc(-c3c[nH]c4cc(F)ccc34)cc2o1] | 6-(6-fluoro-1H-indol-3-yl)-2- methylbenzo[d]oxazole |
| 55 | 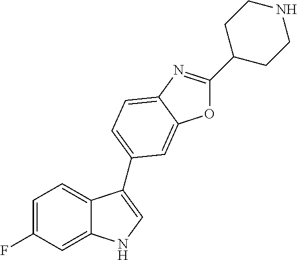 [SMILES: Fc1ccc2c(-c3ccc4nc(C5CCNCC5)oc4c3)c[nH]c2c1] | 6-(6-fluoro-1H-indol-3-yl)-2- (piperidin-4-yl)benzo[d]oxazole |
| 56 | 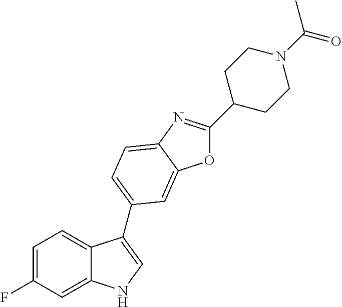 [SMILES: CC(=O)N1CCC(c2nc3ccc(-c4c[nH]c5cc(F)ccc45)cc3o2)CC1] | 1-(4-(6-(6-fluoro-1H-indol-3- yl)benzo[d]oxazol-2- yl)piperidin-1-yl)ethanone |
| 57 | 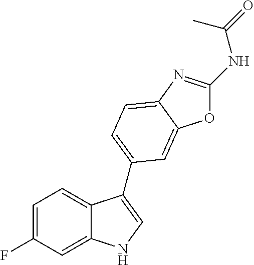 [SMILES: CC(=O)Nc1nc2ccc(-c3c[nH]c4cc(F)ccc34)cc2o1] | N-(6-(6-fluoro-1H-indol-3- yl)benzo[d]oxazol-2- yl)acetamide |
| 58 | 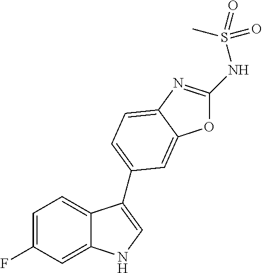 [SMILES: CS(=O)(=O)Nc1nc2ccc(-c3c[nH]c4cc(F)ccc34)cc2o1] | N-(6-(6-fluoro-1H-indol-3- yl)benzo[d]oxazol-2- yl)methanesulfonamide |
| 59 | 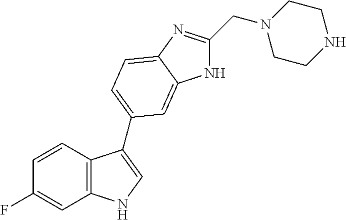 [SMILES: Fc1ccc2c(-c3ccc4nc(CN5CCNCC5)[nH]c4c3)c[nH]c2c1] | 6-(6-fluoro-1H-indol-3-yl)-2- (piperazin-1-ylmethyl)-1H- benzo[d]imidazole |
| 60 | 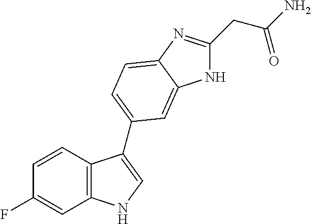 [SMILES: NC(=O)Cc1nc2ccc(-c3c[nH]c4cc(F)ccc34)cc2[nH]1] | 2-(6-(6-fluoro-1H-indol-3-yl)- 1H-benzo[d]imidazol-2- yl)acetamide |
| 61 | 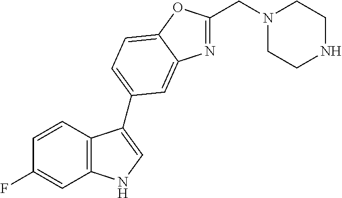 [SMILES: Fc1ccc2c(-c3ccc4oc(CN5CCNCC5)nc4c3)c[nH]c2c1] | 5-(6-fluoro-1H-indol-3-yl)-2- (piperazin-1- ylmethyl)benzo[d]oxazole |
| 62 | 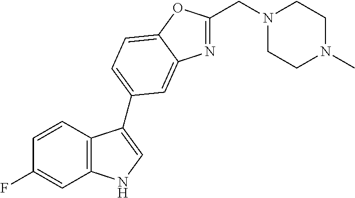 [SMILES: CN1CCN(Cc2nc3cc(-c4c[nH]c5cc(F)ccc45)ccc3o2)CC1] | 5-(6-fluoro-1H-indol-3-yl)-2- ((4-methylpiperazin-1- yl)methyl)benzo[d]oxazole |
| 63 | 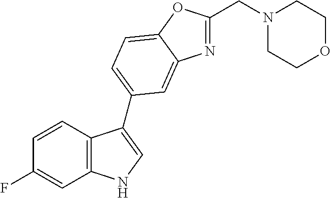 [SMILES: Fc1ccc2c(-c3ccc4oc(CN5CCOCC5)nc4c3)c[nH]c2c1] | 5-(6-fluoro-1H-indol-3-yl)-2- (morpholinomethyl)benzo[d]oxazole |
| 64 | 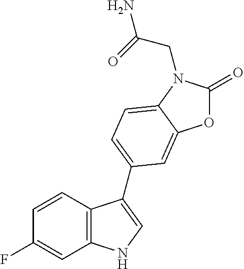 [SMILES: NC(=O)Cn1c(=O)oc2cc(-c3c[nH]c4cc(F)ccc34)ccc21] | 2-(6-(6-fluoro-1H-indol-3-yl)-2- oxobenzo[d]oxazol-3(2H)- yl)acetamide |
| 65 | 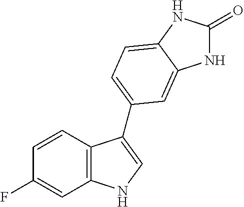 [SMILES: O=c1[nH]c2ccc(-c3c[nH]c4cc(F)ccc34)cc2[nH]1] | 5-(6-fluoro-1H-indol-3-yl)-1H- benzo[d]imidazol-2(3H)-one |
| 66 | 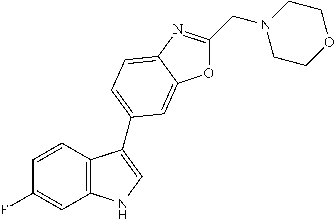 [SMILES: Fc1ccc2c(-c3ccc4nc(CN5CCOCC5)oc4c3)c[nH]c2c1] | 6-(6-fluoro-1H-indol-3-yl)-2- (morpholinomethyl)benzo[d]oxazole |
| 67 | 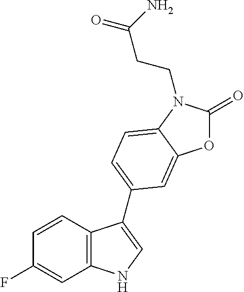 [SMILES: NC(=O)CCn1c(=O)oc2cc(-c3c[nH]c4cc(F)ccc34)ccc21] | 3-(6-(6-fluoro-1H-indol-3-yl)-2- oxobenzo[d]oxazol-3(2H)- yl)propanamide |
| 68 | 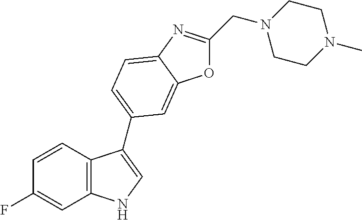 [SMILES: CN1CCN(Cc2nc3ccc(-c4c[nH]c5cc(F)ccc45)cc3o2)CC1] | 6-(6-fluoro-1H-indol-3-yl)-2- ((4-methylpiperazin-1- yl)methyl)benzo[d]oxazole |
| 69 | 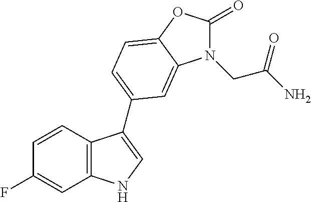 [SMILES: NC(=O)Cn1c(=O)oc2ccc(-c3c[nH]c4cc(F)ccc34)cc21] | 2-(5-(6-fluoro-1H-indol-3-yl)-2- oxobenzo[d]oxazol-3(2H)- yl)acetamide |
| 70 | 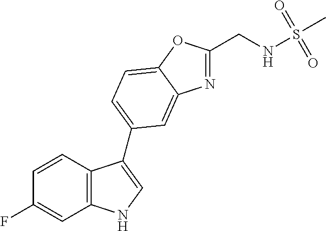 [SMILES: CS(=O)(=O)NCc1nc2cc(-c3c[nH]c4cc(F)ccc34)ccc2o1] | N-((5-(6-fluoro-1H-indol-3- yl)benzo[d]oxazol-2- yl)methyl)methanesulfonamide |
| 71 | 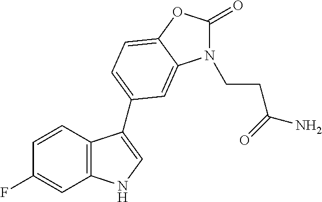 [SMILES: NC(=O)CCn1c(=O)oc2ccc(-c3c[nH]c4cc(F)ccc34)cc21] | 3-(5-(6-fluoro-1H-indol-3-yl)-2- oxobenzo[d]oxazol-3(2H)- yl)propanamide |
| 72 | 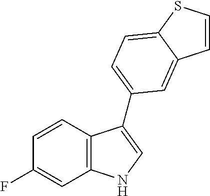 [SMILES: Fc1ccc2c(-c3ccc4sccc4c3)c[nH]c2c1] | 3-(benzo[b]thiophen-5-yl)-6- fluoro-1H-indole |
| 73 | 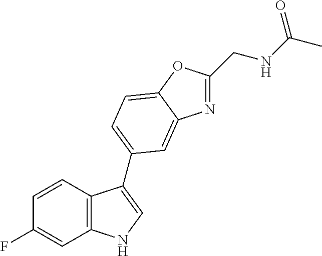 [SMILES: CC(=O)NCc1nc2cc(-c3c[nH]c4cc(F)ccc34)ccc2o1] | N-((5-(6-fluoro-1H-indol-3- yl)benzo[d]oxazol-2- yl)methyl)acetamide |
| 74 | 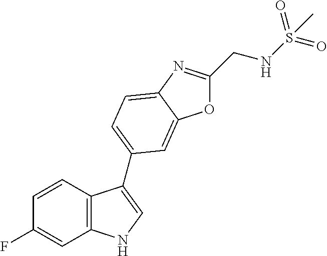 [SMILES: CS(=O)(=O)NCc1nc2ccc(-c3c[nH]c4cc(F)ccc34)cc2o1] | N-((6-(6-fluoro-1H-indol-3- yl)benzo[d]oxazol-2- yl)methyl)methanesulfonamide |
| 75 | 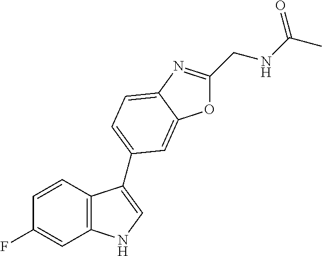 [SMILES: CC(=O)NCc1nc2ccc(-c3c[nH]c4cc(F)ccc34)cc2o1] | N-((6-(6-fluoro-1H-indol-3- yl)benzo[d]oxazol-2- yl)methyl)acetamide |
| 76 | 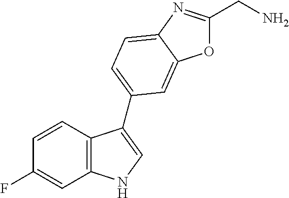 [SMILES: NCc1nc2ccc(-c3c[nH]c4cc(F)ccc34)cc2o1] | (6-(6-fluoro-1H-indol-3- yl)benzo[d]oxazol-2- yl)methanamine |
| 77 | 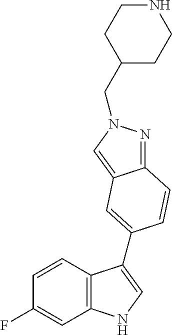 [SMILES: Fc1ccc2c(-c3ccc4nn(CC5CCNCC5)cc4c3)c[nH]c2c1] | 5-(6-fluoro-1H-indol-3-yl)-2- (piperidin-4-ylmethyl)-2H- indazole |
| 78 | 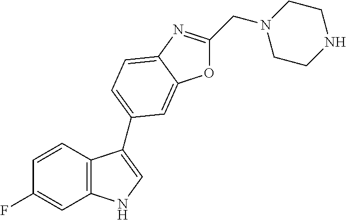 [SMILES: Fc1ccc2c(-c3ccc4nc(CN5CCNCC5)oc4c3)c[nH]c2c1] | 6-(6-fluoro-1H-indol-3-yl)-2- (piperazin-1- ylmethyl)benzo[d]oxazole |
| 79 | 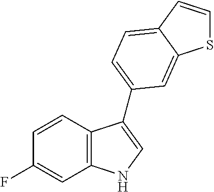 [SMILES: Fc1ccc2c(-c3ccc4ccsc4c3)c[nH]c2c1] | 3-(benzo[b]thiophen-6-yl)-6- fluoro-1H-indole |
| 80 | 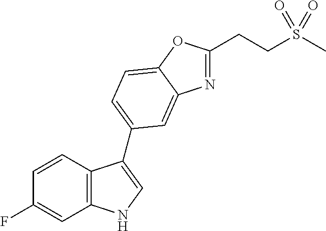 [SMILES: CS(=O)(=O)CCc1nc2cc(-c3c[nH]c4cc(F)ccc34)ccc2o1] | 5-(6-fluoro-1H-indol-3-yl)-2-(2- (methylsulfonyl)ethyl)benzo[d] oxazole |
| 81 | 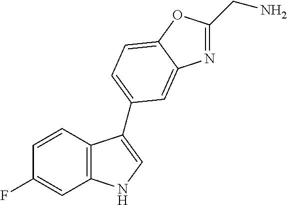 [SMILES: NCc1nc2cc(-c3c[nH]c4cc(F)ccc34)ccc2o1] | (5-(6-fluoro-1H-indol-3- yl)benzo[d]oxazol-2- yl)methanamine |
| 82 | 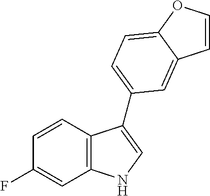 [SMILES: Fc1ccc2c(-c3ccc4occc4c3)c[nH]c2c1] | 3-(benzofuran-5-yl)-6-fluoro- 1H-indole |
| 83 | 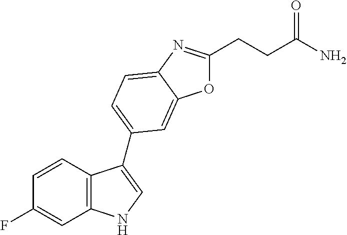 [SMILES: NC(=O)CCc1nc2ccc(-c3c[nH]c4cc(F)ccc34)cc2o1] | 3-(6-(6-fluoro-1H-indol-3- yl)benzo[d]oxazol-2- yl)propanamide |
| 84 | 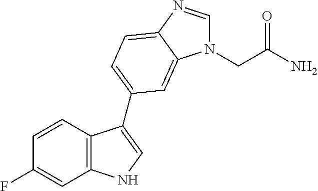 [SMILES: NC(=O)Cn1cnc2ccc(-c3c[nH]c4cc(F)ccc34)cc21] | 2-(6-(6-fluoro-1H-indol-3-yl)- 1H-benzo[d]imidazol-1- yl)acetamide |
| 85 | 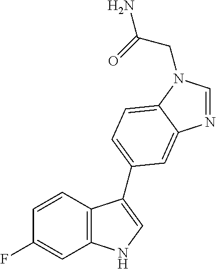 [SMILES: NC(=O)Cn1cnc2cc(-c3c[nH]c4cc(F)ccc34)ccc21] | 2-(5-(6-fluoro-1H-indol-3-yl)- 1H-benzo[d]imidazol-1- yl)acetamide |
| 86 | 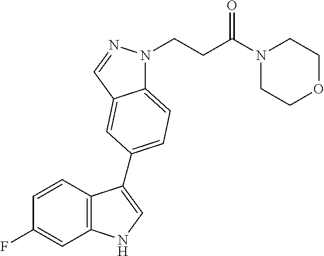 [SMILES: O=C(CCn1ncc2cc(-c3c[nH]c4cc(F)ccc34)ccc21)N1CCOCC1] | 3-(5-(6-fluoro-1H-indol-3-yl)- 1H-indazol-1-yl)-1- morpholinopropan-1-one |
| 87 | 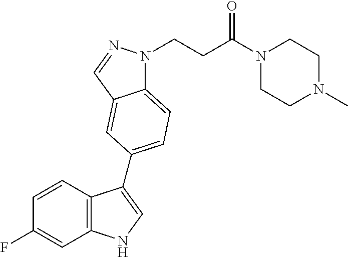 [SMILES: CN1CCN(C(=O)CCn2ncc3cc(-c4c[nH]c5cc(F)ccc45)ccc32)CC1] | 3-(5-(6-fluoro-1H-indol-3-yl)- 1H-indazol-1-yl)-1-(4- methylpiperazin-1-yl)propan-1- one |
| 88 | 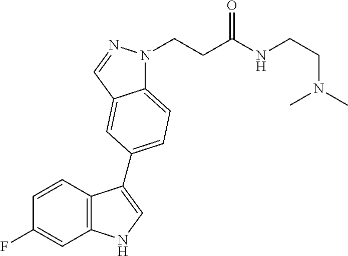 [SMILES: CN(C)CCNC(=O)CCn1ncc2cc(-c3c[nH]c4cc(F)ccc34)ccc21] | N-(2-(dimethylamino)ethyl)-3- (5-(6-fluoro-1H-indol-3-yl)-1H- indazol-1-yl)propanamide |
| 89 | 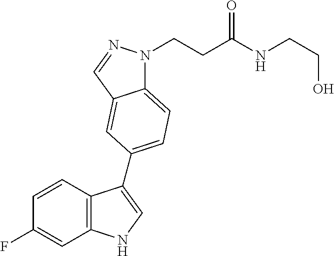 [SMILES: O=C(CCn1ncc2cc(-c3c[nH]c4cc(F)ccc34)ccc21)NCCO] | 3-(5-(6-fluoro-1H-indol-3-yl)- 1H-indazol-1-yl)-N-(2- hydroxyethyl)propanamide |
| 90 | 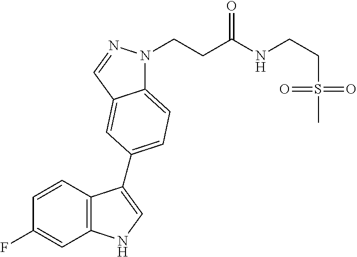 [SMILES: CS(=O)(=O)CCNC(=O)CCn1ncc2cc(-c3c[nH]c4cc(F)ccc34)ccc21] | 3-(5-(6-fluoro-1H-indol-3-yl)- 1H-indazol-1-yl)-N-(2- (methylsulfonyl)ethyl)propanamide |
| 91 | 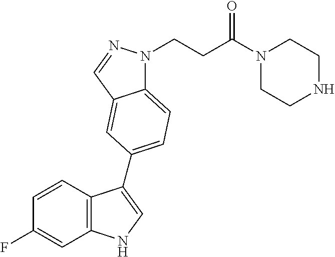 [SMILES: O=C(CCn1ncc2cc(-c3c[nH]c4cc(F)ccc34)ccc21)N1CCNCC1] | 3-(5-(6-fluoro-1H-indol-3-yl)- 1H-indazol-1-yl)-1-(piperazin- 1-yl)propan-1-one |
| 92 | 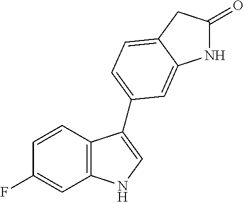 [SMILES: O=C1Cc2ccc(-c3c[nH]c4cc(F)ccc34)cc2N1] | 6-(6-fluoro-1H-indol-3- yl)indolin-2-one |
| 93 | 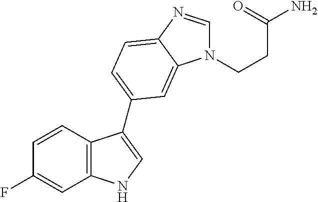 [SMILES: NC(=O)CCn1cnc2ccc(-c3c[nH]c4cc(F)ccc34)cc21] | 3-(6-(6-fluoro-1H-indol-3-yl)- 1H-benzo[d]imidazol-1- yl)propanamide |
| 94 | 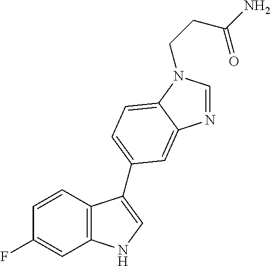 [SMILES: NC(=O)CCn1cnc2cc(-c3c[nH]c4cc(F)ccc34)ccc21] | 3-(5-(6-fluoro-1H-indol-3-yl)- 1H-benzo[d]imidazol-1- yl)propanamide |
| 95 | 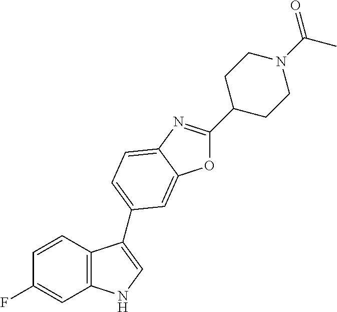 [SMILES: CC(=O)N1CCC(c2nc3ccc(-c4c[nH]c5cc(F)ccc45)cc3o2)CC1] | 1-(4-(6-(6-fluoro-1H-indol-3- yl)benzo[d]oxazol-2- yl)piperidin-1-yl)ethanone |
| 96 | 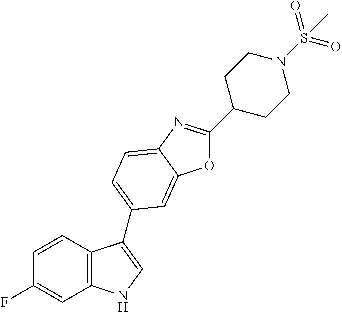 [SMILES: CS(=O)(=O)N1CCC(c2nc3ccc(-c4c[nH]c5cc(F)ccc45)cc3o2)CC1] | 6-(6-fluoro-1H-indol-3-yl)-2-(1- (methylsulfonyl)piperidin-4- yl)benzo[d]oxazole |
| 97 | 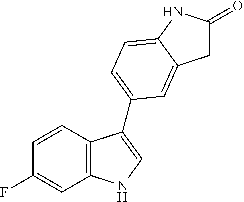 [SMILES: O=C1Cc2cc(-c3c[nH]c4cc(F)ccc34)ccc2N1] | 5-(6-fluoro-1H-indol-3- yl)indolin-2-one |
| 98 | 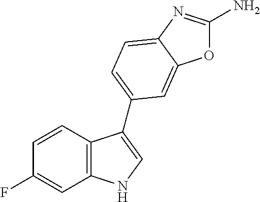 [SMILES: Nc1nc2ccc(-c3c[nH]c4cc(F)ccc34)cc2o1] | 6-(6-fluoro-1H-indol-3- yl)benzo[d]oxazol-2-amine |
| 99 | 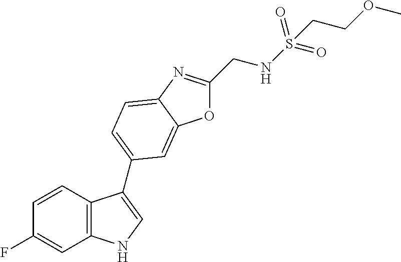 [SMILES: COCCS(=O)(=O)NCc1nc2ccc(-c3c[nH]c4cc(F)ccc34)cc2o1] | N-((6-(6-fluoro-1H-indol-3- yl)benzo[d]oxazol-2-yl)methyl)- 2-methoxyethanesulfonamide |
| 100 | 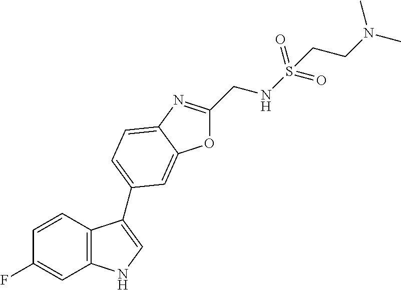 [SMILES: CN(C)CCS(=O)(=O)NCc1nc2ccc(-c3c[nH]c4cc(F)ccc34)cc2o1] | 2-(dimethylamino)-N-((6-(6- fluoro-1H-indol-3- yl)benzo[d]oxazol-2- yl)methyl)ethanesulfonamide |
| 101 | 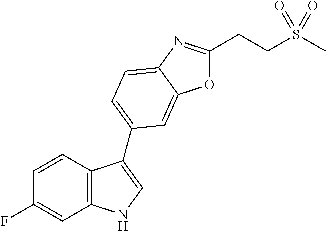 [SMILES: CS(=O)(=O)CCc1nc2ccc(-c3c[nH]c4cc(F)ccc34)cc2o1] | 6-(6-fluoro-1H-indol-3-yl)-2-(2- (methylsulfonyl)ethyl)benzo[d] oxazole |
| 102 | 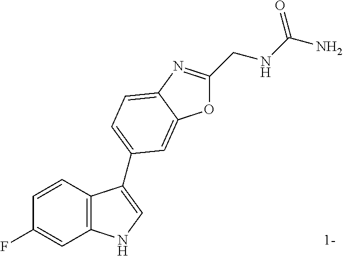 [SMILES: NC(=O)NCc1nc2ccc(-c3c[nH]c4cc(F)ccc34)cc2o1 ; "1-"] | 1-((6-(6-fluoro-1H-indol-3- yl)benzo[d]oxazol-2- yl)methyl)urea |
| 103 | 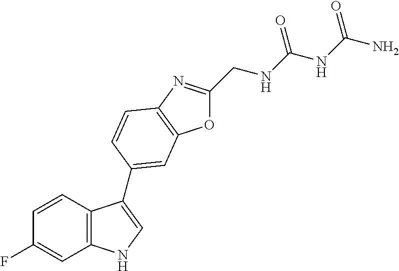 [SMILES: NC(=O)NC(=O)NCc1nc2ccc(-c3c[nH]c4cc(F)ccc34)cc2o1] | 1-carbamoyl-1-((6-(6-fluoro- 1H-indol-3-yl)benzo[d]oxazol- 2-yl)methyl)urea |
| 104 | 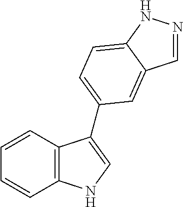 [SMILES: c1ccc2c(-c3ccc4[nH]ncc4c3)c[nH]c2c1] | 5-(1H-indol-3-yl)-1H-indazole |
| 105 | 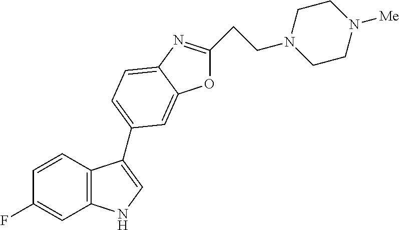 [SMILES: CN1CCN(CCc2nc3ccc(-c4c[nH]c5cc(F)ccc45)cc3o2)CC1] | 6-(6-fluoro-1H-indol-3-yl)-2-(2- (4-methylpiperazin-1- yl)ethyl)benzo[d]oxazole |
| 106 | 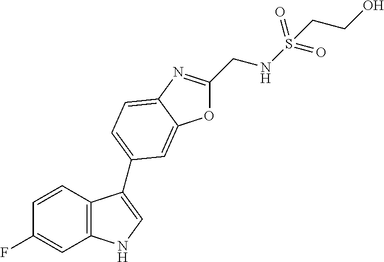 [SMILES: O=S(=O)(CCO)NCc1nc2ccc(-c3c[nH]c4cc(F)ccc34)cc2o1] | N-((6-(6-fluoro-1H-indol-3- yl)benzo[d]oxazol-2-yl)methyl)- 2-hydroxyethanesulfonamide |
| 107 | 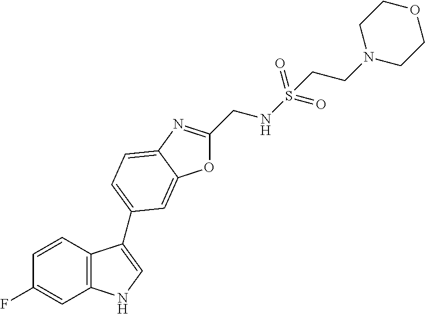 [SMILES: O=S(=O)(CCN1CCOCC1)NCc1nc2ccc(-c3c[nH]c4cc(F)ccc34)cc2o1] | N-((6-(6-fluoro-1H-indol-3- yl)benzo[d]oxazol-2-yl)methyl)- 2-morpholinoethanesulfonamide |
| 108 | 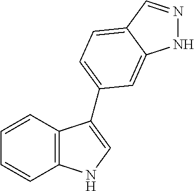 [SMILES: c1ccc2c(-c3ccc4cn[nH]c4c3)c[nH]c2c1] | 6-(1H-indol-3-yl)-1H-indazole |
| 109 | 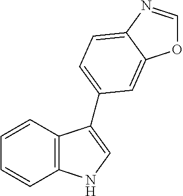 [SMILES: c1ccc2c(-c3ccc4ncoc4c3)c[nH]c2c1] | 6-(1H-indol-3-yl)benzo[d]oxazole |
| 110 | 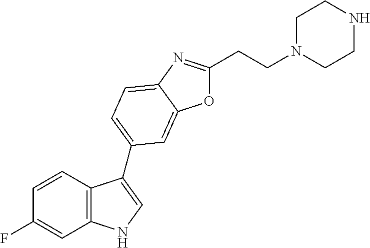 [SMILES: Fc1ccc2c(-c3ccc4nc(CCN5CCNCC5)oc4c3)c[nH]c2c1] | 6-(6-fluoro-1H-indol-3-yl)-2-(2- (piperazin-1- yl)ethyl)benzo[d]oxazole |
| 111 | 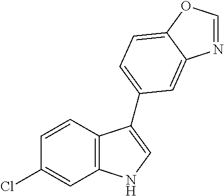 [SMILES: Clc1ccc2c(-c3ccc4ocnc4c3)c[nH]c2c1] | 5-(6-chloro-1H-indol-3- yl)benzo[d]oxazole |
| 112 | 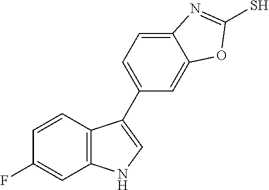 [SMILES: Fc1ccc2c(-c3ccc4nc(S)oc4c3)c[nH]c2c1] | 6-(6-fluoro-1H-indol-3- yl)benzo[d]oxazole-2-thiol |
| 113 | 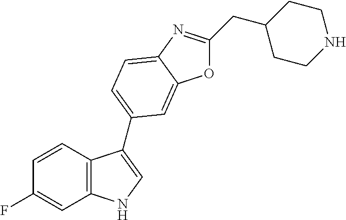 [SMILES: Fc1ccc2c(-c3ccc4nc(CC5CCNCC5)oc4c3)c[nH]c2c1] | 6-(6-fluoro-1H-indol-3-yl)-2- (piperidin-4- ylmethyl)benzo[d]oxazole |
| 114 | 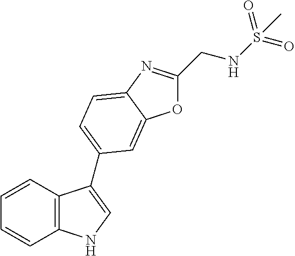 [SMILES: CS(=O)(=O)NCc1nc2ccc(-c3c[nH]c4ccccc34)cc2o1] | N-((6-(1H-indol-3- yl)benzo[d]oxazol-2- yl)methyl)methanesulfonamide |
| 115 | 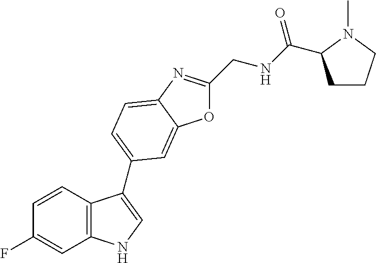 [SMILES: CN1CCC[C@H]1C(=O)NCc1nc2ccc(-c3c[nH]c4cc(F)ccc34)cc2o1] | (S)-N-((6-(6-fluoro-1H-indol-3- yl)benzo[d]oxazol-2-yl)methyl)- 1-methylpyrrolidine-2- carboxamide |
| 116 | 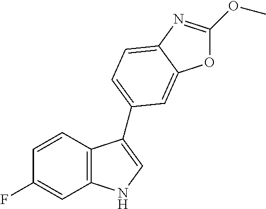 [SMILES: COc1nc2ccc(-c3c[nH]c4cc(F)ccc34)cc2o1] | 6-(6-fluoro-1H-indol-3-yl)-2- methoxybenzo[d]oxazole |
| 117 | 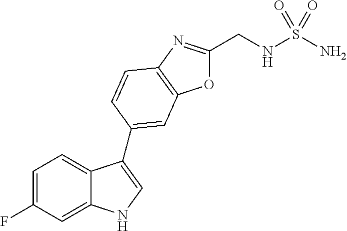 [SMILES: NS(=O)(=O)NCc1nc2ccc(-c3c[nH]c4cc(F)ccc34)cc2o1] | Amino {[6-(6-fluoroindol-3- yl)benzoxazol-2- yl]methyl}sulfonamide |
| 118 | 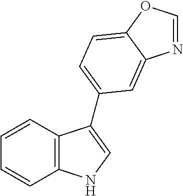 [SMILES: c1ccc2c(-c3ccc4ocnc4c3)c[nH]c2c1] | 5-(1H-indol-3- yl)benzo[d]oxazole |
| 119 | 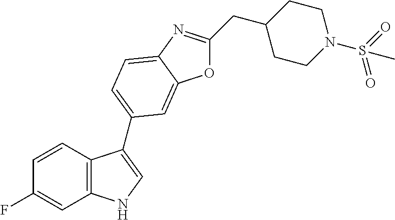 [SMILES: CS(=O)(=O)N1CCC(Cc2nc3ccc(-c4c[nH]c5cc(F)ccc45)cc3o2)CC1] | 6-(6-fluoro-1H-indol-3-yl)-2- ((1-(methylsulfonyl)piperidin-4- yl)methyl)benzo[d]oxazole |
| 120 | 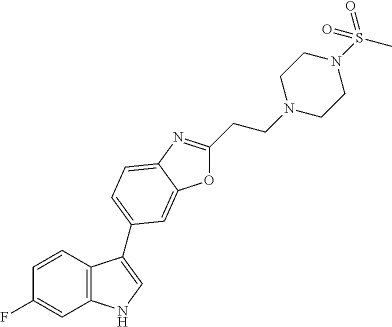 [SMILES: CS(=O)(=O)N1CCN(CCc2nc3ccc(-c4c[nH]c5cc(F)ccc45)cc3o2)CC1] | 6-(6-fluoro-1H-indol-3-yl)-2-(2- (4-(methylsulfonyl)piperazin-1- yl)ethyl)benzo[d]oxazole |
| 121 | 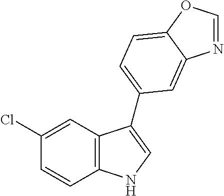 [SMILES: Clc1ccc2[nH]cc(-c3ccc4ocnc4c3)c2c1] | 5-(5-chloro-1H-indol-3- yl)benzo[d]oxazole |
| 122 | 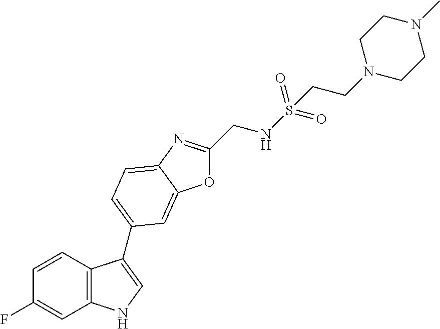 [SMILES: CN1CCN(CCS(=O)(=O)NCc2nc3ccc(-c4c[nH]c5cc(F)ccc45)cc3o2)CC1] | N-((6-(6-fluoro-1H-indol-3- yl)benzo[d]oxazol-2-yl)methyl)- 2-(4-methylpiperazin-1- yl)ethanesulfonamide |
| 123 | 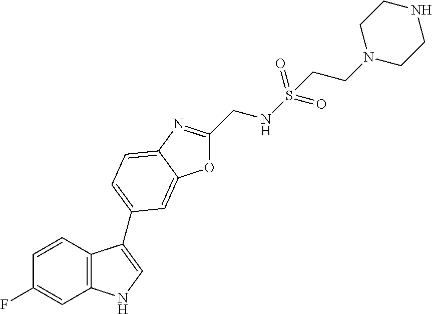 [SMILES: O=S(=O)(CCN1CCNCC1)NCc1nc2ccc(-c3c[nH]c4cc(F)ccc34)cc2o1] | N-((6-(6-fluoro-1H-indol-3- yl)benzo[d]oxazol-2-yl)methyl)- 2-(piperazin-1- yl)ethanesulfonamide |
| 124 | 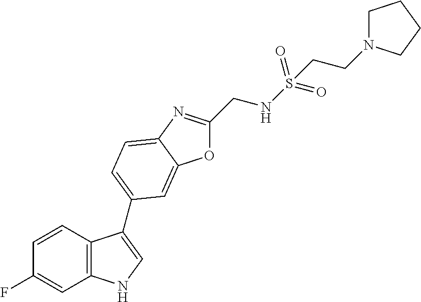 [SMILES: O=S(=O)(CCN1CCCC1)NCc1nc2ccc(-c3c[nH]c4cc(F)ccc34)cc2o1] | N-((6-(6-fluoro-1H-indol-3- yl)benzo[d]oxazol-2-yl)methyl)- 2-(pyrrolidin-1- yl)ethanesulfonamide |
| 125 | 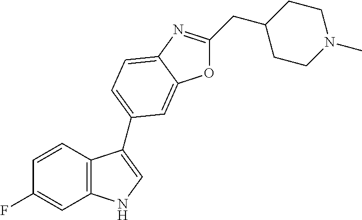 [SMILES: CN1CCC(Cc2nc3ccc(-c4c[nH]c5cc(F)ccc45)cc3o2)CC1] | 6-(6-fluoro-1H-indol-3-yl)-2- ((1-methylpiperidin-4- yl)methyl)benzo[d]oxazole |
| 126 | 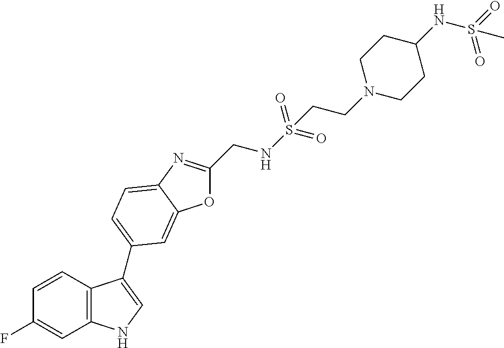 [SMILES: CS(=O)(=O)NC1CCN(CCS(=O)(=O)NCc2nc3ccc(-c4c[nH]c5cc(F)ccc45)cc3o2)CC1] | N-((6-(6-fluoro-1H-indol-3- yl)benzo[d]oxazol-2-yl)methyl)- 2-(4- (methylsulfonamido)piperidin- 1-yl)ethanesulfonamide |
| 127 | 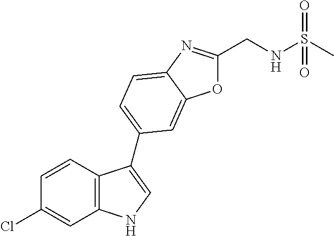 [SMILES: CS(=O)(=O)NCc1nc2ccc(-c3c[nH]c4cc(Cl)ccc34)cc2o1] | N-((6-(6-chloro-1H-indol-3- yl)benzo[d]oxazol-2- yl)methyl)methanesulfonamide |
| 128 | 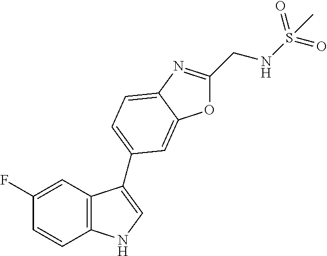 [SMILES: CS(=O)(=O)NCc1nc2ccc(-c3c[nH]c4ccc(F)cc34)cc2o1] | N-((6-(5-fluoro-1H-indol-3- yl)benzo[d]oxazol-2- yl)methyl)methanesulfonamide |
| 129 | 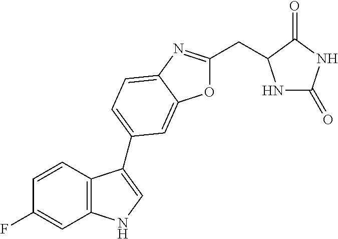 [SMILES: O=C1NC(=O)C(Cc2nc3ccc(-c4c[nH]c5cc(F)ccc45)cc3o2)N1] | 5-((6-(6-fluoro-1H-indol-3- yl)benzo[d]oxazol-2- yl)methyl)imidazolidine-2,4- dione |
| 130 | 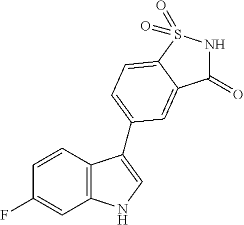 [SMILES: O=C1NS(=O)(=O)c2ccc(-c3c[nH]c4cc(F)ccc34)cc21] | 5-(6-fluoro-1H-indol-3- yl)benzo[d]isothiazol-3(2H)- one 1,1-dioxide |
| 131 | 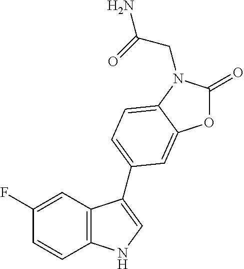 [SMILES: NC(=O)Cn1c(=O)oc2cc(-c3c[nH]c4ccc(F)cc34)ccc21] | 2-(6-(5-fluoro-1H-indol-3-yl)-2- oxobenzo[d]oxazol-3(2H)- yl)acetamide |
| 132 | 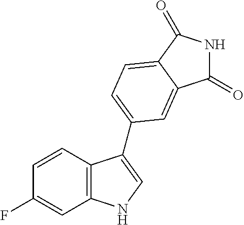 [SMILES: O=C1NC(=O)c2cc(-c3c[nH]c4cc(F)ccc34)ccc21] | 5-(6-fluoro-1H-indol-3- yl)isoindoline-1,3-dione |
| 133 | 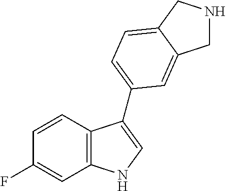 [SMILES: Fc1ccc2c(-c3ccc4c(c3)CNC4)c[nH]c2c1] | 6-fluoro-3-(isoindolin-5-yl)-1H- indole |
| 134 | 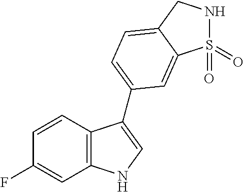 [SMILES: O=S1(=O)NCc2ccc(-c3c[nH]c4cc(F)ccc34)cc21] | 6-(6-fluoro-1H-indol-3- yl)benzo[d]isothiazol-3(2H)- one 1,1-dioxide |
| 135 | 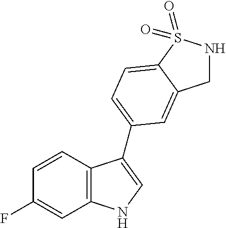 [SMILES: O=S1(=O)NCc2cc(-c3c[nH]c4cc(F)ccc34)ccc21] | 5-(6-fluoro-1H-indol-3-yl)-2,3- dihydrobenzo[d]isothiazole 1,1-dioxide |
| 136 | 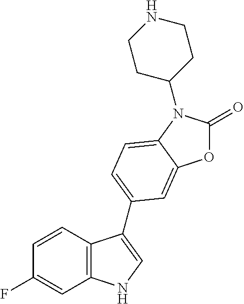 [SMILES: O=c1oc2cc(-c3c[nH]c4cc(F)ccc34)ccc2n1C1CCNCC1] | 6-(6-fluoro-1H-indol-3-yl)-3- (piperidin-4-yl)benzo[d]oxazol- 2(3H)-one |
| 137 | 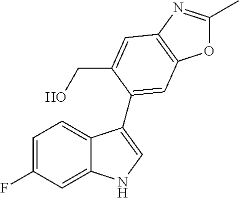 [SMILES: Cc1nc2cc(CO)c(-c3c[nH]c4cc(F)ccc34)cc2o1] | (6-(6-fluoro-1H-indol-3-yl)-2- methylbenzo[d]oxazol-5- yl)methanol |
| 138 | 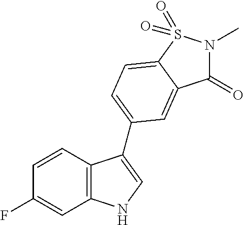 [SMILES: CN1C(=O)c2cc(-c3c[nH]c4cc(F)ccc34)ccc2S1(=O)=O] | 5-(6-fluoro-1H-indol-3-yl)-2- methylbenzo[d]isothiazol- 3(2H)-one 1,1-dioxide |
| 139 | 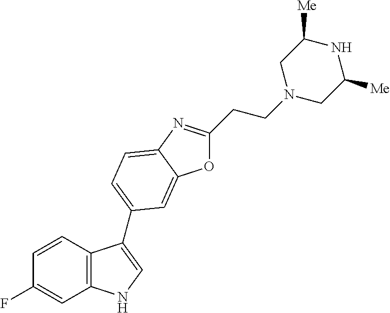 [SMILES: C[C@@H]1CN(CCc2nc3ccc(-c4c[nH]c5cc(F)ccc45)cc3o2)C[C@H](C)N1] | 2-(2-(cis-3,5- dimethylpiperazin-1-yl)ethyl)- 6-(6-fluoro-1H-indol-3- yl)benzo[d]oxazole |
| 140 | 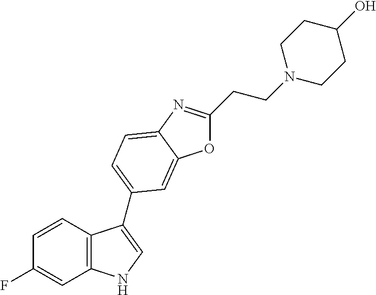 [SMILES: OC1CCN(CCc2nc3ccc(-c4c[nH]c5cc(F)ccc45)cc3o2)CC1] | 1-(2-(6-(6-fluoro-1H-indol-3- yl)benzo[d]oxazol-2- yl)ethyl)piperidin-4-ol |
| 141 | 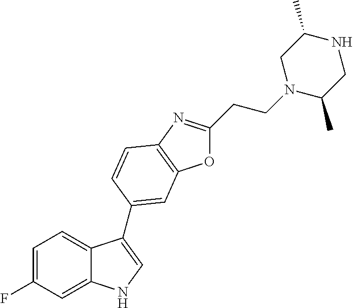 [SMILES: C[C@@H]1CN[C@@H](C)CN1CCc1nc2ccc(-c3c[nH]c4cc(F)ccc34)cc2o1] | 2-(2-{trans-2,5- dimethylpiperazin-1-yl)ethyl)- 6-(6-fluoro-1H-indol-3- yl)benzo[d]oxazole |
| 142 | 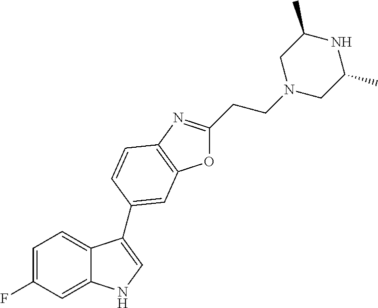 [SMILES: C[C@@H]1CN(CCc2nc3ccc(-c4c[nH]c5cc(F)ccc45)cc3o2)C[C@@H](C)N1] | (+)-2-(2-((3R,5R)-3,5- dimethylpiperazin-1-yl)ethyl)- 6-(6-fluoro-1H-indol-3- yl)benzo[d]oxazole |
| 143 | 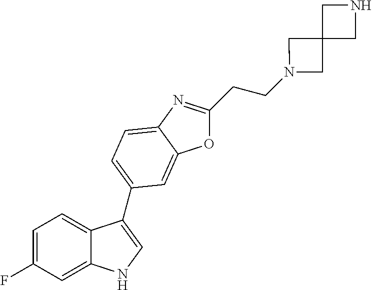 [SMILES: Fc1ccc2c(-c3ccc4nc(CCN5CC6(CNC6)C5)oc4c3)c[nH]c2c1] | 2-(2-(2,6- diazaspiro[3.3]heptan-2- yl)ethyl)-6-(6-fluoro-1H-indol- 3-yl)benzo[d]oxazole |
| 144 | 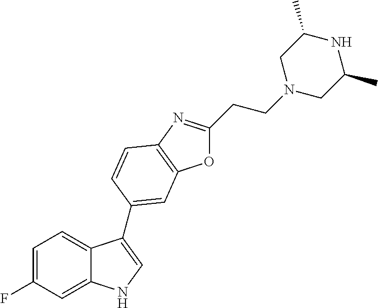 [SMILES: C[C@H]1CN(CCc2nc3ccc(-c4c[nH]c5cc(F)ccc45)cc3o2)C[C@H](C)N1] | (−)-2-(2-((3S,5S)-3,5- dimethylpiperazin-1-yl)ethyl)- 6-(6-fluoro-1H-indol-3- yl)benzo[d]oxazole |
| 145 | 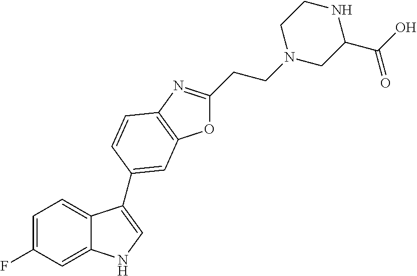 [SMILES: O=C(O)C1CN(CCc2nc3ccc(-c4c[nH]c5cc(F)ccc45)cc3o2)CCN1] | 4-(2-(6-(6-fluoro-1H-indol-3- yl)benzo[d]oxazol-2- yl)ethyl)piperazine-2- carboxylic acid |
| 146 | 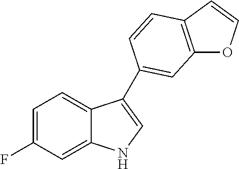 [SMILES: Fc1ccc2c(-c3ccc4ccoc4c3)c[nH]c2c1] | 3-(benzofuran-6-yl)-6-fluoro- 1H-indole |

or pharmaceutically acceptable enantiomers, salts and solvates thereof.

In Table 1, the term “Cpd” means compound.

The compounds of Table 1 were named using ChemBioDraw® Ultra version 12.0 (PerkinElmer).

The compounds of Formula I and subformulae thereof may contain an asymmetric center and thus may exist as different stereoisomeric forms. Accordingly, the present invention includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

The compounds of the invention may be in the form of “pharmaceutically acceptable salts”. Pharmaceutically acceptable salts of the compounds of Formula I include the acid addition and base salts thereof. Suitable acid addition salts are formed from acids which form non-toxic salts. Examples include the acetate, lactobionate, benzenesulfonate, laurate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandalate, bitartrate, methylbromide, bromide, methylnitrate, calcium edetate, mucate, napsylate, chloride, clavulanate, N oleate, edetate, estolate, pantothenate, polygalacuronate, salicylate, glutamate, glycollylarsanilate, sulfate, hexylrosorcinate, subacetate, hydrabamine, hydroxynaphthaloate, etolate, triethiodid, valerate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, ornithine, N,N-dibenzyethelenediamine, piperazine, tri(hydroxymethyl_aminomethane, tetramethylammonium hydroxide, -methylgucamine, ammonium salt, potassium, sodium, tromethamine, 2-(diethylamino)ethanol, ethanolamine, morpholine, 4-(2-hydroxyethyl)morpholine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, and acetate.

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of Formula I may be prepared by one or more of these methods:
(i) by reacting the compound of Formula I with the desired acid;
(ii) by reacting the compound of Formula I with the desired base;
(iii) by removing an acid- or base-labile protecting group from a suitable precursor of the compound of Formula I or by ring-opening a suitable cyclic precursor, for example, a lactone or lactam, using the desired acid; or
(iv) by converting one salt of the compound of Formula I to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The compounds of the present invention may be administered in the form of pharmaceutically acceptable salts, which are as defined above. These salts may be prepared by standard procedures, e.g. by reacting a free acid with a suitable organic or inorganic base. Where a basic group is present, such as amino, an acidic salt, i.e. hydrochloride, hydrobromide, acetate, palmoate, and the like, can be used as the dosage form.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

All references to compounds of Formula I throughout this specification, includes all of ts subformulae, Formula II (including II-1, II-1a, II-1a1, II-1a2, II-1b, II-1b1, II-1b2, II-2, II-2a, II-2a1, II-2a2, II-2b, II-2b1, II-2b2, II-2b3) include references to enantiomers, salts, solvates, polymorphs, multi-component complexes and liquid crystals thereof.

The compounds of the invention include compounds of Formula I as hereinbefore defined, including all polymorphs and crystal habits thereof, prodrugs and isomers thereof (including optical, geometric and tautomeric isomers) and isotopically-labeled compounds of Formula I.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I above.

The invention also generally covers all pharmaceutically acceptable predrugs and prodrugs of the compounds of Formula I.

Process for Manufacturing

The compounds of Formula I can be prepared by different ways with reactions known to a person skilled in the art.

The invention further relates to a first process for manufacturing of compounds of Formula I

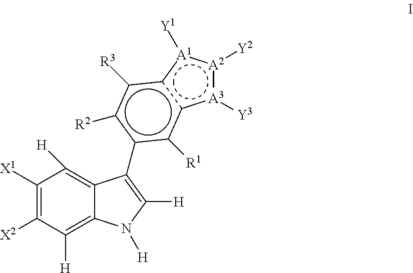

and pharmaceutically acceptable enantiomers, salts and solvates thereof, wherein X¹, X², R¹, R², R³, A¹, A², A³, Y¹, Y² and Y³ are as defined in Formula I;
comprising:
(a1) reacting a compound of Formula (i)

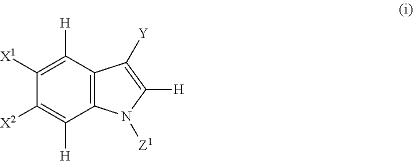

wherein
X¹ and X² are as defined in Formula I;
Z¹ represents H or an amino protecting group such as for example an arylsulphonyl, a tert-butoxy carbonyl, a methoxymethyl, a para-methoxy benzyl, a benzyl or any other suitable protecting group known to those skilled in the art
Y represents an halogen (preferably iodine, bromine or chlorine), an alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy), or any leaving group known to those skilled in the art
with a compound of Formula (ii)

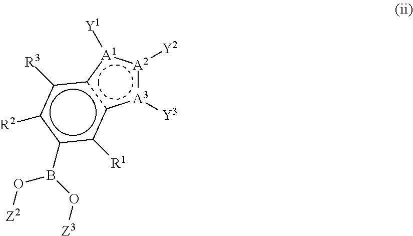

wherein
R¹, R², R³, A¹, A², A³, Y¹, Y² and Y³ are as defined in Formula I;
Z² and Z³ represent H or alkyl groups, with the possibility for Z² and Z³ to form a ring;
so as to obtain a compound of Formula (iii),

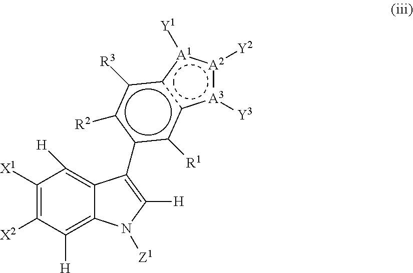

wherein Z¹, X¹, X², R¹, R², R³, A¹, A², A³, Y¹, Y² and Y³ are as defined above; and
(b1) in the case wherein Z¹ is not H, deprotecting the indole amine of compound of Formula (iii), to afford compound of Formula I.

According to one embodiment, step (a1) of the process of the invention may be performed with or without a catalyst such as but not limited to Pd₂(dba)₃, Pd(PPh₃)₄, dichlorobis(triphenylphosphine)palladium(II) or 1,1′-bis(diphenylphosphino)ferrocene-dichloro palladium(II), Pd(OAc)₂, or Pd/C in the presence or absence of an additional ligand, such as but not limited to X-Phos, S-Phos, P(oTol)₃, PPh₃, BINAP, P(tBu)₃ or any other suitable phosphine ligand known to those skilled in the art.

According to one embodiment, step (a1) of the process of the invention may be performed in the presence of bases such as but not limited to K₃PO₄, K₂CO₃, Na₂CO₃.

According to one embodiment, step (a1) of the process of the invention may be performed in the presence of a suitable solvent such as but not limited to dioxane, THF, DMF, water or mixtures thereof, preferably in a mixture of dioxane or THF and water.

According to one embodiment, step (a1) of the process of the invention may be performed at a temperature ranging from 20° C. to about 180° C., with or without microwave irradiation, for a period ranging from 10 minutes to a few hours, e.g. 10 minutes to 24 h.

According to one embodiment, the deprotection (b1) may be performed, depending on the nature of the group Z¹, by treatment with bases, such as but not limited to sodium hydroxide, potassium hydroxide, potassium carbonate. According to one embodiment, the deprotection may be performed in the presence or absence of a suitable solvent such as but not limited to methanol, ethanol, isopropanol, tert-butanol, THF, DMF, Dioxane, water or a mixture thereof. According to one embodiment, the deprotection may be performed at a temperature ranging from 20° C. to 100° C., preferably at about 85° C., for a few hours, e.g. one hour to 24 h.

According to an alternative embodiment, the deprotection (b1) may be performed, depending on the nature of the group Z¹ in the presence of strong acids, such as but not limited to HCl, TFA, HF, HBr. According to one embodiment, the deprotection may be performed in the presence or absence of a suitable solvent such as methanol, ethanol, isopropanol, tert-butanol, THF, DMF, Dioxane, water or a mixture thereof. According to one embodiment, the deprotection may be performed at a temperature between about 20° C. to about 100° C., for a period comprised between 10 minutes and a few hours, e.g. 10 minutes to 24 h.

The invention further relates to a second process of manufacturing of compounds of Formula I

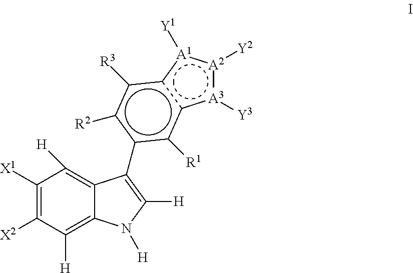

and pharmaceutically acceptable enantiomers, salts and solvates thereof, wherein X¹, X², R¹, R², R³, A¹, A², A³, Y¹, Y² and Y³ are as defined in Formula I;
with the condition that at least one of Y¹, Y² and Y³ is not H or absent;
comprising:
(a2) alkylating a compound of Formula (iv)

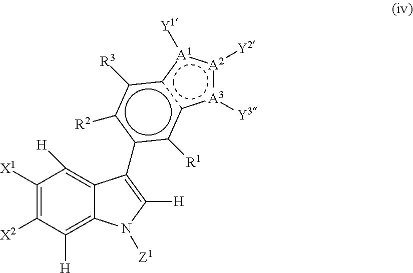

wherein X¹, X², R¹, R², R³, A¹, A², A³, are as defined in Formula I,
Y^{1′}, Y^{2′} and Y^{3′} represent respectively Y¹, Y² and Y³ as defined in Formula I with the condition that at least one of Y^{1′}, Y^{2′} and Y^{3′} is H;
Z¹ represents H or an amino protecting group such as for example an arylsulphonyl, a tert-butoxy carbonyl, a methoxymethyl, a para-methoxy benzyl, a benzyl or any other suitable protecting group known to those skilled in the art;
so as to obtain a compound of Formula (v)

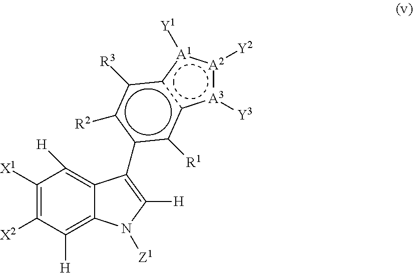

wherein X¹, X², R¹, R², R³, A¹, A², A³, Y¹, Y², Y³ and Z¹ are defined as above with the condition that at least one of Y¹, Y² and Y³ is not H or absent;
and
(b2) in the case wherein Z¹ is not H, deprotecting the indole nitrogen of compound of Formula (v), to afford compound of Formula I.

According to one embodiment, alkylation step (a2) is performed in presence of a compound of Formula (vi)

Y⁴—X
wherein Y⁴ represent Y¹, Y² or Y³, as defined in Formula I, with the condition that Y⁴ is not H or absent;
X represents an halogen (preferably iodine, bromine or chlorine), alkylsulfonyloxy having 1-6 carbon atoms (preferably methylsulfonyloxy or trifluoro-methylsulfonyloxy) or arylsulfonyloxy having 6-10 carbon atoms (preferably phenyl- or p-tolylsulfonyloxy), or any other leaving group known to those skilled in the art.

According to one embodiment, step (a2) of the process of the invention may be performed in the presence of bases such as but not limited to potassium carbonate, sodium carbonate, cesium carbonate, potassium hydroxide, sodium hydroxide, sodium tert-butoxide, potassium tert-butoxide, sodium hydride, lithium diisopropyl amide, buthyl lithium.

According to one embodiment, step (a2) of the process of the invention may be performed in the presence of a suitable solvent such as but not limited to DMF, methanol, ethanol, isopropanol, tert-butanol, THF, dioxane, dichloromethane, water.

According to one embodiment, step (a2) of the process of the invention may be performed in the presence or absence of catalytic amounts of appropriate iodide salts, such as but not limited to tetrabutylammonium iodide.

According to one embodiment, step (a2) of the process of the invention may be performed at a temperature ranging from 20° C. to about 180° C., with or without microwave irradiation.

According to one embodiment, step (a2) of the process of the invention may be performed for a period ranging from 10 minutes and a few hours, e.g. 10 minutes to 24 h.

According to one embodiment, the deprotection step (b2) may be performed in conditions described above for deprotection (b1).

The invention further relates to a third process of manufacturing of compounds of Formula II-2a1 wherein Y¹ is H:

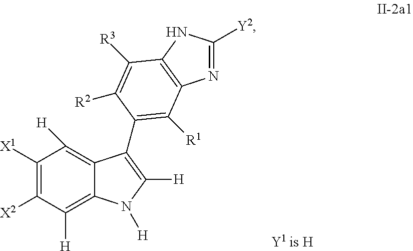

and pharmaceutically acceptable enantiomers, salts and solvates thereof, wherein X¹, X², R¹, R², R³, and Y² are as defined in Formula I;
comprising:
(a3) reacting a compound of Formula (vii)

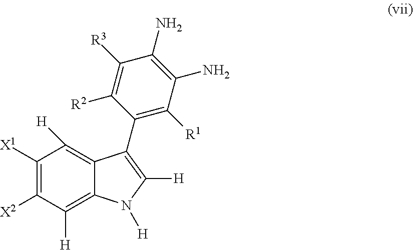

wherein X¹, X², R¹, R², R³ are as defined above; and
Z¹ represents H or an amino protecting group such as for example an arylsulphonyl, a tert-butoxy carbonyl, a methoxymethyl, a para-methoxy benzyl, a benzyl or any other suitable protecting group known to those skilled in the art;
with a compound of Formula (viii)

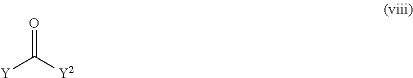

wherein Y² is defined as above; and
Y represents an hydroxyl, halogen (preferably iodine, bromine or chlorine), alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy), or any other leaving group known to those skilled in the art;
so as to obtain a compound of Formula (ix)

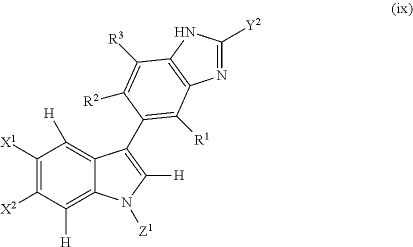

wherein X¹, X², R¹, R², R³, Y² and Z¹ are defined as above;
and
(b3) in the case wherein Z¹ is not H, deprotecting the indole amine of compound of

Formula (ix), to afford compound of II-2a1 wherein Y¹ is H.

According to one embodiment, when Y represents hydroxyl, step (a3) of the process of the invention may be performed in the presence of a suitable amide coupling reagent, such as but not limited to HATU, DCC, DIC, BOP, PyBOP, in the presence or absence of additional additives such as but not limited to HOBt.

According to one embodiment, step (a3) of the process of the invention may be performed in the presence of bases such as but not limited to triethylamine, diisopropylethylamine, DBU, cesium carbonate, sodium carbonate, potassium carbonate, sodium hydroxide, potassium hydroxide.

According to one embodiment, step (a3) of the process of the invention may be performed in the presence of a suitable solvent such as but not limited to Dichloromethane, DMF, THF, dioxane.

According to one embodiment, step (a3) further comprises adding a suitable acid, such as but not limited to acetic acid, wherever necessary to complete cyclization.

According to one embodiment, step (a3) of the process of the invention may be performed at a temperature ranging from 20° C. to about 180° C., with or without microwave irradiation.

According to one embodiment, step (a3) of the process of the invention may be performed for a period ranging from 10 minutes and a few hours, e.g. 10 minutes to 24 h.

According to one embodiment, the deprotection step (b3) may be performed in conditions described above for deprotection (b1).

The invention further relates to a fourth process for manufacturing of compounds of Formula I

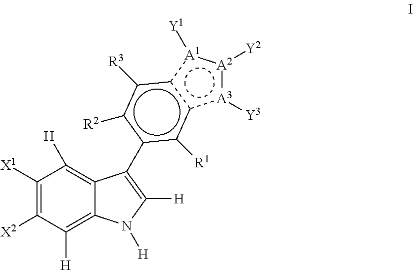

and pharmaceutically acceptable enantiomers, salts and solvates thereof,
wherein X¹, X², R¹, R², R³, A¹, A², A³, Y¹, Y² and Y³ are as defined in Formula I;
comprising:
(a4) reacting a compound of Formula (x)

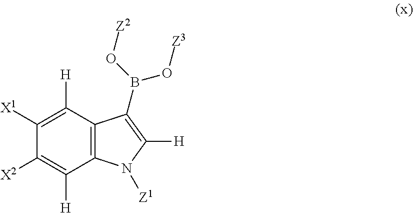

wherein
X¹ and X² are as defined in Formula I;
Z¹ represents H or an amino protecting group such as for example an arylsulphonyl, a tert-butoxy carbonyl, a methoxymethyl, a para-methoxy benzyl, a benzyl or any other suitable protecting group known to those skilled in the art
Z² and Z³ represent H or alkyl groups, with the possibility for Z² and Z³ to form a ring;
with a compound of Formula (xi)

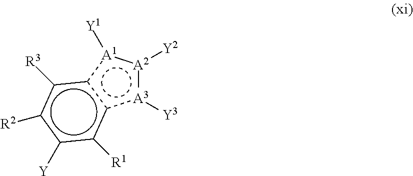

wherein
R¹, R², R³, A¹, A², A³, Y¹, Y² and Y³ are as defined in Formula I;
Y represents an halogen (preferably iodine, bromine or chlorine), an alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy), or any leaving group known to those skilled in the art
so as to obtain a compound of Formula (xii),

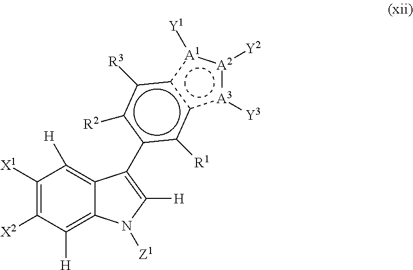

wherein Z¹, X¹, X², R¹, R², R³, A¹, A², A³, Y¹, Y² and Y³ are as defined above; and
(b4) in the case wherein Z¹ is not H, deprotecting the indole amine of compound of Formula (xii), to afford compound of Formula I.

According to one embodiment, step (a4) of the process of the invention may be performed with or without a catalyst such as but not limited to Pd₂(dba)₃, Pd(PPh₃)₄, dichlorobis(triphenylphosphine)palladium(II) or 1,1′-bis(diphenylphosphino)ferrocene-dichloro palladium(II), Pd(OAc)₂, or Pd/C in the presence or absence of an additional ligand, such as but not limited to X-Phos, S-Phos, P(oTol)₃, PPh₃, BINAP, P(tBu)₃ or any other suitable phosphine ligand known to those skilled in the art.

According to one embodiment, step (a4) of the process of the invention may be performed in the presence of bases such as but not limited to K₃PO₄, K₂CO₃, Na₂CO₃.

According to one embodiment, step (a4) of the process of the invention may be performed in the presence of a suitable solvent such as but not limited to dioxane, THF, DMF, water or mixtures thereof, preferably in a mixture of dioxane or THF and water.

According to one embodiment, step (a4) of the process of the invention may be performed at a temperature ranging from 20° C. to about 180° C., with or without microwave irradiation, for a period ranging from 10 minutes to a few hours, e.g. 10 minutes to 24 h.

According to one embodiment, the deprotection step (b4) may be performed in conditions described above for deprotection (b1).

In general, the synthesis pathways for any individual compound of Formula (I) will depend on the specific substituents of each molecule and upon the ready availability of intermediates necessary; again such factors being appreciated by those of ordinary skill in the art.

According to a further general process, compounds of Formula I can be converted to alternative compounds of Formula I, employing suitable interconversion techniques well known by a person skilled in the art.

Compounds of the Formula I and related formulae can furthermore be obtained by liberating compounds of the Formula I from one of their functional derivatives by treatment with a solvolysing or hydrogenolysing agent.

Preferred starting materials for the solvolysis or hydrogenolysis are those which conform to the Formula I and related formulae, but contain corresponding protected amino and/or hydroxyl groups instead of one or more free amino and/or hydroxyl groups, preferably those which carry an amino-protecting group instead of an H atom bonded to an N atom, in particular those which carry an R*—N group, in which R* denotes an amino-protecting group, instead of an HN group, and/or those which carry a hydroxyl-protecting group instead of the H atom of a hydroxyl group, for example those which conform to the Formula I, but carry a —COOR** group, in which R** denotes a hydroxyl-protecting group, instead of a —COOH group.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxy-carbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxy-carbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The term “hydroxyl-protecting group” is likewise known in general terms and relates to groups which are suitable for protecting a hydroxyl group against chemical reactions, but are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the Formula I and related formulae are liberated from their functional derivatives—depending on the protecting group used—for example strong inorganic acids, such as hydrochloric acid, perchloric acid or sulfuric acid, strong organic carboxylic acids, such as trichloroacetic acid, TFA or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50° C., preferably between 15 and 30° C. (room temperature).

The BOC, OtBu and Mtr groups can, for example, preferably be cleaved off using TFA in dichloromethane or using approximately 3 to 5N HCl in dioxane at 15-30° C., and the FMOC group can be cleaved off using an approximately 5 to 50% solution of dimethylamine, diethylamine or piperidine in DMF at 15-30° C.

Protecting groups which can be removed hydrogenolytically (for example CBZ, benzyl or the liberation of the amidino group from the oxadiazole derivative thereof) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° C. and pressures between about 1 and 200 bar, preferably at 20-30° C. and 1-10 bar. Hydrogenolysis of the CBZ group succeeds well, for example, on 5 to 10% Pd/C in methanol or using ammonium formate (instead of hydrogen) on Pd/C in methanol/DMF at 20-30° C.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Esters can be hydrolysed, for example, using HCl, H₂SO₄, or using LiOH, NaOH or KOH in water, water/THF, water/THF/ethanol or water/dioxane, at temperatures between 0 and 100° C.

Free amino groups can furthermore be acylated in a conventional manner using an acyl chloride or anhydride or alkylated using an unsubstituted or substituted alkyl halide, advantageously in an inert solvent, such as dichloromethane or THF and/or in the presence of a base, such as triethylamine or pyridine, at temperatures between −60° C. and +30° C.

For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3rd Edition 1999.

Reaction schemes as described in the example section are illustrative only and should not be construed as limiting the invention in any way.

### Applications

The invention is further directed to the use of the compounds of the invention or pharmaceutically acceptable enantiomers, salts and solvates thereof as TDO2 inhibitors.

Accordingly, in a particularly preferred embodiment, the invention relates to the use of compounds of Formula I and subformulae in particular those of Table 1 above, or pharmaceutically acceptable enantiomers, salts and solvates thereof, as TDO2 inhibitors.

Accordingly, in another aspect, the invention relates to the use of these compounds or enantiomers, salts and solvates thereof for the synthesis of pharmaceutical active ingredients, such as TDO2 inhibitors.

In one embodiment, the invention relates to the use of compounds of Formula I and subformulae in particular those of Table 1 above, or pharmaceutically acceptable enantiomers, salts and solvates thereof, for increasing immune recognition and destruction of the cancer cells.

The compounds of the invention are therefore useful as medicaments, in particular in the prevention and/or treatment of cancer.

In one embodiment, compounds of the invention or pharmaceutically acceptable enantiomers, salts or solvates thereof are for use in the treatment and/or prevention of cancer, neurodegenerative disorders such as Parkinson's disease, Alzheimer's disease and Huntington's disease, chronic viral infections such as HCV and HIV, depression, and obesity.

The invention further relates to a method for treatment or prevention of cancer, neurodegenerative disorders such as Parkinson's disease, Alzheimer's disease and Huntington's disease, chronic viral infections such as HCV and HIV, depression, and obesity, which comprises administering to a mammalian species in need thereof a therapeutically effective amount of the compound according to the invention or a pharmaceutically acceptable enantiomers, salts or solvates thereof.

Various cancers are known in the art. The cancer may be metastatic or non-metastatic.

The cancer may be may be familial or sporadic. In some embodiments, the cancer is selected from the group consisting of: leukemia and multiple myeloma. Additional cancers that can be treated using the methods of the invention include, for example, benign and malignant solid tumours and benign and malignant non-solid tumours.

Examples of solid tumours include, but are not limited to: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumour), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumours (seminomas, and non-seminomas such as teratomas and choriocarcinomas), stromal tumours, germ cell tumours, and thyroid cancer (including thyroid adenocarcinoma and medullary carcinoma).

Examples of non-solid tumours include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

The invention also provides for a method for delaying in patient the onset of cancer comprising the administration of a pharmaceutically effective amount of a compound of Formula I or pharmaceutically acceptable enantiomer, salt and solvate thereof to a patient in need thereof.

Preferably, the patient is a warm-blooded animal, more preferably a human.

The compounds of the invention are especially useful in the treatment and/or prevention of cancer.

In a specific embodiment, the compounds of the invention are especially useful in the treatment and/or prevention of cancer.

The invention further provides the use of a compound of Formula I or a pharmaceutically acceptable enantiomer, salt and solvate thereof for the manufacture of a medicament for treating and/or preventing cancer.

According to a further feature of the present invention there is provided a method for modulating TDO2 activity, in a patient, preferably a warm blooded animal, and even more preferably a human, in need of such treatment, which comprises administering to said patient an effective amount of compound of the present invention, or a pharmaceutically acceptable enantiomer, salt and solvate thereof.

### Formulations

The invention also provides pharmaceutical compositions comprising a compound of Formula I or a pharmaceutically acceptable enantiomer, salt and solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable enantiomer, salt and solvate thereof as active ingredient, additional therapeutic agents and/or active ingredients.

Another object of this invention is a medicament comprising at least one compound of the invention, or a pharmaceutically acceptable enantiomer, salt and solvate thereof, as active ingredient.

According to a further feature of the present invention there is provided the use of a compound of Formula I or a pharmaceutically acceptable enantiomer, salt and solvate thereof for the manufacture of a medicament for modulating TDO2 activity in a patient, in need of such treatment, which comprises administering to said patient an effective amount of compound of the present invention, or a pharmaceutically acceptable enantiomer, salt and solvate thereof.

Generally, for pharmaceutical use, the compounds of the invention may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, the active compound of the invention may be administered as a single daily dose, divided over one or more daily doses, or essentially continuously, e.g. using a drip infusion.

### DEFINITIONS

In the present invention, the following terms have the following meanings:

Where groups may be substituted, such groups may be substituted with one or more substituents, and preferably with one, two or three substituents. Substituents may be selected from but not limited to, for example, the group comprising halogen, hydroxyl, oxo, nitro, amido, carboxy, amino, cyano haloalkoxy, and haloalkyl.

The term “halogen” means fluoro (F), chloro (Cl), bromo (Br), or iodo (I). The following definitions are used in connection with the compounds described herein. In general, the number of carbon atoms present in a given group is designated “Cx to Cy”, where x and y are the lower and upper limits, respectively. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are determined by naming from left to right the terminal portion of the functionality followed by the adjacent functionality toward the point of attachment. As used herein, “optionally substituted” means that at least 1 hydrogen atom of the optionally substituted group has been replaced.

The term “alkyl” by itself or as part of another substituent refers to a hydrocarbyl radical of Formula CₙH₂ₙ₊₁ wherein n is a number greater than or equal to 1. Alkyl groups may contain 1 to 10 carbons (inclusive), i.e., C1, C2, C3, C4, C5, C6, C7, C8, C9 or C10, i.e., C1-C10 alkyl. In certain embodiments, alkyl groups of this invention comprise from 1 to 6 carbon atoms, preferably from 1 to 4 carbon atoms, more preferably from 1 to 3 carbon atoms. Alkyl groups may be linear or branched and may be substituted as indicated herein. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

The term “haloalkyl” alone or in combination, refers to an alkyl radical having the meaning as defined above wherein one or more hydrogens are replaced with a halogen as defined above. Non-limiting examples of such haloalkyl radicals include fluoromethyl, difluoromethyl, trifluoro methyl and the like. In one example, the haloalkyl is a C1 to C6 alkyl group substituted with at least one halogen. In another example, the haloalkyl is a C1 to C4 alkyl group substituted with at least one halogen. Each halogen substitution may be independently selected.

The term “cycloalkyl” as used herein is a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, cyclohexyl, with cyclopropyl being particularly preferred.

The term “heteroatom” refers to a sulfur, nitrogen or oxygen atom.

Where at least one carbon atom in a cycloalkyl group is replaced with a heteroatom, the resultant ring is referred to herein as “heterocyclyl”.

The terms “heterocyclyl” or “heterocycle” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. The heterocycle may contain 3 to 7 carbon atoms (inclusive), or an integer therebetween. Any of the carbon atoms of the heterocyclic group may be substituted by oxo (for example piperidone, pyrrolidinone). The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include piperidinyl, azetidinyl, tetrahydropyranyl, piperazinyl, imidazolinyl, morpholinyl, oxetanyl, pyrazolidinyl imidazolidinyl, isoxazolinyl, oxazolidinyl, isoxazolidinyl, thiazolidinyl, isothiazolidinyl, indolyl, indolinyl, isoindolinyl, tetrahydrofuranyl, tetrahydroquinolinyl, thiomorpholinyl, thiomorpholinylsulfoxide, thiomorpholinylsulfone, pyrrolizinyl.

The term “aryl” as used herein refers to a polyunsaturated, aromatic hydrocarbyl group having a single ring (i.e. phenyl) or multiple aromatic rings fused together (e.g. naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic (carbon-containing ring) systems enumerated herein. Non-limiting examples of aryl comprise phenyl, biphenylyl, biphenylenylnaphthalenyl, indenyl.

The term “heteroaryl” as used herein by itself or as part of another group refers but is not limited to 5 to 12 carbon-atom aromatic rings or ring systems containing 1 to 2 rings which are fused together or linked covalently, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: pyridazinyl, pyridinyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyrimidyl, pyrazinyl, oxazinyl, dioxinyl, thiazinyl, triazinyl, indolyl, indolizinyl, isoindolyl, benzofuranyl, isobenzofuranyl, benzothiophenyl, isobenzothiophenyl, indazolyl, benzimidazolyl, quinolinyl, isoquinolinyl, cinnolinyl, quinazolinyl, quinoxalinyl.

The term “arylalkyl” refers to any group -alkyl-aryl. The term “alkylaryl” refers to any group -aryl-alkyl.

The term “heteroarylalkyl” refers to any group -alkyl-heteroaryl. The term “alkylheteroaryl” refers to any group -heteroaryl-alkyl.

The term “alkoxy” refers to any group O-alkyl. The term“haloalkoxy” refers to any group O-haloalkyl.

The term “oxo” refers to a ═O moiety.

The term “amino” refers to a —NH2 group or any group derived thereof by substitution of one nor two hydrogen atom by an organic aliphatic or aromatic group. Preferably, groups derived from —NH2 are “alkylamino” groups, i.e. N-alkyl groups, comprising monoalkylamino and dialkylamino. Non-limited examples of, the term “amino” include NH2, NHMe or NMe2.

The term “amino-protecting group” refers to a protecting group for an amine function. According to a preferred embodiment, the amino-protecting group is selected in the groups comprising: arylsulphonyl, tert-butoxy carbonyl, methoxymethyl, para-methoxy benzyl or benzyl.

The term “leaving group” refers to a molecular fragment that departs with a pair of electrons in heterolytic bond cleavage. According to a preferred embodiment, the leaving group is selected in the groups comprising: halogen, preferably iodine, bromine or chlorine; alkylsulfonyloxy having 1-6 carbon atoms, preferably methylsulfonyloxy or trifluoromethylsulfonyloxy; or arylsulfonyloxy having 6-10 carbon atoms, preferably phenyl- or p-tolylsulfonyloxy.

The term “solvate” is used herein to describe a compound in this invention that contains stoichiometric or sub-stoichiometric amounts of one or more pharmaceutically acceptable solvent molecule, e.g., ethanol. Typically, a solvate does not significantly alter the physiological activity or toxicity of the compounds, and as such may function as pharmacological equivalents to non-solvate compounds of Formula I and its subformula as defined herein. The term “solvate” as used herein is a combination, physical association and/or solvation of a compound of the present invention with a solvent molecule. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. “Solvate” may encompass solvates of salts of the compounds of Formula I.

The term “hydrate” refers to when the solvent molecule is water and may be an inorganic salt containing nH₂O, wherein n is the number of water molecules per formula unit of the salt. N may be ½, 1½, or an integer from 1 to 10. A hydrate which has lost water

The compounds of the invention include compounds of Formula I as hereinbefore defined, including all polymorphs and crystal habits thereof, prodrugs and prodrugs thereof and isotopically-labeled compounds of Formula I.

The invention also generally covers all pharmaceutically acceptable predrugs and prodrugs of the compounds of Formula I.

The term “prodrug” as used herein means the pharmacologically acceptable derivatives of compounds of Formula I, such as for example esters, whose in vivo biotransformation product generates the biologically active drug. Prodrugs are generally characterized by increased bio-availability and are readily metabolized into biologically active compounds in vivo.

The term “predrug”, as used herein, means any compound that will be modified to form a drug species, wherein the modification may take place either inside or outside of the body, and either before or after the predrug reaches the area of the body where administration of the drug is indicated.

The term “patient” refers to a warm-blooded animal, more preferably a human, who/which is awaiting the receipt of, or is receiving medical care or is/will be the object of a medical procedure.

The term “human” refers to a subject of both genders and at any stage of development (i.e. neonate, infant, juvenile, adolescent, adult).

The terms “treat”, “treating” and “treatment”, as used herein, are meant to include alleviating, attenuating or abrogating a condition or disease and/or its attendant symptoms.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “therapeutically effective amount” (or more simply an “effective amount”) as used herein means the amount of active agent or active ingredient that is sufficient to achieve the desired therapeutic or prophylactic effect in the patient to which/whom it is administered.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

By “pharmaceutically acceptable” is meant that the ingredients of a pharmaceutical composition are compatible with each other and not deleterious to the patient thereof.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

The words “comprise”, “comprises”, and “comprising” are to be interpreted inclusively rather than exclusively. The works “consist”, “consisting”, and its variants, are to be interpreted exclusively, rather than inclusively.

As used herein, the term “about” means a variability of 10% from the reference given, unless otherwise specified.

### EXAMPLES

The present invention will be better understood with reference to the following examples. These examples are intended to representative of specific embodiments of the invention, and are not intended as limiting the scope of the invention.

### I. Chemistry Examples

The mass spectrometry (MS) data provided in the examples described below were obtained as followed: Mass spectrum: LC/MS Agilent 6110 (Electron Spray Ionization, ESI) or a Waters Acquity SQD (ESI) The NMR data provided in the examples described below were obtained as followed:

Bruker Ultrashield™ 400 PLUS and Bruker Fourier 300 MHz and TMS was used as an internal standard.

The microwave chemistry was performed on a single mode microwave reactor Initiator Microwave System EU from Biotage.

Preparative High Performance Liquid Chromatography (HPLC) purifications were performed with a mass directed autopurification Fractionlynx from Waters equipped with a Xbridge™ Prep C18 OBD column 19×150 mm 5 μm, unless otherwise reported. All HPLC purifications were performed with a gradient of CH₃CN/H₂O/NH₄HCO₃ (5 mM), CH₃CN/H₂O/TFA (0.1%), or CH₃CN/H₂O/NH₃H₂O (0.1%).

The following abbreviations are used herein and have the indicated definitions: ACN is acetonitrile; DMSO is dimethylsulfoxide; DCM is dichloromethane; DIPEA is diisopropylethylamine; DMF is N,N-dimethylformamide; dppf is 1,1′-bis(diphenylphosphino)ferrocene; EtOH is ethanol; HATU is 2-(1H-7-azabenzotriazol-1-yl)-1,1,3,3-tetramethyl uronium hexafluorophosphate methanaminium; Hz is hertz; KOAc is potassium acetate; MeOH is methanol; MHz is megahertz; mM is millimolar; mL is milliliter; min is minutes; mol is moles; M is molecular ion; [M+H]⁺ is protonated molecular ion; N is normality; NMR is nuclear magnetic resonance; PPh₃ is triphenylphosphine; psi is pound per square inch; PPM is parts per million; qd po means daily by mouth; rt is room temperature; RT is retention time; TLC is thin layer chromatography; TFA is trifluoroacetic acid; TEA is triethylamine.

### I.1. Synthesis of Intermediates

### Intermediate 1

### 6-fluoro-3-iodo-1-(phenylsulfonyl)-1H-indole

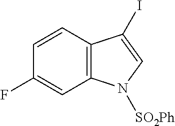

The title compound was prepared using the same procedure as reported (WO2010/136491A1).

### Intermediate 2

### tert-butyl 6-fluoro-3-(4,4,5,5-tetramethyl-1,3,2-dioxaborolan-2-yl)-1H-indole-1-carboxylate

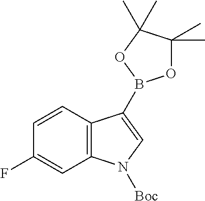 [SMILES: CC(C)(C)OC(=O)n1cc(B2OC(C)(C)C(C)(C)O2)c2ccc(F)cc21]

The title compound was prepared using the same procedure as reported (US2014/256706 A1).

### Intermediate 3

### 6-(4,4,5,5-tetramethyl-1,3,2-dioxaborolan-2-yl)-1H-indazole

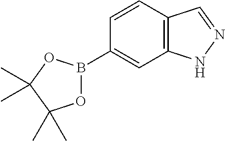 [SMILES: CC1(C)OB(c2ccc3cn[nH]c3c2)OC1(C)C]

A mixture of 6-bromo-1H-indazole (700 mg; 3.55 mmol), 4,4,4′,4′,5,5,5′,5′-octamethyl-2,2′-bi(1,3,2-dioxaborolane) (1.50 g; 5.91 mmol), Pd(dppf)Cl₂.DCM (290 mg; 0.36 mmol) and KOAc (1.04 g; 10.6 mmol) in DMF (20 mL) was stirred at 100° C. for 15 hours under nitrogen. The mixture was concentrated in vacuo, suspended in EtOAc (30 mL), filtered through Celite, and concentrated to afford 866 mg (100%) of the title compound as a brown semi-solid, which was used directly without further purification. LC-MS for C₁₃H₁₇BN₂O₂+H⁺ [M+H]⁺: calcd. 245.1. found: 245.0.

### Intermediate 4

### 6-(6-fluoro-1-(phenylsulfonyl)-1H-indol-3-yl)-1H-indazole

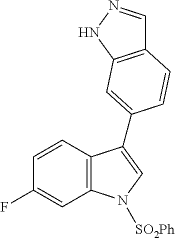

To a mixture of 6-fluoro-3-iodo-1-(phenylsulfonyl)-1H-indole (Intermediate 1; 400 mg; 1.00 mmol), 6-(4,4,5,5-tetramethyl-1,3,2-dioxaborolan-2-yl)-1H-indazole (Intermediate 3; 366 mg; 1.50 mmol) and Cs₂CO₃ (978 mg; 3.00 mmol) in DME (9 mL) and water (3 mL) was added Pd(dppf)Cl₂.DCM (82 mg; 0.1 mmol) under nitrogen. The mixture was heated at 150° C. for 1 hour in a microwave reactor. The mixture was filtered through Celite and diluted with EtOAc (100 mL) and water (100 mL). The aqueous layer was extracted with EtOAc (50 mL). The combined organic layers were washed with brine, dried over anhydrous Na₂SO₄, filtered, concentrated, and purified by a silica gel chromatography (petroleum ether/EtOAc=5/1-2/1) to afford 390 mg (100%) of the title compound as a yellow solid. LC-MS for C₂₁H₁₄FN₃O₂S+H⁺[M+H]⁺: calcd. 392.1. found: 391.8. ¹H NMR (300 MHz, DMSO-d₆) δ [ppm]: 13.13 (s, 1H), 8.20-8.11 (m, 4H), 7.93-7.78 (m, 4H), 7.77-7.70 (m, 1H), 7.65-7.60 (m, 2H), 7.44 (dd, J=9.3, 2.1 Hz, 1H), 7.26 (ddd, J=9.3, 9.0, 2.1 Hz, 1H).

### Intermediate 5

### tert-butyl 4-((6-(6-fluoro-1-(phenylsulfonyl)-1H-indol-3-yl)-1H-indazol-1-yl)methyl)piperidine-1-carboxylate and tert-butyl 4-((6-(6-fluoro-1-(phenylsulfonyl)-1H-indol-3-yl)-2H-indazol-2-yl)methyl)piperidine-1-carboxylate

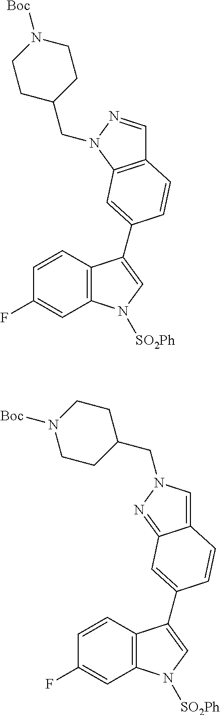

A mixture of 6-fluoro-3-iodo-1-(phenylsulfonyl)-1H-indole (Intermediate 1; 1.00 g; 2.55 mmol), tert-butyl 4-(((methylsulfonyl)oxy)methyl)piperidine-1-carboxylate (1.10 g; 3.75 mmol) and Cs₂CO₃ (1.66 g; 5.09 mmol) in DMF (40 mL) was stirred at 60° C. overnight under nitrogen. The mixture was cooled to room temperature, diluted with EtOAc (100 mL), filtered, concentrated, and purified by a silica gel chromatography (petroleum ether/EtOAc=6/1-2/1) to afford 1.11 g (74%) of a mixture of the title compounds as a yellow solid. LC-MS for C₃₂H₃₃FN₄O₄S+H⁺[M+H]⁺: calcd. 589.2. found: 588.9.

### Intermediate 6

### 6-(6-fluoro-1-(phenylsulfonyl)-1H-indol-3-yl)-1-(piperidin-4-ylmethyl)-1H-indazole hydrochloride and 6-(6-fluoro-1-(phenylsulfonyl)-1H-indol-3-yl)-2-(piperidin-4-ylmethyl)-2H-indazole hydrochloride

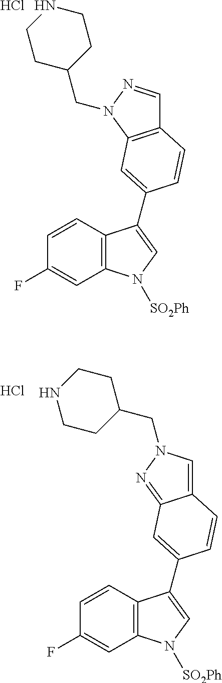

To a solution of tert-butyl 4-((6-(6-fluoro-1-(phenylsulfonyl)-1H-indol-3-yl)-1H-indazol-1-yl)methyl)piperidine-1-carboxylate and tert-butyl 4-((6-(6-fluoro-1-(phenylsulfonyl)-1H-indol-3-yl)-2H-indazol-2-yl)methyl)piperidine-1-carboxylate (Intermediate 5; 1.06 g; 1.80 mmol) in MeOH (40 mL) was added concentrated aqueous HCl (16 mL; 37%). The reaction mixture was stirred for 1 hour and concentrated in vacuo to afford 1.55 g (quant.) of the title compound as a yellow solid, which was used directly without further purification. LC-MS for C₂₇H₂₅FN₄O₂S+H⁺[M+H]⁺: calcd. 489.2. found: 488.9.

### Intermediate 7

### 1-(4-((6-(6-fluoro-1-(phenylsulfonyl)-1H-indol-3-yl)-1H-indazol-1-yl)methyl)piperidin-1-yl)ethanone

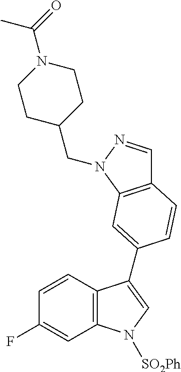

To a solution of 6-(6-fluoro-1-(phenylsulfonyl)-1H-indol-3-yl)-1-(piperidin-4-ylmethyl)-1H-indazole hydrochloride and 6-(6-fluoro-1-(phenylsulfonyl)-1H-indol-3-yl)-2-(piperidin-4-ylmethyl)-2H-indazole hydrochloride (Intermediate 6; 650 mg; 1.24 mmol) and Et₃N (372 mg; 3.68 mmol) in DCM (30 mL) was added Acetyl chloride (116.6 mg; 1.48 mmol) under nitrogen. The reaction mixture was stirred for 1 hour and quenched with saturated aqueous NaHCO₃ (30 mL). The aqueous layer was extracted with DCM (70 mL×2). The combined organic layers were washed with brine (20 mL), dried over anhydrous Na₂SO₄, filtered, concentrated, and purified by preparative TLC (EtOAc) to afford 267 mg (40%) of the title compound as a yellow solid. LC-MS for C₂₉H₂₇FN₄O₃S+H⁺[M+H]⁺: calcd. 531.2. found: 530.9.

### Intermediate 8

### 3-(6-(6-fluoro-1-(phenylsulfonyl)-1H-indol-3-yl)-1H-indazol-1-yl)propanamide

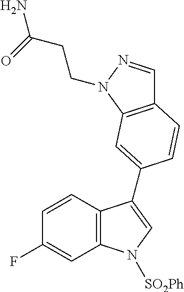

A mixture of 6-(6-fluoro-1-(phenylsulfonyl)-1H-indol-3-yl)-1H-indazole (Intermediate 4; 200 mg; 0.51 mmol), 3-bromopropanamide (389 mg; 2.56 mmol), K₂CO₃ (211 mg; 1.53 mmol) and KI (85 mg; 0.51 mmol) in DMF (6 mL) was heated at 130° C. for 2 hours in a microwave reactor. The mixture was poured into H₂O (20 mL) and the aqueous layer was extracted with EtOAc (50 mL×2). The combined organic layers were washed with brine (100 mL×3), dried over anhydrous Na₂SO₄, filtered, concentrated, and purified by a silica gel chromatography (petroleum ether/EtOAc=2/1-0/1) to afford 95 mg (40%) of the title compound as a yellow solid. LC-MS for C₂₄H₁₉FN₄O₃S+H⁺[M+H]⁺: calcd. 463.1. found: 462.8.

### Intermediate 9

### tert-butyl 4-(6-(6-fluoro-1-(phenylsulfonyl)-1H-indol-3-yl)-1H-indazol-1-yl)piperidine-1-carboxylate and tert-butyl 4-(6-(6-fluoro-1-(phenylsulfonyl)-1H-indol-3-yl)-2H-indazol-2-yl)piperidine-1-carboxylate

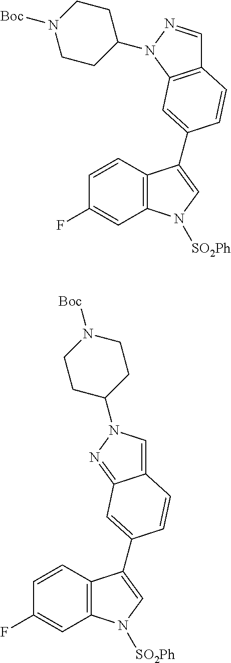

A mixture of 6-fluoro-3-iodo-1-(phenylsulfonyl)-1H-indole (Intermediate 1; 560 mg; 1.43 mmol), tert-butyl 4-((methylsulfonyl)oxy)piperidine-1-carboxylate (0.60 g; 2.15 mmol) and Cs₂CO₃ (1.00 g; 3.07 mmol) in DMF (22 mL) was stirred at 60° C. overnight under nitrogen. The mixture was cooled to room temperature, diluted with EtOAc (100 mL), filtered, and concentrated to afford 820 mg (quant.) of a mixture of the title compounds as a yellow solid, which was used directly without further purification. LC-MS for C31H₃₁FN₄O₄S+H⁺[M+H]⁺: calcd. 575.2. found: 574.8.

### Intermediate 10

### 6-(6-fluoro-1-(phenylsulfonyl)-1H-indol-3-yl)-1-(piperidin-4-yl)-1H-indazole hydrochloride and 6-(6-fluoro-1-(phenylsulfonyl)-1H-indol-3-yl)-2-(piperidin-4-yl)-2H-indazole hydrochloride

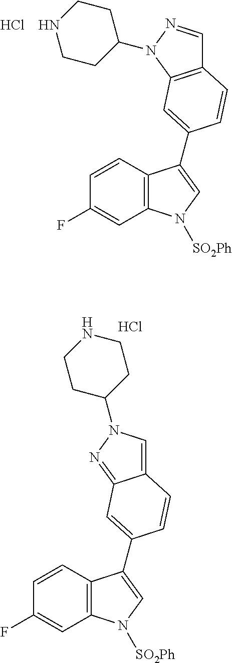

Following the general method as outlined in Intermediate 6, starting from tert-butyl 4-(6-(6-fluoro-1-(phenylsulfonyl)-1H-indol-3-yl)-1H-indazol-1-yl)piperidine-1-carboxylate and tert-butyl 4-(6-(6-fluoro-1-(phenylsulfonyl)-1H-indol-3-yl)-2H-indazol-2-yl)piperidine-1-carboxylate (Intermediate 9; 820 mg; 1.43 mmol), 730 mg (100%) of a mixture of the title compounds was obtained as a yellow solid, which was used directly without further purification. LC-MS for C₂₆H₂₃FN₄O₂S+H⁺[M+H]⁺: calcd. 475.2. found: 474.8.

### Intermediate 11

### 5-(4,4,5,5-tetramethyl-1,3,2-dioxaborolan-2-yl)-1H-indazole

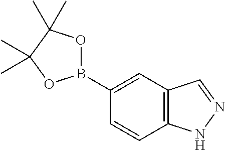 [SMILES: CC1(C)OB(c2ccc3[nH]ncc3c2)OC1(C)C]

A mixture of 5-bromo-1H-indazole (500 mg; 2.54 mmol), 4,4,4′,4′,5,5,5′,5′-octamethyl-2,2′-bi(1,3,2-dioxaborolane) (1.29 g; 5.08 mmol), Pd(dppf)Cl₂.DCM (200 mg; 0.24 mmol) and KOAc (1.24 g; 12.6 mmol) in DMF (10 mL) was stirred at 90° C. for 15 hours under nitrogen. The mixture was concentrated in vacuo, diluted with EtOAc (100 mL), washed with H₂O (50 mL×2), brine (50 mL), dried over anhydrous Na₂SO₄, filtered through Celite, and concentrated to afford 1.18 g (quant.) of the title compound as a yellow oil, which was used directly without further purification. LC-MS for C₁₃H₁₇BN₂O₂+H⁺[M+H]⁺: calcd. 245.1. found: 245.2.

### Intermediate 12

### 5-(6-fluoro-1-(phenylsulfonyl)-1H-indol-3-yl)-1H-indazole

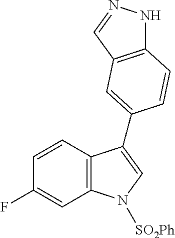

To a mixture of 6-fluoro-3-iodo-1-(phenylsulfonyl)-1H-indole (Intermediate 1; 200 mg; 0.50 mmol), 5-(4,4,5,5-tetramethyl-1,3,2-dioxaborolan-2-yl)-1H-indazole (Intermediate 11; 345 mg crude; 0.74 mmol) and Cs₂CO₃ (488 mg; 1.50 mmol) in DME (6 mL) and water (2 mL) was added Pd(dppf)Cl₂.DCM (40 mg; 0.05 mmol) under nitrogen. The mixture was heated at 150° C. for 30 minutes in a microwave reactor. The mixture was filtered through Celite and diluted with EtOAc (100 mL) and water (100 mL). The aqueous layer was extracted with EtOAc (50 mL). The combined organic layers were washed with brine, dried over anhydrous Na₂SO₄, filtered, and concentrated to afford 300 mg (quant.) of the title compound as a yellow oil, which was used directly without further purification. LC-MS for C₂₁H₁₄FN₃O₂S+H⁺[M+H]⁺: calcd. 392.1. found: 392.0.

### Intermediate 13

### 2-(5-(6-fluoro-1-(phenylsulfonyl)-1H-indol-3-yl)-1H-indazol-1-yl)acetamide and 2-(5-(6-fluoro-1-(phenylsulfonyl)-1H-indol-3-yl)-2H-indazol-2-yl)acetamide

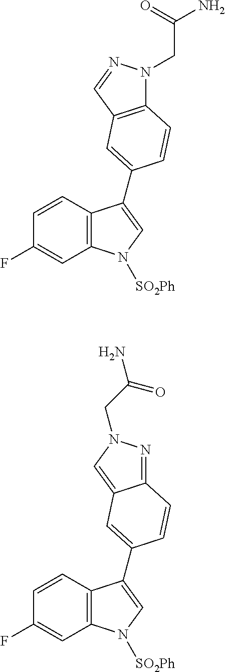

A mixture of 5-(6-fluoro-1-(phenylsulfonyl)-1H-indol-3-yl)-1H-indazole (Intermediate 12; 500 mg; 1.28 mmol), 2-bromoacetamide (900 mg; 6.52 mmol), K₂CO₃ (540 mg; 3.91 mmol) and KI (220 mg; 1.33 mmol) in DMF (12 mL) was heated at 13° C. for 4 hours in a microwave reactor. The mixture was poured into H₂O (10 mL) and extracted with EtOAc (20 mL×4). The combined organic layers were washed with brine (20 mL), dried over anhydrous Na₂SO₄, filtered, and concentrated to afford 353 mg (62%) of a mixture of the title compounds as a brown solid, which was used directly without further purification. LC-MS for C₂₃H₁₇FN₄O₃S+H⁺[M+H]⁺: calcd. 449.1. found: 448.8.

### Intermediate 14

### tert-butyl 4-((5-(6-fluoro-1-(phenylsulfonyl)-1H-indol-3-yl)-1H-indazol-1-yl)methyl)piperidine-1-carboxylate and tert-butyl 4-((5-(6-fluoro-1-(phenylsulfonyl)-1H-indol-3-yl)-2H-indazol-2-yl)methyl)piperidine-1-carboxylate

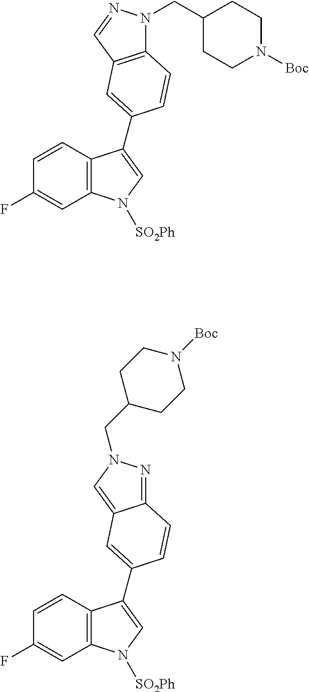

Following the general method as outlined in Intermediate 5, starting from 5-(6-fluoro-1-(phenylsulfonyl)-1H-indol-3-yl)-1H-indazole (Intermediate 12; 1.50 g; 3.83 mmol) and tert-butyl 4-(((methylsulfonyl)oxy)methyl)piperidine-1-carboxylate (1.34 g; 4.57 mmol), 1.97 g (88%) of a mixture of the title compounds was obtained as a brown oil, which was used directly without further purification. LC-MS for C₃₂H₃₃FN₄O₄S+H⁺ [M+H]⁺: calcd. 589.2. found: 588.8.

### Intermediate 15

### tert-butyl 4-((5-(6-fluoro-1H-indol-3-yl)-1H-indazol-1-yl)methyl)piperidine-1-carboxylate

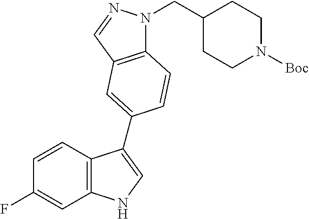 [SMILES: CC(C)(C)OC(=O)N1CCC(Cn2ncc3cc(-c4c[nH]c5cc(F)ccc45)ccc32)CC1]

To a solution of tert-butyl 4-((5-(6-fluoro-1-(phenylsulfonyl)-1H-indol-3-yl)-1H-indazol-1-yl)methyl)piperidine-1-carboxylate and tert-butyl 4-((5-(6-fluoro-1-(phenylsulfonyl)-1H-indol-3-yl)-2H-indazol-2-yl)methyl)piperidine-1-carboxylate (Intermediate 14; 2.00 g; 3.40 mmol) in MeOH (40 mL) was added a solution of NaOH (679 mg; 17.0 mmol) in water (2 mL). The reaction mixture was stirred at 85° C. for 30 minutes, concentrated, and purified by a silica gel chromatography (petroleum ether/EtOAc=3/1) to afford 1.18 g (77%) of the title compound as a yellow solid. LC-MS for C₂₆H₂₉FN₄O₂+H⁺ [M+H]⁺: calcd. 449.2. found: 448.9.

### Intermediate 16

### 3-(5-(6-fluoro-1-(phenylsulfonyl)-1H-indol-3-yl)-1H-indazol-1-yl)propanamide and 3-(5-(6-fluoro-1-(phenylsulfonyl)-1H-indol-3-yl)-2H-indazol-2-yl)propanamide

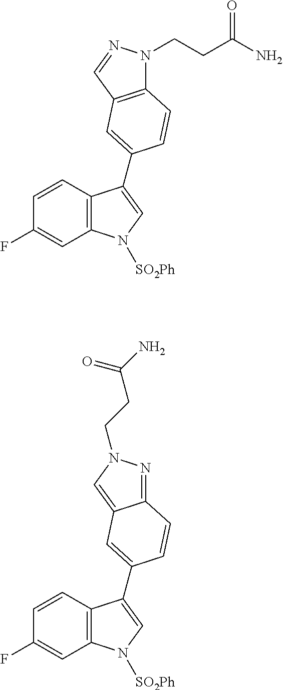

Following the general method as outlined in Intermediate 8, starting from 5-(6-fluoro-1-(phenylsulfonyl)-1H-indol-3-yl)-1H-indazole (Intermediate 12; 490 mg; 1.25 mmol), 492 mg (85%) of a mixture of the title compounds was obtained as a brown solid, which was used directly without further purification. LC-MS for C₂₄H₁₉FN₄O₃S+H⁺ [M+H]⁺: calcd. 463.2. found: 462.8.

### Intermediate 17

### tert-butyl 4-(5-(6-fluoro-1-(phenylsulfonyl)-1H-indol-3-yl)-1H-indazol-1-yl)piperidine-1-carboxylate

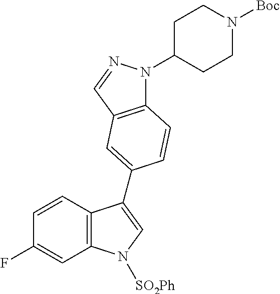

To a mixture of 5-(6-fluoro-1-(phenylsulfonyl)-1H-indol-3-yl)-1H-indazole (Intermediate 12; 1.80 g; 4.60 mmol) and Cs₂CO₃ (3.75 g; 11.5 mmol) in DMF (35 mL) was added tert-butyl 4-(methylsulfonyloxy)piperidine-1-carboxylate (1.93 g; 6.91 mmol) under nitrogen. The reaction mixture was stirred at 70° C. overnight. The mixture was poured into ice-water (180 mL) and extracted with EtOAc (80 mL×3). The combined organic layers were washed with water (50 mL), brine (50 mL), dried over anhydrous Na₂SO₄, filtered, concentrated, and purified by a silica gel chromatography (petroleum ether/EtOAc=5/1-2/1) to afford 1.75 g (66%) of the title compound as a yellow solid. LC-MS for C₃₁H₃₁FN₄O₄S+H⁺ [M+H]⁺: calcd. 575.2. found: 574.9.

### Intermediate 18

### tert-butyl 4-(5-(6-fluoro-1H-indol-3-yl)-1H-indazol-1-yl)piperidine-1-carboxylate

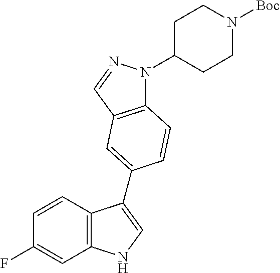 [SMILES: CC(C)(C)OC(=O)N1CCC(n2ncc3cc(-c4c[nH]c5cc(F)ccc45)ccc32)CC1]

To a solution of tert-butyl 4-(5-(6-fluoro-1-(phenylsulfonyl)-1H-indol-3-yl)-1H-indazol-1-yl)piperidine-1-carboxylate (Intermediate 17; 1.75 g; 3.05 mmol) in MeOH (60 mL) was added a solution of NaOH (800 mg; 20.0 mmol) in water (6 mL). The reaction mixture was stirred at 85° C. for 6 hours and concentrated in vacuo. The residual was dissolved in EtOAc (80 mL), washed with water (40 mL), brine (40 mL), dried over anhydrous Na₂SO₄, filtered, concentrated, and purified by a silica gel chromatography (petroleum ether/EtOAc=5/1-2/1) to afford 600 mg (45%) of the title compound as a yellow oil. LC-MS for C₂₅H₂₇FN₄O₂+H⁺-(CH₃)₂C═CH₂ [M+H—(CH₃)₂C═CH₂]⁺: calcd. 379.2. found: 378.9.

### Intermediate 19

### 1-(4-((6-(6-fluoro-1-(phenylsulfonyl)-1H-indol-3-yl)-2H-indazol-2-yl)methyl)piperidin-1-yl)ethanone

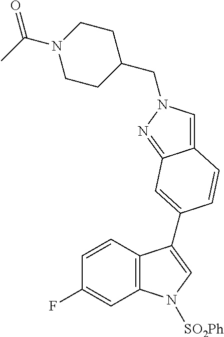

358 mg (54%) of the title compound was obtained as a yellow solid after purification by preparative TLC (EtOAc) as a regio-isomer with intermediate 7. LC-MS for C₂₉H₂₇FN₄O₃S+H [M+H]⁺: calcd. 531.2. found: 530.9.

### Intermediate 20

### 3-(6-(6-fluoro-1-(phenylsulfonyl)-1H-indol-3-yl)-2H-indazol-2-yl)propanamide

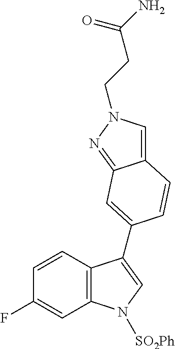

The title compound (70 mg, 29%) was obtained as a yellow solid after purification by silica gel chromatography (petroleum ether/EtOAc=2/1-0/1) during the preparation of Intermediate 8.

### Intermediate 21

### tert-butyl 4-((5-(6-fluoro-1H-indol-3-yl)-2H-indazol-2-yl)methyl)piperidine-1-carboxylate

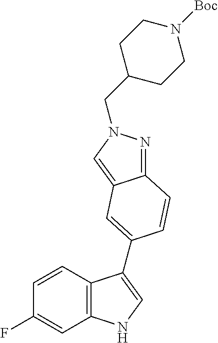 [SMILES: CC(C)(C)OC(=O)N1CCC(Cn2cc3cc(-c4c[nH]c5cc(F)ccc45)ccc3n2)CC1]

The title compound (377 mg, 25%) was obtained as a yellow solid after purification by silica gel chromatography (petroleum ether/EtOAc=3/1) during the preparation of Intermediate 15. LC-MS for C₂₆H₂₉FN₄O₂+H⁺[M+H]⁺: calcd. 449.2. found: 448.9.

### Intermediate 22

### 5-(6-fluoro-1H-indol-3-yl)-2-(piperidin-4-ylmethyl)-2H-indazole

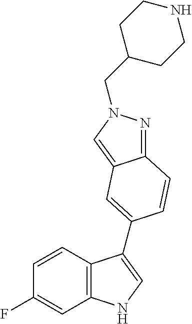 [SMILES: Fc1ccc2c(-c3ccc4nn(CC5CCNCC5)cc4c3)c[nH]c2c1]

A solution of tert-butyl 4-((5-(6-fluoro-1H-indol-3-yl)-2H-indazol-2-yl)methyl)piperidine-1-carboxylate (Intermediate 21; 189 mg; 0.42 mmol) in saturated HCl in 1,4-dioxane (10 mL) was stirred at room temperature for 1 hour. The reaction mixture was concentrated in vacuo, diluted with water (20 mL), and extracted with EtOAc (50 mL×3). The aqueous layer was basified with aqueous NaOH to pH=13 and extracted with EtOAc (80 mL×3). The combined organic layers were dried over anhydrous Na₂SO₄, filtered, concentrated, and purified by preparative TLC (DCM/MeOH=10/1) to afford 136 mg (93%) of the title compound as a yellow solid. LC-MS for C₂₁H₂₁FN₄+H⁺[M+H]⁺: calcd. 349.2. found: 348.9.

### Intermediate 23

### tert-butyl 4-(5-(6-fluoro-1-(phenylsulfonyl)-1H-indol-3-yl)-2H-indazol-2-yl)piperidine-1-carboxylate

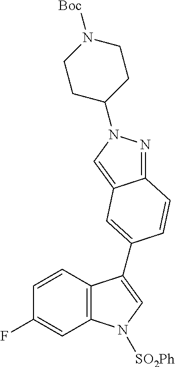

The title compound (380 mg, 14%) was obtained as a yellow solid after purification by silica gel chromatography (petroleum ether/EtOAc=5/1-2/1) during the preparation of Intermediate 17. LC-MS for C₃₁H₃₁FN₄O₄S+H⁺[M+H]⁺: calcd. 575.2. found: 574.9.

### Intermediate 24

### tert-butyl 4-(5-(6-fluoro-1H-indol-3-yl)-2H-indazol-2-yl)piperidine-1-carboxylate

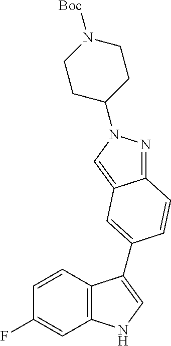 [SMILES: CC(C)(C)OC(=O)N1CCC(n2cc3cc(-c4c[nH]c5cc(F)ccc45)ccc3n2)CC1]

Following the general method as outlined in Intermediate 15, starting from tert-butyl 4-(5-(6-fluoro-1-(phenylsulfonyl)-1H-indol-3-yl)-2H-indazol-2-yl)piperidine-1-carboxylate (Intermediate 23; 1.75 g; 3.04 mmol), 0.6 g (45%, not very pure) of the title compound was obtained as a yellow oil purification by silica gel chromatography (petroleum ether/EtOAc=5/1-2/1). LC-MS for C₂₅H₂₇FN₄O₂+H⁺[M+H]⁺: calcd. 435.5. found: 434.9.

### Intermediate 25

### tert-butyl 5-bromo-1H-benzo[d]imidazole-1-carboxylate and tert-butyl 6-bromo-1H-benzo[d]imidazole-1-carboxylate

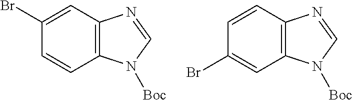 [SMILES: CC(C)(C)OC(=O)n1cnc2cc(Br)ccc21 ; CC(C)(C)OC(=O)n1cnc2ccc(Br)cc21]

To a mixture of 5-bromo-1H-benzo[d]imidazole (1.00 g; 5.08 mmol) in THF (50 mL) was added Boc₂O (1.33 g; 6.09 mmol), Et₃N (770 mg; 7.61 mmol) and DMAP (62 mg; 0.51 mmol). The mixture was stirred at room temperature overnight under nitrogen and concentrated in vacuo. The residue was dissolved in EtOAc (150 mL), washed with H₂O (80 mL×2), brine (80 mL), dried over anhydrous Na₂SO₄, filtered, and concentrated to afford 1.30 g (86%) of a mixture of the title compounds as a yellow oil, which was used directly without further purification. LC-MS for C₁₂H₁₃BrN₂O₂+H⁺—(CH₃)₂C═CH₂ [M+H—(CH₃)₂C═CH₂]⁺: calcd. 241.0. found: 240.9. ¹H NMR (300 MHz, CDCl₃) δ [ppm]: 8.42 (s, 0.5H), 8.40 (s, 0.5H), 8.20 (s, 0.5H), 7.95 (s, 0.5H), 7.88 (d, J=8.7 Hz, 0.5H), 7.66 (d, J=8.7 Hz, 0.5H), 7.55-7.44 (m, 1H), 1.71 (s, 9H) as a mixture of tautomers.

### Intermediate 26

### tert-butyl 5-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate

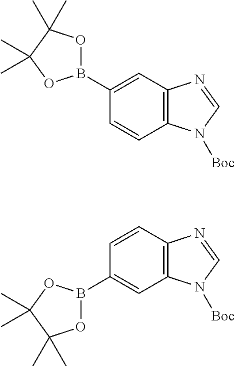 [SMILES: CC(C)(C)OC(=O)n1cnc2cc(B3OC(C)(C)C(C)(C)O3)ccc21 ; CC(C)(C)OC(=O)n1cnc2ccc(B3OC(C)(C)C(C)(C)O3)cc21]

Following the general method as outlined in Intermediate 3, starting from tert-butyl 5-bromo-1H-benzo[d]imidazole-1-carboxylate and tert-butyl 6-bromo-1H-benzo[d]imidazole-1-carboxylate (Intermediate 25; 500 mg; 1.68 mmol), 1.09 g (quant.) of a mixture of the title compounds was obtained as a yellow oil, which was used directly without further purification. LC-MS for C₁₈H₂₅BN₂O₄+H⁺[M+H]⁺: calcd. 345.2. found: 345.0.

### Intermediate 27

### 5-(6-fluoro-1-(phenylsulfonyl)-1H-indol-3-yl)-1H-benzo[d]imidazole

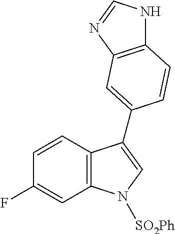

Following the general method as outlined in Intermediate 4, starting from tert-butyl 5-(4,4,5,5-tetramethyl-1,3,2-dioxaborolan-2-yl)-1H-benzo[d]imidazole-1-carboxylate and tert-butyl 6-(4,4,5,5-tetramethyl-1,3,2-dioxaborolan-2-yl)-1H-benzo[d]imidazole-1-carboxylate (Intermediate 26; 730 mg crude; 0.46 mmol) and 6-fluoro-3-iodo-1-(phenylsulfonyl)-1H-indole (Intermediate 1; 300 mg; 0.75 mmol), 550 mg (quant.) of the title compound was obtained as a black oil, which was used directly without further purification. LC-MS for C₂₁H₁₄FN₃O₂S+H⁺[M+H]⁺: calcd. 392.1. found: 391.9.

### Intermediate 28

### tert-butyl 5-bromo-2-methyl-1H-benzo[d]imidazole-1-carboxylate

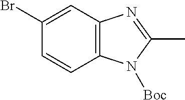 [SMILES: Cc1nc2cc(Br)ccc2n1C(=O)OC(C)(C)C]

Following the general method as outlined in Intermediate 25, starting from 5-bromo-2-methyl-1H-benzo[d]imidazole (3.0 g; 13.51 mmol), 1.14 g (27%) of the title compound was obtained as a white solid after purification by a silica gel chromatography (petroleum ether/EtOAc=8/1-5/1). LC-MS for C₁₃H₁₅BrN₂O₂+H⁺—(CH₃)₂C═CH₂ [M+H—(CH₃)₂C═CH₂]⁺: calcd. 256.0. found: 256.8. ¹H NMR (300 MHz, CDCl₃) δ [ppm]: 7.79 (d, J=8.7 Hz, 1H), 7.79 (d, J=2.0 Hz, 1H), 7.76 (m, 2H), 7.41 (dd, J=8.7, 2.0 Hz, 1H), 2.82 (s, 3H), 1.72 (s, 9H).

### Intermediate 29

### tert-butyl 2-methyl-5-(4,4,5,5-tetramethyl-1,3,2-dioxaborolan-2-yl)-1H-benzo[d]imidazole-1-carboxylate

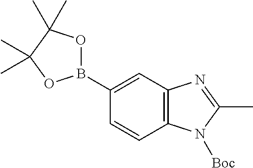

Following the general method as outlined in Intermediate 3, starting from tert-butyl 5-bromo-2-methyl-1H-benzo[d]imidazole-1-carboxylate (Intermediate 28; 1.14 g; 3.67 mmol), 1.51 g (quant.) of the title compound was obtained as a white solid after purification by a silica gel chromatography (petroleum ether/EtOAc=5/1). LC-MS for C₁₉H₂₇BN₂O₄+H⁺[M+H]⁺: calcd. 359.2. found: 359.0. ¹H NMR (400 MHz, DMSO-d₆) δ [ppm]: 7.91 (d, J=8.2 Hz, 1H), 7.84 (s, 1H), 7.64 (d, J=8.2 Hz, 1H), 2.72 (s, 3H), 1.66 (s, 9H), 1.32 (s, 12H).

### Intermediate 30

### 5-(6-fluoro-1-(phenylsulfonyl)-1H-indol-3-yl)-2-methyl-1H-benzo[d]imidazole

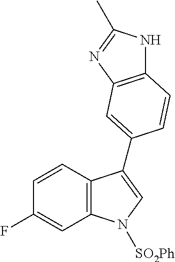

Following the general method as outlined in Intermediate 4, starting from tert-butyl 2-methyl-5-(4,4,5,5-tetramethyl-1,3,2-dioxaborolan-2-yl)-1H-benzo[d]imidazole-1-carboxylate (Intermediate 29; 1.08 g; 3.01 mmol) and 6-fluoro-3-iodo-1-(phenylsulfonyl)-1H-indole (Intermediate 1; 802 mg; 1.99 mmol), 870 mg (quant.) of the title compound was obtained as a black solid after purification by a silica gel chromatography (DCM/MeOH=40/1-20/1). LC-MS for C₂₂H₁₆FN₃O₂S+H⁺ [M+H]⁺: calcd. 406.1. found: 405.8.

### Intermediate 31

### 4-(4,4,5,5-tetramethyl-1,3,2-dioxaborolan-2-yl)benzene-1,2-diamine

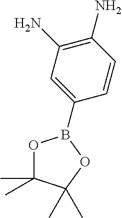 [SMILES: CC1(C)OB(c2ccc(N)c(N)c2)OC1(C)C]

To a mixture of 4-bromobenzene-1,2-diamine (10.8 g; 57.7 mmol), KOAc (17.0 g; 173 mmol) and 44,4,4′,4′,5,5,5′,5′-octamethyl-2,2′-bi(1,3,2-dioxaborolane) (16.1 g; 63.4 mmol) in dioxane (200 mL) was added Pd(dppf)Cl₂.DCM (2.36 g; 2.89 mmol) under nitrogen. The reaction mixture was stirred at 100° C. for 16 hours, cooled to room temperature, filtered, concentrated, and purified by a silica gel chromatography (petroleum ether/EtOAc=5/1-2/1) to afford 11.6 g (86%) of the title compound as a brown oil. LC-MS for C₁₂H₁₉BN₂O₂+H⁺[M+H]⁺: calcd. 235.2. found: 235.0. ¹H NMR (400 MHz, CDCl₃) δ [ppm]: 7.22 (d, J=7.7 Hz, 1H), 7.16 (s, 1H), d, J=7.7 Hz, 1H, 1.32 (s, 12H).

### Intermediate 32

### 4-(6-fluoro-1-(phenylsulfonyl)-1H-indol-3-yl)benzene-1,2-diamine H₂N NH₂

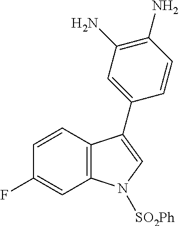

A mixture of 6-fluoro-3-iodo-1-(phenylsulfonyl)-1H-indole (Intermediate 1; 19.9 g; 49.6 mmol), 4-(4,4,5,5-tetramethyl-1,3,2-dioxaborolan-2-yl)benzene-1,2-diamine (Intermediate 31; 11.6 g; 49.6 mmol), Pd(dppf)Cl₂.DCM (2.0 g; 2.45 mmol), K₂CO₃ (20.5 g; 148 mmol) in dioxane (300 mL) and water (60 mL) was stirred at 100° C. for 2 hours under nitrogen. The reaction mixture was cooled to room temperature, filtered, concentrated, and purified by a silica gel chromatography (DCM/MeOH=200/1-20/1) to afford 7.0 g (37%) of the title compound as a brown oil. LC-MS for C₂₀H₁₆FN₃O₂S+H⁺[M+H]⁺: calcd. 382.1. found: 381.8. ¹H NMR (400 MHz, CDCl₃) δ [ppm]: 7.90 (d, J=7.6 Hz, 1H), 7.77 (dd, J=9.7, 2.0 Hz, 1H), 7.68 (dd, J=8.7, 5.3 Hz, 1H), 7.58-7.53 (m, 2H), 7.49-7.43 (m, 2H), 7.01 (ddd, J=9.3, 8.7, 2.3 Hz, 1H), 6.94-6.89 (m, 2H), 6.78 (d, J=7.6 Hz, 1H).

### Intermediate 33

### 5-(6-fluoro-1-(phenylsulfonyl)-1H-indol-3-yl)-2-(2-(methylthio)ethyl)-1H-benzo[d]imidazole

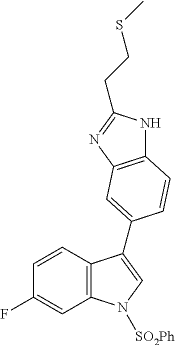

To a mixture of 4-(6-fluoro-1-(phenylsulfonyl)-1H-indol-3-yl)benzene-1,2-diamine (Intermediate 32; 500 mg; 1.31 mmol) and 3-(methylthio)propanoic acid (157 mg; 1.31 mmol) in DCM (10 mL) at 0° C. was added HATU (748 mg; 1.97 mmol) and Et₃N (265 mg; 2.62 mmol) under nitrogen. The reaction mixture was stirred at room temperature for 30 minutes, diluted with DCM (60 mL), washed with water (30 mL), brine (20 mL×2), dried over anhydrous Na₂SO₄, filtered, and concentrated. The residue was diluted with toluene (10 mL) and heated to reflux for 16 hours. The reaction mixture was concentrated in vacuo and purified by a silica gel chromatography (petroleum ether/EtOAc=100/1-6/1) to afford 460 mg (75%) of the title compound as a brown oil, which was used directly without further purification. LC-MS for C₂₄H₂₀FN₃O₂S₂+H⁺[M+H]⁺: calcd. 466.1. found: 465.8.

### Intermediate 34

### 5-(6-fluoro-1-(phenylsulfonyl)-1H-indol-3-yl)-2-(2-(methylsulfonyl)ethyl)-1H-benzo[d]imidazole

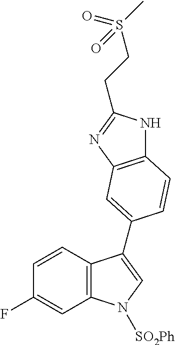

To a solution of 5-(6-fluoro-1-(phenylsulfonyl)-1H-indol-3-yl)-2-(2-(methylthio)ethyl)-1H-benzo[d]imidazole (Intermediate 33; 460 mg; 0.99 mmol) in DCM (15 mL) at 0° C. was added mCPBA (500 mg; 2.46 mmol; 85%) in portions. The reaction mixture was slowly warmed to room temperature and stirred for 30 minutes. The mixture was washed with saturated aqueous NaHSO₃ (20 mL) and the aqueous layer was extracted with DCM (30 mL×2). The combined organic layers were washed with brine (20 mL), dried over anhydrous Na₂SO₄, filtered, and concentrated to afford 170 mg (34%) of the title compound as a brown solid, which was used directly without further purification. LC-MS for O₂₄H₂₀FN₃O₄S₂—H⁻ [M−H]⁻: calcd. 496.1. found: 495.9. ¹H NMR (300 MHz, CDCl₃) δ [ppm]: 7.97-7.89 (m, 2H), 7.83-7.75 (m, 1H), 7.75-7.66 (m, 3H), 7.65-7.52 (m, 2H), 7.51-7.41 (m, 3H), 7.09-6.98 (m, 1H), 3.70-3.61 (m, 2H), 3.59-3.48 (m, 2H), 2.97 (s, 3H).

### Intermediate 35

### tert-butyl 2-(2-amino-5-(6-fluoro-1-(phenylsulfonyl)-1H-indol-3-yl)phenylamino)-2-oxoethyl(methyl)carbamate and tert-butyl 2-(2-amino-4-(6-fluoro-1-(phenylsulfonyl)-1H-indol-3-yl)phenylamino)-2-oxoethyl(methyl)carbamate

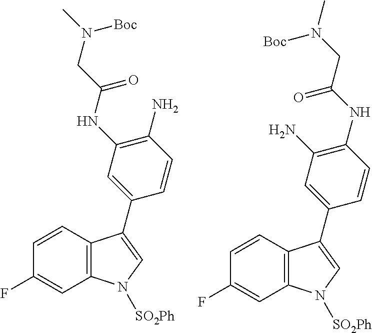

To a solution of 2-(tert-butoxycarbonyl(methyl)amino)acetic acid (165 mg, 0.87 mmol) and HATU (364 mg, 0.96 mmol) in THF (10 mL) and DMF (1 mL) was added DIPEA (187 mg, 1.45 mmol). The reaction mixture was stirred for 10 minutes before 4-(6-fluoro-1-(phenylsulfonyl)-1H-indol-3-yl)benzene-1,2-diamine (Intermediate 32, 500 mg, 1.31 mmol) was added. The mixture was stirred at room temperature for 60 minutes. The mixture was concentrated in vacuo, diluted with EtOAc (30 mL), washed with water (30 mL×3), dried over anhydrous Na₂SO₄, filtered, and concentrated to afford 670 mg (>100%) of the title compound as a black solid, which was used directly without further purification. LC-MS for C₂₂H₁₇ClFN₃O₃S+H⁺—(CH₃)₂C═CH₂ [M+H—(CH₃)₂C═CH₂]⁺: calcd. 497.1. found: 496.8.

### Intermediate 36

### tert-butyl(5-(6-fluoro-1-(phenylsulfonyl)-1H-indol-3-yl)-1H-benzo[d]imidazol-2-yl)methyl(methyl)carbamate

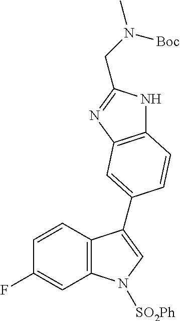

A mixture of tert-butyl 2-(2-amino-5-(6-fluoro-1-(phenylsulfonyl)-1H-indol-3-yl)phenylamino)-2-oxoethyl(methyl)carbamate and tert-butyl 2-(2-amino-4-(6-fluoro-1-(phenylsulfonyl)-1H-indol-3-yl)phenylamino)-2-oxoethyl(methyl)carbamate (Intermediate 35, 670 mg crude, 0.87 mmol) in AcOH (10 mL) was stirred at 50° C. for 6 hours. The mixture was cooled to room temperature, neutralized with saturated aqueous Na₂CO₃ to pH=8, and extracted with EtOAc (50 mL×3). The combined organic layer was dried over anhydrous Na₂SO₄, filtered, and concentrated to afford 640 mg (100%) of the title compound as a black solid, which was used directly without further purification. LC-MS for C₂₈H₂₇FN₄O₄S+H⁺[M+H]⁺: calcd. 535.2. found: 534.8.

### Intermediate 37

### tert-butyl(5-(6-fluoro-1H-indol-3-yl)-1H-benzo[d]imidazol-2-yl)methyl(methyl)carbamate

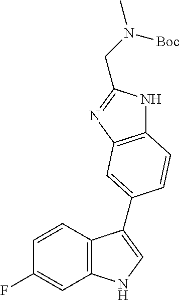 [SMILES: CN(Cc1nc2cc(-c3c[nH]c4cc(F)ccc34)ccc2[nH]1)C(=O)OC(C)(C)C]

Following the general method as outlined in Intermediate 15, starting from tert-butyl(5-(6-fluoro-1-(phenylsulfonyl)-1H-indol-3-yl)-1H-benzo[d]imidazol-2-yl)methyl(methyl)carbamate (Intermediate 36, 640 mg crude, 0.87 mmol), 470 mg (100%) of the title compound was obtained as a black solid, which was used directly without further purification. LC-MS for C₂₂H₂₃FN₄O₂+H⁺ [M+H]⁺: calcd. 395.2. found: 394.9.

### Intermediate 38

### N-(2-amino-5-(6-fluoro-1-(phenylsulfonyl)-1H-indol-3-yl)phenyl)-2-chloroacetamide and N-(2-amino-4-(6-fluoro-1-(phenylsulfonyl)-1H-indol-3-yl)phenyl)-2-chloroacetamide

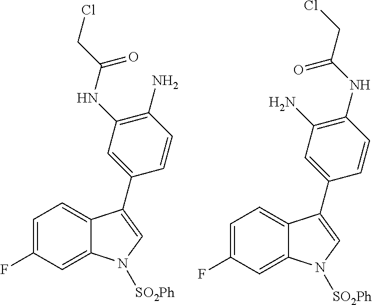

To a stirred solution of 4-(6-fluoro-1-(phenylsulfonyl)-1H-indol-3-yl)benzene-1,2-diamine (Intermediate 32, 3.81 g, 10.0 mmol), chloroacetic acid (945 mg, 10.0 mmol) and Et₃N (2.02 g, 20.0 mmol) in DCM (40 mL) at 0° C. was added HATU (3.80 g, 10.0 mmol). The reaction mixture was stirred at room temperature for 1 hour, diluted with water (50 mL), and concentrated in vacuo to remove most DCM. The aqueous mixture was extracted with EtOAc (40 mL×3). The combined organic layer was washed with brine (30 mL×2), dried over anhydrous Na₂SO₄, filtered, and concentrated to afford 3.96 g (87%) of the title compound as a brown oil, which was used directly without further purification.

LC-MS for C₂₂H₁₇ClFN₃O₃S+H⁺[M+H]⁺: calcd. 458.1. found: 457.7.

### Intermediate 39

### 2-(chloromethyl)-5-(6-fluoro-1-(phenylsulfonyl)-1H-indol-3-yl)-1H-benzo[d]imidazole

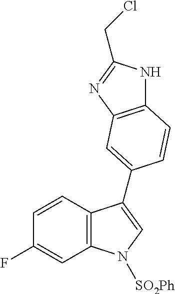

A mixture of N-(2-amino-5-(6-fluoro-1-(phenylsulfonyl)-1H-indol-3-yl)phenyl)-2-chloroacetamide and N-(2-amino-4-(6-fluoro-1-(phenylsulfonyl)-1H-indol-3-yl)phenyl)-2-chloroacetamide (Intermediate 38, 3.96 g, 8.65 mmol) in AcOH (20 mL) was stirred at 50° C. for 16 hours. The mixture was concentrated in vacuo to remove most AcOH. The residue was diluted with EtOAc (100 mL), washed with saturated aqueous NaHCO₃ (30 mL×2), brine (30 mL×2), dried over anhydrous Na₂SO₄, filtered, concentrated, and purified by silica gel chromatography (DCM/MeOH=200/1-50/1) to afford 3.20 g (84%) of the title compound as a brown solid. LC-MS for C₂₂H₁₅ClFN₃O₂S+H⁺ [M+H]⁺: calcd. 440.1. found: 439.8.

### Intermediate 40

### 3-(5-(6-fluoro-1-(phenylsulfonyl)-1H-indol-3-yl)-1H-benzo[d]imidazol-2-yl)propanoic acid

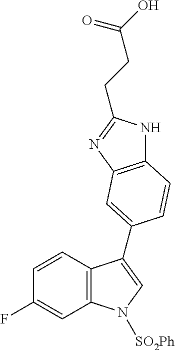

A solution of 4-(6-fluoro-1-(phenylsulfonyl)-1H-indol-3-yl)benzene-1,2-diamine (Intermediate 32, 500 mg, 1.31 mmol) and succinic anhydride (160 mg, 1.60 mmol) in 1,4-dioxane (8 mL) was heated at 80° C. for 48 hours. The mixture was concentrated and purified by silica gel chromatography (DCM/MeOH=18/1) to afford 0.60 g (99%) of the title compound as a white solid. LC-MS for C₂₄H₁₈FN₃O₄S+H⁺[M+H]⁺: calcd. 464.1. found: 464.0. ¹H NMR (400 MHz, DMSO-d₆) δ [ppm]: 12.30 (s, 1H), 8.15 (d, J=7.7 Hz, 2H), 8.06 (s, 1H), 7.87-7.80 (m, 2H), 7.77-7.70 (m, 2H), 7.66-7.60 (m, 2H), 7.57 (d, J=8.3 Hz, 1H), 7.44 (d, J=8.3 Hz, 1H), 7.24 (ddd, J=9.3, 8.7, 2.2 Hz, 1H), 3.57 (s, 1H), 3.07 (t, J=7.1 Hz, 2H), 2.82 (t, J=7.1 Hz, 2H).

### Intermediate 41

### 6-(6-fluoro-1-(phenylsulfonyl)-1H-indol-3-yl)-2,3-dihydro-1H-benzo[d]pyrrolo[1,2-a]imidazol-1-one

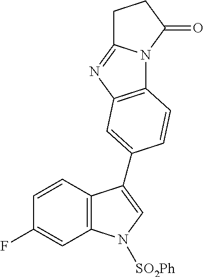

To a solution of 3-(5-(6-fluoro-1-(phenylsulfonyl)-1H-indol-3-yl)-1H-benzo[d]imidazol-2-yl)propanoic acid (Intermediate 40, 521 mg, 1.12 mmol), HATU (855 mg, 2.25 mmol) and Et₃N (454 mg, 4.49 mmol) in THF (15 mL) was added NH₄Cl (120 mg, 2.24 mmol) under nitrogen. The mixture was stirred at room temperature for 2 hours. The mixture was diluted with water (30 mL) and extracted with DCM (50 mL×2) and EtOAc (50 mL×2). The combined organic layer was washed with brine (40 mL), dried over anhydrous Na₂SO₄, filtered, and concentrated to afford 556 mg (>100%) of the title compound as a yellow solid, which was used directly without further purification. LC-MS for C₂₄H₁₆FN₃O₃S+H⁺ [M+H]⁺: calcd. 446.1. found: 446.0.

### Intermediate 42

### 5-(4,4,5,5-tetramethyl-1,3,2-dioxaborolan-2-yl)benzo[d]oxazole

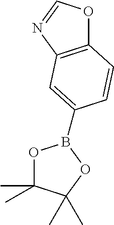 [SMILES: CC1(C)OB(c2ccc3ocnc3c2)OC1(C)C]

Following the general method as outlined in Intermediate 3, starting from 5-bromobenzo[d]oxazole (500 mg, 2.52 mmol), 576 mg (93%) of the title compound was obtained as a white solid after purification by silica gel chromatography (petroleum ether/EtOAc=100/1-30/1). LC-MS for C₁₃H₁₆BNO₃+H⁺[M+H]⁺: calcd. 246.1. found: 246.0. ¹H NMR (400 MHz, CDCl₃) δ [ppm]: 8.26 (s, 1H), 8.10 (s, 1H), 7.86 (d, J=8.0 Hz, 1H), 7.58 (d, J=8.0 Hz, 1H), 1.37 (s, 12H).

### Intermediate 43

### 5-(6-fluoro-1-(phenylsulfonyl)-1H-indol-3-yl)benzo[d]oxazole

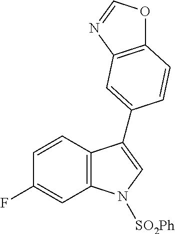

Following the general method as outlined in Intermediate 4, starting from 5-(4,4,5,5-tetramethyl-1,3,2-dioxaborolan-2-yl)benzo[d]oxazole (Intermediate 42, 330 mg, 1.35 mmol) and 6-fluoro-3-iodo-1-(phenylsulfonyl)-1H-indole (Intermediate 1,540 mg, 1.35 mmol), 423 mg (80%) of the title compound was obtained as a white solid after purification by recrystallization with MeOH. LC-MS for C₂₁H₁₃FN₂O₃S+H⁺ [M+H]⁺: calcd. 393.1. found: 392.8. ¹H NMR (400 MHz, CDCl₃) δ [ppm]: 8.16 (s, 1H), 7.99-7.93 (m, 3H), 7.81 (dd, J=9.5, 2.1 Hz, 1H), 7.73-7.65 (m, 3H), 7.62-7.57 (m, 2H), 7.53-7.47 (m, 2H), 7.07 (ddd, J=9.2, 8.7, 2.2 Hz, 1H).

### Intermediate 44

### 2-methyl-5-(4,4,5,5-tetramethyl-1,3,2-dioxaborolan-2-yl)benzo[d]oxazole

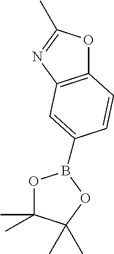 [SMILES: Cc1nc2cc(B3OC(C)(C)C(C)(C)O3)ccc2o1]

Following the general method as outlined in Intermediate 3, starting from 5-bromo-2-methylbenzo[d]oxazole (564 mg, 2.66 mmol), 688 mg (100%) of the title compound was obtained as a brown semi-solid, which was used directly without further purification. LC-MS for C₁₄H₁₈BNO₃+H⁺[M+H]⁺: calcd. 260.1. found: 260.0.

### Intermediate 45

### 5-(6-fluoro-1-(phenylsulfonyl)-1H-indol-3-yl)-2-methylbenzo[d]oxazole

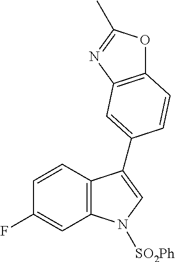

Following the general method as outlined in Intermediate 4, starting from 2-methyl-5-(4,4,5,5-tetramethyl-1,3,2-dioxaborolan-2-yl)benzo[d]oxazole (Intermediate 44, 688 mg, 2.66 mmol) and 6-fluoro-3-iodo-1-(phenylsulfonyl)-1H-indole (Intermediate 1, 711 mg, 1.77 mmol), 718 mg (100%) of the title compound was obtained as a yellow solid after purification by silica gel chromatography (petroleum ether/EtOAc=5/1-2/1). LC-MS for C₂₂H₁₅FN₂O₃S+H⁺[M+H]⁺: calcd. 407.1. found: 406.8. ¹H NMR (400 MHz, DMSO-d₆) δ [ppm]: 8.20-8.14 (m, 3H), 7.97 (d, J=0.8 Hz, 1H), 7.88-7.81 (m, 2H), 7.79-7.70 (m, 2H), 7.69-7.60 (m, 3H), 7.23 (ddd, J=9.2, 8.8, 2.4 Hz, 1H), 2.65 (s, 3H).

### Intermediate 46

### tert-butyl(2-((2-amino-5-(6-fluoro-1-(phenylsulfonyl)-1H-indol-3-yl)phenyl)amino)-2-oxoethyl)carbamate

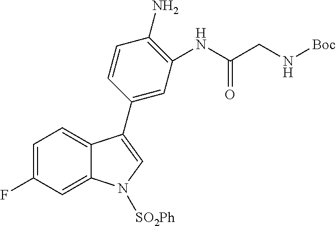

To a stirred solution of tert-butoxycarbonylamino-acetic acid (153 mg, 0.87 mmol) and DIEA (188.14 mg, 1.46 mmol) in anhydrous THF/DMF (10 mL/1 mL) was added HATU (363.66 mg, 0.96 mmol) at 0° C. The mixture was stirred for 10 mins and 4-(1-Benzenesulfonyl-6-fluoro-1H-indol-3-yl)-benzene-1,2-diamine (Intermediate 32, 500 mg, 1.31 mmol) was added. The reaction mixture was stirred at room temperature for 1 hour. The mixture was quenched with water (20 mL) and extracted with EtOAc (30 mL×3). The combined organic layer was washed with brine (20 mL×3), dried over anhydrous Na₂SO₄ and filtered. The filtrate was concentrated to afford 678 mg (96%) of the title compound as a black semi-solid, which was used directly without further purification. LC-MS for C₂₇H₂₇FN₄O₅S+H+: [M+H]⁺: calcd. 539.2. found: 538.8.

### Intermediate 47

### tert-butyl((6-(6-fluoro-1-(phenylsulfonyl)-1H-indol-3-yl)-1H-benzo[d]imidazol-2-yl)methyl)carbamate

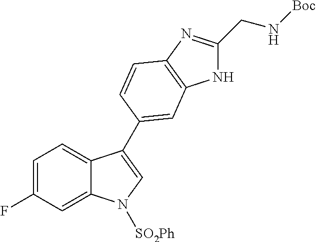

A mixture of tert-butyl(2-((2-amino-5-(6-fluoro-1-(phenylsulfonyl)-1H-indol-3-yl)phenyl)amino)-2-oxoethyl)carbamate (Intermediate 46, 678 mg, 1.31 mmol) in AcOH (10 mL) was stirred at 51° C. for 6 hours under nitrogen. The mixture was neutralized with aqueous of Na₂CO₃ to pH=8 and extracted with EtOAc (50 mL×3). The combined organic layer was washed with brine (100 mL), dried over anhydrous Na₂SO₄ and filtered. The filtrate was concentrated to afford 637 mg (93%) of the title compound as a black semi-solid, which was used directly without further purification. LC-MS for C₂₇H₂₅FN₄O₄S+H⁺[M+H]⁺: calcd. 521.2. found: 520.8. ¹H NMR (400 MHz, DMSO-d₆) δ [ppm]: 12.29 (d, J=20.0 Hz, 1H), 8.16 (d, J=7.8 Hz, 2H), 8.08 (d, J=11.5 Hz, 1H), 7.87-7.80 (m, 2H), 7.74 (ddd, J=11.4, 3.6, 1.2 Hz, 1H), 7.69-7.60 (m, 2H), 7.57-7.44 (m, 3H), 7.29-7.21 (m, 1H), 4.38 (d, J=5.7 Hz, 2H), 2.70 (d, J=8.2 Hz, 1H), 1.42 (s, 9H).

### Intermediate 48

### tert-butyl((6-(6-fluoro-1H-indol-3-yl)-1H-benzo[d]imidazol-2-yl)methyl)carbamate

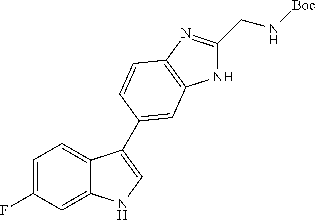 [SMILES: CC(C)(C)OC(=O)NCc1nc2ccc(-c3c[nH]c4cc(F)ccc34)cc2[nH]1]

To a solution of ((6-(6-fluoro-1-(phenylsulfonyl)-1H-indol-3-yl)-1H-benzo[d]imidazol-2-yl)methyl)carbamate (Intermediate 47, 587 mg, 1.13 mmol) in MeOH (30 mL) was added NaOH (451 mg, 11.28 mmol). The mixture was stirred at 85° C. for 2 hours. The reaction mixture was cooled to room temperature and concentrated to afford 429 mg (100%) of the title compound as a black semi-solid, which was used directly without further purification. LC-MS for C₂₁H₂₁FN₄O₂+H⁺[M+H]⁺: calcd. 381.2. found: 380.9.

### Intermediate 49

### N-(2-amino-5-(6-fluoro-1-(phenylsulfonyl)-1H-indol-3-yl)phenyl)-2-(dimethylamino)acetamide

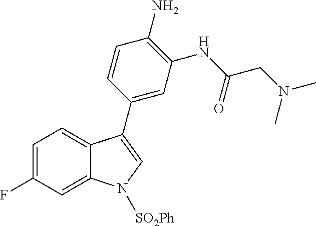

To a stirred solution of dimethylamino-acetic acid (110 mg, 0.79 mmol) and DIEA (201 mg, 1.56 mmol) in anhydrous THF (10 mL) was added HATU (385 mg, 1.01 mmol) at 0° C. The mixture was stirred for 10 mins and 4-(1-benzenesulfonyl-6-fluoro-1H-indol-3-yl)-benzene-1,2-diamine (Intermediate 32, 300 mg, 0.78 mmol) was added. The reaction mixture was stirred at room temperature for 1 hour. The mixture was quenched with water (20 mL) and extracted with EtOAc (30 mL×3). The combined organic layer was washed with brine (20 mL×3), dried over anhydrous Na₂SO₄ and filtered. The filtrate was concentrated to afford 363 mg (100%) of the title compound as a black semi-solid, which was used directly without further purification. LC-MS for C₂₄H₂₃FN₄O₃S+H⁺[M+H]⁺: calcd. 467.2. found: 466.8.

### Intermediate 50

### 1-(6-(6-fluoro-1-(phenylsulfonyl)-1H-indol-3-yl)-1H-benzo[d]imidazol-2-yl)-N,N-dimethylmethanamine

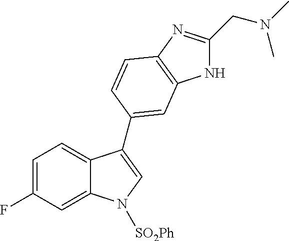

A mixture of N-[2-amino-5-(1-benzenesulfonyl-6-fluoro-1H-indol-3-yl)-phenyl]-2-dimethylamino-acetamide (Intermediate 49, 363 mg, 0.78 mmol) in AcOH (15 mL) was stirred at 51° C. for 5 hours under nitrogen. The mixture was neutralized with aqueous of Na₂CO₃ to pH=8 and extracted with EtOAc (50 mL×3). The combined organic layer was washed with brine (100 mL), dried over anhydrous Na₂SO₄ and filtered. The filtrate was concentrated to afford 441 mg (93%) of the title compound as a black semi-solid, which was used directly without further purification. LC-MS for C₂₄H₂₁FN₄O₂S+H⁺[M+H]⁺: calcd. 449.1. found: 449.0.

### Intermediate 51

### tert-butyl(2-(((6-(6-fluoro-1H-indol-3-yl)-1H-benzo[d]imidazol-2-yl)methyl)amino)-2-oxoethyl)carbamate

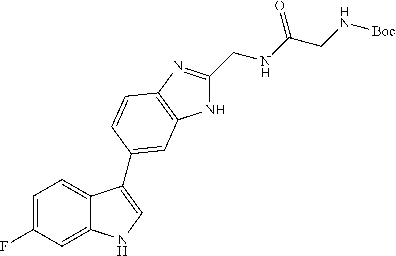 [SMILES: CC(C)(C)OC(=O)NCC(=O)NCc1nc2ccc(-c3c[nH]c4cc(F)ccc34)cc2[nH]1]

To a stirred solution of tert-butoxycarbonylamino-acetic acid (250 mg, 1.43 mmol) and DIEA (308 mg, 2.39 mmol) in anhydrous THF/DMF (20/2 mL) was added HATU (651 mg, 1.71 mmol) at 0° C. The mixture was stirred for 10 min and (6-(6-fluoro-1H-indol-3-yl)-1H-benzo[d]imidazol-2-yl)methanamine (Compound 27, 400 mg, 1.43 mmol) was added. The reaction mixture was stirred at room temperature for 1 hour. The reaction mixture was quenched with water (20 mL) and extracted with EtOAc (30 mL×3). The combined organic layer was washed with brine (20 mL×3) and dried over anhydrous Na₂SO₄ and filtered. The filtrate was concentrated to afford 624 mg (100%) of the title compound as a black semi-oil, which was used directly without further purification. LC-MS for C₂₃H₂₄FN₅O₃+H⁺[M+H]⁺: calcd. 438.2. found: 437.9.

### Intermediate 52

### 2-acetamido-N-(2-amino-5-(6-fluoro-1-(phenylsulfonyl)-1H-indol-3-yl)phenyl)acetamide

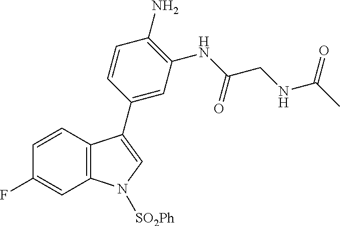

To a solution of N-acetylglycine (102 mg, 0.87 mmol) and DIPEA (226 mg, 1.75 mmol) in THF (10 mL) and DMF (1 mL) was added HATU (365 mg, 0.96 mmol). The reaction mixture was stirred at 0° C. for 20 minutes before 4-(6-fluoro-1-(phenylsulfonyl)-1H-indol-3-yl)benzene-1,2-diamine (Intermediate 32, 500 mg, 1.31 mmol) was added. The mixture was stirred at room temperature for 2 hours, concentrated and diluted with EtOAc (50 mL) and water (40 mL). The organic layer was dried over anhydrous Na₂SO₄, filtered and concentrated to afford 458 mg (>100%) of the crude title compound as a brown solid, which was used directly without further purification. LC-MS for C₂₄H₂₁FN₄O₄S+H⁺[M+H]⁺: calcd. 481.1. found: 480.8.

### Intermediate 53

### N-((6-(6-fluoro-1-(phenylsulfonyl)-1H-indol-3-yl)-1H-benzo[d]imidazol-2-yl)methyl)acetamide

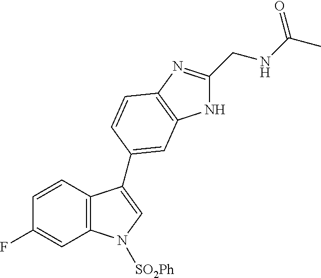

A mixture of 2-acetamido-N-(2-amino-5-(6-fluoro-1-(phenylsulfonyl)-1H-indol-3-yl)phenyl)acetamide (Intermediate 52, 458 mg crude, 0.87 mmol) in HOAc (15 mL) was stirred at 50° C. for 2.5 hours under nitrogen. The reaction mixture was cooled to room temperature, adjusted to pH=7 with saturate aqueous NaHCO₃ and extracted with EtOAc (20 mL). The organic layer was dried over anhydrous Na₂SO₄, filtered and concentrated to afford 470 mg (100%) of the crude title compound as a black solid, which was used directly without further purification. LC-MS for C₂₄H₁₉FN₄O₃S+H⁺ [M+H]⁺: calcd. 463.1. found: 462.8.

### Intermediate 54

### tert-butyl 4-((2-amino-5-(6-fluoro-1-(phenylsulfonyl)-1H-indol-3-yl)phenyl)carbamoyl)piperidine-1-carboxylate

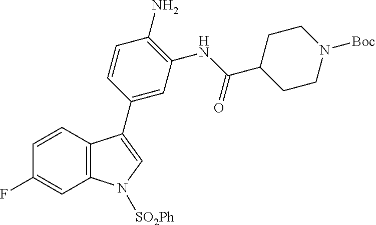

A mixture of 1-(tert-butoxycarbonyl)piperidine-4-carboxylic acid (430 mg, 1.88 mmol), DIPEA (520 mg, 4.02 mmol) and HATU (1.0 g, 2.63 mmol) in THF (10 mL) and DMF (1 mL) was stirred at room temperature for 15 minutes before 4-(6-fluoro-1-(phenylsulfonyl)-1H-indol-3-yl)benzene-1,2-diamine (Intermediate 32, 1.0 g, 2.62 mmol) was added. The mixture was stirred at room temperature for 12 hours before it was concentrated. The mixture was quenched with water and extracted with EtOAc. The combined organic layer was dried over anhydrous Na₂SO₄, filtered and concentrated to afford 1.6 g (>100%) of the crude title compound as a black oil, which was used directly without further purification. LC-MS for C₃₁H₃₃FN₄O₅S+H⁺[M+H]⁺: calcd. 593.2. found: 592.9.

### Intermediate 55

### tert-butyl 4-(6-(6-fluoro-1-(phenylsulfonyl)-1H-indol-3-yl)-1H-benzo[d]imidazol-2-yl)piperidine-1-carboxylate

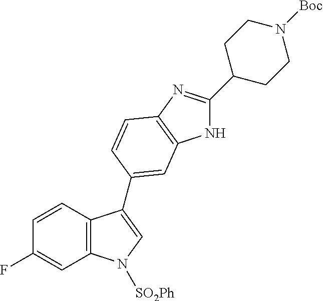

A mixture of tert-butyl 4-((2-amino-5-(6-fluoro-1-(phenylsulfonyl)-1H-indol-3-yl)phenyl)carbamoyl)piperidine-1-carboxylate (Intermediate 54, 1.60 g crude, 1.88 mmol) in HOAc (20 mL) was stirred at 50° C. for 4 hours under nitrogen. The reaction mixture was cooled to room temperature, concentrated, adjusted to pH=11 with aqueous K₂CO₃ and extracted with EtOAc. The combined organic layer was dried over anhydrous Na₂SO₄, filtered and concentrated to afford 660 mg (60%) of the crude title compound as a yellow solid, which was used directly without further purification. LC-MS for C₃₁H₃₁FN₄O₄S+H⁺[M+H]⁺: calcd. 575.2. found: 574.9.

### Intermediate 56

### tert-butyl 4-(6-(6-fluoro-1H-indol-3-yl)-1H-benzo[d]imidazol-2-yl)piperidine-1-carboxylate

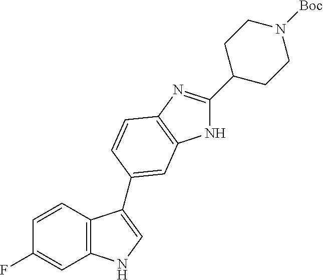 [SMILES: CC(C)(C)OC(=O)N1CCC(c2nc3ccc(-c4c[nH]c5cc(F)ccc45)cc3[nH]2)CC1]

Following the general method as outlined in Compound 1, starting from tert-butyl 4-(6-(6-fluoro-1-(phenylsulfonyl)-1H-indol-3-yl)-1H-benzo[d]imidazol-2-yl)piperidine-1-carboxylate (Intermediate 55, 660 mg, 1.15 mmol), 600 mg (>100%) of the crude title compound was obtained as a black oil, which was used directly without further purification. LC-MS for C₂₅H₂₇FN₄O₂+H⁺[M+H]⁺: calcd. 435.2. found: 434.9.

### Intermediate 57

### 2-amino-5-(6-fluoro-1H-indol-3-yl)phenol

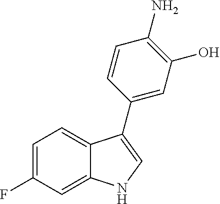 [SMILES: Nc1ccc(-c2c[nH]c3cc(F)ccc23)cc1O]

To a solution of 6-(1-benzenesulfonyl-6-fluoro-1H-indol-3-yl)-benzooxazole (702 mg, 1.81 mmol) in MeOH (100 mL) was added NaOH (212 mg, 5.31 mmol). The mixture was stirred at 65° C. for 3 hours. The mixture was concentrated to dryness in vacuo to give 500 mg of the title compound as a brown solid, which was used directly without further purification. LC-MS for C₁₄H₁₁FN₂O+H⁺[M+H]⁺: calcd. 243.1. found: 243.2.

### Intermediate 58

### N-[2-amino-5-(1-benzenesulfonyl-6-fluoro-1H-indol-3-yl)-phenyl]-2-chloro-acetamide & N-[2-amino-4-(1-benzenesulfonyl-6-fluoro-1H-indol-3-yl)-phenyl]-2-chloro-acetamide

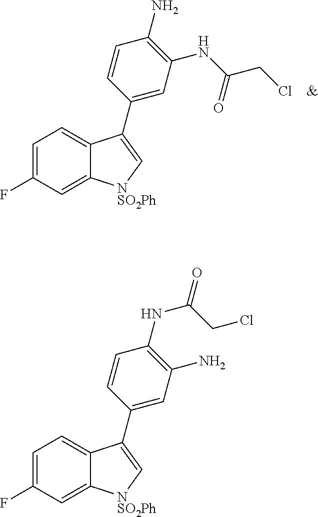

To a stirred solution of 4-(1-benzenesulfonyl-6-fluoro-1H-indol-3-yl)-benzene-1,2-diamine (3.81 g, 10.0 mmol), chloro-acetic acid (945 mg, 10.0 mmol) and TEA (2.0 g, 20.0 mmol) in DCM (40 mL) was added HATU (3.8 g, 10.0 mmol) at 0° C. The reaction mixture was stirred at room temperature for 1 hour, quenched with water (50 mL) and concentrated to remove most of the DCM. The aqueous mixture was extracted with EtOAc (40 mL×3). The combined organic layer was washed with brine (30 mL×2), dried over anhydrous Na₂SO₄ and filtered. The filtrate was concentrated to afford 3.96 g (87%) of the title compound as a brown oil, which was used to next step without further purification. LC-MS for C₂₂H₁₇ClFN₃O₃S+H⁺[M+H]⁺: calcd. 458.1. found: 457.7.

### Intermediate 59

### 6-(1-benzenesulfonyl-6-fluoro-1H-indol-3-yl)-2-chloromethyl-1H-benzoimidazole

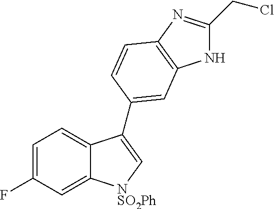

A mixture of N-[2-amino-5-(1-benzenesulfonyl-6-fluoro-1H-indol-3-yl)-phenyl]-2-chloro-acetamide & N-[2-amino-4-(1-benzenesulfonyl-6-fluoro-1H-indol-3-yl)-phenyl]-2-chloro-acetamide (3.96 g, 8.66 mmol) in AcOH (20 mL) was stirred at 50° C. for 16 hours and concentrated to remove most AcOH. The residue was diluted with EtOAc (100 mL), washed with aqueous NaHCO₃ (30 mL×2), brine (30 mL×2), dried over anhydrous Na₂SO₄ and filtered. The filtrate was concentrated to residue, which was purified by silica gel chromatography (DCM/MeOH=200/1-50/1) to afford 3.20 g (84%) of the title compound as a brown solid. LC-MS for C₂₂H₁₅ClFN₃O₂S+H⁺[M+H]⁺: calcd. 440.1. found: 439.8.

### Intermediate 60

### 6-(6-fluoro-1-(phenylsulfonyl)-1H-indol-3-yl)-2-((4-methylpiperazin-1-yl)methyl)-1H-benzo[d]imidazole

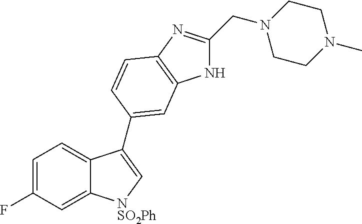

A mixture of 6-(1-benzenesulfonyl-6-fluoro-1H-indol-3-yl)-2-chloromethyl-1H-benzoimidazole (Intermediate 59, 200 mg, 0.456 mmol), 1-methyl-piperazine (73 mg, 0.729 mmol) and TEA (92 mg, 0.91 mmol) in DMF (3 mL) was stirred at 50° C. for 1 hour. The mixture was diluted with water (20 mL), extracted with DCM (30 mL×3), washed with brine (30 mL×2), dried over anhydrous Na₂SO₄ and filtered. The filtrate was concentrated to dryness to afford 290 mg (>100%) of the crude title compound as a brown oil. LC-MS for C₂₇H₂₆FN₅O₂S+H⁺ [M+H]⁺: calcd. 504.2. found: 503.8.

### Intermediate 61

### 2-(((6-(6-fluoro-1-(phenylsulfonyl)-1H-indol-3-yl)-1H-benzo[d]imidazol-2-yl)methyl)amino)ethanol

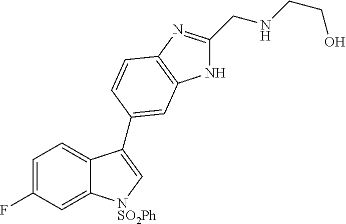

A mixture of 6-(1-benzenesulfonyl-6-fluoro-1H-indol-3-yl)-2-chloromethyl-1H-benzoimidazole (Intermediate 59, 200 mg, 0.456 mmol), 2-amino-ethanol (55 mg, 0.911 mmol) and TEA (92 mg, 0.911 mmol) in DMF (3 mL) was stirred at 50° C. for 2 hours.

Then it was diluted with EtOAc (50 mL), washed with brine (30 mL×2), dried over anhydrous Na₂SO₄ and filtered. The filtrate was concentrated to dryness to afford 116 mg (55%) of the crude title compound as a brown solid. LC-MS for C₂₄H₂₁FN₄O₃S+H⁺[M+H]⁺: calcd. 465.1. found: 464.9.

### Intermediate 62

### 6-(6-fluoro-1-(phenylsulfonyl)-1H-indol-3-yl)-2-(piperazin-1-ylmethyl)-1H-benzo[d]imidazole

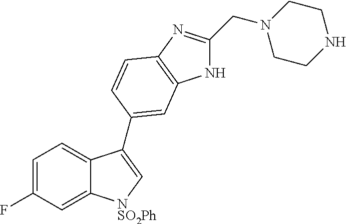

A mixture of 6-(1-benzenesulfonyl-6-fluoro-1H-indol-3-yl)-2-chloromethyl-1H-benzoimidazole (Intermediate 59, 439 mg, 1.0 mmol) and piperazine (258 mg, 3.0 mmol) in DMF (5 mL) was stirred at 60° C. for 0.5 hour. Then the mixture was diluted with water (50 mL), extracted with EtOAc (50 mL×2). The organic layer was washed with brine (30 mL×2), dried over anhydrous Na₂SO₄ and filtered. The filtrate was concentrated and purified by a silica gel column chromatography (DCM/MeOH=20/1 to 5/1) to afford 176 mg (36%) of the title compound as a yellow solid.

LC-MS for C₂₆H₂₄FN₅O₂S+H⁺[M+H]⁺: calcd. 490.2. found: 490.5.

### Intermediate 63

### tert-butyl(2-(6-(6-fluoro-1-(phenylsulfonyl)-1H-indol-3-yl)-1H-benzo[d]imidazol-2-yl)ethyl)carbamate

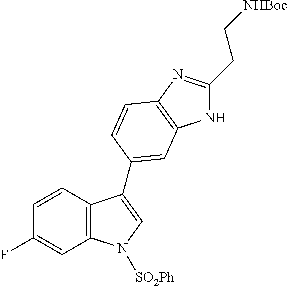

The solution of 3-tert-Butoxycarbonylamino-propionic acid (2.0 g, 10.5 mmol), HATU (4.5 g, 11.5 mmol) and DIPEA (17.8 g, 17.8 mmol) in THF (100 mL) was stirred at room temperature for 10 min before (4-(6-fluoro-1-(phenylsulfonyl)-1H-indol-3-yl)benzene-1,2-diamine (Intermediate 32, 6.0 g, 15.8 mmol) was added. The mixture was stirred at r. t for 2 h. Then it was diluted with EtOAc (100 mL), washed with water (100 mL×3), dried over Na₂SO₄ and concentrated to give the crude product which is used for next step without further purification.

The crude product was dissolved in AcOH (10 mL) and stirred at 50° C. for 6 hrs before it was quenched with aq NaHCO₃. The pH was adjusted to 7. The mixture was extracted with EtOAc (100 mL×3), dried over anhydrous Na₂SO₄ and concentrated to give the crude product (4.17 g) which is used for next step without further purification.

### Intermediate 64

### 2-(6-(6-fluoro-1-(phenylsulfonyl)-1H-indol-3-yl)-1H-benzo[d]imidazol-2-yl)ethanamine

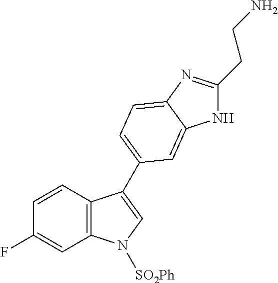

{2-[6-(1-Benzenesulfonyl-6-fluoro-1H-indol-3-yl)-1H-benzoimidazol-2-yl]-ethyl}-carbamic acid tert-butyl ester (Intermediate 63, 4.17 g, 7.80 mmol) was dissolved in HCl/CH₃OH (40 mL) and stirred at r. t for 1 h. The mixture was diluted with EtOAc (100 mL), washed with brine (50 mL×3), dried over Na₂SO₄ and concentrated to give the crude product which is purified by prep-HPLC to afford the title compound (1.80 g, 53%) as a yellow solid. ¹H NMR (400 MHz, CDCl₃) δ [ppm]: 7.93 (d, J=8.8 Hz, 2H), 7.80 (dd, J=9.6, 2.0 Hz, 1H), 7.74-7.67 (m, 1H), 7.65 (s, 1H), 7.59-7.55 (m, 2H), 7.49-7.39 (m, 4H), 7.04 (td, J=8.8, 1.6 Hz, 1H), 3.24 (t, J=5.6 Hz, 2H), 3.06 (t, J=5.2 Hz, 2H).

### Intermediate 65

### N-{2-[6-(1-Benzenesulfonyl-6-fluoro-1H-indol-3-yl)-1H-benzoimidazol-2-yl]-ethyl}-methanesulfonamide

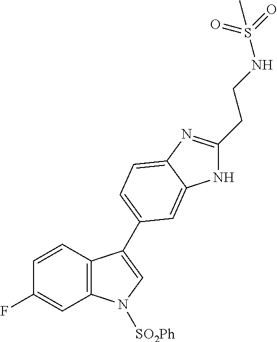

To a solution of 2-[6-(1-Benzenesulfonyl-6-fluoro-1H-indol-3-yl)-1H-benzoimidazol-2-yl]-ethylamine (Intermediate 64, 700 mg, 1.61 mmol) in DCM (20 mL) was added TEA (488 mg, 4.83 mmol) and the mixture was stirred at r.t for 10 min. Then MsCl (147 mg, 1.29 mmol) was added to the reaction mixture. The reaction was stirred at room temperature overnight and then diluted with DCM (50 mL). The mixture was washed with water (50 mL×3), dried over anhydrous Na₂SO₄ and concentrated to give the product (430 mg, 52%).

### Intermediate 66

### N-(2-(6-(6-fluoro-1-(phenylsulfonyl)-1H-indol-3-yl)-1H-benzo[d]imidazol-2-yl)ethyl)acetamide

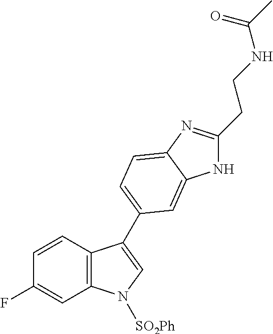

To a solution of 2-[6-(1-Benzenesulfonyl-6-fluoro-1H-indol-3-yl)-1H-benzoimidazol-2-yl]-ethylamine (Intermediate 64, 700 mg, 1.61 mmol) in DCM (20 mL) was added TEA (488 mg, 4.83 mmol) and the mixture was stirred at r.t for 10 min before AcCl (101 mg, 1.29 mmol) was added to the reaction mixture. The reaction was stirred at room temperature overnight and then diluted with DCM (50 mL). The mixture was washed with water (50 mL×3), dried over anhydrous Na₂SO₄, filtered and concentrated to give the product (429 mg, 57%).

### Intermediate 67

### 6-(6-fluoro-1-(phenylsulfonyl)-1H-indol-3-yl)-2-((4-(methylsulfonyl)piperazin-1-yl)methyl)-1H-benzo[d]imidazole

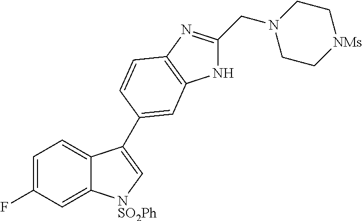

To a stirred solution of 6-(1-benzenesulfonyl-6-fluoro-1H-indol-3-yl)-2-piperazin-1-ylmethyl-1H-benzoimidazole (Intermediate 62, 96 mg, 0.196 mmol) and TEA (59 mg, 0.588 mmol) in DCM (10 mL) was added MsCl (27 mg, 0.236 mmol) at 0° C. The mixture was stirred at room temperature for 0.5 hour, diluted with EtOAc (50 mL), washed with water (30 mL) and brine (30 mL×2), dried over anhydrous Na₂SO₄ and filtered. The filtrate was concentrated and purified by a silica gel column chromatography (DCM/MeOH=100/1 to 20/1) to afford 80 mg (72%) of the title compound as a brown solid.

### Intermediate 68

### 2-(5-(6-fluoro-1H-indol-3-yl)-1H-benzo[d]imidazol-2-yl)acetonitrile

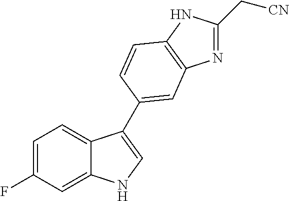 [SMILES: N#CCc1nc2cc(-c3c[nH]c4cc(F)ccc34)ccc2[nH]1]

To a stirred solution of 4-(6-fluoro-1H-indol-3-yl)-benzene-1,2-diamine (Intermediate 32, 296 mg, 1.23 mmol), cyano-acetic acid (104 mg, 1.23 mmol) and TEA (248 mg, 2.46 mmol) in DCM (10 mL) was added HATU (467 mg, 1.23 mmol) at 0° C. The mixture was stirred at room temperature for 1 hour and then concentrated. The residue was diluted with EtOAc (50 mL), washed with brine (30 mL×2), dried over anhydrous Na₂SO₄ and filtered. The filtrate was concentrated and AcOH (10 mL) was added. The mixture was stirred at 65° C. for 3 hours. The mixture was cooled and AcOH was removed. The mixture was basified with aqueous Na₂CO₃ (20 mL, 1M) and extracted with EtOAc (30 mL×2). The combined organic layer was washed with brine (30 mL×2), dried over anhydrous Na₂SO₄ and filtered. The filtrate was concentrated and purified by silica gel column chromatography (DCM/MeOH=100/1 to 20/1) to afford 186 mg (52%) of the title compound as a brown solid. LC-MS for C₁₇H₁₁FN₄+H⁺[M+H]⁺: calcd. 291.1. found: 291.3.

### Intermediate 69

### 2-amino-5-(6-fluoro-1-(phenylsulfonyl)-1H-indol-3-yl)phenol

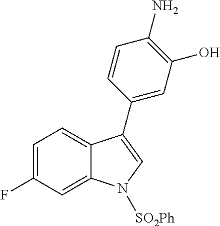

Following the general method as outlined in Intermediate 32, starting from 6-fluoro-3-iodo-1-(phenylsulfonyl)-1H-indole (Intermediate 1, 6.0 g, 25.53 mmol) and 2-amino-5-(4,4,5,5-tetramethyl-1,3,2-dioxaborolan-2-yl)phenol (6.83 g, 17.02 mmol), 4.3 g (66%) of the title compound was obtained as a white solid. LC-MS for C₂₀H₁₅FN₂O₃S—H⁻ [M−H]⁻: calcd. 381.1. found: 381.3. ¹H NMR (300 MHz, DMSO-d₆) δ [ppm]: 9.20 (s, 1H), 8.10 (d, J=7.5 Hz, 2H), 7.89-7.66 (m, 4H), 7.61 (t, J=7.5 Hz, 2H), 7.22 (t, J=8.1 Hz, 1H), 6.95 (s, 1H), 6.89 (d, J=7.9 Hz, 1H), 6.67 (d, J=8.1 Hz, 1H), 4.77 (s, 2H).

### Intermediate 70

### 2-amino-4-(6-fluoro-1-(phenylsulfonyl)-1H-indol-3-yl)phenol

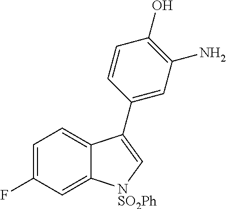

Following the general method as outlined in Intermediate 32, starting from 2-amino-4-(4,4,5,5-tetramethyl-[1,3,2]dioxaborolan-2-yl)-phenol (11.0 g, 46.7 mmol) and 6-fluoro-3-iodo-1-(phenylsulfonyl)-1H-indole (Intermediate 1, 12.5 g, 31.2 mmol), 7.2 g (60%) of the title compound was obtained as a brown solid. ¹H NMR (400 MHz, CDCl₃) δ [ppm]: 7.91 (s, 1H), 7.89 (s, 1H), 7.77 (dd, J=10.0, 2.0 Hz, 1H), 7.66 (dd, J=8.8, 4.8 Hz, 1H), 7.59-7.53 (m, 2H), 7.51-7.44 (m, 2H), 7.02 (td, J=8.8, 2.0 Hz, 1H), 6.94 (s, 1H), 6.89-6.85 (m, 1H), 6.83-6.79 (m, 1H), 4.99 (brs, 1H), 3.80-3.69 (m, 2H).

### Intermediate 71

### 2-(chloromethyl)-5-(6-fluoro-1-(phenylsulfonyl)-1H-indol-3-yl)benzo[d]oxazole

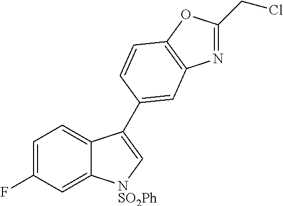

To a stirred mixture of 2-amino-4-(1-benzenesulfonyl-6-fluoro-1H-indol-3-yl)-phenol (Intermediate 70, 2.5 g, 6.54 mmol) in DCM (25 mL) at 0° C. was added chloro-acetyl chloride (887 mg, 7.85 mmol) and then TEA (990 mg, 9.81 mmol) slowly. The mixture was stirred at room temperature for 30 minutes before it was concentrated to afford brown oil. The above residue was diluted with AcOH (20 mL) and refluxed under nitrogen for 16 hours. Then it was cooled to room temperature and concentrated. The residue was suspended with sat.NaHCO₃ (50 mL) and extracted with EtOAc (50 mL×2). The combined organic layer was washed with brine (30 mL×2), dried over anhydrous Na₂SO₄ and filtered. The filtrate was concentrated and purified by a silica gel column chromatography (petroleum ether/EtOAc=10/1-5/1) to afford 1.1 g (38%) of the title compound as a brown solid. LC-MS for C₂₂H₁₄ClFN₂O₃S+H⁺ [M+H]⁺: calcd. 441.0. found: 441.4.

### Intermediate 72

### 5-(6-fluoro-1-(phenylsulfonyl)-1H-indol-3-yl)-2-(piperazin-1-ylmethyl)benzo[d]oxazole

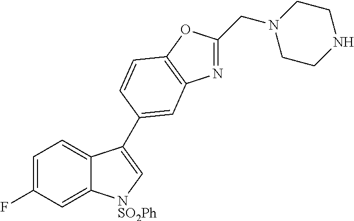

To a stirred solution of piperazine (Intermediate 71, 195 mg, 2.27 mmol) in DMF (5 mL) at 60° C. was added 2-(chloromethyl)-5-(6-fluoro-1-(phenylsulfonyl)-1H-indol-3-yl)benzo[d]oxazole (Intermediate 71, 100 mg, 0.23 mmol). The mixture was stirred for 30 minutes. Then it was cooled to room temperature and diluted with EtOAc (50 mL), washed with brine (30 mL×3), dried over anhydrous Na₂SO₄ and filtered. The filtrate was concentrated to dryness to afford 80 mg (72%) of the title compound as a brown oil. LC-MS for C₂₆H₂₃FN₄O₃S+H⁺[M+H]⁺: calcd. 491.2. found: 491.6.

### Intermediate 73

### 5-(6-fluoro-1-(phenylsulfonyl)-1H-indol-3-yl)-2-((4-methylpiperazin-1-yl)methyl)benzo[d]oxazole

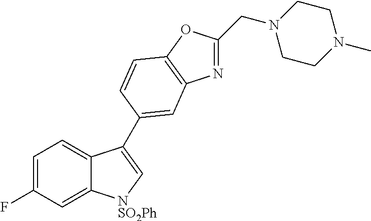

2-(chloromethyl)-5-(6-fluoro-1-(phenylsulfonyl)-1H-indol-3-yl)benzo[d]oxazole (Intermediate 71, 200 mg, 0.45 mmol) was added to a stirred solution of 1-methyl-piperazine (450 mg, 4.5 mmol) in DMF (3 mL) at 60° C. The mixture was stirred for 30 minutes, cooled and diluted with EtOAc (50 mL). The organic layer was washed with brine (30 mL×3), dried over anhydrous Na₂SO₄ and filtered. The filtrate was concentrated to dryness to afford 160 mg (70%) of the title compound as a brown solid. LC-MS for C₂₇H₂₂FN₄O₃S+H⁺[M+H]⁺: calcd. 505.2. found: 505.5.

### Intermediate 74

### 5-(6-fluoro-1-(phenylsulfonyl)-1H-indol-3-yl)-2-(morpholinomethyl)benzo[d]oxazole

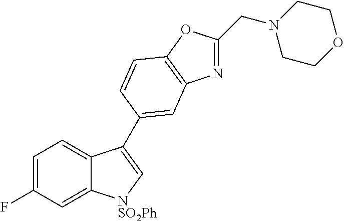

To a stirred solution of morpholine (391 mg, 4.5 mmol) in DMF (5 mL) was added 2-(chloromethyl)-5-(6-fluoro-1-(phenylsulfonyl)-1H-indol-3-yl)benzo[d]oxazole (Intermediate 71, 200 mg, 0.45 mmol) at 60° C. The mixture was stirred for 30 minutes, cooled and diluted with EtOAc (50 mL). The organic layer was washed with brine (30 mL×3), dried over anhydrous Na₂SO₄ and filtered. The filtrate was concentrated to dryness to afford 100 mg (45%) of the title compound as a brown solid. LC-MS for C₂₆H₂₂FN₃O₄S+H⁺[M+H]⁺: calcd. 492.1. found: 492.5.

### Intermediate 75

### 6-(6-fluoro-1-(phenylsulfonyl)-1H-indol-3-yl)benzo[d]oxazol-2(3H)-one

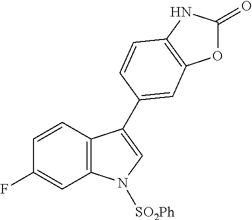

To a stirred solution of 2-amino-5-(6-fluoro-1-(phenylsulfonyl)-1H-indol-3-yl)phenol (Intermediate 69, 500 mg, 1.3 mmol) and CDl (233 mg, 1.43 mmol) in anhydrous THF (20 mL) was added TEA (1.08 mL) under nitrogen. The mixture was stirred at room temperature for 8 hours under nitrogen. The reaction mixture was diluted with water (30 mL) and extracted with EtOAc (60 mL×3). The combined organic layer was washed with water (60 mL×3), dried over anhydrous Na₂SO₄ and filtered. The filtrate was concentrated to afford 531 mg (100%) of the title compound as a red solid. LC-MS for C₂₁H₁₃FN₂O₄S—H⁻ [M−H]⁻: calcd. 407.1. found: 406.8. ¹H NMR (400 MHz, DMSO-d₆) δ [ppm]: 11.94 (s, 1H), 8.14 (d, J=8.5 Hz, 1H), 7.87-7.78 (m, 1H), 7.76-7.70 (m, OH), 7.68-7.60 (m, 2H), 7.49 (d, J=7.9 Hz, 0H), 7.23 (td, J=9.1, 2.2 Hz, 0H), 7.18 (d, J=7.8 Hz, 1H), 7.02 (s, 2H).

### Intermediate 76

### 2-(6-(6-fluoro-1-(phenylsulfonyl)-1H-indol-3-yl)-2-oxobenzo[d]oxazol-3(2H)-yl)acetamide

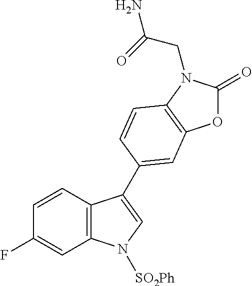

To a stirred solution of 6-(6-fluoro-1-(phenylsulfonyl)-1H-indol-3-yl)benzo[d]oxazol-2(3H)-one (Intermediate 75, 200 mg, 0.48 mmol) and K₂CO₃ (246 mg, 1.78 mmol) in NMP (6 mL) was added 2-bromoacetamide (61 mg, 0.45 mmol). The mixture was stirred at 60° C. for 6 hours. The reaction mixture was filtered. The filtrate was diluted with water (60 mL) and extracted with EtOAc (60 mL×3). The combined organic layer was washed with water (60 mL×3), dried over anhydrous Na₂SO₄ and filtered. The filtrate was concentrated to afford 183 mg (80%) of the title compound as a yellow solid. LC-MS for C₂₃H₁₆FN₃O₅S+H⁺[M+H]⁺: calcd. 466.1. found: 465.8.

### Intermediate 77

### 5-(1-Benzenesulfonyl-6-fluoro-1H-indol-3-yl)-1,3-dihydro-benzoimidazol-2-one

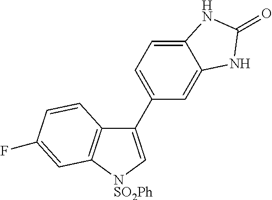

To a solution of 4-(1-Benzenesulfonyl-6-fluoro-1H-indol-3-yl)-benzene-1,2-diamine (Intermediate 32, 200 mg, 0.50 mmol) in THF was added CDl (170 mg, 1.05 mmol). The mixture was stirred overnight at room temperature. The solvent was removed to afford 185 mg (88%) of the title compound as a brown oil. LC-MS for C₂₁H₁₄FN₃O₃S+H⁺[M+H]⁺: calcd. 408.1. found: 408.4.

### Intermediate 78

### 2-chloro-N-(4-(6-fluoro-1-(phenylsulfonyl)-1H-indol-3-yl)-2-hydroxyphenyl)acetamide

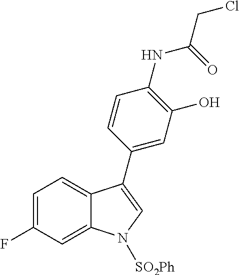

To a stirred solution of 2-amino-5-(6-fluoro-1-(phenylsulfonyl)-1H-indol-3-yl)phenol (Intermediate 69, 2.0 g, 5.2 mmol) in THF (200 mL) at 00° C. was added TEA (787 mg, 7.8 mmol) followed by chloro-acetyl chloride (712 mg, 6.3 mmol). The mixture was stirred at room temperature for 2 hours before it was concentrated. The residue was extracted with EtOAc, washed with brine, dried over Na₂SO₄ and filtered. The filtrate was concentrated to dryness to afford 1.4 g (61%) of the title compound as a brown solid.

### Intermediate 79

### 2-(chloromethyl)-6-(6-fluoro-1-(phenylsulfonyl)-1H-indol-3-yl)benzo[d]oxazole

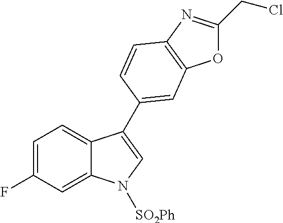

To a stirred solution of 2-chloro-N-(4-(6-fluoro-1-(phenylsulfonyl)-1H-indol-3-yl)-2-hydroxyphenyl)acetamide (Intermediate 78, 1.22 g, 2.7 mmol) in xylem (5 mL) was added PPTS (0.67 g, 2.7 mmol). The mixture was stirred at 150° C. for 4 hours. The reaction mixture was cooled to room temperature and concentrated. The residue was diluted with water (100 mL) and extracted with EtOAc (50 mL×3). The organic phase was dried over anhydrous Na₂SO₄ and filtered. The filtrate was concentrated and purified by silica gel column (Petrol Ether/Ethyl Acetate=2/1) to afford 746 mg (64%) of the title compound as a brown solid. LC-MS for C₂₂H₁₄ClFN₂O₃S+H⁺[M+H]⁺: calcd. 441.0. found: 441.0.

### Intermediate 80

### 6-(6-fluoro-1-(phenylsulfonyl)-1H-indol-3-yl)-2-(morpholinomethyl)benzo[d]oxazole

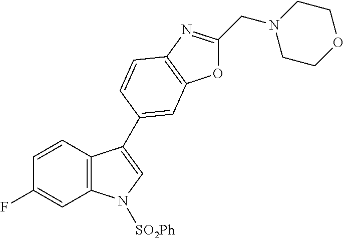

To a stirred solution of 2-(chloromethyl)-6-(6-fluoro-1-(phenylsulfonyl)-1H-indol-3-yl)benzo[d]oxazole (Intermediate 79, 150 mg, 0.34 mmol) in DMF (20 mL) at 60° C. was added dropwise morpholine (59 mg, 0.68 mmol) in DMF. The mixture was stirred at 60° C. for 1 hour, cooled to room temperature and concentrated. The residue was diluted with EtOAc (50 mL) and washed with water (40 mL×4). The organic phase was dried over anhydrous Na₂SO₄ and filtered. The filtrate was concentrated to afford 130 mg (80%) of the title compound as a brown solid. LC-MS for C₂₆H₂₂FN₃O₄S+H⁺[M+H]⁺: calcd. 492.1. found: 492.1.

### Intermediate 81

### 3-(6-(6-fluoro-1-(phenylsulfonyl)-1H-indol-3-yl)-2-oxobenzo[d]oxazol-3(2H)-yl)propanamide

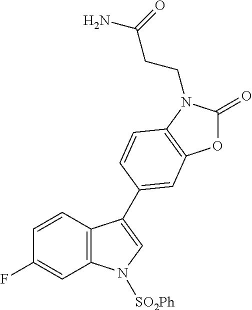

To a stirred solution of 6-(6-fluoro-1-(phenylsulfonyl)-1H-indol-3-yl)benzo[d]oxazol-2(3H)-one (Intermediate 75, 300 mg, 0.73 mmol) and K₂CO₃ (406 mg, 2.94 mmol) in NMP (6 mL) was added 3-bromopropanamide (101 mg, 0.67 mmol). The mixture was stirred at 63° C. for 6 hours. The reaction was filtered. The filtrate was diluted with water (60 mL) and extracted with EtOAc (60 mL×3). The combined organic layer was dried over anhydrous Na₂SO₄ and filtered. The filtrate was concentrated to afford 280 mg (80%) of the title compound as a yellow solid. LC-MS for C₂₄H₁₈FN₃O₅S—H⁻ [M−H]⁻: calcd. 478.1. found: 477.7.

### Intermediate 82

### 6-(6-fluoro-1-(phenylsulfonyl)-1H-indol-3-yl)-2-((4-methylpiperazin-1-yl)methyl)benzo[d]oxazole

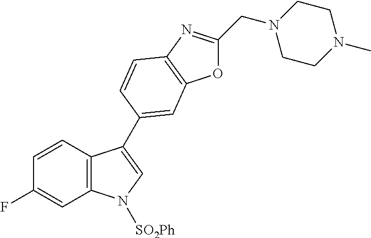

To a stirred solution of 2-(chloromethyl)-6-(6-fluoro-1-(phenylsulfonyl)-1H-indol-3-yl)benzo[d]oxazole (Intermediate 79, 300 mg, 0.68 mmol) in DMF (20 mL) at 60° C. was added dropwise 1-methyl-piperazine (136 mg, 1.36 mmol) in DMF. The mixture was stirred at 60° C. for 1 hours, cooled to room temperature and concentrated. The residue was diluted with EtOAc (50 mL) and washed with water (40 mL×4). The organic phase was dried over anhydrous Na₂SO₄ and filtered. The filtrate was concentrated to afford 285 mg (84%) of the title compound as a brown solid. LC-MS for C₂₇H₂₅FN₄O₃S+H⁺[M+H]⁺: calcd. 505.2. found: 504.9.

### Intermediate 83

### 5-(6-fluoro-1-(phenylsulfonyl)-1H-indol-3-yl)benzo[d]oxazol-2(3H)-one

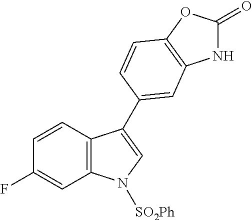

A solution of 2-amino-4-(6-fluoro-1-(phenylsulfonyl)-1H-indol-3-yl) phenol (Intermediate 70, 1.00 g, 2.61 mmol) and CDl (0.63 g, 3.89 mmol) in THF (50 mL) was stirred at room temperature for 3 hours. The mixture was concentrated to afford 1.60 g (>100%) of the crude title compound as a black oil, which was used directly without further purification. LC-MS for C₂₁H₁₃FN₂O₄S+H⁺[M+H]⁺: calcd. 409.1. found: 408.8.

### Intermediate 84

### 2-(5-(6-fluoro-1-(phenylsulfonyl)-1H-indol-3-yl)-2-oxobenzo[d]oxazol-3(2H)-yl)acetamide

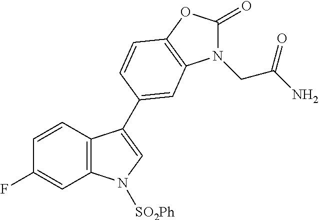

A mixture of 5-(6-fluoro-1-(phenylsulfonyl)-1H-indol-3-yl)benzo[d]oxazol-2(3H)-one (Intermediate 83, 800 mg crude, 1.3 mmol), 2-bromoacetamide (215 mg, 1.56 mmol) and K₂CO₃ (550 mg, 3.98 mmol) in DMF (10 mL) was stirred at room temperature for 12 hours. The mixture was added into water and extracted with EtOAc. The combined organic layer was concentrated to afford 600 mg (99%) of the crude title compound as yellow oil, which was used directly without further purification. LC-MS for C₂₃H₁₆FN₃O₅S+H⁺[M+H]⁺: calcd. 466.1. found: 465.8.

### Intermediate 85

### 6-(6-fluoro-1-(phenylsulfonyl)-1H-indol-3-yl)-1H-benzo[d]imidazol-2-amine

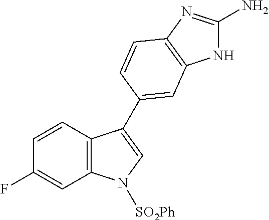

Following the general method as outlined in Intermediate 32, starting from 6-fluoro-3-iodo-1-(phenylsulfonyl)-1H-indole (Intermediate 1, 1.55 g, 3.86 mmol) and 6-(4,4,5,5-tetramethyl-1,3,2-dioxaborolan-2-yl)-1H-benzo[d]imidazol-2-amine (1.30 g, 5.02 mmol; prepared as described in US20070117818), 260 mg (17%) of the title compound was obtained as a brown solid. LC-MS for C₂₁H₁₅FN₄O₂S+H [M+H]⁺: calcd. 407.1. found: 407.4.

### Intermediate 86

### tert-butyl((5-(6-fluoro-1-(phenylsulfonyl)-1H-indol-3-yl)benzo[d]oxazol-2-yl)methyl)(methylsulfonyl)carbamate

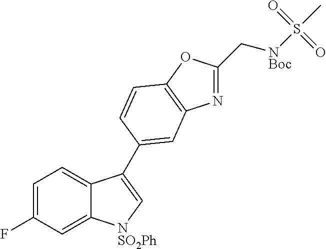

A mixture of (tert-butoxy)-N-(methylsulfonyl) carboxamide (53 mg, 0.273 mmol), 2-(chloromethyl)-5-(6-fluoro-1-(phenylsulfonyl)-1H-indol-3-yl)benzo[d]oxazole (Intermediate 71, 100 mg, 0.227 mmol) and K₂CO₃ (78 mg, 0.568 mmol) in DMF (3 mL) was stirred at 50° C. for 2 hours. Then it was cooled to room temperature and quenched with water (10 mL). The mixture was extracted with EtOAc (10 mL×3), washed with brine (10 mL×3), dried over anhydrous Na₂SO₄ and filtered. The filtrate was concentrated and purified by a preparative TLC (petroleum ether/EtOAc=4/1) to afford 50 mg (37%) of the title compound as a yellow solid. LC-MS for C₂₈H₂₆FN₃O₇S₂+H⁺[M+H]⁺: calcd. 600.1. found: 600.5.

### Intermediate 87

### N-((5-(6-fluoro-1-(phenylsulfonyl)-1H-indol-3-yl)benzo[d]oxazol-2-yl)methyl)methanesulfonamide

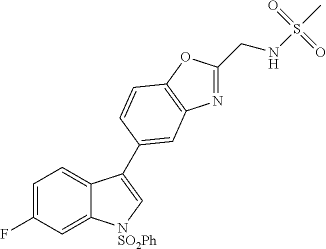

To a solution of N-[5-(1-benzenesulfonyl-6-fluoro-1H-indol-3-yl)-benzooxazol-2-ylmethyl]-methanesulfonamide (Intermediate 86, 100 mg, 0.17 mmol) in EtOAc (2 mL) was added HCl/EtOAc (2 mL, 4M). The reaction was stirred at room temperature for 16 hours then the mixture was concentrated to dryness to afford 83 mg (100%) of the title compound as a yellow solid. LC-MS for C₂₃H₁₈FN₃O₅S₂+H⁺[M+H]⁺: calcd. 500.1. found: 499.9.

### Intermediate 88

### 3-(5-(6-fluoro-1-(phenylsulfonyl)-1H-indol-3-yl)-2-oxobenzo[d]oxazol-3(2H)-yl)propanamide

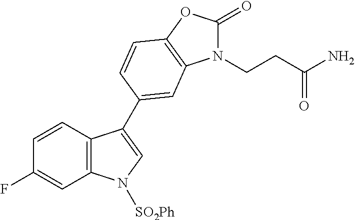

The mixture of 5-(1-benzenesulfonyl-6-fluoro-1H-indol-3-yl)-3H-benzooxazol-2-one (Intermediate 83, 370 mg, 0.91 mmol), 3-bromo-propionamide (127 mg, 0.91 mmol) and K₂CO₃(458 mg, 3.31 mmol) in NMP (8 mL) was stirred at 65° C. overnight. The reaction mixture was diluted with EA and washed with water (50 mL×4) and brine. The solvent was removed to give 270 mg (62%) as an orange solid. LC-MS for C₂₄H₁₈FN₃O₅S—H⁻ [M−H]⁻: calcd. 408.1. found: 477.8.

### Intermediate 89

### 2-Benzo[b]thiophen-5-yl-4,4,5,5-tetramethyl-[1,3,2]dioxaborolane

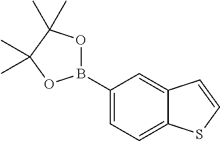 [SMILES: CC1(C)OB(c2ccc3sccc3c2)OC1(C)C]

Following the general method as outlined in Intermediate 3, starting from 5-bromo-benzo[b]thiophene (500 mg, 2.35 mmol) and 4,4,5,5,4′,4′,5′,5′-Octamethyl-[2,2′]bi[[1,3,2]dioxaborolanyl](776 mg, 3.06 mmol), the tile compound (720 mg, >100%, crude) was obtained as a yellow oil.

¹H NMR (400 MHz, CDCl₃) δ 8.31 (s, 1H), 7.89 (d, J=8.0 Hz, 1H), 7.75 (d, J=8.0 Hz, 1H), 7.41 (d, J=5.4 Hz, 1H), 7.35 (d, J=5.2 Hz, 1H), 1.37 (s, 12H).

### Intermediate 90

### 3-(benzo[b]thiophen-5-yl)-6-fluoro-1-(phenylsulfonyl)-1H-indole

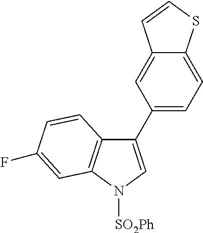

The mixture of 6-fluoro-3-iodo-1-(phenylsulfonyl)-1H-indole (Intermediate 1, 401 mg, 1.0 mmol), 2-benzo[b]thiophen-5-yl-4,4,5,5-tetramethyl-[1,3,2]dioxaborolane (423 mg, 1.30 mmol, crude), and K₃PO₄ (636 mg, 3.0 mmol) in dioxane (12 mL) and water (3 mL) was bubbled with nitrogen for 5 mins. Pd(dppf)Cl₂ (73 mg, 0.1 mmol) was added and the mixture was bubbled with nitrogen for another 5 mins. The mixture was placed into microwave reactor and stirred at 90° C. under nitrogen for 1.5 h. The solvent was removed. The residue was dissolved in PE/EA (100 mL, PE/EA=3/1) and filtered through a short plug of silica. The solvent was removed to get 500 mg of the title compound as a yellow oil.

### Intermediate 91

### tert-butyl((6-(6-fluoro-1-(phenylsulfonyl)-1H-indol-3-yl)benzo[d]oxazol-2-yl)methyl)(methylsulfonyl)carbamate

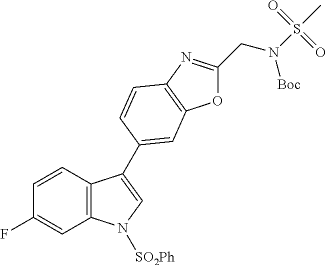

To a stirred solution of tert-butyl methylsulfonylcarbamate (155 mg, 0.82 mmol), K₂CO₃ (235 mg, 1.70 mmol) in DMF (15 mL) was added 2-(chloromethyl)-6-(6-fluoro-1-(phenylsulfonyl)-1H-indol-3-yl)benzo[d]oxazole (Intermediate 79, 300 mg, 0.68 mmol). The mixture was stirred at 50° C. for 5 hours. The reaction mixture was cooled to room temperature and filtered. The filtrate was diluted with water (60 mL) and extracted with EtOAc (60 mL×3). The combined organic layer was washed with water (20 mL×3), dried over anhydrous Na₂SO₄ and filtered. The filtrate was concentrated to afford 198 mg (49%) of the title compound as a yellow solid. LC-MS for C₂₈H₂₆FN₃O₇S₂+H⁺[M+H]⁺: calcd. 600.1. found: 599.8.

### Intermediate 92

### tert-butyl((6-(6-fluoro-1H-indol-3-yl)benzo[d]oxazol-2-yl)methyl)(methylsulfonyl)carbamate

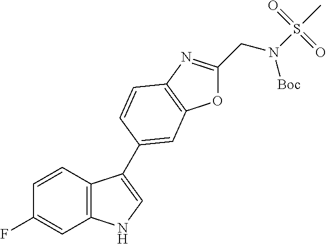 [SMILES: CC(C)(C)OC(=O)N(Cc1nc2ccc(-c3c[nH]c4cc(F)ccc34)cc2o1)S(C)(=O)=O]

To a stirred solution of tert-butyl((6-(6-fluoro-1-(phenylsulfonyl)-1H-indol-3-yl)benzo[d]oxazol-2-yl)methyl)(methylsulfonyl)carbamate (Intermediate 91, 148 mg, 0.25 mmol) in MeOH (15 mL) was added NaOH (99 mg, 2.5 mmol). The mixture was stirred at 85° C. for 1 hour. The reaction mixture was cooled to room temperature and neutralized with HCl (12 M). The mixture was diluted with water (60 mL) and extracted with EtOAc (60 mL×3). The combined organic layer was washed with brine (30 mL), dried over anhydrous Na₂SO₄ and filtered. The filtrate was concentrated to afford 114 mg (100%) of the title compound as a black solid. LC-MS for C₂₂H₂₂FN₃O₅S+H⁺[M-Boc+H]⁺: calcd. 360.1. found: 360.0.

### Intermediate 93

### tert-butyl N-({6-[1-(benzenesulfonyl)-6-fluoro-1H-indol-3-yl]-1,3-benzoxazol-2-yl}methyl)-N-[(tert-butoxy)carbonyl]carbamate

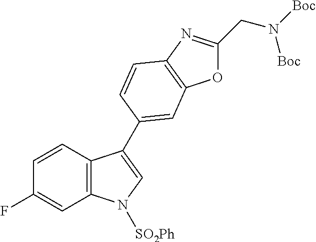

To a stirred solution of di-tert-butyl iminodicarboxylate (640 mg, 2.95 mmol), K₂CO₃ (900 mg, 6.5 mmol) and KI (34 mg, 0.2 mmol) in DMF (30 mL) was added 2-(chloromethyl)-6-(6-fluoro-1-(phenylsulfonyl)-1H-indol-3-yl)benzo[d]oxazole (Intermediate 79, 1 g, 2.27 mmol) at 60° C. The mixture was stirred at 60° C. for 5 hours. The reaction was neutralized with HCl (12 M) and filtered. The filtrate was diluted with water (60 mL) and extracted with EtOAc (60 mL×3). The combined organic layer was washed with water (60 mL×3), dried over anhydrous Na₂SO₄ and filtered. The filtrate was concentrated to afford 1.4 g (100%) of the title compound as a black oil. LC-MS for C₃₂H₃₂FN₃O₇S₂+H⁺[M-Boc+H]⁺: calcd. 522.2. found: 522.9.

### Intermediate 94

### tert-butyl N-[(tert-butoxy)carbonyl]-N-{[6-(6-fluoro-1H-indol-3-yl)-1,3-benzoxazol-2-yl]methyl}carbamate

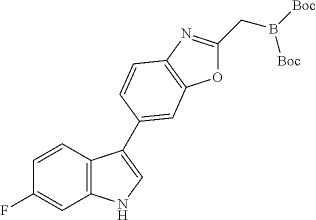 [SMILES: CC(C)(C)OC(=O)B(Cc1nc2ccc(-c3c[nH]c4cc(F)ccc34)cc2o1)C(=O)OC(C)(C)C]

To a stirred solution of tert-butyl N-({6-[1-(benzenesulfonyl)-6-fluoro-1H-indol-3-yl]-1,3-benzoxazol-2-yl}methyl)-N-[(tert-butoxy)carbonyl]carbamate (Intermediate 93; 1.36 g, 2.20 mmol) in MeOH (50 mL) was added NaOH (878 mg, 21.96 mmol). The mixture was stirred at 85° C. for 1 hour. The reaction mixture was cooled to room temperature and neutralized with HCl (12 M) and filtered. The filtrate was diluted with water (60 mL) and extracted with EtOAc (70 mL×3). The combined organic layer was washed with brine (30 mL), dried over anhydrous Na₂SO₄ and filtered. The filtrate was concentrated and purified by silica gel chromatography (petroleum ether/EtOAc=6/1-2/1) to afford 406 mg (39%) of the title compound as a yellow solid. LC-MS for C₂₆H₂₈FN₃O₅+H⁺[M-Boc+H]⁺: calcd. 382.2. found: 381.9.

### Intermediate 95

### 6-(6-fluoro-1-(phenylsulfonyl)-1H-indol-3-yl)-2-(piperazin-1-ylmethyl)benzo[d]oxazole

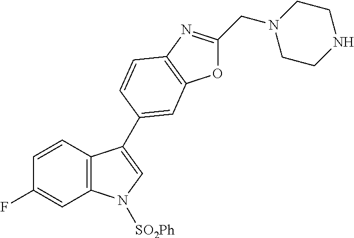

To a stirred solution of 2-(chloromethyl)-6-(6-fluoro-1-(phenylsulfonyl)-1H-indol-3-yl)benzo[d]oxazole (Intermediate 79, 500 mg, 1.13 mmol) in DMF (20 mL) at 60° C., was added dropwise piperazine (195 mg, 2.26 mmol) in DMF. The reaction was stirred at 60° C. for 1 hour before it was cooled to room temperature and concentrated. The residue was diluted with water (20 mL) and extracted with EtOAc (20 mL×4). The organic phase was dried over anhydrous Na₂SO₄ and filtered. The filtrate was concentrated to afford 277 mg (50%) of the title compound as a brown solid. LC-MS for C₂₆H₂₃FN₄O₃S+H⁺[M+H]⁺: calcd. 491.2. found: 490.8.

### Intermediate 96

### 2-(benzo[b]thiophen-6-yl)-4,4,5,5-tetramethyl-1,3,2-dioxaborolane

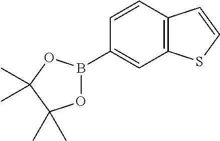 [SMILES: CC1(C)OB(c2ccc3ccsc3c2)OC1(C)C]

Following the general method as outlined in Intermediate 3, starting from 6-bromo-benzo[b]thiophene (1.0 g, 4.7 mmol) and 4,4,4′,4′,5,5,5′,5′-octamethyl-2,2′-bi(1,3,2-dioxaborolane (1.78 g, 7.0 mmol), 2.0 g (crude) of the title compound was obtained as a black solid, which was used directly without further purification.

### Intermediate 97

### 3-(benzo[b]thiophen-6-yl)-6-fluoro-1-(phenylsulfonyl)-1H-indole

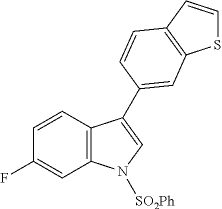

Following the general method as outlined in Intermediate 90, starting from 6-fluoro-3-iodo-1-(phenylsulfonyl)-1H-indole (Intermediate 1,500 mg, 1.2 mmol) and 2-(benzo[b]thiophen-6-yl)-4,4,5,5-tetramethyl-1,3,2-dioxaborolane (Intermediate 96, 420 mg, 1.6 mmol), 338 mg (70%) of the title compound was obtained as a white solid.

### Intermediate 98

### 5-(6-fluoro-1-(phenylsulfonyl)-1H-indol-3-yl)-2-(2-(methylthio)ethyl)benzo[d]oxazole

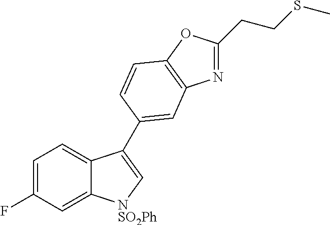

To a stirred solution of 3-methylsulfanyl-propionic acid (198 mg, 1.65 mmol) in DCM (15 mL) at −10° C. was added TEA (317 mg, 3.14 mmol), followed by isobutyl chloroformate (321 mg, 2.36 mmol). The white suspension was stirred at 0° C. for 20 minutes before 2-amino-4-(1-benzenesulfonyl-6-fluoro-1H-indol-3-yl)-phenol (Intermediate 70, 600 mg, 1.57 mmol) was added. The mixture was stirred at room temperature for 30 minutes before it was concentrated to dryness. The residue was diluted with AcOH (10 mL), refluxed for 16 hours, cooled to room temperature and concentrated. The residue was suspended with aqueous NaHCO₃ (30 mL), extracted with EtOAc (40 mL×2). The combined organic layer was washed with brine (30 mL×2), dried over anhydrous Na₂SO₄ and filtered. The filtrate was evaporated and purified by silica gel column chromatography (petroleum ether/EtOAc=20/1 to 5/1) to afford 320 mg (44%) of the title compound as a brown solid. LC-MS for C₂₄H₁₉FN₂O₃S₂+H⁺[M+H]⁺: calcd. 467.1. found: 467.5. ¹H NMR (400 MHz, CDCl₃) δ [ppm]: 7.97-7.92 (m, 2H), 8.85 (d, J=1.6 Hz, 1H), 7.80 (dd, J=9.6, 2.4 Hz, 1H), 7.71-7.66 (m, 2H), 7.62-7.55 (m, 2H), 7.53-7.46 (m, 3H), 7.06 (td, J=8.8, 2.4 Hz, 1H), 3.28 (t, J=7.2 Hz, 2H), 3.06 (t, J=7.2 Hz, 2H), 2.19 (s, 3H).

### Intermediate 99

### 5-(6-fluoro-1H-indol-3-yl)-2-(2-(methylthio)ethyl)benzo[d]oxazole

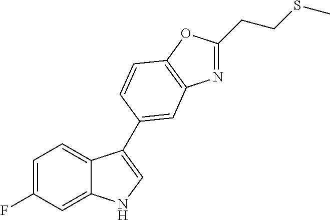 [SMILES: CSCCc1nc2cc(-c3c[nH]c4cc(F)ccc34)ccc2o1]

A mixture of 5-(1-benzenesulfonyl-6-fluoro-1H-indol-3-yl)-2-(2-methylsulfanyl-ethyl)-benzooxazole (320 mg, 0.687 mmol) and NaOH (137 mg, 3.43 mmol) in MeOH (10 mL) was stirred at 65° C. for 1 hour before it was cooled to room temperature. The mixture was diluted with water (30 mL) and extracted with EtOAc (30 mL×2). The combined organic layer was washed with brine (20 mL×2), dried over anhydrous Na₂SO₄ and filtered. The filtrate was evaporated and purified by silica gel column chromatography (petroleum ether/EtOAc=20/1 to 5/1) to afford 70 mg (31%) of the title compound as a yellow solid. LC-MS for C₁₈H₁₅FN₂OS+H⁺[M+H]⁺: calcd. 327.1. found: 327.4. ¹H NMR (400 MHz, DMSO-d₆) δ [ppm]: 8.28 (brs, 1H), 7.91 (s, 1H), 7.82 (dd, J=8.8, 5.2 Hz, 1H), 7.59-7.53 (m, 2H), 7.34 (d, J=2.4 Hz, 1H), 7.13 (dd, J=9.6, 2.4 Hz, 1H), 6.96 (td, J=9.2, 2.0 Hz, 1H), 3.93 (t, J=6.4 Hz, 2H), 3.42 (s, 3H), 3.25 (t, J=6.4 Hz, 2H).

### Intermediate 100

### tert-butyl 6-fluoro-3-(2-(2-(methylthio)ethyl)benzo[d]oxazol-5-yl)-1H-indole-1-carboxylate

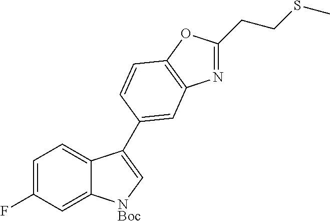 [SMILES: CSCCc1nc2cc(-c3cn(C(=O)OC(C)(C)C)c4cc(F)ccc34)ccc2o1]

To a solution of 5-(6-fluoro-1H-indol-3-yl)-2-(2-methylsulfanyl-ethyl)-benzooxazole (70 mg, 0.215 mmol) in DCM (10 mL) was added Boc₂O (93 mg, 0.429 mmol) at 0° C., followed by DMAP (8 mg, 0.0064 mmol). The mixture was stirred for 30 minutes before it was quenched with ice water (20 mL) and extracted with DCM (30 mL×2). The combined organic layer was washed with brine (20 mL×2), dried over anhydrous Na₂SO₄ and filtered. The filtrate was concentrated to dryness to afford 56 mg (61%) of the title compound as a white solid. LC-MS for C₂₃H₂₃FN₂O₃S+H⁺[M+H]⁺: calcd. 427.1. found: 427.6.

### Intermediate 101

### tert-butyl 6-fluoro-3-(2-(2-(methylsulfonyl)ethyl)benzo[d]oxazol-5-yl)-1H-indole-1-carboxylate

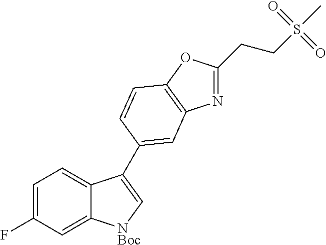 [SMILES: CC(C)(C)OC(=O)n1cc(-c2ccc3oc(CCS(C)(=O)=O)nc3c2)c2ccc(F)cc21]

To a stirred solution of 6-fluoro-3-[2-(2-methylsulfanyl-ethyl)-benzooxazol-5-yl]-indole-1-carboxylic acid tert-butyl ester (56 mg, 0.13 mmol) in DCM (5 mL) was added m-CPBA (80 mg, 0.39 mmol, 85%) at 0° C. The reaction was stirred at r.t. for 20 minutes. The mixture was diluted with aqueous NaHCO₃ (20 mL) and extracted with EtOAc (20 mL×2). The combined organic layer was washed with brine (20 mL×2), dried over anhydrous Na₂SO₄ and filtered. The filtrate was concentrated and purified by preparative TLC (petroleum ether/EtOAc=3/1) to afford 45 mg (75%) of the title compound as a white solid. LC-MS for C₂₃H₂₃FN₂O₅S+H⁺[M+H]⁺: calcd. 459.1. found: 459.6. ¹H NMR (400 MHz, CDCl₃) δ [ppm]: 8.18-8.00 (m, 1H), 7.99-7.90 (m, 1H), 7.88 (s, 1H), 7.72-7.66 (m, 1H), 7.58-7.50 (m, 1H), 7.40-7.30 (m, 1H), 7.04 (td, J=8.8, 2.0 Hz, 1H), 3.69 (t, J=7.2 Hz, 2H), 3.54 (t, J=7.2 Hz, 2H), 3.03 (s, 3H), 1.68 (s, 9H).

### Intermediate 102

### tert-butyl N-({5-[1-(benzenesulfonyl)-6-fluoro-1H-indol-3-yl]-1,3-benzoxazol-2-yl}methyl)-N-[(tert-butoxy)carbonyl]carbamate

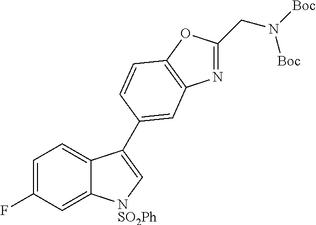

A mixture of 5-(1-benzenesulfonyl-6-fluoro-1H-indol-3-yl)-2-chloromethyl-benzooxazole (Intermediate 71, 100 mg, 0.227 mmol), tert-butyl[(tert-butoxy)carbonylamino]formate (148 mg, 0.682 mmol), KI (38 mg, 0.227 mmol) and K₂CO₃ (94 mg, 0.682 mmol) in DMF (5 mL) was stirred at 60° C. for 5 hours. Then it was cooled to room temperature, diluted with EtOAc (30 mL), washed with brine (20 mL×2), dried over anhydrous Na₂SO₄ and filtered. The filtrate was concentrated and purified by a silica gel column chromatography (petroleum ether/EtOAc=50/1 to 15/1) to afford 114 mg (81%) of the title compound as a yellow solid. LC-MS for C₃₂H₃₂FN₃O₇S+H⁺[M+H]⁺: calcd. 622.2. found: 622.7.

### Intermediate 103

### tert-butyl((5-(6-fluoro-1H-indol-3-yl)benzo[d]oxazol-2-yl)methyl)carbamate

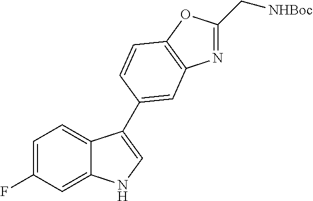 [SMILES: CC(C)(C)OC(=O)NCc1nc2cc(-c3c[nH]c4cc(F)ccc34)ccc2o1]

To a solution of tert-butyl N-({5-[1-(benzenesulfonyl)-6-fluoro-1H-indol-3-yl]-1,3-benzoxazol-2-yl}methyl)-N-[(tert-butoxy)carbonyl]carbamate (Intermediate 102, 114 mg, 0.183 mmol) in MeOH (8 mL) was added NaOH (73 mg, 1.83 mmol). The mixture was refluxed for 4 hours before it was cooled to room temperature. The solvent was removed. The residue was diluted with water (20 mL) and extracted with EtOAc (20 mL×2). The combined organic layer was washed with brine (20 mL×2), dried over anhydrous Na₂SO₄ and filtered. The filtrate was concentrated and purified by a silica gel column chromatography (petroleum ether/EtOAc=10/1 to 3/1) to afford 63 mg (90%) of the title compound as a brown oil. ¹H NMR (300 MHz, CDCl₃) δ [ppm]: 8.31 (brs, 1H), 7.92 (s, 1H), 7.82 (dd, J=9.3, 5.4 Hz, 1H), 7.63-7.54 (m, 2H), 7.35 (d, J=2.4 Hz, 1H), 7.13 (dd, J=9.3, 2.4 Hz, 1H), 6.97 (td, J=9.3, 2.4 Hz, 1H), 4.66 (d, J=5.7 Hz, 2H), 1.49 (s, 9H).

### Intermediate 104

### 3-(benzofuran-5-yl)-6-fluoro-1-(phenylsulfonyl)-1H-indole

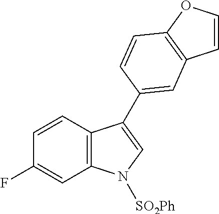

Following the general method as outlined in Intermediate 90, starting from 6-fluoro-3-iodo-1-(phenylsulfonyl)-1H-indole (Intermediate 1,412 mg, 1.03 mmol) and benzofuran-5-ylboronic acid (250 mg, 1.54 mmol), 340 mg (85%) of the title compound was obtained as a white solid. LC-MS for C₂₂H₁₄FNO₃S+H⁺[M+H]⁺: calcd. 392.1. found: 391.8. ¹H NMR (400 MHz, DMSO-d₆) δ [ppm]: 8.17-8.13 (m, 2H), 8.12 (s, 1H), 8.06 (d, J=2.2 Hz, 1H), 7.99 (d, J=1.8 Hz, 1H), 7.87 (dd, J=8.8, 5.3 Hz, 1H), 7.82 (dd, J=9.9, 2.4 Hz, 1H), 7.76-7.69 (m, 2H), 7.67-7.60 (m, 3H), 7.24 (ddd, J=9.5, 8.8, 2.4 Hz, 1H), 7.02 (d, J=2.0 Hz, 1H).

### Intermediate 105

### tert-butyl 4-(6-(6-fluoro-1-(phenylsulfonyl)-1H-indol-3-yl)benzo[d]oxazol-2-yl)piperidine-1-carboxylate

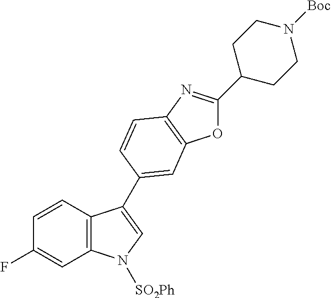

To a solution of 4-carbamoyl-piperidine-1-carboxylic acid tert-butyl ester (394 mg, 1.73 mmol) in dry DCM (10 mL) was added trimethyloxonium tetrafluoroborate (256 mg, 1.73 mmol) in portions at room temperature under nitrogen. The mixture was stirred at room temperature for 24 hours. Then 2-amino-5-(1-benzenesulfonyl-6-fluoro-1H-indol-3-yl)-phenol (Intermediate 69, 726 mg, 1.90 mmol) was added in one portion. The mixture was stirred at room temperature for 4 hours before it was diluted with aqueous NaHCO₃ (20 mL) and extracted with DCM (30 mL×3). The combined organic layer was washed with brine (50 mL), dried over anhydrous Na₂SO₄ and filtered. The filtrate was concentrated and purified by silica gel chromatography (PE/EtOAc=2/1) to afford 540 mg (54%) of the title compound as a brown solid. LC-MS for C₃₁H₃₀FN₃O₅S+H [M+H]⁺: calcd. 576.2. found: 576.7. ¹H NMR (400 MHz, DMSO-d₆) δ [ppm]: 8.21 (s, 1H), 8.15 (d, J=7.6 Hz, 2H), 8.04 (s, 1H), 7.89 (dd, J=4.8, 8.8 Hz, 1H), 7.84-7.78 (m, 2H), 7.75-7.68 (m, 2H), 7.63 (t, J=7.6 Hz, 2H), 7.24 (td, J=1.6, 8.8 Hz, 1H), 3.97 (d, J=12.8 Hz, 2H), 3.32-3.25 (m, 1H), 3.01 (brs, 2H), 2.11 (d, J=10.8 Hz, 2H), 1.75-1.65 (m, 2H), 1.42 (s, 9H).

### Intermediate 106

### tert-butyl 4-(6-(6-fluoro-1H-indol-3-yl)benzo[d]oxazol-2-yl)piperidine-1-carboxylate

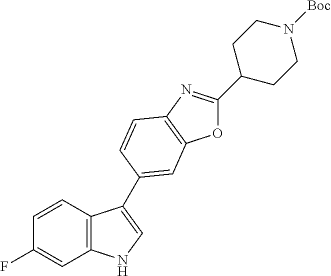 [SMILES: CC(C)(C)OC(=O)N1CCC(c2nc3ccc(-c4c[nH]c5cc(F)ccc45)cc3o2)CC1]

To a solution of tert-butyl 4-(6-(6-fluoro-1-(phenylsulfonyl)-1H-indol-3-yl)benzo[d]oxazol-2-yl)piperidine-1-carboxylate (Intermediate 105, 1.62 g, 2.82 mmol) in methanol (100 mL) was added NaOH (1.13 g, 28.2 mmol) at room temperature. The mixture was stirred at 60° C. for 2 hours. The mixture was cooled to room temperature and concentrated. The residue was diluted with water (100 mL) and extracted with EtOAc (100 mL×3). The combined organic layer was washed with brine (100 mL), dried over anhydrous Na₂SO₄ and filtered. The filtrate was concentrated to afford 1.23 g (99%) of the title compound as a gray solid. LC-MS for C₂₅H₂₆FN₃O₃+H⁺[M+H]⁺: calcd. 436.2. found: 436.6.

### Intermediate 107

### methyl 3-(6-(6-fluoro-1-(phenylsulfonyl)-1H-indol-3-yl)benzo[d]oxazol-2-yl)propanoate

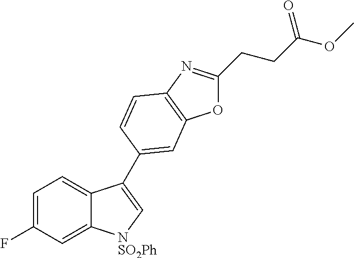

To a stirred solution of succinamic acid methyl ester (500 mg, 3.82 mmol) in 1,2-dichloro-ethane (10 mL) was added Meerwein's reagent (621 mg, 4.20 mmol) dropwise at room temperature. The mixture was stirred for 16 hours before a solution of 2-amino-5-(1-benzenesulfonyl-6-fluoro-1H-indol-3-yl)-phenol (Intermediate 69, 1.46 g, 3.82 mmol) in anhydrous MeOH (15 mL) was added. The mixture was stirred for a further 3 hours before it was quenched with aqueous NaHCO₃ (50 mL) and extracted with EtOAc (30 mL×3). The combined organic layer was washed with brine (20 mL×2), dried over anhydrous Na₂SO₄ and filtered. The filtrate was concentrated and purified by a silica gel column chromatography (petroleum ether/EtOAc=10/1 to 5/1) to afford 110 mg (6%) of the title compound as a brown solid. LC-MS for C₂₅H₁₉FN₂O₅S+H⁺[M+H]⁺: calcd. 479.1. found: 479.6. ¹H NMR (400 MHz, DMSO-d₆) δ [ppm]: 8.21 (s, 1H), 8.16 (d, J=8.1 Hz, 2H), 8.04 (s, 1H), 7.93-7.59 (m, 7H), 7.23 (td, J=9.0, 1.8 Hz, 1H), 3.62 (s, 3H), 3.24 (t, J=9.2 Hz, 2H), 2.94 (t, J=9.2 Hz, 2H).

### Intermediate 108

### 3-(6-(6-fluoro-1H-indol-3-yl)benzo[d]oxazol-2-yl)propanoic acid

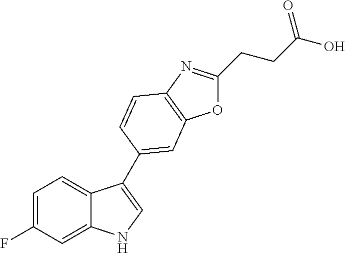 [SMILES: O=C(O)CCc1nc2ccc(-c3c[nH]c4cc(F)ccc34)cc2o1]

A mixture of 3-[6-(1-benzenesulfonyl-6-fluoro-1H-indol-3-yl)-benzooxazol-2-yl]-propionic acid methyl ester (Intermediate 107, 60 mg, 0.126 mmol) and NaOH (50 mg, 1.26 mmol) in EtOH (8 mL) was stirred at 76° C. for 1 hour. Then it was cooled to room temperature and concentrated. The mixture was diluted with water (10 mL) and suspended with petroleum ether/EtOAc=1/1 (10 mL, v/v). The aqueous layer was acidified with 1M HCl to pH=4 and extracted with EtOAc (20 mL×3). The combined organic layer was washed with brine (20 mL×2), dried over anhydrous Na₂SO₄ and filtered. The filtrate was evaporated to dryness to afford 52 mg (70%) of the title compound as a brown oil. LC-MS for C₁₈H₁₃FN₂O₃+H⁺[M+H]⁺: calcd. 325.1. found: 325.3.

### Intermediate 109

### 2-(6-(6-fluoro-1-(phenylsulfonyl)-1H-indol-3-yl)-1H-benzo[d]imidazol-1-yl)acetamide & 2-(5-(6-fluoro-1-(phenylsulfonyl)-1H-indol-3-yl)-1H-benzo[d]imidazol-1-yl)acetamide

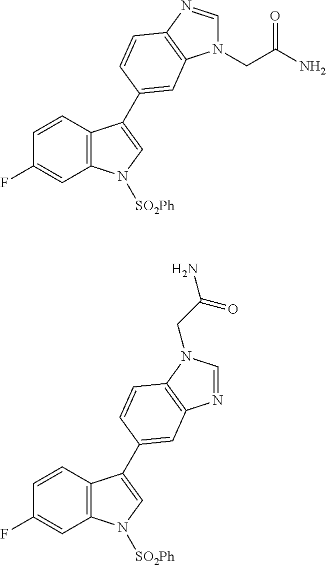

A mixture of 5-(6-fluoro-1-(phenylsulfonyl)-1H-indol-3-yl)-1H-benzo[d]imidazole (Intermediate 27, 1.00 g, 2.55 mmol), 2-bromoacetamide (450 mg, 3.26 mmol) and K₂CO₃ (700 mg, 5.06 mmol) in NMP (30 mL) was stirred at room temperature for 48 hours. The mixture was added into water dropwise. The yellow precipitate was collected by vacuum filtration. The solid was dissolved in EtOAc, washed with brine, dried over anhydrous Na₂SO₄ and filtered. The filtrate was concentrated and purified by silica gel chromatography (DCM-DCM/MeOH=9/1, v/v) to afford 400 mg (35%) of a mixture of the title compound and its region-isomer as a yellow solid. It was further purified by chiral preparative HPLC (SFC) to afford 100 mg (9%) the title compound as a white solid and 60 mg (5%) of the other regio-isomer 2-(5-(6-fluoro-1-(phenylsulfonyl)-1H-indol-3-yl)-1H-benzo[d]imidazol-1-yl)acetamide as a white solid. LC-MS for C₂₃H₁₇FN₄O₃S+H⁺[M+H]⁺: calcd. 449.1. found: 448.8.

### Intermediate 110

### ethyl 3-(5-(6-fluoro-1-(phenylsulfonyl)-1H-indol-3-yl)-1H-indazol-1-yl)propanoate

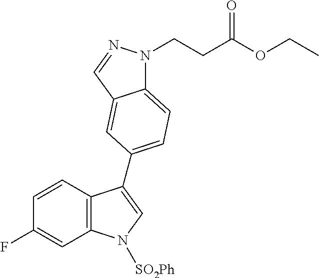

A mixture of 5-(1-benzenesulfonyl-6-fluoro-1H-indol-3-yl)-1H-indazole (Intermediate 12, 6.5 g, 1.66 mmol), acrylic acid ethyl ester (3.32 g, 33.2 mmol) and Cs₂CO₃ (16.2 g, 49.8 mmol) in DMF (60 mL) was stirred at room temperature for 3 hrs. Then the reaction mixture was poured into water and extracted with EtOAc (50 mL×3). The combined organic layer was washed with brine, dried over anhydrous Na₂SO₄, filtered and concentrated. The residue was purified by silica gel chromatography (petroleum ether/EtOAc=5/1-1/1) to afford 3.5 g (43%) of the title compound as a white solid. LC-MS for C₂₆H₂₂FN₃O₄S+H⁺[M+H]⁺: calcd. 492.1. found: 491.8. ¹H NMR (400 MHz, DMSO-d₆) δ [ppm]: 8.16-8.13 (m, 4H), 8.08 (s, 1H), 7.91-7.87 (m, 1H), 7.85-7.79 (m, 2H), 7.75-7.70 (m, 2H), 7.66-7.61 (m, 2H), 7.25 (td, J=9.2, 2.4 Hz, 1H), 4.68 (t, J=6.4 Hz, 2H), 4.00 (q, J=7.2 Hz, 2H), 2.95 (t, J=6.4 Hz, 2H), 1.10 (t, J=7.2 Hz, 3H).

### Intermediate 111

### 3-(5-(6-fluoro-1H-indol-3-yl)-1H-indazol-1-yl)propanoic acid

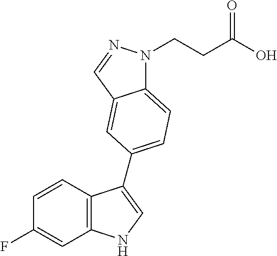 [SMILES: O=C(O)CCn1ncc2cc(-c3c[nH]c4cc(F)ccc34)ccc21]

A mixture of NaOH (855 mg, 21.4 mmol) and 3-[5-(1-Benzenesulfonyl-6-fluoro-1H-indol-3-yl)-indazol-1-yl]-propionic acid ethyl ester (Intermediate 110, 3.5 g, 7.13 mmol) in 50 mL of methanol was stirred at 75° C. for 30 mins. The mixture was concentrated. The residue was neutralized with 1 N HCl. The mixture was concentrated to give 4.2 g of the title compound as a yellow solid which was used for the next step without further purification. LC-MS for C₁₈H₁₄FN₃O₂+H⁺ [M+H]⁺: calcd. 324.1. found: 323.9.

### Intermediate 112

### tert-butyl 4-(3-(5-(6-fluoro-1H-indol-3-yl)-1H-indazol-1-yl)propanoyl)piperazine-1-carboxylate

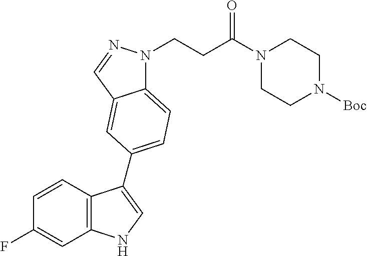 [SMILES: CC(C)(C)OC(=O)N1CCN(C(=O)CCn2ncc3cc(-c4c[nH]c5cc(F)ccc45)ccc32)CC1]

A mixture of 3-[5-(6-fluoro-1H-indol-3-yl)-indazol-1-yl]-propionic acid (Intermediate 111, 300 mg, crude, 0.93 mmol), Piperazine-1-carboxylic acid tert-butyl ester (173 mg, 0.93 mmol), HATU (530 mg, 1.4 mmol) and DIEA (360 mg, 2.8 mmol) in DMF (5 mL) was stirred at room temperature for 2 hrs. Then the mixture was poured into water, extracted with EtOAc (50 mL×3). The combined organic layer was washed with brine, dried over anhydrous Na₂SO₄, filtered and concentrated. The residue was purified by preparative HPLC (CH₃CN/H₂O=30%-75%, NH₄HCO₃) to afford 98 mg (21%) of the title compound as a yellow solid. LC-MS for C₂₇H₃₀FN₅O₃+H⁺[M+H]⁺: calcd. 492.2. found: 491.9.

### Intermediate 113

### 6-(6-fluoro-1-(phenylsulfonyl)-1H-indol-3-yl)indolin-2-one

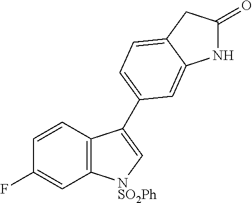

To a mixture of 6-fluoro-3-iodo-1-(phenylsulfonyl)-1H-indole (Intermediate 1, 600 mg, 1.50 mmol), 6-(4,4,5,5-tetramethyl-1,3,2-dioxaborolan-2-yl)indolin-2-one (259 mg, 1.00 mmol, prepared as described in WO2014140075) and K₂CO₃ (276 mg, 2.00 mmol) in dioxane (10 mL) and water (1 mL) was added Pd(PPh₃)₄ (116 mg, 0.10 mmol). The mixture was stirred at 120° C. for 2 hrs before it was concentrated. The residue was purified by silica gel chromatography (petroleum ether/EtOAc=5/1, v/v) to afford 400 mg (98%) of the title compound as a yellow solid. ¹H NMR (300 MHz, CDCl₃) δ [ppm]: 7.96-7.90 (m, 2H), 7.81 (dd, J=9.6, 2.4 Hz, 1H), 7.70-7.63 (m, 3H), 7.61-7.56 (m, 1H), 7.54-7.45 (m, 2H), 7.31 (d, J=7.7 Hz, 1H), 7.22 (dd, J=7.7, 1.5 Hz, 1H), 7.10-7.01 (m, 2H), 3.59 (s, 2H).

### Intermediate 114

### 3-(6-(6-fluoro-1-(phenylsulfonyl)-1H-indol-3-yl)-1H-benzo[d]imidazol-1-yl)propanamide & 3-(5-(6-fluoro-1-(phenylsulfonyl)-1H-indol-3-yl)-1H-benzo[d]imidazol-1-yl)propanamide

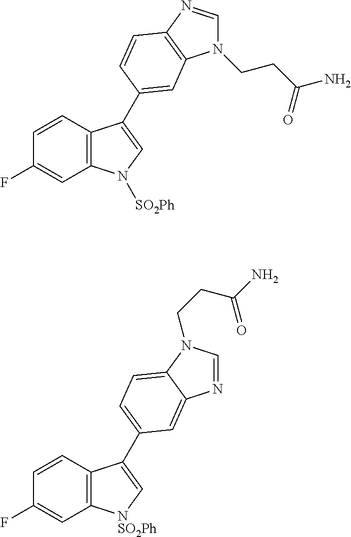

A mixture of 5-(6-fluoro-1-(phenylsulfonyl)-1H-indol-3-yl)-1H-benzo[d]imidazole (Intermediate 27, 1.66 g, 4.24 mmol), 3-bromopropionamide (970 mg, 6.38 mmol), KI (100 mg, 0.60 mmol) and K₂CO₃ (876 mg, 6.34 mmol) in NMP (10 mL) was stirred at 60° C. for 12 hrs. Another batch of 3-bromopropionamide (2 mg, 12.7 mmol) and K₂CO₃ (2 mg, 12.7 mmol) were added and the mixture was stirred at 60° C. for another 24 hours. The mixture was poured into water (150 mL) and extracted with EtOAc (50 mL×3). The organic layer was washed with brine, dried over anhydrous Na₂SO₄, filtered, concentrated, and purified by silica gel chromatography (DCM-DCM/MeOH=10/1) to afford 700 mg (37%) of a mixture of the title compound and its region-isomer as colorless oil, which was further purified by chiral preparative HPLC (SFC) to afford 140 mg (7%) the title compound as a white solid and 180 mg (9%) of the other regio-isomer 3-(5-(6-fluoro-1-(phenylsulfonyl)-1H-indol-3-yl)-1H-benzo[d]imidazol-1-yl)propanamide as a white solid. LC-MS for C₂₄H₁₉FN₄O₃S+H⁺[M+H]⁺: calcd. 463.1. found: 462.8.

### Intermediate 115

### 5-(4,4,5,5-tetramethyl-1,3,2-dioxaborolan-2-yl)indolin-2-one

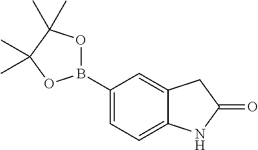 [SMILES: CC1(C)OB(c2ccc3c(c2)CC(=O)N3)OC1(C)C]

Following the general method as outlined in Intermediate 3, starting from 5-Bromo-1,3-dihydro-indol-2-one (1.00 g, 4.72 mmol) and 4,4,5,5,4′,4′,5′,5′-Octamethyl-20 [2,2′]bi[[1,3,2]dioxaborolanyl](1.56 g, 6.14 mmol), the title compound (1.20 g, crude) was obtained as a yellow solid. LC-MS for C₁₄H₁₈BNO₃+H⁺[M+H]⁺: calcd. 2620.1. found: 260.4.

### Intermediate 116

### 1-Benzenesulfonyl-6-fluoro-1′,3′-dihydro-1H-[3,5′]biindolyl-2′-one

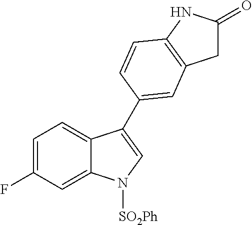

Following the general method as outlined in Intermediate 32, starting from 6-fluoro-3-iodo-1-(phenylsulfonyl)-1H-indole (Intermediate 1, 0.96 g, 2.4 mmol) and 5-(4,4,5,5-Tetramethyl-[1,3,2]dioxaborolan-2-yl)-1,3-dihydro-indol-2-one (0.84 g, 2.0 mmol), the title compound (440 mg, 45%) was obtained as a yellow solid. LC-MS for C₂₂H₁₅FN₂O₃S—H—[M−H]⁻: calcd. 405.1. found: 405.4. ¹H NMR (400 MHz, DMSO) δ 10.49 (s, 1H), 8.12 (d, J=7.6 Hz, 2H), 8.01 (s, 1H), 7.86-7.76 (m, 2H), 7.72 (t, J=7.5 Hz, 1H), 7.62 (t, J=7.7 Hz, 2H), 7.55 (s, 1H), 7.51 (d, J=7.9 Hz, 1H), 7.22 (td, J=9.1, 2.3 Hz, 1H), 6.91 (d, J=8.0 Hz, 1H), 3.54 (s, 2H).

### Intermediate 117

### 6-(4,4,5,5-tetramethyl-1,3,2-dioxaborolan-2-yl)benzo[d]oxazol-2-amine

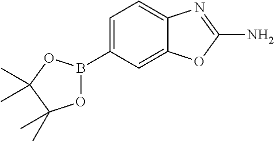 [SMILES: CC1(C)OB(c2ccc3nc(N)oc3c2)OC1(C)C]

Following the general method as outlined in Intermediate 3, starting from 6-Bromo-benzooxazol-2-ylamine (2.40 g, 11.3 mmol) and 4,4,5,5,4′,4′,5′,5′-Octamethyl-[2,2′]bi[[1,3,2]dioxaborolanyl](3.73 g, 14.7 mmol), the title compound (2.66 g, 90%) was obtained as a yellow solid. LC-MS for C₁₃H₁₇BN₂O₃+H⁺[M+H]⁺: calcd. 261.1. found: 261.4. ¹H NMR (300 MHz, DMSO-d₆) δ [ppm]: 7.58 (s, 2H), 7.48 (s, 1H), 7.46-7.43 (m, 1H), 7.18 (d, J=7.8 Hz, 1H), 1.29 (s, 12H).

### Intermediate 118

### 6-(6-fluoro-1-(phenylsulfonyl)-1H-indol-3-yl)benzo[d]oxazol-2-amine

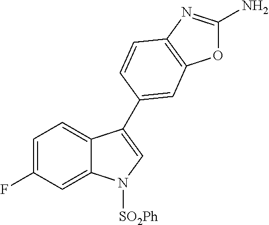

Following the general method as outlined in Intermediate 90, starting from 6-fluoro-3-iodo-1-(phenylsulfonyl)-1H-indole (Intermediate 1, 2.77 g, 6.90 mmol) and 6-(4,4,5,5-Tetramethyl-[1,3,2]dioxaborolan-2-yl)-benzooxazol-2-ylamine (1.50 g, 5.77 mmol), the title compound (1.50 g, 64%) was obtained as a yellow solid. LC-MS for C₂₁H₁₄FN₃O₃S+H⁺[M+H]⁺: calcd. 408.1. found: 408.5.

### Intermediate 119

### N-((6-(6-fluoro-1H-indol-3-yl)benzo[d]oxazol-2-yl)methyl)ethenesulfonamide

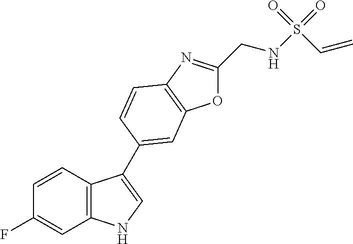 [SMILES: C=CS(=O)(=O)NCc1nc2ccc(-c3c[nH]c4cc(F)ccc34)cc2o1]

To a stirred solution of (6-(6-fluoro-1H-indol-3-yl)benzo[d]oxazol-2-yl)methanamine hydrochloride (Compound 76, 400 mg, 1.26 mmol), TEA (0.5 mL, 3.78 mmol) in DCM (30 mL) was added 2-chloroethanesulfonyl chloride (0.17 mL, 1.30 mmol) at 0° C. under nitrogen. The mixture was stirred at room temperature for 3 hours under nitrogen. The reaction mixture was diluted with EtOAc (60 mL) and filtered. The filtrate was concentrated and purified by silica gel column (petroleum ether/EtOAc=2/1) to afford 147 mg (32%) of the title compound as yellow oil. LC-MS for C₁₈H₁₄FN₃O₃S+H⁺ [M+H]⁺: calcd. 372.1. found: 372.5.

### Intermediate 120

### 6-(4,4,5,5-tetramethyl-1,3,2-dioxaborolan-2-yl)-2-vinylbenzo[d]oxazole

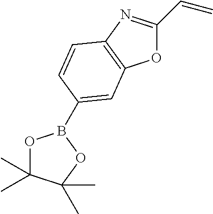 [SMILES: C=Cc1nc2ccc(B3OC(C)(C)C(C)(C)O3)cc2o1]

Following the general method as outlined in Intermediate 3, starting from 6-bromo-2-(2-chloro-ethyl)-benzooxazole (950 mg, 3.67 mmol) and B₂Pin₂ (1.12 g, 4.4 mmol), 700 mg (70%) of the title compound was obtained as a colorless oil.

¹H NMR (400 MHz, CDCl₃) δ [ppm]: 7.95 (s, 1H), 7.78 (d, J=8.0 Hz, 1H), 7.69 (d, J=8.0 Hz, 1H), 6.76 (dd, J=17.6, 11.2 Hz, 1H), 6.50 (dd, J=17.6, 0.8 Hz, 1H), 5.88 (dd, J=11.6, 0.8 Hz, 1H), 1.37 (s, 12H).

### Intermediate 121

### tert-butyl 6-fluoro-3-(2-vinylbenzo[d]oxazol-6-yl)-1H-indole-1-carboxylate

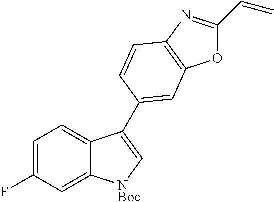 [SMILES: C=Cc1nc2ccc(-c3cn(C(=O)OC(C)(C)C)c4cc(F)ccc34)cc2o1]

Following the general method as outlined in Intermediate 32, starting from 6-(4,4,5,5-tetramethyl-[1,3,2]dioxaborolan-2-yl)-2-vinyl-benzooxazole (700 mg, 2.58 mmol) and 3-bromo-6-fluoro-indole-1-carboxylic acid tert-butyl ester (970 mg, 3.10 mmol), 520 mg (53%) of the title compound was obtained as a white solid. ¹H NMR (400 MHz, CDCl₃) δ [ppm]: 7.96 (d, J=8.8 Hz, 1H), 7.78 (dd, J=8.4, 0.4 Hz, 1H), 7.76-7.70 (m, 3H), 7.59 (dd, J=8.0, 1.6 Hz, 1H), 7.07 (td, J=8.8, 2.4 Hz, 1H), 6.79 (dd, J=17.6, 11.2 Hz, 1H), 6.50 (dd, J=17.6, 0.8 Hz, 1H), 5.89 (dd, J=11.2, 0.8 Hz, 1H), 1.70 (s, 9H).

### Intermediate 122

### tert-butyl 6-fluoro-3-(2-(2-(methylthio)ethyl)benzo[d]oxazol-6-yl)-H-indole-1-carboxylate

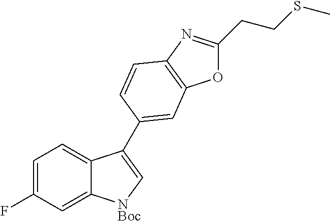 [SMILES: CSCCc1nc2ccc(-c3cn(C(=O)OC(C)(C)C)c4cc(F)ccc34)cc2o1]

To a solution of 6-fluoro-3-(2-vinyl-benzooxazol-6-yl)-indole-1-carboxylic acid tert-butyl ester (Intermediate 121, 520 mg, 1.37 mmol) in anhydrous THF (6 mL) was added NaSMe (144 mg, 2.06 mmol) at 0° C. The mixture was slowly warmed to r.t. and heated at 60° C. for 30 minutes. The mixture was cooled, diluted with EtOAc (50 mL), washed with water (20 mL), brine (20 mL×2), dried over anhydrous Na₂SO₄ and filtered. The filtrate was concentrated and purified by silica gel column chromatography (petroleum ether/EtOAc=20/1 to 10/1) to afford 480 mg (82%) of the title compound as a white solid. ¹H NMR (400 MHz, CDCl₃) δ [ppm]: 7.96 (d, J=9.2 Hz, 1H), 7.78-7.69 (m, 4H), 7.57 (dd, J=8.0, 1.2 Hz, 1H), 7.06 (td, J=8.8, 2.4 Hz, 1H), 3.29 (t, J=7.6 Hz, 2H), 3.06 (t, J=7.6 Hz, 2H), 2.20 (s, 3H), 1.70 (s, 9H).

### Intermediate 123

### tert-butyl 6-fluoro-3-(2-(2-(methylsulfonyl)ethyl)benzo[d]oxazol-6-yl)-1H-indole-1-carboxylate

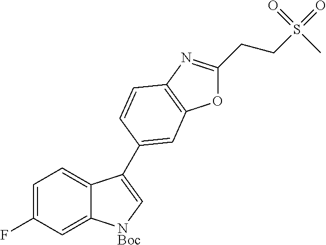 [SMILES: CC(C)(C)OC(=O)n1cc(-c2ccc3nc(CCS(C)(=O)=O)oc3c2)c2ccc(F)cc21]

To a solution of 6-fluoro-3-[2-(2-methylsulfanyl-ethyl)-benzooxazol-6-yl]-indole-1-carboxylic acid tert-butyl ester (480 mg, 1.13 mmol) in DCM (10 mL) was added m-CPBA (391 mg, 2.26 mmol) at 0° C. The mixture was stirred at r.t. for 20 minutes, poured into ice water (30 mL), extracted with EtOAc (30 mL×2). The combined organic layer was washed with brine (20 mL×2), dried over anhydrous Na₂SO₄ and filtered. The filtrate was evaporated and purified by silica gel column chromatography (petroleum ether/EtOAc=10/1 to 3/1) to afford 390 mg (75%) of the title compound as a yellow solid. LC-MS for C₂₃H₂₃FN₂O₅S+H⁺ [M+H]⁺: calcd. 459.1. found: 459.6. ¹H NMR (300 MHz, CDCl₃) δ [ppm]: 7.96 (d, J=8.7 Hz, 1H), 7.78-7.68 (m, 4H), 7.59 (dd, J=8.4, 1.5 Hz, 1H), 7.07 (td, J=9.0, 2.4 Hz, 1H), 3.70 (t, J=7.8 Hz, 2H), 3.54 (t, J=7.8 Hz, 2H), 3.03 (s, 3H), 1.70 (s, 9H).

### Intermediate 124

### N-(6-(6-fluoro-1-(phenylsulfonyl)-1H-indol-3-yl)benzo[d]oxazol-2-yl)methanesulfonamide

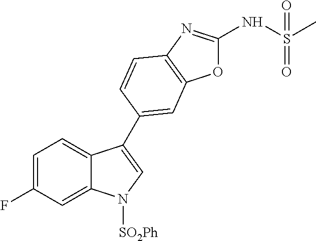

To a solution of 6-(1-benzenesulfonyl-6-fluoro-1H-indol-3-yl)-benzooxazol-2-ylamine (Intermediate 118, 400 mg, 0.98 mmol) in pyridine (8 mL) was added Methanesulfonyl chloride (224 mg, 1.96 mmol) and the mixture was stirred at room temperature overnight. The solvent was removed and the residue was redissolved in dichloromethane and washed with saturated NH₄Cl aqueous (20 mL×2) and brine (20 mL). The organic layer was dried and concentrated to afford 350 mg of the title compound (74%) as a black solid. LC-MS for C₂₂H₁₆FN₃O₅S₂—H⁻ [M−H]⁻: calcd. 484.1. found: 484.4.

### Intermediate 125

### 2-(2-chloroethyl)-6-(6-fluoro-1-(phenylsulfonyl)-1H-indol-3-yl)benzo[d]oxazole

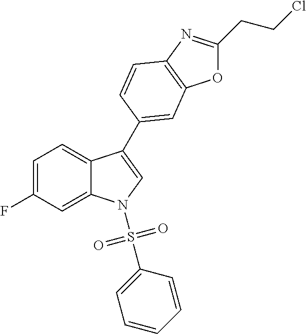 [SMILES: O=S(=O)(c1ccccc1)n1cc(-c2ccc3nc(CCCl)oc3c2)c2ccc(F)cc21]

A solution of 2-amino-5-(6-fluoro-1-(phenylsulfonyl)-1H-indol-3-yl) phenol (Intermediate 69, 1.6 g, 4.19 mmol) and 3-chloro-propionimidic acid methyl ester (3.6 g, 20.94 mmol) in DCM (10 mL) was stirred at room temperature overnight under nitrogen. The reaction mixture was concentrated and diluted with water (50 mL), extracted with EtOAc (100 mL×3) and the organic layer was concentrated and purified by flash chromatography on silica gel (Petroleum Ether/EtOAc=5/1) to afford 280 mg (15%) of the title compound as a yellow solid. LC-MS for C₂₃H₁₆ClFN₂O₃S+H⁺[M+H]⁺: calcd. 454.1. found: 455.5. ¹H NMR (400 MHz, CDCl₃) δ [ppm]: 7.95 (d, J=7.9 Hz, 2H), 7.81 (dd, J=9.2, 1.6 Hz, 1H), 7.77 (d, J=8.0 Hz, 1H), 7.73-7.66 (m, 3H), 7.59 (t, J=7.3 Hz, 1H), 7.54-7.48 (m, 3H), 7.07 (td, J=9.2, 2.4 Hz, 1H), 4.04 (t, J=6.8 Hz, 2H), 3.45 (t, J=6.8 Hz, 2H).

### Intermediate 126

### 6-(4,4,5,5-tetramethyl-1,3,2-dioxaborolan-2-yl)benzo[d]oxazole

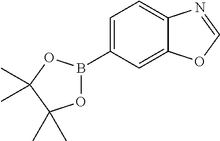 [SMILES: CC1(C)OB(c2ccc3ncoc3c2)OC1(C)C]

Following the general method as outlined in Intermediate 3, starting from 6-bromo-benzooxazole (510 mg, 2.58 mmol) and 4,4,5,5,4′,4′,5′,5′-Octamethyl-[2,2′]bi[[1,3,2]dioxaborolanyl](856 mg, 3.37 mmol), the title compound (740 mg, >100%) was obtained as a yellow solid. LC-MS for C₁₃H₁₆BNO₃+H⁺[M+H]⁺: calcd. 246.1. found: 246.4. ¹H NMR (300 MHz, CDCl₃) δ 8.15 (s, 1H), 8.05 (s, 1H), 7.84 (dd, J=8.0, 0.8 Hz, 1H), 7.80 (dd, J=8.0, 0.5 Hz, 1H), 1.39 (s, 12H).

### Intermediate 127

### 6-(1-(phenylsulfonyl)-1H-indol-3-yl)benzo[d]oxazole

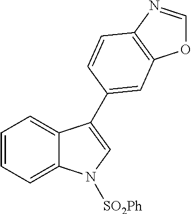

Following the general method as outlined in Intermediate 90, starting from 1-Benzenesulfonyl-3-iodo-1H-indole (500 mg, 1.30 mmol) and 6-(4,4,5,5-Tetramethyl-[1,3,2]dioxaborolan-2-yl)-benzooxazole (442 mg, 1.43 mmol), the title compound (340 mg, 70%) was obtained as a black solid. LC-MS for C₂₁H₁₄N₂O₃S+H⁺[M+H]⁺: calcd. 375.1. found: 375.5.

### Intermediate 128

### 6-(6-fluoro-1-(phenylsulfonyl)-1H-indol-3-yl)-2-vinylbenzo[d]oxazole

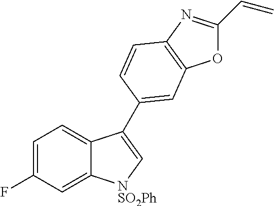

Following the general method as outlined in Intermediate 32, starting from 6-(4,4,5,5-tetramethyl-1,3,2-dioxaborolan-2-yl)-2-vinylbenzo[d]oxazole (3.0 g, 11.07 mmol) and 6-fluoro-3-iodo-1-(phenylsulfonyl)-1H-indole (Intermediate 1, 4.8 g, 12.18 mmol), 2.3 g (50%) of the title compound was obtained as a white solid. LC-Ms for C₂₃H₁₅FN₂O₃S+H [M+H]⁺: calcd. 419.1. found: 419.6. ¹H NMR (400 MHz, CDCl₃) δ [ppm]: 7.95 (d, J=7.4 Hz, 2H), 7.85-7.74 (m, 2H), 7.72 (q, J=5.1 Hz, 3H), 7.60 (t, J=7.5 Hz, 1H), 7.57-7.45 (m, 3H), 7.08 (td, J=8.9, 2.4 Hz, 1H), 6.78 (dd, J=17.6, 11.1 Hz, 1H), 6.51 (d, J=17.6 Hz, 1H), 5.90 (d, J=11.2 Hz, 1H).

### Intermediate 129

### 5-(6-chloro-1-(phenylsulfonyl)-1H-indol-3-yl)benzo[d]oxazole

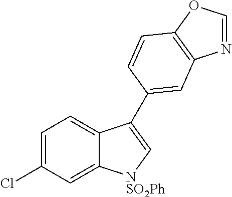

Following the general method as outlined in Intermediate 32, starting from 1-benzenesulfonyl-6-chloro-3-iodo-1H-indole (568 mg, 1.36 mmol) and 5-(4,4,5,5-tetramethyl-1,3,2-dioxaborolan-2-yl)benzo[d]oxazole (Intermediate 42, 500 mg, 2.04 mmol), 340 mg (61%) of the title compound was obtained as a white solid. LC-MS for C₂₁H₁₃ClN₂O₃S+H⁺[M+H]⁺: calcd. 409.0. found: 409.5. ¹H NMR (300 MHz, DMSO-d₆) δ [ppm]: 8.82 (s, 1H), 8.26 (s, 1H), 8.16 (s, 1H), 8.13 (d, J=1.5 Hz, 2H), 8.03 (d, J=1.8 Hz, 1H), 7.88 (m, 2H), 7.85 (dd, J=8.4, 1.7 Hz, 1H), 7.73 (d, J=7.5 Hz, 1H), 7.64 (t, J=7.6 Hz, 2H), 7.41 (dd, J=8.6, 1.9 Hz, 1H).

### Intermediate 130

### 6-(6-fluoro-1-(phenylsulfonyl)-1H-indol-3-yl)benzo[d]oxazole-2-thiol

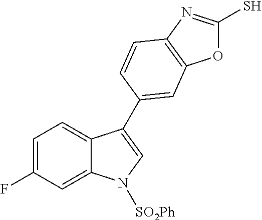

The mixture of 2-amino-5-(1-benzenesulfonyl-6-fluoro-1H-indol-3-yl)-phenol (Intermediate 69, 300 mg, 0.78 mmol) and KOH (63 mg, 0.95 mmol, 85%) in ethanol (20 mL) was added CS₂(2 mL, 33.16 mmol). The mixture was stirred at 60° C. overnight. The solvent was removed to obtain 450 mg (crude) of the title compound as a yellow solid. LC-MS for C₂₁H₁₃FN₂O₃S₂—H⁻ [M−H]⁻: calcd. 423.1. found: 423.3.

### Intermediate 131

### tert-butyl 4-((6-(6-fluoro-1-(phenylsulfonyl)-1H-indol-3-yl)benzo[d]oxazol-2-yl)methyl)piperidine-1-carboxylate

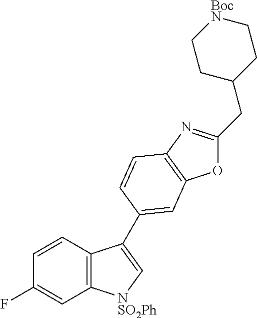

To a solution of tert-butyl 4-(2-amino-2-oxoethyl)piperidine-1-carboxylate (500 mg, 2.07 mmol) in DCM (25 mL) was added Me₃OBF₄ (306 mg, 2.07 mmol. The mixture was stirred at room temperature under N₂ overnight. Before 2-amino-5-(6-fluoro-1-(phenylsulfonyl)-1H-indol-3-yl)phenol (Intermediate 69, 872 mg, 2.28 mmol) was added. The mixture was stirred at 40° C. for 2 hrs and then at room temperature overnight. The mixture was concentrated and the residue was purified by silica gel column with PE-EA (5/1-1/1) to afford 350 mg of the title compound (28%) as a yellow solid. LC-MS for C₃₂H₃₂FN₃O₅S+H [M+H]⁺: calcd. 590.2. found: 590.2.

### Intermediate 132

### 6-(6-fluoro-1-(phenylsulfonyl)-1H-indol-3-yl)-2-(piperidin-4-ylmethyl)benzo[d]oxazole

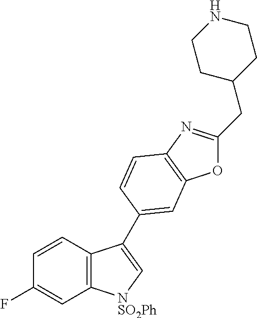

To a solution of tert-butyl 4-((6-(6-fluoro-1-(phenylsulfonyl)-1H-indol-3-yl)benzo[d]oxazol-2-yl)methyl)piperidine-1-carboxylate (300 mg, 0.51 mmol) in EA (2 mL) was added EA-HCl (2 mL, 3N). The mixture was stirred at room temperature for 1 hr. The mixture was concentrated to afford the title product (249 mg, 100%) as a yellow solid. LC-MS for C₂₇H₂₄FN₃O₃S+H [M+H]⁺: calcd. 490.2. found: 490.2.

### Intermediate 133

### tert-butyl methylsulfonyl((6-(1-(phenylsulfonyl)-1H-indol-3-yl)benzo[d]oxazol-2-yl)methyl)carbamate

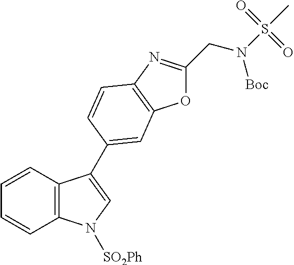

To a solution of tert-butyl methylsulfonylcarbamate (900 mg, 4.62 mmol), K₂CO₃ (1.3 g, 9.63 mmol) in DMF (60 mL) was added 2-(chloromethyl)-6-(1-(phenylsulfonyl)-1H-indol-3-yl)benzo[d]oxazole (Intermediate 79, 1.49 g, 3.52 mmol). The mixture was stirred at 50° C. for 9 hrs. The reaction mixture was filtered. The filtrate was diluted with water (60 mL) and extracted with EtOAc (60 mL×3). The combined organic layer was washed with water (60 mL×3), dried over anhydrous Na₂SO₄ and filtered. The filtrate was concentrated and purified by silica gel chromatography (petroleum ether/EtOAc=6/1-3/1) to afford 1.25 g (61%) of the title compound as a yellow solid. LC-MS for C₂₈H₂₇N₃O₇S₂+H [M+H]⁺: calcd. 582.1. found: 582.7. ¹H NMR (400 MHz, DMSO-d₆) δ [ppm]: 8.22 (s, 1H), 8.13 (d, J=1.1 Hz, 1H), 8.11 (s, 1H), 8.09 (d, J=1.3 Hz, 1H), 8.05 (d, J=8.3 Hz, 1H), 7.89 (d, J=7.9 Hz, 1H), 7.85 (d, J=8.3 Hz, 1H), 7.76 (dd, J=8.3, 1.5 Hz, 1H), 7.73-7.68 (m, 1H), 7.61 (t, J=7.7 Hz, 2H), 7.47-7.42 (m, 1H), 7.39-7.34 (m, 1H), 5.17 (s, 2H), 3.57 (s, 3H), 1.43 (s, 9H).

### Intermediate 134

### 6-(6-fluoro-1-(phenylsulfonyl)-1H-indol-3-yl)-2-methoxybenzo[d]oxazole

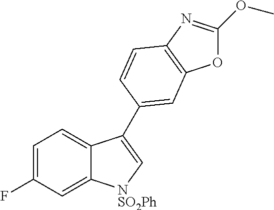

A mixture of 2-amino-5-(1-benzenesulfonyl-6-fluoro-1H-indol-3-yl)-phenol (Intermediate 69, 200 mg, 0.52 mmol), tetramethoxy-methane (1.40 g, 10.5 mmol) and toluene-4-sulfonic acid (20 mg, cat.) was stirred at 100° C. for 4 hours. The mixture was cooled, concentrated and purified by a silica gel column chromatography (petroleum ether/EtOAc=20/1 to 5/1) to afford 120 mg (54%) of the title compound as a white solid. LC-MS for C₂₂H₁₅FN₂O₄S+H⁺ [M+H]⁺: calcd. 423.1. found: 423.6. ¹H NMR (400 MHz, CDCl₃) δ [ppm]: 7.95 (s, 1H), 7.92 (d, J=1.6 Hz, 1H), 7.80 (dd, J=9.6, 2.4 Hz, 1H), 7.72-7.67 (m, 1H), 7.66 (s, 1H), 7.60-7.54 (m, 3H), 7.52-7.48 (m, 2H), 7.45 (dd, J=8.0, 1.6 Hz, 1H), 7.06 (td, J=9.2, 2.4 Hz, 1H), 4.26 (s, 3H).

### Intermediate 135

### tert-Butoxycarbonylamino{[6-(6-fluoroindol-3-yl)benzoxazol-2-yl]methyl}Sulfonamide

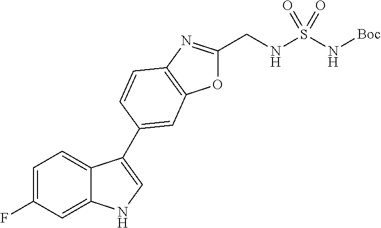 [SMILES: CC(C)(C)OC(=O)NS(=O)(=O)NCc1nc2ccc(-c3c[nH]c4cc(F)ccc34)cc2o1]

The solution of chloride sulfonyl isocyanate (0.95 mL, 1.1 mmol) in dichloromethane (0.87 mL) was cooled in ice bath. Tert-butanol (0.11 mL, 1.2 mmol) was added and then it was stirred at room temperature for 1 hr. A flask was charged with C-[6-(6-Fluoro-1H-indol-3-yl)-benzooxazol-2-yl]-methylamine hydrochloride (Compound 76, 317 mg, 1.0 mmol) and dichloromethane (5 mL). After being cooled to −15° C. with ice salt bath, the solution of tertbuyl[chlorosulfonyl]carbamate (prepared as above) was added. After stirring for 10 mins, triethylamine (606 mg, 6.6 mmol) was added. The cold bath was removed, and the reaction was allowed to warm to 10° C. The reaction was monitored by TLC. When it's done, the mixture was diluted with dichloromethane and washed with water and brine. The organic layer was dried and concentrated. The residue was purified by silica gel column chromatography (DCM/Methanol=40/1-20/1) to afford 240 mg (52%) as a yellow solid. LC-MS for C₂₁H₂₁FN₄O₅S+H⁺[M+H]⁺: calcd. 461.1. found: 461.6. ¹H NMR (400 MHz, DMSO) δ 11.48 (s, 1H), 11.02 (s, 1H), 8.55 (t, J=6.0 Hz, 1H), 7.92 (d, J=1.0 Hz, 1H), 7.87 (dd, J=8.8, 5.4 Hz, 1H), 7.80-7.70 (m, 2H), 7.69 (dd, J=8.3, 1.5 Hz, 1H), 7.24 (dd, J=9.9, 2.4 Hz, 1H), 6.97 (td, J=9.6, 2.4 Hz, 1H), 4.49 (d, J=6.0 Hz, 2H), 1.34 (s, 9H).

### Intermediate 136

### 5-(1-(phenylsulfonyl)-1H-indol-3-yl)benzo[d]oxazole

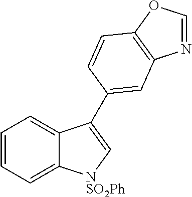

Following the general method as outlined in Intermediate 32, starting from 3-iodo-1-(phenylsulfonyl)-1H-indole (625 mg, 1.63 mmol) and 5-(4,4,5,5-Tetramethyl-15 [1,3,2]dioxaborolan-2-yl)-benzooxazole (400 mg, 1.63 mmol), 525 mg (86%) of the title compound was obtained as a yellow solid.

LC-MS for C₂₁H₁₄N₂O₃S+H⁺[M+H]⁺: calcd. 375.1. found: 375.4.

¹H NMR (300 MHz, DMSO-d₆) δ [ppm]: 8.81 (s, 1H), 8.17 (s, 1H), 8.15-8.07 (m, 3H), 8.04 (d, J=8.2 Hz, 1H), 7.90-7.81 (m, 2H), 7.79 (dd, J=8.4, 1.8 Hz, 1H), 7.70-7.58 (m, 3H), 7.43-7.38 (m, 1H), 7.37-7.35 (m, 1H).

### Intermediate 137

### 6-(6-fluoro-1-(phenylsulfonyl)-1H-indol-3-yl)-2-((1-(methylsulfonyl)piperidin-4-yl)methyl)benzo[d]oxazole

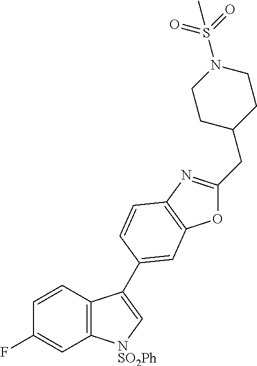

To a solution of 6-(6-fluoro-1-(phenylsulfonyl)-1H-indol-3-yl)-2-(piperidin-4-ylmethyl)benzo[d]oxazole (Intermediate 132, 300 mg, 0.613 mmol) in DCM (6 mL), TEA (186 mg, 1.84 mmol) was added at room temperature. Then methanesulfonyl chloride (84 mg, 0.74 mmol) was added into the mixture at 0° C. The mixture was stirred at 0° C. for 2 hrs. H₂O (10 mL) was added. The mixture was extracted with DCM (10 mL×3). The organic layer was dried over Na₂SO₄, filtered and concentrated. The residue was purified by silica gel column with PE-EA (4/1-1/1) to afford 275 mg (68%) of the title compound as a white solid. LC-MS for C₂₈H₂₆FN₃O₅S₂+H⁺[M+H]⁺: calcd. 568.1. found: 568.1.

### Intermediate 138

### 6-(6-fluoro-1-(phenylsulfonyl)-1H-indol-3-yl)-2-((1-methylpiperidin-4-yl)methyl)benzo[d]oxazole

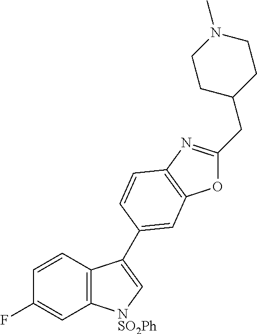

To a solution of 6-(6-fluoro-1-(phenylsulfonyl)-1H-indol-3-yl)-2-(piperidin-4-ylmethyl)benzo[d]oxazole (Intermediate 132, 330 mg, 0.67 mmol) in DCM/MeOH (6/3 mL) was added AcOH (122 mg, 2.02 mmol), CH₂O/H₂O (37%, 271 mg, 3.35 mmol) and NaBH(OAc)₃ (428 mg, 2.02 mmol). The mixture was stirred at room temperature for 3 hrs before it was quenched with H₂O (10 mL). The mixture was extracted with DCM (15 mL×3). The organic layer was dried over Na₂SO₄, filtered and concentrated. The residue was purified by silica gel column with DCM-MeOH (100/2) to afford 250 mg (74%) of the title compound as a yellow solid. LC-MS for C₂₈H₂₆FN₃O₃S+H⁺[M+H]⁺: calcd. 504.2. found: 504.2.

### Intermediate 139

### N-(2,4-dibromophenyl)-2-(2,5-dioxoimidazolidin-4-yl)acetamide

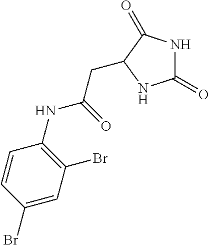 [SMILES: O=C(CC1NC(=O)NC1=O)Nc1ccc(Br)cc1Br]

A mixture of hydantoin-5-acetic acid (1.00 g, 6.32 mmol), HATU (2.86 g, 7.52 mmol) and 2,4,6-collidine (1.42 g, 11.7 mmol) in DMF (50 mL) was stirred at room temperature for 0.5 hour before 2,4-dibromoaniline (1.45 g, 5.78 mmol) was added. The mixture was stirred at room temperature for 12 hours before it was poured into water and EtOAc. The precipitate was collected by vacuum filtration to afford 450 mg (20%) of the crude title compound as a white solid, which was used directly without further purification. LC-MS for C₁₁H₉Br₂N₃O₃+H⁺[M+H]⁺: calcd. 389.9. found: 389.6. ¹H NMR (300 MHz, DMSO-d₆) δ [ppm]: 10.62 (br s, 1H), 9.64 (br s, 1H), 7.90 (s, 2H), 7.64-7.56 (m, 2H), 4.31 (td, J=6.6, 4.2 Hz, 1H), 2.89-2.83 (m, 1H), 2.75-2.50 (m, 1H).

### Intermediate 140

### 5-((6-bromobenzo[d]oxazol-2-yl)methyl)imidazolidine-2,4-dione

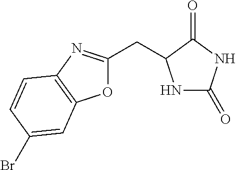 [SMILES: O=C1NC(=O)C(Cc2nc3ccc(Br)cc3o2)N1]

A mixture of N-(2,4-dibromophenyl)-2-(2,5-dioxoimidazolidin-4-yl)acetamide (Intermediate 141, 200 mg, 0.51 mmol), K₂CO₃ (106 mg, 0.77 mmol), CuBr (150 mg, 1.05 mmol) and pyridine (0.5 mL) in DMF (5 mL) was stirred at 140° C. for 2 hours in a microwave reactor. The mixture was partitioned between EtOAc and saturated aqueous NaHCO₃. The organic layer was dried over anhydrous Na₂SO₄, filtered, concentrated and purified by silica gel chromatography (petroleum ether/EtOAc=1/1-EtOAc) to afford 75 mg (47%) of the title compound as a yellow semisolid. LC-MS for C₁₁H₈BrN₃O₃—H⁻ [M−H]⁻: calcd. 308.0. found: 307.8.

### Intermediate 141

### tert-butyl 3-(2-((2,5-dioxoimidazolidin-4-yl)methyl)benzo[d]oxazol-6-yl)-6-fluoro-1H-indole-1-carboxylate

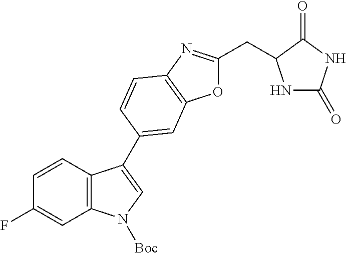 [SMILES: CC(C)(C)OC(=O)n1cc(-c2ccc3nc(CC4NC(=O)NC4=O)oc3c2)c2ccc(F)cc21]

Following the general method as outlined in Intermediate 32, starting from 5-((6-bromobenzo[d]oxazol-2-yl)methyl)imidazolidine-2,4-dione (Intermediate 142, 310 mg, 1.00 mmol) and tert-butyl 6-fluoro-3-(4,4,5,5-tetramethyl-1,3,2-dioxaborolan-2-yl)-1H-indole-1-carboxylate (Intermediate 2, 543 mg, 1.50 mmol), 50 mg (11%) of the title compound was obtained as a yellow solid.

LC-MS for C₂₄H₂₁FN₄O₅+H⁺[M+H]⁺: calcd. 465.2. found: 464.8. ¹H NMR (400 MHz, MeOH-d₄) δ [ppm]: 7.94 (dd, J=10.1, 2.3 Hz, 1H), 7.86 (d, J=1.6 Hz, 1H), 7.81 (s, 1H). 7.80 (dd, J=8.7, 5.4 Hz, 1H), 7.76 (d, J=8.4 Hz, 1H), 7.66 (dd, J=8.4, 1.6 Hz, 1H), 7.11 (ddd, J=9.6, 8.7, 2.3 Hz, 1H), 4.70 (dd, J=7.2, 4.6 Hz, 1H), 3.54 (dd, J=16.3, 4.6 Hz, 1H), 3.40 (dd, J=16.3, 7.2 Hz, 1H), 1.71 (s, 9H).

### Intermediate 142

### 6-(5-fluoro-1-(phenylsulfonyl)-1H-indol-3-yl)benzo[d]oxazol-2(3H)-one

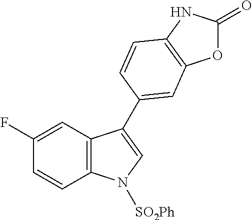

To a stirred solution of 2-amino-5-(1-benzenesulfonyl-5-fluoro-1H-indol-3-yl)-phenol (770 mg, 2.01 mmol) and CDl (359 mg, 2.21 mmol) in anhydrous THF (30 mL) was added TEA (1.68 mL) under nitrogen. The mixture was stirred at room temperature for 12 hours under nitrogen. The reaction was diluted with water (60 mL) and extracted with EtOAc (60 mL×3). The combined organic layer was washed with water (60 mL×3), dried over anhydrous Na₂SO₄ and filtered. The filtrate was concentrated to afford 820 mg (100%) of the title compound as a yellow solid. LC-MS for C₂₁H₁₃FN₂O₄S—H⁻ [M−H]⁻: calcd. 407.1. found: 407.5. ¹H NMR (400 MHz, DMSO-d₆) δ [ppm]: 11.79 (s, 1H), 8.19 (s, 1H), 8.08 (d, J=7.6 Hz, 2H), 8.03 (dd, J=9.1, 4.5 Hz, 1H), 7.75-7.67 (m, 2H), 7.65-7.57 (m, 3H), 7.50 (dd, J=8.0, 1.2 Hz, 1H), 7.28 (td, J=9.1, 2.5 Hz, 1H), 7.18 (d, J=8.1 Hz, 1H).

### Intermediate 143

### 2-(6-(5-fluoro-1-(phenylsulfonyl)-1H-indol-3-yl)-2-oxobenzo[d]oxazol-3(2H)-yl)acetamide

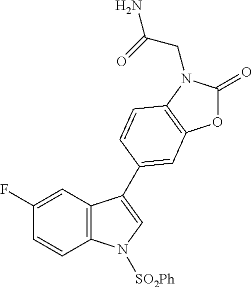

To a solution of 6-(5-fluoro-1-(phenylsulfonyl)-1H-indol-3-yl)benzo[d]oxazol-2(3H)-one (400 mg, 0.98 mmol) and K₂CO₃ (542 mg, 3.92 mmol) in NMP (20 mL) was added 2-bromoacetamide (135 mg, 0.98 mmol). The mixture was stirred at 60° C. for 6 hours. The reaction mixture was cooled to room temperature and filtered. The filtrate was diluted with water (60 mL) and extracted with EtOAc (60 mL×3). The combined organic layer was washed with water (60 mL×3), dried over anhydrous Na₂SO₄ and filtered. The filtrate was concentrated to afford 183 mg (81%) of the title compound as a yellow solid. LC-MS for C₂₃H₁₆FN₃O₅S—H⁻ [M−H]⁻: calcd. 464.1. found: 464.4. ¹H NMR (400 MHz, DMSO-d₆) δ [ppm]: 8.22 (s, 1H), 8.13-8.07 (m, 2H), 8.04 (dd, J=9.2, 4.4 Hz, 1H), 7.82-7.75 (m, 2H), 7.72 (t, J=7.2 Hz, 1H), 7.66-7.55 (m, 4H), 7.40 (s, 1H), 7.33-7.26 (m, 2H), 4.50 (s, 2H).

### Intermediate 144

### dimethyl 4-(1-(tert-butoxycarbonyl)-6-fluoro-1H-indol-3-yl)phthalate

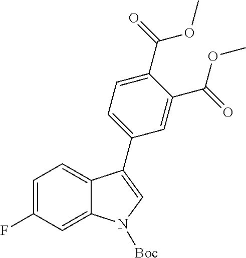 [SMILES: COC(=O)c1ccc(-c2cn(C(=O)OC(C)(C)C)c3cc(F)ccc23)cc1C(=O)OC]

Following the general method as outlined in Intermediate 32, starting from 4-bromo-phthalic acid dimethyl ester (362 mg, 1.33 mmol) and 6-fluoro-3-(4,4,5,5-tetramethyl-[1,3,2]dioxaborolan-2-yl)-indole-1-carboxylic acid tert-butyl ester (Intermediate 2, 400 mg, 1.10 mmol), the title compound (400 mg, 72%) was obtained as a red solid. LC-MS for C₂₃H₂₂FNO₆+H⁺[M+H]⁺: calcd. 428.1. found: 428.6. ¹H NMR (400 MHz, CDCl₃) δ [ppm]: 7.97 (d, J=10.0 Hz, 1H), 7.91 (d, J=2.0 Hz, 1H), 7.88 (d, J=8.4 Hz, 1H), 7.78 (dd, J=8.0, 2.0 Hz, 1H), 7.77 (s, 1H), 7.70 (dd, J=8.8, 5.2 Hz, 1H), 7.07 (td, J=8.8, 2.4 Hz, 1H), 3.95 (s, 3H), 3.94 (s, 3H), 1.70 (s, 9H).

### Intermediate 145

### 4-(6-fluoro-1H-indol-3-yl)phthalic acid

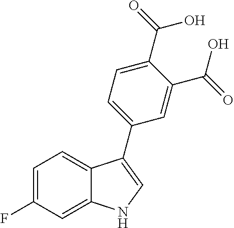 [SMILES: O=C(O)c1ccc(-c2c[nH]c3cc(F)ccc23)cc1C(=O)O]

To the solution of 6-fluoro-3-(4,4,5,5-tetramethyl-[1,3,2]dioxaborolan-2-yl)-indole-1-carboxylic acid tert-butyl ester (Intermediate 146, 360 mg, 0.84 mmol) in methanol (10 mL) was added NaOH (135 mg, 3.37 mmol) and the mixture was stirred at room temperature. After 2 hrs NaOH (135 mg, 3.37 mmol) was added and the mixture was stirred for another 4 hrs. The solvent was removed. The residue was diluted with water (10 mL) and the pH was adjusted to 1-2. The mixture was extracted with EA (20 mL×3). The combined organic layer was washed with brine, dried over Na₂SO₄ and filtered. The solvent was removed to afford 180 mg (72%) of the title compound as a yellow solid. LC-MS for C₁₆H₁₀FNO₄—H⁻ [M−H]⁻: calcd. 298.1. found: 298.4. ¹H NMR (400 MHz, DMSO-d₆) δ [ppm]: 12.99 (brs, 2H), 11.64 (s, 1H), 7.92 (d, J=2.4 Hz, 1H), 7.88 (dt, J=6.6, 3.0 Hz, 3H), 7.79 (d, J=8.0 Hz, 1H), 7.25 (dd, J=10.0, 2.4 Hz, 1H), 7.01 (td, J=9.4, 2.4 Hz, 1H).

### Intermediate 146

### tert-butyl 3-(2-(tert-butoxycarbonyl)isoindolin-5-yl)-6-fluoro-1H-indole-1-carboxylate

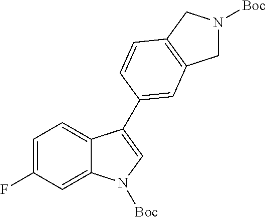 [SMILES: CC(C)(C)OC(=O)N1Cc2ccc(-c3cn(C(=O)OC(C)(C)C)c4cc(F)ccc34)cc2C1]

Following the general method as outlined in Intermediate 32, starting from tert-butyl 6-fluoro-3-(4,4,5,5-tetramethyl-1,3,2-dioxaborolan-2-yl)-1H-indole-1-carboxylate (Intermediate 2, 260 mg, 0.72 mmol) and 5-bromo-1,3-dihydro-isoindole-2-carboxylic acid tert-butyl ester (278 mg, 0.93 mmol), 178 mg (55%) of the title compound was obtained as a white solid. LC-MS for C₂₆H₂₉FN₂O₄+H⁺-56 [M+H-56]⁺: calcd. 397.2. found: 397.6. ¹H NMR (400 MHz, CDCl₃) δ [ppm]: 7.97-7.93 (m, 1H), 7.74-7.59 (m, 2H), 7.53-7.45 (m, 2H), 7.37-7.28 (m, 1H), 7.07-7.02 (m, 1H), 4.76-4.71 (m, 4H), 1.69 (s, 9H), 1.54 (s, 9H).

### Intermediate 147

### tert-butyl 3-(1,1-dioxido-2,3-dihydrobenzo[d]isothiazol-6-yl)-6-fluoro-1H-indole-1-carboxylate

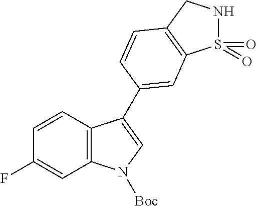 [SMILES: CC(C)(C)OC(=O)n1cc(-c2ccc3c(c2)S(=O)(=O)NC3)c2ccc(F)cc21]

Following the general method as outlined in Intermediate 32, starting from 6-bromo-2,3-dihydro-benzo[d]isothiazole 1,1-dioxide (70 mg, 0.28 mmol) and 6-Fluoro-3-(4,4,5,5-tetramethyl-[1,3,2]dioxaborolan-2-yl)-indole-1-carboxylic acid tert-butyl ester (Intermediate 2, 139 mg, 0.42 mmol), 50 mg (29%) of the title compound was obtained as a yellow solid. ¹H NMR (400 MHz, DMSO-d₆) δ [ppm]: 8.13 (d, J=8.7 Hz, 2H), 8.03 (dd, J=8.4, 1.2 Hz, 1H), 7.92-7.83 (m, 3H), 7.67 (d, J=8.1 Hz, 1H), 7.24 (td, J=9.0, 1.5 Hz, 1H), 4.46 (s, 2H), 1.66 (s, 9H).

### Intermediate 148

### 4-(1-(tert-butoxycarbonyl)-6-fluoro-1H-indol-3-yl)-2-(N-methylsulfamoyl)benzoic acid

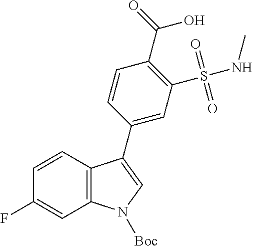 [SMILES: CNS(=O)(=O)c1cc(-c2cn(C(=O)OC(C)(C)C)c3cc(F)ccc23)ccc1C(=O)O]

Following the general method as outlined in Intermediate 32, starting from 6-bromo-2-methylbenzo[d]isothiazol-3(2H)-one 1,1-dioxide (400 mg, 1.4 mmol) and 6-fluoro-3-(4,4,5,5-tetramethyl-[1,3,2]dioxaborolan-2-yl)-indole-1-carboxylic acid tert-butyl ester (Intermediate 2, 790 mg, 2.2 mmol), 150 mg (25%) of the title compound was obtained as a white solid. ¹H NMR (400 MHz, DMSO-d₆) δ [ppm]: 9.65 (s, 1H), 7.98-7.75 (m, 6H), 7.27 (td, J=9.2, 2.8 Hz, 1H), 2.39 (s, 3H), 1.66 (s, 9H).

### Intermediate 149

### tert-butyl 6-fluoro-3-(2-oxo-2,3-dihydrobenzo[d]oxazol-6-yl)-1H-indole-1-carboxylate

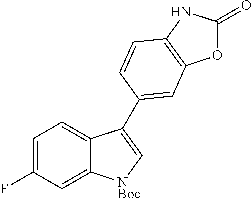 [SMILES: CC(C)(C)OC(=O)n1cc(-c2ccc3[nH]c(=O)oc3c2)c2ccc(F)cc21]

Following the general method as outlined in Intermediate 32, starting from 6-bromo-3H-benzooxazol-2-one (600 mg, 2.80 mmol) and 6-fluoro-3-(4,4,5,5-tetramethyl-[1,3,2]dioxaborolan-2-yl)-indole-1-carboxylic acid tert-butyl ester (Intermediate 2, 1.21 g, 3.36 mmol), 450 mg (45%) of the title compound was obtained as a white solid. ¹H NMR (400 MHz, CDCl₃) δ [ppm]: 8.56 (s, 1H), 7.95 (d, J=9.6 Hz, 1H), 7.69-7.64 (m, 1H), 7.46 (s, 1H), 7.42 (dd, J=8.0, 1.6 Hz, 1H), 7.16 (d, J=8.0 Hz, 1H), 7.06 (td, J=8.8, 2.4 Hz, 1H), 3.71 (s, 1H), 1.70 (s, 9H).

### Intermediate 150

### tert-butyl 3-(3-(1-(tert-butoxycarbonyl)piperidin-4-yl)-2-oxo-2,3-dihydrobenzo[d]oxazol-6-yl)-6-fluoro-1H-indole-1-carboxylate

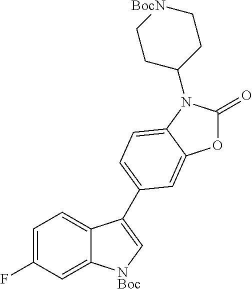 [SMILES: CC(C)(C)OC(=O)N1CCC(n2c(=O)oc3cc(-c4cn(C(=O)OC(C)(C)C)c5cc(F)ccc45)ccc32)CC1]

To a stirred solution of 6-fluoro-3-(2-oxo-2,3-dihydro-benzooxazol-6-yl)-indole-1-carboxylic acid tert-butyl ester (Intermediate 151, 450 mg, 1.22 mmol), 4-hydroxy-piperidine-1-carboxylic acid tert-butyl ester (369 mg, 1.83 mmol) and PPh₃ (480 mg, 1.83 mmol) in anhydrous THF (10 mL) was added DIAD (370 mg, 1.83 mmol) at 0° C. The mixture was stirred at room temperature for 16 hours under N₂ before it was diluted with EtOAc (50 mL), washed with water (20 mL) and brine (20 mL×2). The organic layer was dried over anhydrous Na₂SO₄ and filtered. The filtrate was concentrated and purified by a silica gel column chromatography (petroleum ether/EtOAc=30/1 to 10/1) and preparative TLC (petroleum ether/EtOAc=10/1) to afford 125 mg (17%) of the title compound as a white solid. LC-MS for C₃₀H₃₄FN₃O₆+H⁺[M+H]⁺: calcd. 552.2. found: 552.8. ¹H NMR (400 MHz, CDCl₃) δ [ppm]: 7.94 (d, J=10.0 Hz, 1H), 7.69-7.61 (m, 2H), 7.46 (s, 1H), 7.40 (dd, J=8.0, 2.0 Hz, 1H), 7.18 (d, J=8.0 Hz, 1H), 7.06 (td, J=8.8, 2.4 Hz, 1H), 4.43-4.30 (m, 3H), 2.95-2.83 (m, 2H), 2.36-2.24 (m, 2H), 1.95-1.88 (m, 2H), 1.70 (s, 9H), 1.51 (s, 9H).

### Intermediate 151

### tert-butyl 6-fluoro-3-(2-methyl-1,1-dioxido-3-oxo-2,3-dihydrobenzo[d]isothiazol-5-yl)-1H-indole-1-carboxylate

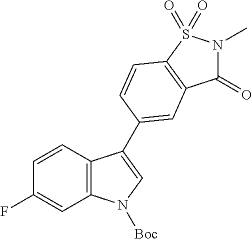 [SMILES: CN1C(=O)c2cc(-c3cn(C(=O)OC(C)(C)C)c4cc(F)ccc34)ccc2S1(=O)=O]

To a solution of tert-butyl 3-(1,1-dioxido-3-oxo-2,3-dihydrobenzo[d]isothiazol-5-yl)-6-fluoro-1H-indole-1-carboxylate (Compound 130 Step 1, 246 mg, 0.59 mmol) and K₂CO₃ (245 mg, 1.77 mmol) in acetone (10 mL) was added MeI (126 mg, 0.89 mmol). The reaction mixture was stirred at 40° C. overnight. The reaction was filtered, concentrated and triturated in MeOH to afford 60 mg (24%) of the title compound as a yellow solid. ¹H NMR (400 MHz, DMSO-d₆) δ [ppm]: 8.43-8.38 (m, 2H), 8.35 (s, 1H), 8.33 (s, 1H), 7.97-7.91 (m, 2H), 7.28 (td, J=9.2, 2.4 Hz, 1H), 3.21 (s, 3H), 1.67 (s, 9H).

### Intermediate 152

### tert-butyl 6-(2-(6-(6-fluoro-1H-indol-3-yl)benzo[d]oxazol-2-yl)ethyl)-2,6-diazaspiro[3.3]heptane-2-carboxylate

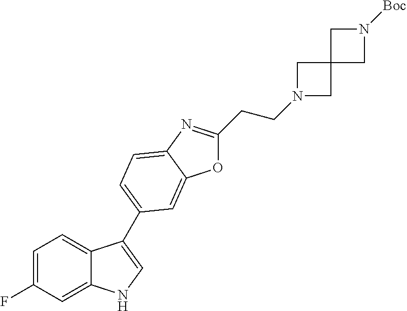 [SMILES: CC(C)(C)OC(=O)N1CC2(CN(CCc3nc4ccc(-c5c[nH]c6cc(F)ccc56)cc4o3)C2)C1]

To a stirred solution of 6-(6-fluoro-1-(phenylsulfonyl)-1H-indol-3-yl)-2-vinylbenzo[d]oxazole (Intermediate 128, 100 mg, 0.24 mmol) in dry MeOH (10 mL) at 50° C. was added 2,6-diaza-spiro[3.3]heptane-2-carboxylic acid tert-butyl ester (146 mg, 0.6 mmol; made according to the procedures reported by Org. lett., 2008, 10, 3525). The mixture was stirred at 50° C. overnight. The solvent was removed. The residue was diluted with water (30 mL) and extracted with EA (10 mL×3). The combined organic layer was washed with brine (20 mL), dried over anhydrous Na₂SO₄ and concentrated to afford 200 mg of the title compound as a white solid, which was used for next step without further purification.

### Intermediate 153

### tert-butyl 3-(benzofuran-6-yl)-6-fluoro-1H-indole-1-carboxylate

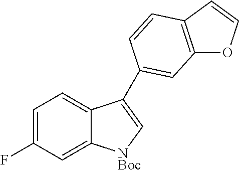 [SMILES: CC(C)(C)OC(=O)n1cc(-c2ccc3ccoc3c2)c2ccc(F)cc21]

To a stirred solution of 6-bromobenzo[b]furan (100 mg, 0.507 mmol) in 1,4-dioxane (15 mL), tert-butyl 6-fluoro-3-(4,4,5,5-tetramethyl-1,3,2-dioxaborolan-2-yl)-1H-indole-1-carboxylate (Intermediate 2, 180 mg, 0.507 mmol), K₂CO₃ (210 mg, 1.52 mmol) and Pd(dppf)Cl₂ (19 mg, 0.025 mmol) were added. The mixture was stirred at 80° C. for 15 hrs. The solvent was removed and the residue was purified by prep-TLC (EtOAc/Petroleum Ether=1/10) to afford 112.8 mg (63%) of the title compound. LC-MS for C₂₁H₁₈FNO₃+H⁺[M+H]⁺: calcd: 351.1. found: 351.8. ¹H NMR (400 MHz, CDCl₃) δ [ppm]: 7.96 (d, J=4.8 Hz, 1H), 7.79-7.76 (m, 2H), 7.71-7.67 (m, 3H), 7.50 (dd, J=8.0 Hz, 1.6 Hz, 1H), 7.06 (dd, J=8.8, 2.4 Hz, 1H), 6.82 (d, J=1.0 Hz, 1H), 1.70 (s, 9H).

### I.2. Synthesis of Final Compounds

### Compound 1

### 6-(6-fluoro-1H-indol-3-yl)-1H-indazole

To a solution of 6-(6-fluoro-1-(phenylsulfonyl)-1H-indol-3-yl)-1H-indazole (Intermediate 4; 390 mg; 1.00 mmol) in MeOH (15 mL) was added a solution of NaOH (200 mg; 5.00 mmol) in water (2 mL). The reaction mixture was stirred at 80° C. for 2 hours, concentrated, and purified by a silica gel chromatography (petroleum ether/EtOAc=5/1-2/1) to afford 50 mg (20%) of the title compound as a white solid. LC-MS for C₁₅H₁₀FN₃+H [M+H]⁺: calcd. 252.1. found: 252.1. ¹H NMR (300 MHz, DMSO-d₆) δ [ppm]: 12.94 (s, 1H), 11.45 (s, 1H), 8.05 (s, 1H), 7.88 (dd, J=8.8, 5.4 Hz, 1H), 7.79 (d, J=8.4 Hz, 1H), 7.77-7.73 (m, 2H), 7.44 (d, J=8.4 Hz, 1H), 7.24 (dd, J=10.0, 2.2 Hz, 1H), 6.99 (ddd, J=9.3, 8.8, 2.2 Hz, 1H).

### Compound 2

### 2-(6-(6-fluoro-1H-indol-3-yl)-1H-indazol-1-yl)acetamide

A mixture of 6-(6-fluoro-1-(phenylsulfonyl)-1H-indol-3-yl)-1H-indazole (Intermediate 4; 300 mg; 0.77 mmol), 2-bromoacetamide (1.05 g; 7.61 mmol), KI (290 mg; 1.75 mmol), K₂CO₃ (986 mg; 7.13 mmol) in DMF (16.5 mL) was stirred at 60° C. for 48 hours under nitrogen. The mixture was cooled to room temperature, diluted with EtOAc (100 mL), filtered, concentrated, and purified by preparative HPLC to afford 25 mg (11%) of the title compound as a yellow solid. LC-MS for C₁₇H₁₃FN₄O+H⁺[M+H]⁺: calcd. 309.1. found: 308.9. ¹H NMR (400 MHz, DMSO-d₆) δ [ppm]: 11.46 (s, 1H), 8.03 (s, 1H), 7.96 (dd, J=8.7, 5.4 Hz, 1H), 7.80-7.77 (m, 2H), 7.76-7.73 (m, 2H), 7.55 (br s, 1H), 7.46 (dd, J=8.3, 1.3 Hz, 1H), 7.26 (br s, 1H), 7.22 (dd, J=9.9, 2.4 Hz, 1H), 6.96 (ddd, J=9.6, 8.8, 2.4, 1H), 5.11 (s, 2H).

### Compound 3

### 6-(6-fluoro-1H-indol-3-yl)-1-(piperidin-4-ylmethyl)-1H-indazole hydrochloride

Following the general method as outlined in Compound 1, starting from 6-(6-fluoro-1-(phenylsulfonyl)-1H-indol-3-yl)-1-(piperidin-4-ylmethyl)-1H-indazole hydrochloride and 6-(6-fluoro-1-(phenylsulfonyl)-1H-indol-3-yl)-2-(piperidin-4-ylmethyl)-2H-indazole hydrochloride (Intermediate 6; 136 mg; 0.26 mmol), 3 mg (3%) of the title compound was obtained as a yellow solid after purification by preparative HPLC with 0.1% HCl as buffer. LC-MS for C₂₁H₂₁FN₄+H⁺[M+H]⁺: calcd. 349.2. found: 349.0. ¹H NMR (300 MHz, DMSO-d₆) δ [ppm]: 11.52 (s, 1H), 9.26 (br s, 1H), 8.90 (br s, 1H), 8.39 (s, 1H), 7.87 (dd, J=8.8, 5.4 Hz, 1H), 7.80 (s, 1H), 7.77-771 (m, 2H), 7.41 (d, J=8.6 Hz, 1H), 7.24 (d, J=9.9, 2.3 Hz, 1H), 6.97 (ddd, J=9.4, 8.8, 2.3 Hz, 1H), 4.38 (d, J=6.9 Hz, 2H), 3.18-3.18 (m, 2H), 2.91-2.72 (m, 2H), 2.38-2.22 (m, 1H), 1.73-1.59 (m, 2H), 1.58-1.40 (m, 2H).

### Compound 4

### 1-(4-((6-(6-fluoro-1H-indol-3-yl)-1H-indazol-1-yl)methyl)piperidin-1-yl)ethanone

Following the general method as outlined in Compound 1, starting from 1-(4-((6-(6-fluoro-1-(phenylsulfonyl)-1H-indol-3-yl)-1H-indazol-1-yl)methyl)piperidin-1-yl)ethanone (Intermediate 7; 267 mg; 0.50 mmol), 12 mg (6%) of the title compound was obtained as a white solid after purification by preparative HPLC. LC-MS for C₂₃H₂₃FN₄O—H⁻ [M−H]⁻: calcd. 389.2. found: 389.0. ¹H NMR (400 MHz, DMSO-d₆) δ [ppm]: 11.51 (s, 1H), 8.05 (s, 1H), 7.95 (dd, J=8.8, 5.4 Hz, 1H), 7.87 (s, 1H), 7.80-7.76 (m, 2H), 7.47 (d, J=8.4 Hz, 1H), 7.25 (dd, J=9.9, 2.0 Hz, 1H), 6.99 (ddd, J=9.4, 8.8, 2.0 Hz, 1H), 4.40-4.30 (m, 3H), 3.82-3.73 (m, 1H), 2.99-2.89 (m, 1H), 2.50-2.40 (m, 1H), 2.26-2.14 (m, 1H), 1.95 (s, 3H), 1.56-1.44 (m, 2H), 1.30-1.16 (m, 2H).

### Compound 5

### 3-(6-(6-fluoro-1H-indol-3-yl)-1H-indazol-1-yl)propanamide

Following the general method as outlined in Compound 1, starting from 3-(6-(6-fluoro-1-(phenylsulfonyl)-1H-indol-3-yl)-1H-indazol-1-yl)propanamide (Intermediate 8; 95 mg; 0.21 mmol), 35 mg (53%) of the title compound was obtained as a white solid after purification by preparative TLC (EtOAc).

LC-MS for C₁₈H₁₅FN₄O+H⁺[M+H]⁺: calcd. 323.1. found: 323.1. ¹H NMR (300 MHz, DMSO-d₆) δ [ppm]: 11.48 (s, 1H), 8.05 (dd, J=8.8, 5.6 Hz, 1H), 8.03 (s, 1H), 7.89 (s, 1H), 7.79-7.73 (m, 2H), 7.45 (d, J=8.4 Hz, 1H), 7.40 (br s, 1H), 7.24 (dd, J=9.9, 2.2 Hz, 1H), 6.99 (ddd, J=9.4, 8.8, 2.2 Hz, 1H), 6.87 (br s, 1H), 4.64 (t, J=6.6 Hz, 2H), 2.70 (t, J=6.6 Hz, 2H).

### Compound 6

### 6-(6-fluoro-1H-indol-3-yl)-1-(piperidin-4-yl)-1H-indazole

Following the general method as outlined in Compound 1, starting from 6-(6-fluoro-1-(phenylsulfonyl)-1H-indol-3-yl)-1-(piperidin-4-yl)-1H-indazole hydrochloride and 6-(6-fluoro-1-(phenylsulfonyl)-1H-indol-3-yl)-2-(piperidin-4-yl)-2H-indazole hydrochloride (Intermediate 10; 700 mg crude; 1.37 mmol), 12.3 mg (3%) of the title compound was obtained as a yellow solid after purification by preparative HPLC. LC-MS for C₂₀H₁₉FN₄+H⁺[M+H]⁺: calcd. 335.2. found: 334.9. ¹H NMR (300 MHz, DMSO-d₆) δ [ppm]: 11.49 (s, 1H), 8.04 (s, 1H), 7.94 (dd, J=9.0, 5.4 Hz, 1H), 7.88 (s, 1H), 7.80-7.75 (m, 2H), 7.46 (dd, J=8.4, 1.3 Hz, 1H), 7.25 (dd, J=9.8, 2.4 Hz, 1H), 6.99 (ddd, J=9.7, 8.9, 2.4 Hz, 1H), 4.87-4.75 (m, 1H), 3.20-3.10 (m, 2H), 2.86-2.75 (m, 2H), 2.10-2.00 (m, 2H), 2.00-1.92 (m, 2H).

### Compound 7

### 1-(4-(6-(6-fluoro-1H-indol-3-yl)-1H-indazol-1-yl)piperidin-1-yl)ethanone

To a solution of 6-(6-fluoro-1H-indol-3-yl)-1-(piperidin-4-yl)-1H-indazole (Compound 6; 300 mg; 0.90 mmol) and Et₃N (5.0 mL; 36 mmol) in DCM (30 mL) was added AcCl (84.6 mg; 1.08 mmol) under nitrogen. The mixture was stirred for 1 hour and quenched with saturated aqueous NaHCO₃ (30 mL). The aqueous layer was extracted with DCM (60 mL×2). The combined organic layers were washed with brine (20 mL), dried over anhydrous Na₂SO₄, filtered, concentrated, and purified by preparative HPLC to afford 15 mg (4%) of the title compound as a yellow solid. LC-MS for C₂₂H₂₁FN₄O+H⁺ [M+H]⁺: calcd. 377.2. found: 376.9. ¹H NMR (400 MHz, DMSO-d₆) δ [ppm]: 11.49 (s, 1H), 8.05 (s, 1H), 7.96 (dd, J=8.8, 5.4 Hz, 1H), 7.91 (s, 1H), 7.81-7.76 (m, 2H), 7.47 (dd, J=8.4, 1.2 Hz, 1H), 7.25 (dd, J=9.9, 2.4 Hz, 1H), 6.99 (ddd, J=9.6, 8.8, 2.4 Hz, 1H), 5.08-4.95 (m, 1H), 4.58-4.48 (m, 1H), 4.03-3.93 (m, 1H), 3.33-3.27 (m, 1H), 2.90-2.78 (m, 1H), 2.07 (s, 3H), 2.10-1.88 (m, 4H).

### Compound 8

### 5-(6-fluoro-1H-indol-3-yl)-1H-indazole

Following the general method as outlined in Compound 1, starting from 5-(6-fluoro-1-(phenylsulfonyl)-1H-indol-3-yl)-1H-indazole (Intermediate 12; 270 mg crude; 0.45 mmol), 40 mg (32%) of the title compound was obtained as a yellow solid after purification by preparative HPLC. LC-MS for C₁₅H₁₀FN₃+H⁺[M+H]⁺: calcd. 252.1. found: 252.1. ¹H NMR (300 MHz, DMSO-d₆) δ [ppm]: 13.02 (s, 1H), 11.34 (s, 1H), 8.08 (s, 1H), 7.99 (s, 1H), 7.86 (dd, J=8.8, 5.4 Hz, 1H), 7.66 (dd, J=9.0, 1.5 Hz, 1H), 7.64 (d, J=1.5 Hz, 1H), 7.59 (d, J=9.0 Hz, 1H), 7.22 (dd, J=10.0, 2.4 Hz, 1H), 6.95 (ddd, J=9.7, 8.8, 2.4 Hz, 1H).

### Compound 9

### 2-(5-(6-fluoro-1H-indol-3-yl)-1H-indazol-1-yl)acetamide

Following the general method as outlined in Compound 1, starting from 2-(5-(6-fluoro-1-(phenylsulfonyl)-1H-indol-3-yl)-1H-indazol-1-yl)acetamide and 2-(5-(6-fluoro-1-(phenylsulfonyl)-1H-indol-3-yl)-2H-indazol-2-yl)acetamide (Intermediate 13; 353 mg; 0.79 mmol), 34.1 mg (14%) of the title compound was obtained as a yellow solid after purification by preparative HPLC and chiral preparative HPLC. LC-MS for C₁₇H₁₃FN₄O+H⁺[M+H]⁺: calcd. 309.1. found: 308.9. ¹H NMR (300 MHz, DMSO-d₆) δ [ppm]: 11.39 (s, 1H), 8.08 (s, 1H), 7.99 (s, 1H), 7.87 (dd, J=8.8, 5.3 Hz, 1H), 7.72-7.61 (m, 3H), 7.58 (br s, 1H), 7.30 (br s, 1H), 7.23 (dd, J=10.0, 2.3 Hz, 2H), 6.99 (dd, J=9.5, 8.8, 2.3 Hz, 1H), 5.08 (s, 2H).

### Compound 10

### 5-(6-fluoro-1H-indol-3-yl)-1-(piperidin-4-ylmethyl)-1H-indazole

A solution of tert-butyl 4-((5-(6-fluoro-1H-indol-3-yl)-1H-indazol-1-yl)methyl)piperidine-1-carboxylate (Intermediate 15; 1.18 g; 2.63 mmol) in saturated HCl in 1,4-dioxane (20 mL) was stirred at room temperature for 1 hour. The reaction mixture was concentrated in vacuo, diluted with water (100 mL), and extracted with EtOAc (50 mL×3). The aqueous layer was basified with aqueous NaOH to pH=13 and extracted with EtOAc (100 mL×3). The combined organic layers were dried over anhydrous Na₂SO₄, filtered, concentrated, and purified by preparative TLC (DCM/MeOH=8/1) to afford 31 mg (33%) of the title compound as a yellow solid. LC-MS for C₂₁H₂₁FN₄+H⁺[M+H]⁺: calcd. 349.2. found: 349.0. ¹H NMR (300 MHz, DMSO-d₆) δ [ppm]: 11.42 (s, 1H), 8.60 (br s, 1H), 8.09 (s, 1H), 8.00 (s, 1H), 7.86 (dd, J=8.8, 5.3 Hz, 1H), 7.79-7.65 (m, 3H), 7.30 (dd, J=10.0, 2.4 Hz, 1H), 6.96 (ddd, J=9.6, 8.8, 2.4 Hz, 1H), 4.38 (d, J=6.7 Hz, 2H), 3.26-3.15 (m, 2H), 2.86-2.71 (m, 2H), 2.30-2.13 (m, 1H), 1.72-1.60 (m, 2H), 1.55-1.36 (m, 2H).

### Compound 11

### 1-(4-((5-(6-fluoro-1H-indol-3-yl)-1H-indazol-1-yl)methyl)piperidin-1-yl)ethanone

Following the general method as outlined in Compound 7, starting from 5-(6-fluoro-1H-indol-3-yl)-1-(piperidin-4-ylmethyl)-1H-indazole (Compound 10; 204 mg; 0.59 mmol), 65 mg (28%) of the title compound was obtained as a white solid after purification by preparative HPLC. LC-MS for C₂₃H₂₃FN₄O+H⁺ [M+H]⁺: calcd. 391.2. found: 390.9. ¹H NMR (300 MHz, DMSO-d₆) δ [ppm]: 11.38 (s, 1H), 8.08 (s, 1H), 7.99 (s, 1H), 7.87 (dd, J=8.9, 5.5 Hz, 1H), 7.76 (d, J=8.8 Hz, 1H), 7.71-7.66 (m, 2H), 7.22 (dd, J=10.0, 2.4 Hz, 1H), 6.96 (ddd, J=9.6, 8.9, 2.3 Hz, 1H), 4.39-4.30 (m, 3H), 3.83-3.73 (m, 1H), 3.00-2.87 (m, 1H), 2.50-2.40 (m, 1H), 2.26-2.10 (m, 1H), 1.96 (s, 3H), 1.54-1.43 (m, 2H), 1.30-1.00 (m, 2H).

### Compound 12

### 3-(5-(6-fluoro-1H-indol-3-yl)-1H-indazol-1-yl)propanamide

Following the general method as outlined in Compound 1, starting from 3-(5-(6-fluoro-1-(phenylsulfonyl)-1H-indol-3-yl)-1H-indazol-1-yl)propanamide and 3-(5-(6-fluoro-1-(phenylsulfonyl)-1H-indol-3-yl)-2H-indazol-2-yl)propanamide (Intermediate 16; 492 mg; 1.06 mmol), 33.9 mg (10%) of the title compound was obtained as a white solid after purification by preparative HPLC and chiral preparative HPLC. LC-MS for C₁₈H₁₅FN₄O+H⁺[M+H]⁺: calcd. 323.1. found: 322.9. ¹H NMR (300 MHz, DMSO-d₆) δ [ppm]: 11.34 (s, 1H), 8.27 (d, J=0.8 Hz, 1H), 7.93-7.91 (m, 1H), 7.88 (dd, J=8.8, 5.4 Hz, 1H), 7.67-7.61 (m, 2H), 7.56 (dd, J=9.0, 1.6 Hz, 1H), 7.43 (br s, 1H), 7.21 (dd, J=10.0, 2.3 Hz, 1H), 6.95 (ddd, J=9.6, 8.8, 2.3 Hz, 1H), 6.92 (br s, 1H), 4.62 (t, J=6.8 Hz, 2H), 2.79 (t, J=6.9 Hz, 2H).

### Compound 13

### 5-(6-fluoro-1H-indol-3-yl)-1-(piperidin-4-yl)-1H-indazole

To a solution of tert-butyl 4-(5-(6-fluoro-1H-indol-3-yl)-1H-indazol-1-yl)piperidine-1-carboxylate (Intermediate 18; 180 mg; 0.41 mmol) in 1,4-dioxane (20 mL) was added saturated HCl in 1,4-dioxane (10 mL). The reaction mixture was stirred overnight. The resulting precipitate was collected by filtration and purified by preparative HPLC to afford 56 mg (40%) of the title compound as a yellow solid. LC-MS for C₂₀H₁₉FN₄+H⁺[M+H]⁺: calcd. 335.2. found: 334.9. ¹H NMR (300 MHz, DMSO-d₆) δ [ppm]: 11.36 (s, 1H), 8.07 (s, 1H), 7.98 (s, 1H), 7.86 (dd, J=8.8, 5.4 Hz, 1H), 7.77 (d, J=8.8 Hz, 1H), 7.67 (dd, J=8.8, 1.6 Hz, 1H), 7.66 (d, J=1.6 Hz, 1H), 7.22 (dd, J=10.0, 2.4 Hz, 1H), 6.95 (ddd, J=9.6, 8.8, 2.4 Hz, 1H), 4.73-4.60 (m, 1H), 3.14-3.04 (m, 2H), 2.76-2.64 (m, 2H), 2.20 (br s, 1H), 2.05-1.93 (m, 2H), 1.91-1.82 (m, 2H).

### Compound 14

### 1-(4-(5-(6-fluoro-1H-indol-3-yl)-1H-indazol-1-yl)piperidin-1-yl)ethanone

Following the general method as outlined in Compound 7, starting from 5-(6-fluoro-1H-indol-3-yl)-1-(piperidin-4-yl)-1H-indazole (Compound 13; 53 mg; 0.16 mmol), 16 mg (27%) of the title compound was obtained as a yellow solid after purification by preparative HPLC. LC-MS for C₂₂H₂₁FN₄O+H⁺[M+H]⁺: calcd. 377.2. found: 376.9. ¹H NMR (300 MHz, MeOH-d₄) δ [ppm]: 8.25 (s, 1H), 7.92-7.89 (m, 1H), 7.83 (dd, J=8.8, 5.3 Hz, 1H), 7.66-7.63 (m, 2H), 7.46 (s, 1H), 7.12 (dd, J=10.0, 2.3 Hz, 1H), 6.88 (ddd, J=9.6, 8.8, 2.3 Hz, 1H), 4.86-4.66 (m, 2H), 4.16-4.06 (m, 1H), 3.42-3.32 (m, 1H), 2.93-2.81 (m, 1H), 2.32-2.00 (m, 4H), 2.18 (s, 3H).

### Compound 15

### 2-(6-(6-fluoro-1H-indol-3-yl)-2H-indazol-2-yl)acetamide

A mixture of 6-(6-fluoro-1-(phenylsulfonyl)-1H-indol-3-yl)-1H-indazole (Intermediate 4; 300 mg; 0.77 mmol), 2-bromoacetamide (1.05 g; 7.61 mmol), KI (290 mg; 1.75 mmol) and K₂CO₃ (986 mg; 7.13 mmol) in DMF (16.5 mL) was stirred at 60° C. for 48 hours under nitrogen. The mixture was cooled to room temperature, diluted with EtOAc (100 mL), filtered, concentrated, and purified by preparative HPLC to afford 50 mg (21%) of the title compound as a yellow solid. LC-MS for C₁₇H₁₃FN₄O+H⁺[M+H]⁺: calcd. 309.1. found: 308.9. ¹H NMR (400 MHz, DMSO-d₆) δ [ppm]: 11.42 (s, 1H), 8.32 (s, 1H), 7.90 (dd, J=8.8, 5.4 Hz, 1H), 7.80 (s, 1H), 7.81-7.72 (m, 2H), 7.66 (br s, 1H), 7.39 (dd, J=8.7, 1.4 Hz, 1H), 7.35 (br s, 1H), 7.24 (dd, J=10.0, 2.4 Hz, 1H), 6.97 (ddd, J=9.6, 8.8, 2.4 Hz, 1H), 5.09 (s, 2H).

### Compound 16

### 1-(4-((6-(6-fluoro-1H-indol-3-yl)-2H-indazol-2-yl)methyl)piperidin-1-yl)ethanone

Following the general method as outlined in Compound 1, starting from 1-(4-((6-(6-fluoro-1-(phenylsulfonyl)-1H-indol-3-yl)-2H-indazol-2-yl)methyl)piperidin-1-yl)ethanone (Intermediate 19; 358 mg; 0.67 mmol), 10 mg (4%) of the title compound was obtained as a white solid after purification by preparative HPLC.

LC-MS for C₂₃H₂₃FN₄O+H⁺[M+H]⁺: calcd. 389.9. found: 389.9. ¹H NMR (400 MHz, DMSO-d₆) δ [ppm]: 11.43 (s, 1H), 8.32 (s, 1H), 7.90 (dd, J=8.8, 5.4 Hz, 1H), 7.80 (s, 1H), 7.77-7.71 (m, 2H), 7.39 (d, J=8.6 Hz, 1H), 7.23 (dd, J=10.0, 2.3 Hz, 1H), 6.97 (dd, J=9.6, 8.8, 2.3 Hz, 1H), 4.40-4.30 (m, 3H), 3.85-3.75 (m, 1H), 3.05-2.90 (m, 1H), 2.50-2.42 (m, 1H), 2.31-2.22 (m, 1H), 1.97 (s, 3H), 1.60-1.40 (m, 2H), 1.30-1.00 (m, 2H).

### Compound 17

### 3-(6-(6-fluoro-1H-indol-3-yl)-2H-indazol-2-yl)propanamide

Following the general method as outlined in Compound 1, starting from 3-(6-(6-fluoro-1-(phenylsulfonyl)-1H-indol-3-yl)-2H-indazol-2-yl)propanamide (Intermediate 20; 75 mg; 0.16 mmol), 20 mg (38%) of the title compound was obtained as a white solid after purification by preparative TLC (EtOAc). LC-MS for C₁₈H₁₅FN₄O+H⁺ [M+H]⁺: calcd. 323.1. found: 323.1. ¹H NMR (300 MHz, MeOH-d₄) δ [ppm]: 8.16 (s, 1H), 7.87 (dd, J=8.8, 5.3 Hz, 1H), 7.81 (s, 1H), 7.70 (d, J=8.7 Hz, 1H), 7.52 (s, 1H), 7.41 (d, J=8.7 Hz, 1H), 7.13 (dd, J=9.8, 2.3 Hz, 1H), 6.90 (ddd, J=9.6, 8.8, 2.3 Hz, 1H), 4.73 (t, J=6.7 Hz, 2H), 2.94 (t, J=6.7 Hz, 2H).

### Compound 18

### 6-(6-fluoro-1H-indol-3-yl)-2-(piperidin-4-yl)-2H-indazole

The title compound (26 mg, 5%) was obtained as a yellow solid after purification by preparative HPLC during the preparation of Compound 6. LC-MS for C₂₀H₁₉FN₄+H⁺[M+H]⁺: calcd. 335.2. found: 334.9. ¹H NMR (400 MHz, DMSO-d₆) δ [ppm]: 11.41 (s, 1H), 8.36 (s, 1H), 7.91 (dd, J=8.6, 5.4 Hz, 1H), 7.83 (s, 1H), 7.75-7.70 (m, 2H), 7.39 (d, J=8.6, 1.4 Hz, 1H), 7.24 (dd, J=10.0, 2.4 Hz, 1H), 6.97 (ddd, J=9.6, 8.8, 2.4 Hz, 1H), 4.62-4.50 (m, 1H), 3.22-3.13 (m, 2H), 2.82-2.70 (m, 2H), 2.16-1.93 (m, 4H).

### Compound 19

### 1-(4-(6-(6-fluoro-1H-indol-3-yl)-2H-indazol-2-yl)piperidin-1-yl)ethanone

Following the general method as outlined in Compound 7, starting from 6-(6-fluoro-1H-indol-3-yl)-2-(piperidin-4-yl)-2H-indazole (Compound 18; 300 mg; 0.90 mmol), 20 mg (6%) of the title compound was obtained as a yellow solid after purification by preparative HPLC. LC-MS for C₂₂H₂₁FN₄O+H⁺[M+H]⁺: calcd. 377.2. found: 376.9. ¹H NMR (400 MHz, DMSO-d₆) δ [ppm]: 11.42 (s, 1H), 8.40 (s, 1H), 7.89 (dd, J=8.7, 5.4 Hz, 1H), 7.80 (s, 1H), 7.75-7.71 (m, 2H), 7.39 (d, J=8.5 Hz, 1H), 7.23 (dd, J=10.0, 2.3 Hz, 1H), 6.96 (dd, J=9.4, 8.7, 2.3 Hz, 1H), 4.81-4.71 (m, 1H), 4.56-4.43 (m, 1H), 4.02-3.94 (m, 1H), 3.32-3.22 (m, 1H), 2.82-2.73 (m, 1H), 2.21-1.86 (m, 4H), 2.07 (s, 3H).

### Compound 20

### 2-(5-(6-fluoro-1H-indol-3-yl)-2H-indazol-2-yl)acetamide

The title compound (71 mg, 29%) was obtained as a yellow solid after purification by preparative HPLC and chiral preparative HPLC as a regio-isomer with Compound 9. LC-MS for C₁₇H₁₃FN₄O+H⁺[M+H]⁺: calcd. 309.1. found: 308.9. ¹H NMR (300 MHz, DMSO-d₆) δ [ppm]: 11.38 (s, 1H), 8.31 (s, 1H), 7.97 (s, 1H), 7.90 (dd, J=8.7, 5.5 Hz, 1H), 7.70-7.55 (m, 4H), 7.37 (s, 1H), 7.22 (dd, J=9.9, 2.3 Hz, 1H), 6.96 (ddd, J=9.6, 8.7 2.3 Hz, 1H), 5.08 (s, 2H).

### Compound 21

### 1-(4-((5-(6-fluoro-1H-indol-3-yl)-2H-indazol-2-yl)methyl)piperidin-1-yl)ethanone

Following the general method as outlined in Compound 7, starting from 5-(6-fluoro-1H-indol-3-yl)-2-(piperidin-4-ylmethyl)-2H-indazole (Intermediate 22; 100 mg; 0.29 mmol), 5 mg (4%) of the title compound was obtained as an off-white solid after purification by preparative HPLC. LC-MS for C₂₃H₂₃FN₄O+H⁺[M+H]⁺: calcd. 391.2. found: 390.9. ¹H NMR (400 MHz, MeOH-d₄) δ [ppm]: 8.14 (s, 1H), 7.90 (s, 1H), 7.84 (dd, J=8.8, 5.4 Hz, 1H), 7.68-7.61 (m, 2H), 7.46 (s, 1H), 7.12 (dd, J=9.8, 2.3 Hz, 1H), 6.89 (dd, J=9.7, 8.8, 2.3 Hz, 1H), 4.54-4.47 (m, 1H), 4.11 (d, J=7.0 Hz, 2H), 3.92-3.85 (m, 1H), 3.09-3.00 (m, 1H), 2.62-2.53 (m, 1H), 2.37-2.24 (m, 1H), 2.06 (s, 3H), 1.63-1.54 (m, 2H), 1.34-1.13 (m, 2H).

### Compound 22

### 3-(5-(6-fluoro-1H-indol-3-yl)-2H-indazol-2-yl)propanamide

The title compound (48 mg, 14%) was obtained as a white solid after purification by preparative HPLC and chiral preparative HPLC during the preparation of Compound 12. LC-MS for C₁₈H₁₅FN₄O+H⁺ [M+H]⁺: calcd. 323.1. found: 322.9. ¹H NMR (300 MHz, DMSO-d₆) δ [ppm]: 11.39 (s, 1H), 8.07 (s, 1H), 7.98 (s, 1H), 7.87 (dd, J=8.7, 5.4 Hz, 1H), 7.74-7.66 (m, 3H), 7.43 (s, 1H), 7.23 (dd, J=10.0, 2.0 Hz, 1H), 6.96 (ddd, J=9.3, 8.7 2.0 Hz, 1H), 6.89 (s, 1H), 4.61 (t, J=6.8 Hz, 2H), 2.70 (t, J=6.8 Hz, 2H).

### Compound 23

### 5-(6-fluoro-1H-indol-3-yl)-2-(piperidin-4-yl)-2H-indazole

To the solution of tert-butyl 4-(5-(6-fluoro-1H-indol-3-yl)-2H-indazol-2-yl)piperidine-1-carboxylate (Intermediate 24; 180 mg; 0.41 mmol) in 1,4-dioxane (20 mL) was added saturated HCl in 1,4-dioxane (10 mL). The reaction mixture was stirred at room temperature overnight, filtered, and purified by preparative HPLC to afford 59 mg (43%) of the title compound as a yellow solid. LC-MS for C₂₀H₁₉FN₄+H⁺ [M+H]⁺: calcd. 335.4. found: 334.9. ¹H NMR (300 MHz, DMSO-d₆) δ [ppm]: 11.37 (s, 1H), 8.36 (s, 1H), 7.92 (s, 1H), 7.87 (dd, J=8.8, 5.4 Hz, 1H), 7.68-7.63 (m, 2H), 7.55 (dd, J=9.0, 1.6 Hz, 1H), 7.22 (dd, J=10.0, 2.4 Hz, 1H), 6.96 (ddd, J=9.7, 8.8, 2.4 Hz, 1H), 4.59-4.43 (m, 1H), 3.11-3.02 (m, 2H), 2.70-2.59 (m, 2H), 2.26 (br s, 1H), 2.10-1.89 (m, 4H).

### Compound 24

### 1-(4-(5-(6-fluoro-1H-indol-3-yl)-2H-indazol-2-yl)piperidin-1-yl)ethanone

Following the general method as outlined in Compound 7, starting from 5-(6-fluoro-1H-indol-3-yl)-2-(piperidin-4-yl)-2H-indazole (Compound 23; 53 mg; 0.16 mmol), 16 mg (27%) of the title compound was obtained as an off-white solid after purification by preparative HPLC. LC-MS for C₂₂H₂₁FN₄O+H⁺[M+H]⁺: calcd. 377.4. found: 376.9. ¹H NMR (300 MHz, DMSO-d₆) δ [ppm]: 8.25 (s, 1H), 7.90 (s, 1H), 7.83 (dd, J=8.8, 5.3 Hz, 1H), 7.65-7.61 (m, 2H), 7.45 (s, 1H), 7.11 (dd, J=9.8, 2.4 Hz, 1H), 6.88 (ddd, J=9.6, 8.8, 2.4 Hz, 1H), 4.78-4.67 (m, 2H), 4.15-4.06 (m, 1H), 3.40-3.34 (m, 1H), 2.91-2.80 (m, 1H), 2.30-2.00 (m, 4H), 2.17 (s, 3H).

### Compound 25

### 5-(6-fluoro-1H-indol-3-yl)-1H-benzo[d]imidazole

Following the general method as outlined in Compound 1, starting from 5-(6-fluoro-1-(phenylsulfonyl)-1H-indol-3-yl)-1H-benzo[d]imidazole (Intermediate 27; 550 mg crude; 0.46 mmol), 5 mg (3%) of the title compound was obtained as a yellow solid after purification by preparative HPLC. LC-MS for C₁₅H₁₀FN₃+H⁺ [M+H]⁺: calcd. 252.1. found: 252.0. ¹H NMR (300 MHz, DMSO-d₆) δ [ppm]: 12.44-12.33 (m, 1H), 11.38-11.30 (m, 1H), 8.22-8.17 (m, 1H), 7.88-7.72 (m, 2H), 7.71-7.44 (m, 3H), 7.25-7.18 (m, 1H), 7.01-6.90 (m, 1H).

### Compound 26

### 5-(6-fluoro-1H-indol-3-yl)-2-methyl-1H-benzo[d]imidazole

Following the general method as outlined in Compound 1, starting from 5-(6-fluoro-1-(phenylsulfonyl)-1H-indol-3-yl)-2-methyl-1H-benzo[d]imidazole (Intermediate 30; 870 mg crude; 1.99 mmol), 240 mg (45%) of the title compound was obtained as an off-white solid after purification by preparative HPLC. LC-MS for C₁₆H₁₂FN₃+H⁺ [M+H]⁺: calcd. 266.1. found: 265.9. ¹H NMR (400 MHz, DMSO-d₆) δ [ppm]: 12.16 (s, 1H), 11.34 (s, 1H), 7.82 (dd, J=8.7, 5.5 Hz, 1H), 7.67 (s, 1H), 7.62 (s, 1H), 7.51 (d, J=8.2 Hz, 1H), 7.41 (d, J=8.2 Hz, 1H), 7.22 (dd, J=10.0, 1.9 Hz, 1H), 6.95 (ddd, J=9.6, 8.7, 1.9 Hz, 1H), 2.50 (s, 3H).

### Compound 27

### (6-(6-fluoro-1H-indol-3-yl)-1H-benzo[d]imidazol-2-yl)methanamine

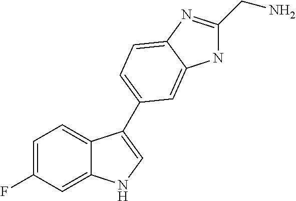 [SMILES: NCc1nc2ccc(-c3c[nH]c4cc(F)ccc34)cc2[nH]1]

To a solution of tert-butyl((6-(6-fluoro-1H-indol-3-yl)-1H-benzo[d]imidazol-2-yl)methyl)carbamate (Intermediate 48, 429 mg, 1.13 mmol) in THF (30 mL) was added HCl (12 M, 3 mL). The resulting mixture was stirred at 51° C. for 3 hours. The reaction mixture was cooled to room temperature and concentrated. The residue was diluted with water (80 mL) and extracted with EtOAc (30 mL×2). The aqueous phase was basified with NH₄OH to pH=13 and extracted with EtOAc (80 mL×3). The combined organic layer was washed with brine (30 mL), dried over anhydrous Na₂SO₄ and filtered. The filtrate was concentrated and purified by preparative HPLC to afford 70 mg (22%) of the title compound as a white solid. LC-MS for C₁₆H₁₃FN₄+H⁺[M+H]⁺: calcd. 281.1. found: 280.9. ¹H NMR (400 MHz, DMSO-d₆) δ [ppm]: 11.34 (s, 1H), 7.82 (dd, J=8.8, 5.5 Hz, 1H), 7.70 (s, 1H), 7.61 (s, 1H), 7.54 (d, J=8.2 Hz, 1H), 7.42 (d, J=8.3 Hz, 1H), 7.22 (dd, J=9.9, 2.3 Hz, 1H), 6.95 (td, J=9.4, 2.1 Hz, 1H), 3.94 (s, 2H).

### Compound 28

### 1-(6-(6-fluoro-1H-indol-3-yl)-1H-benzo[d]imidazol-2-yl)-N,N-dimethylmethanamine

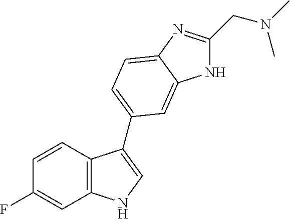 [SMILES: CN(C)Cc1nc2ccc(-c3c[nH]c4cc(F)ccc34)cc2[nH]1]

Following the general method as outlined in Compound 1, starting from 1-(6-(6-fluoro-1-(phenylsulfonyl)-1H-indol-3-yl)-1H-benzo[d]imidazol-2-yl)-N,N-dimethylmethanamine (Intermediate 50, 391 mg, 0.87 mmol), 10 mg (4%) of the title compound was obtained as a white solid. LC-MS for C₁₈H₁₇FN₄+H⁺ [M+H]⁺: Calcd. 309.1. found: 309.1. ¹H NMR (400 MHz, CDCl₃) δ [ppm]: 10.06-9.46 (m, 1H), 8.31 (s, 1H), 7.86 (dd, J=8.6, 5.3 Hz, 1H), 7.80-7.62 (m, 1H), 7.52 (d, J=6.8 Hz, 1H), 7.35 (d, J=2.3 Hz, 1H), 7.12 (dd, J=9.5, 2.2 Hz, 1H), 6.96 (td, J=9.3, 2.3 Hz, 1H), 3.80 (s, 2H), 2.38 (s, 6H).

### Compound 30

### N-((6-(6-fluoro-1H-indol-3-yl)-1H-benzo[d]imidazol-2-yl)methyl)acetamide

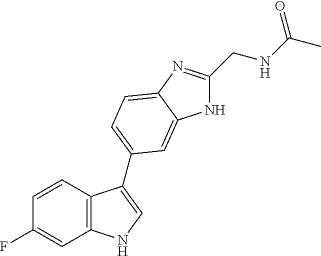 [SMILES: CC(=O)NCc1nc2ccc(-c3c[nH]c4cc(F)ccc34)cc2[nH]1]

Following the general method as outlined in Compound 1, starting from N-((6-(6-fluoro-1-(phenylsulfonyl)-1H-indol-3-yl)-1H-benzo[d]imidazol-2-yl)methyl)acetamide (Intermediate 53, 400 mg crude, 0.74 mmol), 20 mg (9%) of the title compound was obtained as a white solid after purification by preparative HPLC. LC-MS for C₁₈H₁₅FN₄O+H⁺[M+H]⁺: calcd. 323.1. found: 322.1. ¹H NMR (400 MHz, MeOH-d₄) 6 [ppm]: 7.76 (dd, J=8.8, 5.2 Hz, 1H), 7.71 (s, 1H), 7.53 (d, J=8.4 Hz, 1H), 7.48 (d, J=8.4 Hz, 1H), 7.38 (s, 1H), 7.07 (d, J=9.6 Hz, 1H), 6.83 (td, J=9.2, 2.4 Hz, 1H), 4.59 (s, 2H), 2.03 (s, 3H).

### Compound 31

### 2-amino-N-((6-(6-fluoro-1H-indol-3-yl)-1H-benzo[d]imidazol-2-yl)methyl)acetamide

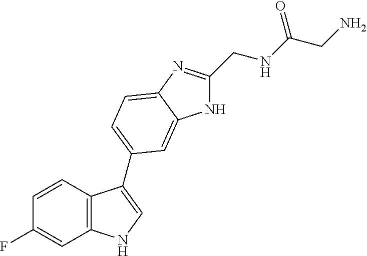 [SMILES: NCC(=O)NCc1nc2ccc(-c3c[nH]c4cc(F)ccc34)cc2[nH]1]

To a stirred solution of tert-butyl(2-(((6-(6-fluoro-1H-indol-3-yl)-1H-benzo[d]imidazol-2-yl)methyl)amino)-2-oxoethyl)carbamate (Intermediate 51, 650 mg, 1.43 mmol) in THF (20 mL) was added HCl (12 M, 3 mL). The resulting mixture was stirred for 40 mins at room temperature. The solvents was removed under reduced pressure. The residue was diluted with water (80 mL) and extracted with EtOAc (30 mL×2). The aqueous phase was basified with NH₄OH till pH=13 and extracted with EtOAc (80 mL×3). The combined organic layer was washed with brine (30 mL), dried over anhydrous Na₂SO₄ and filtered. The filtrate was concentrated and purified by preparative HPLC to afford 70 mg (15%) of the title compound as a white solid. LC-MS for C₁₈H₁₆FN₅O+H⁺ [M+H]⁺: calcd. 338.1. found: 337.9. ¹H NMR (400 MHz, DMSO-d₆) δ [ppm]: 11.36 (s, 1H), 8.55 (s, 1H), 7.83 (dd, J=8.7, 5.4 Hz, 1H), 7.73 (s, 1H), 7.63 (d, J=1.7 Hz, 1H), 7.56 (d, J=8.3 Hz, 1H), 7.45 (d, J=8.3 Hz, 1H), 7.22 (dd, J=10.0, 2.2 Hz, 1H), 6.95 (td, J=9.5, 2.2 Hz, 1H), 4.55 (s, 2H), 3.22 (s, 2H).

### Compound 32

### N-((6-(6-fluoro-1H-indol-3-yl)-1H-benzo[d]imidazol-2-yl)methyl)methanesulfonamide

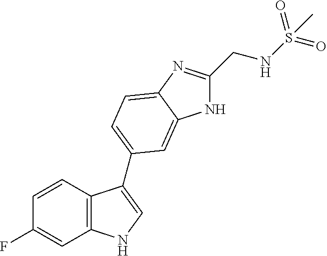 [SMILES: CS(=O)(=O)NCc1nc2ccc(-c3c[nH]c4cc(F)ccc34)cc2[nH]1]

To a stirred solution of c-[6-(6-fluoro-1H-indol-3-yl)-1H-benzoimidazol-2-yl]-methylamine (Compound 27, 300 mg, 1.07 mmol) and Et₃N (270 mg, 2.68 mmol) in anhydrous DCM (24 mL) was added MsCl (123 mg, 1.07 mmol) at −10° C. under nitrogen. The reaction mixture was stirred at 5° C. overnight. The reaction mixture was diluted with EtOAc (80 mL) and washed with saturated aqueous Na₂CO₃. The combined organic layers was dried over anhydrous Na₂SO₄ and filtered. The filtrate was concentrated and purified by preparative HPLC to afford 20 mg (44%) of the title compound as a yellow solid. LC-MS for C₁₇H₁₅FN₄O₂S—H⁻ [M−H]⁻: calcd. 357.1. found: 356.9. ¹H NMR (400 MHz, MeOD) 6 [ppm]: 7.74 (dd, J=8.8, 5.3 Hz, 1H), 7.70 (s, 1H), 7.52 (d, J=8.4 Hz, 1H), 7.46 (dd, J=8.4, 1.2 Hz, 1H), 7.35 (s, 1H), 7.04 (dd, J=9.8, 2.3 Hz, 1H), 6.80 (td, J=9.3, 2.3 Hz, 1H), 4.46 (s, 2H), 2.92 (s, 3H).

### Compound 33

### 6-(6-fluoro-1H-indol-3-yl)-2-((4-methylpiperazin-1-yl)methyl)-1H-benzo[d]imidazole

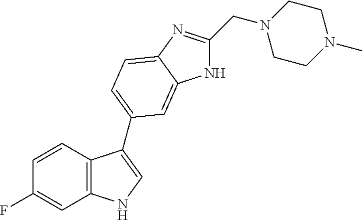 [SMILES: CN1CCN(Cc2nc3ccc(-c4c[nH]c5cc(F)ccc45)cc3[nH]2)CC1]

Following the general method as outlined in Compound 1, starting from 6-(1-benzenesulfonyl-6-fluoro-1H-indol-3-yl)-2-(4-methyl-piperazin-1-ylmethyl)-1H-benzoimidazole (Intermediate 60, 290 mg, 0.45 mmol, crude), 18 mg (11%) of the title compound was obtained as a brown solid. LC-MS for C₂₁H₂₂FN₅+H⁺ [M+H]⁺: calcd. 364.2. found: 364.2. ¹H NMR (400 MHz, DMSO-d₆) δ [ppm]: 11.69 (br s, 1H), 11.10 (br s, 1H), 7.99 (s, 1H), 7.86-7.84 (m, 4H), 7.28 (dd, J=9.8, 2.0 Hz, 1H), 7.02 (td, J=9.6, 2.0 Hz, 1H), 4.26 (s, 2H), 3.43-3.40 (m, 2H), 3.18-3.10 (m, 4H), 2.84-2.76 (m, 5H).

### Compound 34

### 5-(6-fluoro-1H-indol-3-yl)-2-((4-(methylsulfonyl)piperazin-1-yl)methyl)-1H-benzo[d]imidazole

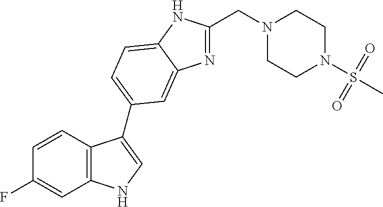 [SMILES: CS(=O)(=O)N1CCN(Cc2nc3cc(-c4c[nH]c5cc(F)ccc45)ccc3[nH]2)CC1]

Following the general method as outlined in Compound 1, starting from 6-(1-benzenesulfonyl-6-fluoro-1H-indol-3-yl)-2-(4-methanesulfonyl-piperazin-1-ylmethyl)-1H-benzoimidazole (Intermediate 67, 80 mg, 0.14 mmol), 15 mg (25%) of the title compound was obtained as a white solid. LC-MS for C₂₁H₂₂FN₅O₂S+H⁺[M+H]⁺: calcd. 428.2. found: 428.2. ¹H NMR (400 MHz, CD₃OD) δ [ppm]: 7.72 (dd, J=8.8, 5.2 Hz, 1H), 7.67 (s, 1H), 7.49 (d, J=8.8 Hz, 1H), 7.43 (dd, J=8.4, 0.9 Hz, 1H), 7.33 (s, 1H), 7.02 (dd, J=9.6, 2.0 Hz, 1H), 7.78 (d, J=8.8, 2.0 Hz, 1H), 3.77 (s, 2H), 3.28-3.20 (m, 4H), 2.75 (s, 3H), 2.62-2.52 (m, 4H).

### Compound 35

### 2-(6-(6-fluoro-1H-indol-3-yl)-1H-benzo[d]imidazol-2-yl)ethanamine

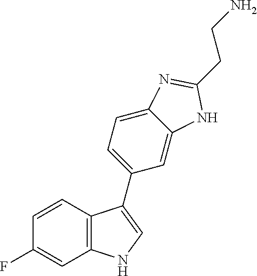 [SMILES: NCCc1nc2ccc(-c3c[nH]c4cc(F)ccc34)cc2[nH]1]

Following the general method as outlined in Compound 1, starting from 2-[6-(1-Benzenesulfonyl-6-fluoro-1H-indol-3-yl)-1H-benzoimidazol-2-yl]-ethylamine (Intermediate 64, 400 mg, 0.920 mmol), the title compound (40 mg, 15%) was obtained as a white solid. LC-MS for C₁₇H₁₅FN₄+H⁺[M+H]⁺: calcd. 295.1. found: 295.1. ¹H NMR (400 MHz, CD₄O-d₄) δ [ppm]: 7.86-7.82 (m, 2H), 7.61-7.53 (m, 2H), 7.46 (s, 1H), 7.14 (dd, J=10.0, 2.4 Hz, 1H), 6.91 (td, J=9.2, 2.4 Hz, 1H), 3.28-3.22 (m, 2H), 3.16-3.08 (m, 2H).

### Compound 36

### N-(2-(6-(6-fluoro-1H-indol-3-yl)-1H-benzo[d]imidazol-2-yl)ethyl)acetamide

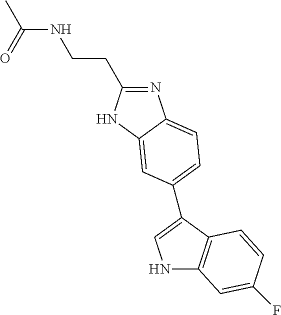 [SMILES: CC(=O)NCCc1nc2ccc(-c3c[nH]c4cc(F)ccc34)cc2[nH]1]

Following the general method as outlined in Compound 1, starting from N-{2-[6-(1-Benzenesulfonyl-6-fluoro-1H-indol-3-yl)-1H-benzoimid-azol-2-yl]-ethyl}-acetamide (Intermediate 66, 424 mg, 0.890 mmol), the title compound (86 mg, 29%) was obtained as a white solid. LC-MS for C₁₉H₁₇FN₄O+H⁺[M+H]⁺: calcd. 337.1. found: 337.1. ¹H NMR (400 MHz, CD₄O-d₄) δ [ppm]: 7.70 (dd, J=8.8, 5.6 Hz, 2H), 7.64 (s, 1H), 7.47-7.40 (m, 2H), 7.32 (s, 1H), 7.02 (dd, J=9.6, 2.2 Hz, 1H), 6.77 (td, J=9.2, 2.4 Hz, 1H), 3.54 (t, J=6.8 Hz, 2H), 2.99 (t, J=6.8 Hz, 2H).

### Compound 37

### N-(2-(6-(6-fluoro-1H-indol-3-yl)-1H-benzo[d]imidazol-2-yl)ethyl)methanesulfonamide

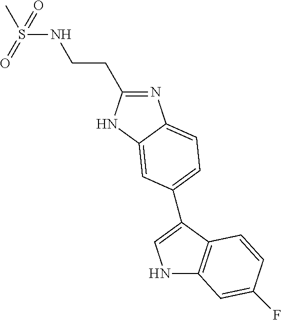 [SMILES: CS(=O)(=O)NCCc1nc2ccc(-c3c[nH]c4cc(F)ccc34)cc2[nH]1]

Following the general method as outlined in Compound 1, starting from N-{2-[6-(1-Benzenesulfonyl-6-fluoro-1H-indol-3-yl)-1H-benzoimida-zol-2-yl]-ethyl}-methanesulfonamide (Intermediate 65, 430 mg, 0.840 mmol), the title compound (36 mg, 11%) was botained as a white solid. LC-MS for C₁₈H₁₇FN₄O₂S+H⁺[M+H]⁺: calcd. 373.1. found: 373.1. ¹H NMR (400 MHz, CD₄O-d₄) δ [ppm]: 7.73-7.66 (m, 2H), 7.51-7.44 (m, 2H), 7.34 (s, 1H), 7.02 (dd, J=10.0, 2.0 Hz, 1H), 6.78 (td, J=8.8, 2.0 Hz, 1H), 3.48 (t, J=7.0 Hz, 2H), 3.11 (m, J=7.0 Hz, 2H), 2.81 (s, 3H).

### Compound 38

### 5-(6-fluoro-1H-indol-3-yl)-2-(2-(methylsulfonyl)ethyl)-1H-benzo[d]imidazole

Following the general method as outlined in Compound 1, starting from 5-(6-fluoro-1-(phenylsulfonyl)-1H-indol-3-yl)-2-(2-(methylsulfonyl)ethyl)-1H-benzo[d]imidazole (Intermediate 34; 170 mg; 0.34 mmol), 20 mg (16%) of the title compound was obtained as a white solid after purification by preparative TLC (DCM/MeOH=10/1) and preparative HPLC. LC-MS for C₁₈H₁₆FN₃O₂S+H⁺[M+H]⁺: calcd. 358.1. found: 358.1. ¹H NMR (400 MHz, DMSO-d₆) δ [ppm]: 12.35 (s, 0.45H), 12.35 (s, 0.55H), 11.37 (s, 0.55H), 11.33 (s, 0.45H), 7.85-7.79 (m, 1H), 7.77 (s, 0.45H), 7.66-7.63 (m, 1H), 7.62-7.57 (m, 1H), 7.52-7.41 (m, 1.55H), 7.24-7.19 (m, 1H), 6.99-6.91 (m, 1H), 3.71-3.65 (m, 2H), 3.32-3.27 (m, 2H), 2.32 (s, 3H) as a mixture of tautomers.

### Compound 39

### 6-(6-fluoro-1H-indol-3-yl)-2-(piperidin-4-yl)-1H-benzo[d]imidazole

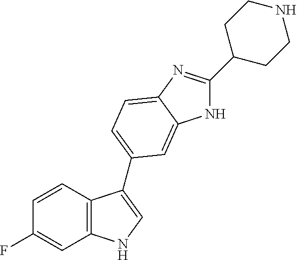 [SMILES: Fc1ccc2c(-c3ccc4nc(C5CCNCC5)[nH]c4c3)c[nH]c2c1]

To a solution of tert-butyl 4-(6-(6-fluoro-1H-indol-3-yl)-1H-benzo[d]imidazol-2-yl)piperidine-1-carboxylate (Intermediate 56, 550 mg crude, 1.05 mmol) in THF (5 mL) was added concentrated aqueous HCl (1 mL). The reaction mixture was stirred at 50° C. for 3 hours before it was concentrated. The mixture was poured into brine and extracted with EtOAc. The combined organic layer was dried over anhydrous Na₂SO₄, filtered and concentrated to afford 200 mg (57%) of the title compound as a yellow solid. LC-MS for C₂₀H₁₉FN₄+H [M+H]⁺: calcd. 335.2. found: 335.1. ¹H NMR (400 MHz, MeOH-d₄) 6 [ppm]: 7.84 (dd, J=8.8, 5.2 Hz, 1H), 7.79 (s, 1H), 7.62 (d, J=8.4 Hz, 1H), 7.56 (d, J=8.4 Hz, 1H), 7.46 (s, 1H), 7.15 (dd, J=9.8, 2.2 Hz, 1H), 6.91 (td, J=9.2, 2.0 Hz, 1H), 3.54-3.51 (m, 2H), 3.31-3.28 (m, 1H), 3.21-3.15 (m, 2H), 2.38-2.35 (m, 2H), 2.20-2.13 (m, 2H).

### Compound 40

### 6-(6-fluoro-1H-indol-3-yl)-2-(1-(methylsulfonyl)piperidin-4-yl)-1H-benzo[d]imidazole

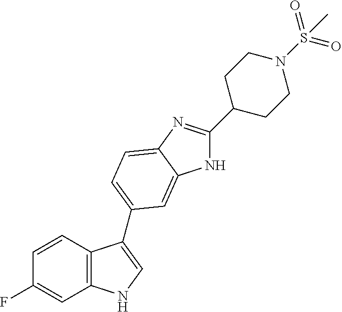 [SMILES: CS(=O)(=O)N1CCC(c2nc3ccc(-c4c[nH]c5cc(F)ccc45)cc3[nH]2)CC1]

To a solution of 6-(6-fluoro-1H-indol-3-yl)-2-(piperidin-4-yl)-1H-benzo[d]imidazole (Compound 39, 100 mg, 0.30 mmol) and Et₃N (290 mg, 2.9 mmol) in DCM (2 mL) was added MsCl (16 mg, 0.14 mmol) dropwise. The reaction mixture was stirred at room temperature for 2 hours, washed with brine, concentrated and purified by preparative HPLC to afford 8 mg (6%) of the title compound as a white solid. LC-MS for C₂₁H₂₁FN₄O₂S+H [M+H]⁺: calcd. 413.1. found: 413.1. ¹H NMR (400 MHz, MeOH-d₄) 6 [ppm]: 7.72 (dd, J=8.4, 5.2 Hz, 1H), 7.65 (s, 1H), 7.47 (d, J=8.4 Hz, 1H), 7.41 (d, J=8.4, 1H), 7.33 (s, 1H), 7.02 (dd, J=9.8, 2.4 Hz, 1H), 6.78 (td, J=9.2, 2.0 Hz, 1H), 3.75 (d, J=12.0 Hz, 2H), 3.02 (tt, J=11.7, 3.6 Hz, 1H), 2.86-2.77 (m, 2H), 2.73 (s, 3H), 2.13-2.09 (m, 2H), 1.96-1.86 (m, 2H).

### Compound 41

### 6-(6-fluoro-1H-indol-3-yl)-1H-benzo[d]imidazol-2-amine

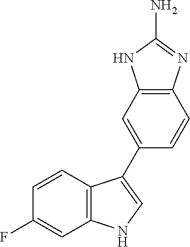 [SMILES: Nc1nc2ccc(-c3c[nH]c4cc(F)ccc34)cc2[nH]1]

Following the general method as outlined in Compound 1, starting from 6-(6-fluoro-1-(phenylsulfonyl)-1H-indol-3-yl)-1H-benzo[d]imidazol-2-amine (Intermediate 85, 260 mg, 0.64 mmol), 60 mg (35%) of the title compound was obtained as a yellow solid after purification by preparative HPLC. LC-MS for C₁₅H₁₁FN₄+H⁺ [M+H]⁺: calcd. 267.1. found: 267.1. ¹H NMR (400 MHz, DMSO-d₆) δ [ppm]: 11.21 (br s, 1H), 10.61 (br s, 1H), 7.78 (dd, J=8.8, 5.5 Hz, 1H), 7.49 (d, J=2.0 Hz, 1H), 7.34 (s, 1H), 7.18 (dd, J=10.1, 2.2 Hz, 1H), 7.16-7.11 (m, 2H), 6.92 (ddd, J=9.6, 8.6, 2.2 Hz, 1H), 6.11 (br s, 2H).

### Compound 44

### 1-(5-(6-fluoro-1H-indol-3-yl)-1H-benzo[d]imidazol-2-yl)-N-methylmethanamine

To a solution of tert-butyl(5-(6-fluoro-1H-indol-3-yl)-1H-benzo[d]imidazol-2-yl)methyl(methyl)carbamate (Intermediate 37; 470 mg crude; 0.87 mmol) in THF (30 mL) was added concentrated aqueous HCl (3 mL; 37%). The reaction mixture was stirred at 51° C. for 3 hours, concentrated in vacuo, diluted with water (80 mL), and extracted with EtOAc (50 mL×3). The aqueous layer was basified with aqueous NaOH to pH=13 and extracted with EtOAc (80 mL×3). The combined organic layers were dried over anhydrous Na₂SO₄, filtered, concentrated, and purified by preparative TLC (DCM/MeOH=10/1) and preparative HPLC to afford 40 mg (16%) of the title compound as a white solid. LC-MS for C₁₇H₁₅FN₄+H⁺[M+H]⁺: calcd. 295.1. found: 294.9. ¹H NMR (400 MHz, DMSO-d₆) δ [ppm]: 11.34 (s, 1H), 7.82 (dd, J=8.7, 5.4 Hz, 1H), 7.70 (s, 1H), 7.61 (s, 1H), 7.53 (d, J=8.7 Hz, 1H), 7.42 (d, J=8.7 Hz, 1H), 7.22 (dd, J=10.0, 2.3 Hz, 1H), 6.95 (ddd, J=9.4, 8.7, 2.3 Hz, 1H), 3.87 (s, 2H), 2.34 (s, 3H).

### Compound 45

### 4-((5-(6-fluoro-1H-indol-3-yl)-1H-benzo[d]imidazol-2-yl)methyl)morpholine

### Step 1

### 4-((5-(6-fluoro-1-(phenylsulfonyl)-1H-indol-3-yl)-1H-benzo[d]imidazol-2-yl)methyl)morpholine

A solution of 2-(chloromethyl)-5-(6-fluoro-1-(phenylsulfonyl)-1H-indol-3-yl)-1H-benzo[d]imidazole (Intermediate 39; 100 mg; 0.23 mmol) and morpholine (59 mg; 0.68 mmol) in DMF (2 mL) was stirred at 80° C. for 2 hours. The mixture was diluted with EtOAc (50 mL), washed with H₂O (20 mL), brine (30 mL×2), dried over anhydrous Na₂SO₄, filtered, concentrated, and purified by preparative TLC (DCM/MeOH=20/1) to afford 56 mg (50%) of the title compound as a brown solid. LC-MS for C₂₆H₂₃FN₄O₃S+H⁺[M+H]⁺: calcd. 491.2. found: 490.9.

### Step 2

Following the general method as outlined in Compound 1, starting from 4-((5-(6-fluoro-1-(phenylsulfonyl)-1H-indol-3-yl)-1H-benzo[d]imidazol-2-yl)methyl)morpholine (Step 1; 56 mg; 0.11 mmol), 13 mg (32%) of the title compound was obtained as a white solid after purification by preparative TLC (DCM/MeOH=20/1). LC-MS for C₂₀H₁₉FN₄O+H⁺[M+H]⁺: calcd. 351.2. found: 351.2. ¹H NMR (400 MHz, MeOH-d₄) δ [ppm]: 7.71 (dd, J=8.4, 5.2 Hz, 1H), 7.67 (s, 1H), 7.49 (d, J=8.4 Hz, 1H), 7.41 (d, J=8.4 Hz, 1H), 7.33 (s, 1H), 7.01 (dd, J=10.0, 2.4 Hz, 1H), 6.78 (ddd, J=9.2, 8.4, 2.4 Hz, 1H), 3.72 (s, 1H), 3.66-3.61 (m, 4H), 2.50-2.44 (m, 4H).

### Compound 46

### 3-(5-(6-fluoro-1H-indol-3-yl)-1H-benzo[d]imidazol-2-yl)propanamide

### Step 1

### 3-(5-(6-fluoro-1-(phenylsulfonyl)-1H-indol-3-yl)-1H-benzo[d]imidazol-2-yl)propanamide

A solution of 6-(6-fluoro-1-(phenylsulfonyl)-1H-indol-3-yl)-2,3-dihydro-1H-benzo[d]pyrrolo[1,2-a]imidazol-1-one (Intermediate 41; 456 mg crude; 0.92 mmol) in saturated NH₃ in THF (100 mL) was stirred at 137° C. for 24 hours in an autoclave. The reaction mixture was cooled to room temperature and concentrated to afford 480 mg (100%) of the title compound as a yellow solid, which was used directly without further purification. LC-MS for C₂₄H₁₉FN₄O₃S+H⁺[M+H]⁺: calcd. 463.1. found: 462.8.

### Step 2

To a solution of 3-(5-(6-fluoro-1-(phenylsulfonyl)-1H-indol-3-yl)-1H-benzo[d]imidazol-2-yl)propanamide (Step 1; 480 mg crude; 0.92 mmol) in EtOH (10 mL) was added KOH (116 mg; 2.07 mmol). The reaction mixture was stirred at 50° C. for 4 hours. The mixture was neutralized with 2 M aqueous HCl, concentrated, and purified by preparative HPLC to afford 50 mg (17%) of the title compound as a white solid. LC-MS for C₁₈H₁₅FN₄O+H⁺[M+H]⁺: calcd. 323.1. found: 322.9. ¹H NMR (400 MHz, DMSO-d₆) δ [ppm]: 12.15 (s, 1H), 11.33 (s, 1H), 7.82 (dd, J=8.7, 5.5 Hz, 1H), 7.67 (s, 1H), 7.61 (d, J=1.9 Hz, 1H), 7.51 (d, J=8.1 Hz, 1H), 7.48-7.38 (m, 2H), 7.22 (dd, J=10.0, 2.0 Hz, 1H), 6.95 (ddd, J=9.6, 8.7, 2.0 Hz, 1H), 6.86 (s, 1H), 3.03 (t, J=7.6 Hz, 2H), 2.64 (t, J=7.6 Hz, 2H).

### Compound 47

### 5-(6-fluoro-1H-indol-3-yl)benzo[d]oxazole

Following the general method as outlined in Compound 1, starting from 5-(6-fluoro-1-(phenylsulfonyl)-1H-indol-3-yl)benzo[d]oxazole (Intermediate 43; 423 mg; 1.08 mmol), 60 mg (22%) of the title compound was obtained as a white solid after purification by a silica gel chromatography (petroleum ether/EtOAc=100/1-6/1). LC-MS for C₁₅H₉FN₂O+H⁺[M+H]⁺: calcd. 253.1. found: 253.0. ¹H NMR (400 MHz, CDCl₃) δ [ppm]: 8.30 (br s, 1H), 8.14 (s, 1H), 8.03 (s, 1H), 7.84 (dd, J=8.8, 5.2 Hz, 1H), 7.69-7.63 (m, 2H), 7.37 (d, J=2.4 Hz, 1H), 7.14 (dd, J=9.4, 2.2 Hz, 1H), 6.98 (ddd, J=9.4, 8.8, 2.2 Hz, 1H).

### Compound 48

### 5-(6-fluoro-1H-indol-3-yl)-2-methylbenzo[d]oxazole

Following the general method as outlined in Compound 1, starting from 5-(6-fluoro-1-(phenylsulfonyl)-1H-indol-3-yl)-2-methylbenzo[d]oxazole (Intermediate 45; 718 mg; 1.77 mmol), 184 mg (39%) of the title compound was obtained as a brown solid after purification by recrystallization in petroleum ether/EtOAc=4/1. LC-MS for C₁₆H₁₁FN₂O+H⁺[M+H]⁺: calcd. 267.1. found: 267.0. ¹H NMR (400 MHz, DMSO-d₆) δ [ppm]: 11.45 (s, 1H), 7.87 (s, 1H), 7.83 (dd, J=8.8, 5.2 Hz, 1H), 7.73-7.68 (m, 2H), 7.63 (d, J=8.0 Hz, 1H), 7.50 (dd, J=10.0, 2.4 Hz, 1H), 7.23 (ddd, J=9.2, 8.8, 2.4 Hz, 1H), 2.63 (s, 3H).

### Compound 53

### 6-(6-fluoro-1H-indol-3-yl)benzo[d]oxazole

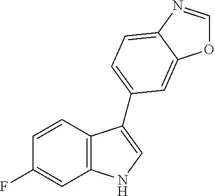 [SMILES: Fc1ccc2c(-c3ccc4ncoc4c3)c[nH]c2c1]

A mixture of 2-amino-5-(6-fluoro-1H-indol-3-yl)-phenol (Intermediate 57, 500 mg, 2.66 mmol) in triethyl orthoformate (15 mL) was heated at 120° C. for 0.5 hour. The mixture was concentrated, suspended in water (20 mL) and filtered. The cake was recrystalized from a mixture of EtOAc (5 mL) and petroleum ether (20 mL) to afford 160 mg (35%) of the title compound as a white solid. LC-MS for C₁₅H₉FN₂O+H⁺ [M+H]⁺: calcd. 253.1. found: 253.0. ¹H NMR (400 MHz, DMSO-d₆) δ [ppm]: 8.43 (s, 1H), 7.89 (s, 1H), 7.84-7.82 (m, 1H), 7.75 (d, J=8.0 Hz, 1H), 7.70 (dd, J=8.4, 1.6 Hz, 1H), 7.52 (s, 1H), 7.12 (dd, J=10, 2.4 Hz, 1H), 6.90 (m, 1H).

### Compound 54

### 6-(6-fluoro-1H-indol-3-yl)-2-methylbenzo[d]oxazole

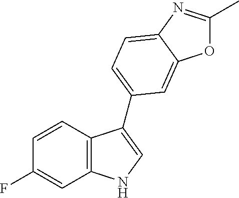 [SMILES: Cc1nc2ccc(-c3c[nH]c4cc(F)ccc34)cc2o1]

A mixture of 2-amino-5-(6-fluoro-1H-indol-3-yl)-phenol (Intermediate 57, 500 mg; 2.66 mmol) in 1,1,1-triethoxyethane (15 mL) was heated at 120° C. for 0.5 hour. The mixture was concentrated, suspended in water (20 mL) and filtered. The cake was recrystalized from a mixture of EtOAc (5 mL) and petroleum ether (20 mL) to afford 140 mg (30%) of the title compound as a white solid. LC-MS for C₁₆H₁₁FN₂O+H⁺[M+H]⁺: calcd. 267.1. found: 267.1. ¹H NMR (400 MHz, DMSO-d₆) δ [ppm]: 7.85 (dd, J=8.8, 5.6 Hz, 1H), 7.82 (s, 1H), 7.66 (s, 2H), 7.53 (s, 1H), 7.16 (dd, J=9.6, 2.4 Hz, 1H), 6.93 (td, J=9.2, 2.0 Hz, 1H), 2.67 (s, 3H).

### Compound 55

### 6-(6-fluoro-1H-indol-3-yl)-2-(piperidin-4-yl)benzo[d]oxazole

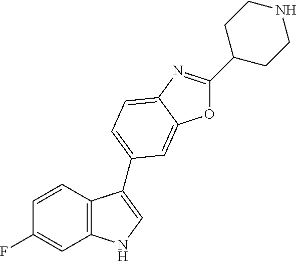 [SMILES: Fc1ccc2c(-c3ccc4nc(C5CCNCC5)oc4c3)c[nH]c2c1]

A mixture of tert-butyl 4-(6-(6-fluoro-1H-indol-3-yl)benzo[d]oxazol-2-yl)piperidine-1-carboxylate (Intermediate 106, 1.23 g, 2.82 mmol) in HCl/ether (40 mL) was stirred at room temperature for 1 hour. The mixture was concentrated, diluted with saturated aqueous NaHCO₃ (50 mL) and extracted with EtOAc (50 mL×3). The combined organic layer was washed with brine (50 mL), dried over anhydrous Na₂SO₄ and filtered. The filtrate was concentrated and purified by recystallization to afford 783 mg (83%) of the title compound as a gray solid. LC-MS for C₂₀H₁₈FN₃O+H⁺[M+H]⁺: calcd. 335.1. found: 336.1. ¹H NMR (400 MHz, DMSO-d₆) δ [ppm]: 11.49 (br s, 1H), 7.91 (s, 1H), 7.89 (dd, J=8.8, 3.2 Hz, 1H), 7.75 (d, J=1.6 Hz, 1H), 7.73 (d, J=8.4 Hz, 1H), 7.66 (dd, J=8.6, 1.4 Hz, 1H), 7.25 (dd, J=9.8, 2.6 Hz, 1H), 6.97 (td, J=10.6, 2.5 Hz, 1H), 3.13-3.02 (m, 3H), 2.64 (t, J=11.2 Hz, 2H), 2.03 (d, J=10.8 Hz, 2H), 1.72 (td, J=12.2, 3.0 Hz, 2H).

### Compound 56

### 1-(4-(6-(6-fluoro-1H-indol-3-yl)benzo[d]oxazol-2-yl)piperidin-1-yl)ethanone

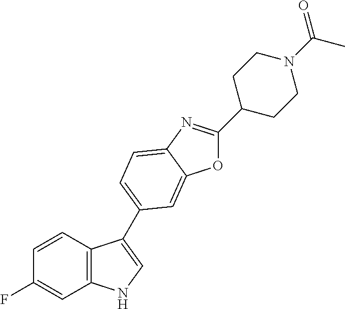 [SMILES: CC(=O)N1CCC(c2nc3ccc(-c4c[nH]c5cc(F)ccc45)cc3o2)CC1]

To a stirred solution of 6-(6-fluoro-1H-indol-3-yl)-2-(piperidin-4-yl)benzo[d]oxazole (Compound 55, 200 mg, 0.60 mmol) and TEA (182 mg, 1.80 mmo) in DCM (10 mL) at room temperature, was added dropwise acetyl chloride (57 mg, 0.72 mmol). The mixture was stirred at room temperature overnight. The mixture was diluted with DCM (10 mL), washed with water (20 mL×2), dried over Na₂SO₄ and filtered. The filtrate was concentrated and purified by preparative HPLC to afford 121 mg (54%) of the title compound as a yellow solid. LC-MS for C₂₂H₂₀FN₃O₂+H⁺: [M+H]⁺: calcd. 378.2. found: 378.1. ¹H NMR (400 MHz, DMSO-d₆) δ [ppm]: 11.48 (s, 1H), 7.92 (s, 1H), 7.89 (dd, J=5.6, 8.8 Hz, 1H), 7.75 (d, J=2.8 Hz, 1H), 7.73 (d, J=7.2 Hz, 1H), 7.66 (dd, J=1.6, 8.0 Hz, 1H), 7.23 (dd, J=2.4, 10.0 Hz, 1H), 6.97 (td, J=2.4, 9.6 Hz, 1H), 4.32 (d, J=13.6 Hz, 1H), 3.87 (d, J=13.6 Hz, 1H), 3.35-3.27 (m, 2H), 2.88 (t, J=11.2 Hz, 1H), 2.13 (t, J=14.0 Hz, 2H), 2.04 (s, 3H), 1.88-1.77 (m, 1H), 1.72-1.62 (m, 1H).

### Compound 58

### N-(6-(6-fluoro-1H-indol-3-yl)benzo[d]oxazol-2-yl)methanesulfonamide

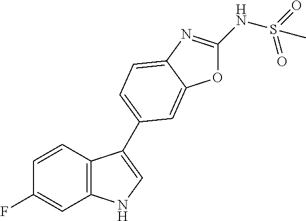 [SMILES: CS(=O)(=O)Nc1nc2ccc(-c3c[nH]c4cc(F)ccc34)cc2o1]

To the mixture of N-[6-(1-Benzenesulfonyl-6-fluoro-1H-indol-3-yl)-benzooxazol-2-yl]-methanesulfonamide (Intermediate 124, 350 mg 0.72 mmol) in ethanol (20 mL) was added potassium hydroxide (55 mg, 2.88 mmol). The mixture was stirred at 50° C. for 4 hrs. The solvent was removed. The residue was redissolved in EA and washed with water and brine. The organic layer was dried, filtered and concentrated. The residue was purified by preparative TLC (DCM/methanol=10/1) and further purified by trituration with EA to afford 8 mg (3%) of the title compound as a black solid. LC-MS for C₁₆H₁₂FN₃O₃S+H⁺[M+H]⁺: calcd. 346.1. found: 346.0. ¹H NMR (400 MHz, DMSO-d₆) δ [ppm]: 12.51 (s, 1H), 11.44 (s, 1H), 7.86 (dd, J=9.2, 5.6 Hz, 1H), 7.76 (s, 1H), 7.72 (d, J=2.0 Hz, 1H), 7.59 (d, J=8.0 Hz, 1H), 7.36 (d, J=8.4 Hz, 1H), 7.22 (dd, J=10.0, 2.4 Hz, 1H), 6.98-6.94 (m, 1H), 3.08 (s, 3H).

### Compound 59

### 6-(6-fluoro-1H-indol-3-yl)-2-(piperazin-1-ylmethyl)-1H-benzo[d]imidazole

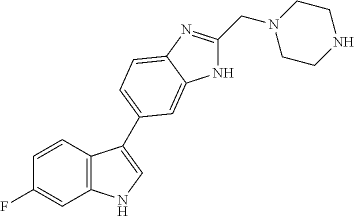 [SMILES: Fc1ccc2c(-c3ccc4nc(CN5CCNCC5)[nH]c4c3)c[nH]c2c1]

Following the general method as outlined in Compound 1, starting from 6-(1-benzenesulfonyl-6-fluoro-1H-indol-3-yl)-2-piperazin-1-ylmethyl-1H-benzoimidazole (Intermediate 62, 80 mg, 0.16 mmol), 35 mg (62%) of the title compound was obtained as a white solid. LC-MS for C₂₀H₂₀FN₅+H [M+H]⁺: calcd. 350.2. found: 350.1. ¹H NMR (400 MHz, CD₃OD) δ [ppm]: 7.73 (dd, J=8.8, 5.2 Hz, 1H), 7.70 (s, 1H), 7.50 (d, J=8.8 Hz, 1H), 7.44 (d, J=8.4 Hz, 1H), 7.34 (s, 1H), 7.03 (dd, J=9.6, 2.0 Hz, 1H), 7.79 (d, J=8.8, 2.0 Hz, 1H), 3.74 (s, 2H), 2.89 (t, J=4.8 Hz, 4H), 2.52 (t, J=4.8 Hz, 4H).

### Compound 60

### 2-(6-(6-fluoro-1H-indol-3-yl)-1H-benzo[d]imidazol-2-yl)acetamide

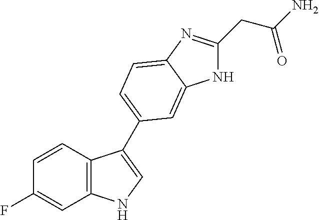 [SMILES: NC(=O)Cc1nc2ccc(-c3c[nH]c4cc(F)ccc34)cc2[nH]1]

To a stirred solution of [5-(6-fluoro-1H-indol-3-yl)-1H-benzoimidazol-2-yl]-acetonitrile (Intermediate 68, 186 mg, 0.628 mmol) in DMSO (5 mL) at 0° C. was added K₂CO₃ (173 mg, 1.26 mmol) and then aqueous H₂O₂ (0.7 mL, 30% w/w). The mixture was stirred at room temperature for 1 hour before it was diluted with water (50 mL) and extracted with EtOAc (50 mL×2). The combined organic layer was washed with brine (30 mL×2), dried over anhydrous Na₂SO₄ and filtered. The filtrate was concentrated and purified by a silica gel column chromatography (DCM/MeOH=20/1 to 5/1) to give the solid. The solid was suspended with DCM/MeOH=5/1 (3 mL) and filtered. The filtered cake was evaporated to dryness to afford 40 mg (21%) of the title compound as an off-white solid. LC-MS for C₁₇H₁₁FN₄O+H⁺[M+H]⁺: calcd. 309.1. found: 309.3. ¹H NMR (400 MHz, DMSO-d₆) δ [ppm]: 12.19 (d, J=24.0 Hz, 1H), 11.33 (d, J=12.8 Hz, 1H), 7.82 (dd, J=8.4, 5.2 Hz, 1H), 7.76-7.66 (m, 2H), 7.61 (dd, J=9.6, 2.4 Hz, 1H), 7.59-7.47 (m, 1H), 7.43 (td, J=9.6, 2.4 Hz, 1H), 7.25-7.16 (m, 2H), 6.99-6.91 (m, 1H), 3.73 (s, 2H).

### Compound 61

### 5-(6-fluoro-1H-indol-3-yl)-2-(piperazin-1-ylmethyl)benzo[d]oxazole

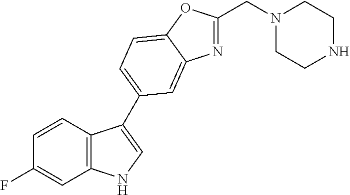 [SMILES: Fc1ccc2c(-c3ccc4oc(CN5CCNCC5)nc4c3)c[nH]c2c1]

Following the general method as outlined in Compound 1, starting from 5-(1-benzenesulfonyl-6-fluoro-1H-indol-3-yl)-2-piperazin-1-ylmethyl-benzooxazole (Intermediate 72, 80 mg, 0.16 mmol) in MeOH (10 mL), 30 mg (52%) of the title compound was obtained as a white solid. LC-MS for C₂₀H₁₉FN₄O+H⁺[M+H]⁺: calcd. 351.2. found: 351.1. ¹H NMR (400 MHz, CD₃OD) δ [ppm]: 7.77 (s, 1H), 7.67 (dd, J=8.8, 5.2 Hz, 1H), 7.54 (dd, J=8.8, 1.2 Hz, 1H), 7.49 (d, J=8.4 Hz, 1H), 7.36 (s, 1H), 7.03 (dd, J=9.6, 2.0 Hz, 1H), 6.78 (td, J=9.6, 2.0 Hz, 1H), 3.77 (s, 2H), 2.83 (t, J=4.8 Hz, 4H), 2.56 (t, J=4.8 Hz, 4H).

### Compound 62

### 5-(6-fluoro-1H-indol-3-yl)-2-((4-methylpiperazin-1-yl)methyl)benzo[d]oxazole

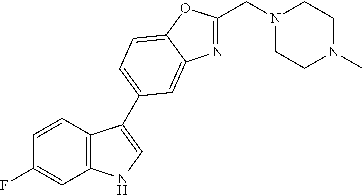 [SMILES: CN1CCN(Cc2nc3cc(-c4c[nH]c5cc(F)ccc45)ccc3o2)CC1]

Following the general method as outlined in Compound 1, starting from 5-(1-benzenesulfonyl-6-fluoro-1H-indol-3-yl)-2-(4-methyl-piperazin-1-ylmethyl)-benzooxazole (Intermediate 73, 160 mg, 0.32 mmol), 27 mg (23%) of the title compound was botained as a white solid. LC-MS for C₂₁H₂₁FN₄O+H⁺[M+H]⁺: calcd. 365.2. found: 365.1. ¹H NMR (400 MHz, CD₃OD) δ [ppm]: 7.89 (d, J=0.8 Hz, 1H), 7.80 (dd, J=8.8, 5.2 Hz, 1H), 7.67 (dd, J=8.8, 1.6 Hz, 1H), 7.62 (d, J=8.4 Hz, 1H), 7.47 (s, 1H), 7.13 (dd, J=9.6, 2.0 Hz, 1H), 6.89 (td, J=9.6, 2.0 Hz, 1H), 3.90 (s, 2H), 2.78-2.68 (m, 4H), 2.68-2.39 (m, 4H), 2.28 (s, 3H).

### Compound 63

### 5-(6-fluoro-1H-indol-3-yl)-2-(morpholinomethyl)benzo[d]oxazole

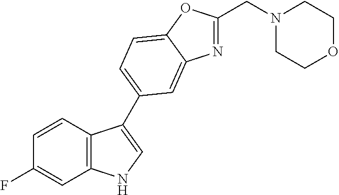 [SMILES: Fc1ccc2c(-c3ccc4oc(CN5CCOCC5)nc4c3)c[nH]c2c1]

Following the general method as outlined in Compound 1, starting from 5-(1-benzenesulfonyl-6-fluoro-1H-indol-3-yl)-2-morpholin-4-ylmethyl-benzooxazole (Intermediate 74, 100 mg, 0.204 mmol), 30 mg (42%) of the title compound was obtained as a white solid. LC-MS for C₂₀H₁₈FN₃O₂+H⁺[M+H]⁺: calcd. 352.1. found: 352.1. ¹H NMR (400 MHz, CD₃OD) δ [ppm]: 7.86 (s, 1H), 7.75 (dd, J=8.8, 5.6 Hz, 1H), 7.62 (d, J=8.4 Hz, 1H), 7.56 (d, J=8.4 Hz, 1H), 7.44 (s, 1H), 7.12 (dd, J=9.6, 1.6 Hz, 1H), 6.87 (td, J=9.6, 2.0 Hz, 1H), 3.82 (s, 2H), 3.69 (t, J=4.4 Hz, 4H), 2.59 (t, J=4.4 Hz, 4H).

### Compound 64

### 2-(6-(6-fluoro-1H-indol-3-yl)-2-oxobenzo[d]oxazol-3(2H)-yl)acetamide

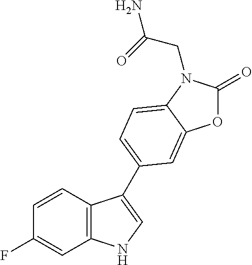 [SMILES: NC(=O)Cn1c(=O)oc2cc(-c3c[nH]c4cc(F)ccc34)ccc21]

Following the general method as outlined in Compound 1, starting from 2-(6-(6-fluoro-1-(phenylsulfonyl)-1H-indol-3-yl)-2-oxobenzo[d]oxazol-3(2H)-yl)acetamide (Intermediate 76, 183 mg, 0.4 mmol), 50 mg (38%) of the title compound was obtained as a white solid. LC-MS for C₁₇H₁₂FN₃O₃+H][M+H]⁺: calcd. 326.1. found: 325.8. ¹H NMR (400 MHz, DMSO-d₆) δ [ppm]: 11.43 (br s, 1H), 10.97 (br s, 1H), 9.81 (br s, 1H), 7.81 (dd, J=8.4, 5.2 Hz, 1H), 7.65 (d, J=2.4 Hz, 1H), 7.31 (d, J=8.0 Hz, 1H), 7.24-7.21 (m, 2H), 7.12 (dd, J=8.0, 1.2 Hz, 1H), 6.97 (td, J=9.2, 2.0 Hz, 1H), 4.28 (s, 2H).

### Compound 65

### 5-(6-fluoro-1H-indol-3-yl)-1H-benzo[d]imidazol-2(3H)-one

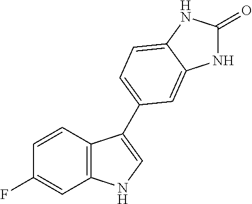 [SMILES: O=c1[nH]c2ccc(-c3c[nH]c4cc(F)ccc34)cc2[nH]1]

Following the general method as outlined in Compound 1, starting from 5-(1-Benzenesulfonyl-6-fluoro-1H-indol-3-yl)-, 3-dihydro-benzoimidazol-2-one (Intermediate 77, 185 mg, 0.45 mmol), 52 mg (43%) of the title compound was obtained as a yellow solid. LC-MS for C₁₅H₁₀FN₃O—H⁻ [M−H]: calcd. 266.1. found: 265.9. ¹H NMR (400 MHz, MeOD-d₄) δ [ppm]: 11.30 (s, 1H), 10.59 (s, 2H), 7.76 (dd, J₁=8.4 Hz, J₂=5.6 Hz, 1H), 7.56 (d, J=2.0 Hz, 1H), 7.23-7.19 (m, 2H), 7.15 (s, 1H), 6.98 (d, J=7.6 Hz, 1H), 6.94 (td, J₁=9.2 Hz, J₂=2.0 Hz, 1H).

### Compound 66

### 6-(6-fluoro-1H-indol-3-yl)-2-(morpholinomethyl)benzo[d]oxazole

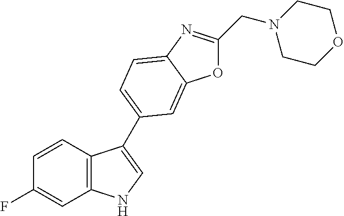 [SMILES: Fc1ccc2c(-c3ccc4nc(CN5CCOCC5)oc4c3)c[nH]c2c1]

Following the general method as outlined in Compound 1, starting from 6-(6-fluoro-1-(phenylsulfonyl)-1H-indol-3-yl)-2-(morpholinomethyl)benzo[d]oxazole (Intermediate 80, 240 mg, 0.48 mmol), 63 mg (44%) of the title compound was obtained as a tan solid. LC-MS for C₂₀H₁₈FN₃O₂+H [M+H]⁺: calcd. 352.1. found: 352.1. ¹H NMR (400 MHz, DMSO-d₆) δ [ppm]: 11.51 (s, 1H), 7.96 (s, 1H), 7.91-7.88 (m, 1H), 7.77-7.74 (m, 2H), 7.69-7.67 (m, 1H), 7.24 (dd, J=2.0, 9.6 Hz, 1H), 6.97 (td, J=2.4, 9.6 Hz, 1H), 3.87 (s, 2H), 3.61-3.59 (m, 4H), 2.56-2.54 (m, 4H).

### Compound 67

### 3-(6-(6-fluoro-1H-indol-3-yl)-2-oxobenzo[d]oxazol-3(2H)-yl)propanamide

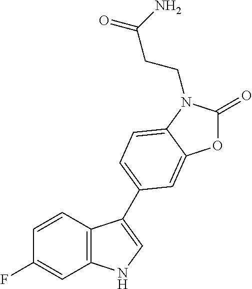 [SMILES: NC(=O)CCn1c(=O)oc2cc(-c3c[nH]c4cc(F)ccc34)ccc21]

Following the general method as outlined in Compound 1, starting from 3-(6-(6-fluoro-1-(phenylsulfonyl)-1H-indol-3-yl)-2-oxobenzo[d]oxazol-3(2H)-yl)propanamide (Intermediate 81, 280 mg, 0.58 mmol), 50 mg (25%) of the title compound was obtained as a white solid. LC-MS for C₁₈H₁₄FN₃O₃—H⁻ [M−H]⁻: calcd. 338.1. found: 337.9. ¹H NMR (400 MHz, DMSO-d₆) δ [ppm]: 11.40 (br s, 1H), 10.27 (br s, 1H), 9.60 (br s, 1H), 7.81 (dd, J=8.8, 5.4 Hz, 1H), 7.63 (d, J=2.8 Hz, 1H), 7.24-7.19 (m, 3H), 7.11 (dd, J=8.0, 1.6 Hz, 1H), 6.97 (td, J=9.6, 2.0 Hz, 1H), 3.63 (t, J=6.6 Hz, 2H), 2.70 (t, J=6.8 Hz, 2H).

### Compound 68

### 6-(6-fluoro-1H-indol-3-yl)-2-((4-methylpiperazin-1-yl)methyl)benzo[d]oxazole

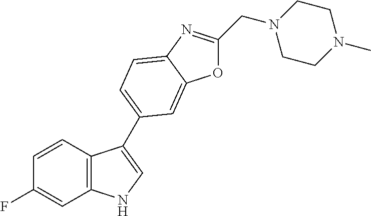 [SMILES: CN1CCN(Cc2nc3ccc(-c4c[nH]c5cc(F)ccc45)cc3o2)CC1]

Following the general method as outlined in Compound 1, starting from 6-(6-fluoro-1-(phenylsulfonyl)-1H-indol-3-yl)-2-((4-methylpiperazin-1-yl)methyl)benzo[d]oxazole (Intermediate 82, 285 mg, 0.56 mmol), 45 mg (22%) of the title compound was obtained as a tan solid. LC-MS for C₂₁H₂₁FN₄O+H⁺ [M+H]⁺: calcd. 365.2. found: 364.9. ¹H NMR (400 MHz, DMSO-d₆) δ [ppm]: 11.54 (s, 1H), 7.95-7.88 (m, 2H), 7.76-7.67 (m, 3H), 7.24 (dd, J=9.6, 1.6 Hz, 1H), 6.99-6.95 (m, 1H), 3.84 (s, 2H), 2.55 (s, 4H), 2.34 (s, 4H), 2.15 (s, 3H)

### Compound 69

### 2-(5-(6-fluoro-1H-indol-3-yl)-2-oxobenzo[d]oxazol-3(2H)-yl)acetamide

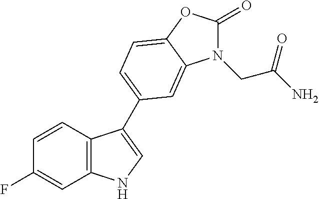 [SMILES: NC(=O)Cn1c(=O)oc2ccc(-c3c[nH]c4cc(F)ccc34)cc21]

Following the general method as outlined in Compound 1, starting from 2-(5-(6-fluoro-1-(phenylsulfonyl)-1H-indol-3-yl)-2-oxobenzo[d]oxazol-3(2H)-yl)acetamide (Intermediate 84, 600 mg crude, 1.29 mmol), 13 mg (3%) of the title compound was obtained as a white solid after purification by preparative HPLC. LC-MS for C₁₇H₁₂FN₃O₃—H⁻ [M−H]⁻: calcd. 324.1. found: 323.9. ¹H NMR (400 MHz, DMSO-d₆) δ [ppm]: 11.31 (br s, 1H), 10.95 (br s, 1H), 9.74 (br s, 1H), 7.80 (dd, J=8.7, 5.4 Hz, 1H), 7.59 (d, J=2.0 Hz, 1H), 7.54 (d, J=2.0 Hz, 1H), 7.46-7.43 (m, 1H), 7.19 (dd, J=10.0, 2.0 Hz, 1H), 6.99-6.90 (m, 2H), 4.32 (s, 2H).

### Compound 70

### N-((5-(6-fluoro-1H-indol-3-yl)benzo[d]oxazol-2-yl)methyl)methanesulfonamide

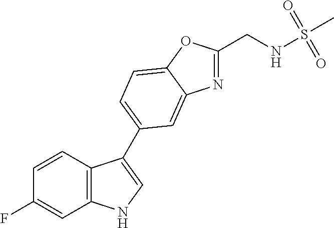 [SMILES: CS(=O)(=O)NCc1nc2cc(-c3c[nH]c4cc(F)ccc34)ccc2o1]

Following the general method as outlined in Compound 1, starting from N-[5-(1-benzenesulfonyl-6-fluoro-1H-indol-3-yl)-benzooxazol-2-ylmethyl]-methanesulfonamide (Intermediate 87, 83 mg, 0.17 mmol), 30 mg (50%) of the title compound was obtained as a white solid. LC-MS for C₁₇H₁₄FN₃O₃S+H⁺ [M+H]⁺: calcd. 360.1. found: 360.0. ¹H NMR (400 MHz, DMSO-d₆) δ [ppm]: 11.45 (br s, 1H), 8.01 (br s, 1H), 7.95 (s, 1H), 7.89-7.81 (m, 1H), 7.78 (d, J=8.0 Hz, 1H), 7.73 (d, J=2.8 Hz, 1H), 7.70 (d, J=8.8 Hz, 1H), 7.23 (dd, J=10.0, 2.0 Hz, 1H), 6.96 (td, J=8.8, 2.0 Hz, 1H), 4.54 (s, 2H), 3.04 (s, 3H).

### Compound 71

### 3-(5-(6-fluoro-1H-indol-3-yl)-2-oxobenzo[d]oxazol-3(2H)-yl)propanamide)

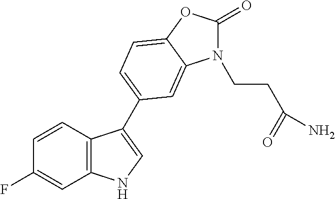 [SMILES: NC(=O)CCn1c(=O)oc2ccc(-c3c[nH]c4cc(F)ccc34)cc21]

Following the general method as outlined in Compound 1, starting from 3-[5-(1-Benzenesulfonyl-6-fluoro-1H-indol-3-yl)-2-oxo-benzooxazol-3-yl]-propionamide (Intermediate 88, 270 mg, 0.56 mmol), the title compound (48 mg, 25%) was obtained as a white solid. LC-MS for C₁₈H₁₄FN₃O₃—H⁻ [M−H]⁻: calcd. 338.1. found: 338.0. ¹H NMR (400 MHz, DMSO) δ 11.30 (s, 1H), 10.31 (s, 1H), 9.56 (s, 1H), 7.78 (dd, J=8.8, 5.6 Hz, 1H), 7.54 (d, J=2.0 Hz, 1H), 7.47 (d, J=2.0 Hz, 1H), 7.43 (dd, J=8.4, 2.2 Hz, 1H), 7.19 (dd, J=9.9, 1.9 Hz, 1H), 6.98 (d, J=8.4 Hz, 1H), 6.99-6.90 (m, 2H), 3.67 (t, J=6.6 Hz, 2H), 2.72 (t, J 5=6.6 Hz, 2H).

### Compound 72

### 3-(benzo[b]thiophen-5-yl)-6-fluoro-1H-indole

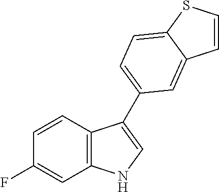 [SMILES: Fc1ccc2c(-c3ccc4sccc4c3)c[nH]c2c1]

Following the general method as outlined in Compound 1, starting from 3-(benzo[b]thiophen-5-yl)-6-fluoro-1-(phenylsulfonyl)-1H-indole (Intermediate 90, 300 mg, 0.74 mmol), 3-Benzo[b]thiophen-5-yl-6-fluoro-1H-indole (70 mg, 36%) was obtained as a white solid. LC-MS for C₁₆H₁₀FNS—H⁻ [M−H]⁻: calcd. 266.1. found: 265.9. ¹H NMR (400 MHz, DMSO) δ 11.44 (s, 1H), 8.18 (s, 1H), 8.03 (d, J=8.0 Hz, 1H), 7.92 (dd, J=8.8, 5.2 Hz, 1H), 7.77 (d, J=5.2 Hz, 1H), 7.74 (s, 1H), 7.68 (d, J=8.8 Hz, 1H), 7.51 (d, J=5.6 Hz, 1H), 7.23 (dd, J=9.8, 2.0 Hz, 1H), 6.97 (td, J=9.2, 2.4 Hz, 1H).

### Compound 73

### N-((5-(6-fluoro-1H-indol-3-yl)benzo[d]oxazol-2-yl)methyl)acetamide

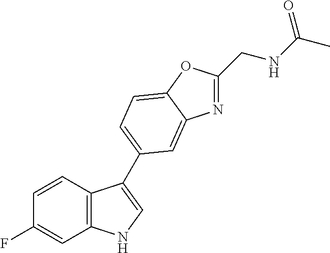 [SMILES: CC(=O)NCc1nc2cc(-c3c[nH]c4cc(F)ccc34)ccc2o1]

To a stirred mixture of C-[5-(6-fluoro-1H-indol-3-yl)-benzooxazol-2-yl]-methylamine hydrochloride (Compound 81, 40 mg, 0.126 mmol) in DCM (6 mL) at room temperature was added TEA (25 mg, 0.252 mmol) and AcCl (49 mg, 0.63 mmol). The mixture was stirred for a further 30 minutes before it was quenched with ice water (20 mL) and extracted with EtOAc (20 mL×2). The combined organic layer was washed with brine (20 mL×2), dried over anhydrous Na₂SO₄ and filtered. The filtrate was concentrated and purified by a preparative TLC (DCM/MeOH=20/1) to afford 13 mg (32%) of the title compound as a yellow solid. LC-MS for C₁₈H₁₄FN₃O₂+H⁺[M+H]⁺: calcd. 324.1. found: 324.1. ¹H NMR (400 MHz, CD₃OD) δ [ppm]: 7.90 (s, 1H), 7.82 (dd, J=8.8, 1.6 Hz, 1H), 7.70 (dd, J=8.4, 1.6 Hz, 1H), 7.66 (d, J=8.4 Hz, 1H), 7.50 (s, 1H), 7.16 (dd, J=9.6, 2.0 Hz, 1H), 6.92 (td, J=9.6, 2.4 Hz, 1H), 4.70 (s, 2H), 2.10 (s, 3H).

### Compound 74

### N-((6-(6-fluoro-1H-indol-3-yl)benzo[d]oxazol-2-yl)methyl)methanesulfonamide

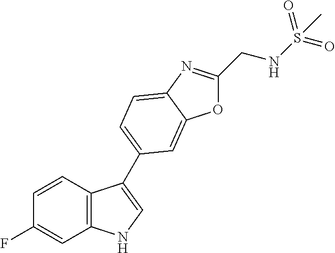 [SMILES: CS(=O)(=O)NCc1nc2ccc(-c3c[nH]c4cc(F)ccc34)cc2o1]

A mixture of tert-butyl tert-butyl((6-(6-fluoro-1H-indol-3-yl)benzo[d]oxazol-2-yl)methyl)(methylsulfonyl)carbamate (Intermediate 92, 114 mg, 0.25 mmol) in HCl/1,4-dioxane (15 mL) was stirred at room temperature for 0.5 hour. The reaction mixture was concentrated and purified by preparative HPLC to afford 22 mg (24%) of the title compound as a yellow solid. LC-MS for C₁₇H₁₄FN₃O₃S+H⁺[M+H]⁺: calcd. 360.1. found: 360.1. ¹H NMR (400 MHz, DMSO-d₆) δ [ppm]: 11.51 (s, 1H), 8.03-7.99 (m, 1H), 7.97 (s, 1H), 7.90 (dd, J=8.8, 5.4 Hz, 1H), 7.80-7.75 (m, 2H), 7.70 (dd, J=8.3, 1.3 Hz, 1H), 7.24 (dd, J=10.2, 2.2 Hz, 1H), 6.97 (td, J=9.2, 2.1 Hz, 1H), 4.54 (d, J=4.4 Hz, 2H), 3.04 (s, 3H).

### Compound 75

### N-((6-(6-fluoro-1H-indol-3-yl)benzo[d]oxazol-2-yl)methyl)acetamide

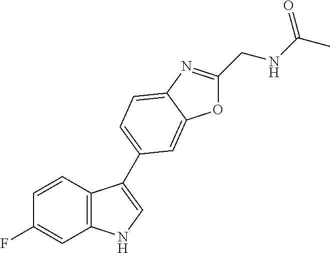 [SMILES: CC(=O)NCc1nc2ccc(-c3c[nH]c4cc(F)ccc34)cc2o1]

To a solution of (6-(6-fluoro-1H-indol-3-yl)benzo[d]oxazol-2-yl)methanamine hydrochloride (Compound 76, 120 mg, 0.38 mmol) and TEA (115 mg, 1.14 mmol) in DCM (20 mL) was added acetyl chloride (36 mg, 0.45 mmol) under nitrogen. The mixture was stirred at room temperature for 1 hour under nitrogen. The reaction was acidified with HCl (12 M) till pH=5. The mixture was diluted with water (60 mL) and extracted with DCM (30 mL). The combined organic layer was washed with water (60 mL×3), dried over anhydrous Na₂SO₄ and filtered. The filtrate was concentrated and purified by preparative HPLC to afford 55 mg (39%) of the title compound as a white solid. LC-MS for C₁₈H₁₄FN₃O₂+H⁺[M+H]⁺: calcd. 324.1. found: 324.5. ¹H NMR (400 MHz, DMSO-d₆) δ [ppm]: 11.50 (s, 1H), 8.69 (t, J=5.6 Hz, 1H), 7.94 (s, 1H), 7.89 (dd, J=9.0, 5.4 Hz, 1H), 7.77 (d, J=2.5 Hz, 1H), 7.74 (d, J=8.2 Hz, 1H), 7.69-7.67 (m, 1H), 7.24 (dd, J=9.9, 2.3 Hz, 1H), 6.97 (td, J=9.2, 2.0 Hz, 1H), 4.57 (d, J=5.6 Hz, 2H), 1.93 (s, 3H).

### Compound 76

### (6-(6-fluoro-1H-indol-3-yl)benzo[d]oxazol-2-yl)methanamine

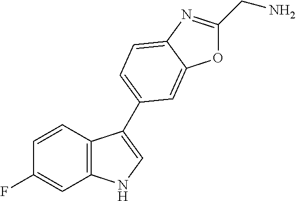 [SMILES: NCc1nc2ccc(-c3c[nH]c4cc(F)ccc34)cc2o1]

A mixture of tert-butyl N-[(tert-butoxy)carbonyl]-N-{[6-(6-fluoro-1H-indol-3-yl)-1,3-benzoxazol-2-yl]methyl}carbamate (Intermediate 94; 376 mg, 0.78 mmol) in HCl/1,4-dioxane (20 mL) was stirred at room temperature for 0.5 hour. The reaction mixture was concentrated to afford 219 mg (100%) of the title compound as a yellow solid. LC-MS for C₁₆H₁₃ClFN₃O+H⁺[M+H]⁺: calcd. 282.1. found: 281.9. ¹H NMR (400 MHz, DMSO-d₆) δ [ppm]: 11.51 (br s, 1H), 7.92 (s, 1H), 7.90-7.87 (m, 1H), 7.77 (s, 1H), 7.74 (d, J=8.0 Hz, 1H), 7.67 (d, J=8.2 Hz, 1H), 7.25 (d, J=9.6 Hz, 1H), 6.98 (t, J=9.0 Hz, 1H), 3.99 (s, 2H).

### Compound 77

### 5-(6-fluoro-1H-indol-3-yl)-2-(piperidin-4-ylmethyl)-2H-indazole

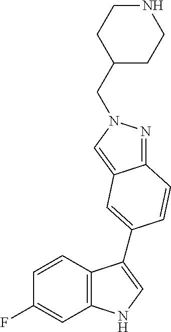 [SMILES: Fc1ccc2c(-c3ccc4nn(CC5CCNCC5)cc4c3)c[nH]c2c1]

A mixture of 4-[5-(6-Fluoro-1H-indol-3-yl)-indazol-2-ylmethyl]-piperidine-1-carboxylic acid tert-butyl ester (Intermediate 5-2, 189 mg, 0.42 mmol) in HCl/1,4-dioxane (10 mL) was stirred at room temperature for 1 hour. The mixture was concentrated and the residue was purified by preparative TLC (dichloro-methane/MeOH=10/1) to afford 136 mg (93%) of the title compound as a yellow solid. LC-MS for C₂₁H₂₁FN₄+H⁺ [M+H]⁺: calcd. 349.2. found: 348.9.

¹H NMR (400 MHz, MeOD-d₄) δ [ppm]: 11.35 (s, 1H), 8.31 (s, 1H), 7.92-7.86 (m, 2H), 7.66-7.63 (m, 2H), 7.55 (dd, J=9.2, 1.6 Hz, 1H), 7.21 (dd, J=9.6, 2.4 Hz, 1H), 6.95 (td, J=9.6, 2.4 Hz, 1H), 4.28 (d, J=7.2 Hz, 2H), 2.91-2.89 (m, 2H), 2.41-2.36 (m, 2H), 1.39 (d, J=10.8 Hz, 2H), 1.12 (td, J=12.2, 3.4 Hz, 2H).

### Compound 78

### 6-(6-fluoro-1H-indol-3-yl)-2-(piperazin-1-ylmethyl)benzo[d]oxazole

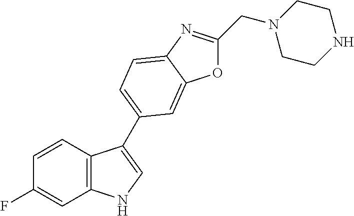 [SMILES: Fc1ccc2c(-c3ccc4nc(CN5CCNCC5)oc4c3)c[nH]c2c1]

Following the general method as outlined in Compound 1, starting from 6-(6-fluoro-1-(phenylsulfonyl)-1H-indol-3-yl)-2-(piperazin-1-ylmethyl)benzo[d]oxazole (Intermediate 95, 277 mg, 0.56 mmol), 84 mg (43%) of the title compound was obtained as a white solid. LC-MS for C₂₀H₁₉FN₄O+H⁺ [M+H]⁺: calcd. 351.2. found: 351.1. ¹H NMR (400 MHz, MeOD) δ [ppm]: 7.89-7.84 (m, 2H), 7.74-7.73 (m, 2H), 7.56 (s, 1H), 7.16 (dd, J=10, 2.4 Hz, 1H), 6.94 (td, J=9.2, 2.4 Hz, 1H), 3.96-3.95 (m, 2H), 2.97 (br s, 4H), 2.71 (br s, 4H)

### Compound 79

### 3-(benzo[b]thiophen-6-yl)-6-fluoro-1H-indole

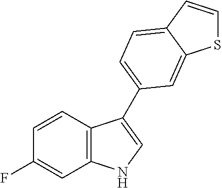 [SMILES: Fc1ccc2c(-c3ccc4ccsc4c3)c[nH]c2c1]

Following the general method as outlined in Compound 1, starting from 3-(benzo[b]thiophen-6-yl)-6-fluoro-1-(phenylsulfonyl)-1H-indole (Intermediate 97, 338 mg, 0.83 mmol) in MeOH (20 mL), 89 mg (40%) of the title compound was obtained as a yellow solid. LC-MS for C₁₆H₁₀FNS—H⁺ [M−H]⁺: calcd. 266.1. found: 266.0. ¹H NMR (400 MHz, DMSO-d₆) δ [ppm]: 11.47 (s, 1H), 8.29 (s, 1H), 7.97-7.91 (m, 2H), 7.78 (d, J=2.4 Hz, 1H), 7.73-7.68 (m, 2H), 7.45 (d, J=5.2 Hz, 1H), 7.24 (dd, J=10.0, 2.0 Hz, 1H), 6.98 (td, J=9.2, 2.4 Hz, 1H).

### Compound 80

### 5-(6-fluoro-1H-indol-3-yl)-2-(2-(methylsulfonyl)ethyl)benzo[d]oxazole

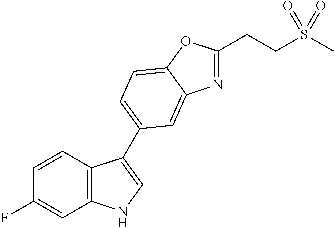 [SMILES: CS(=O)(=O)CCc1nc2cc(-c3c[nH]c4cc(F)ccc34)ccc2o1]

To a solution of 6-fluoro-3-[2-(2-methanesulfonyl-ethyl)-benzooxazol-5-yl]-indole-1-carboxylic acid tert-butyl ester (Intermediate 101, 45 mg, 0.098 mmol) in MeOH (1 mL) was added HCl/Et₂O (5 mL, 4M) at 0° C. The mixture was stirred at room temperature for 16 hours before it was concentrated. The residue was diluted with MeOH (5 mL) and basified with NH₃/THF to pH=8. The mixture was concentrated and purified by a preparative TLC (DCM/MeOH=20/1) to afford 6 mg (17%) of the title compound as an off-white solid. LC-MS for C₁₈H₁₅FN₂O₃S+H⁺[M+H]⁺: calcd. 359.1. found: 359.0. ¹H NMR (400 MHz, CDCl₃) δ [ppm]: 8.26 (brs, 1H), 7.91 (s, 1H), 7.82 (dd, J=8.8, 5.2 Hz, 1H), 7.61 (d, J=8.0 Hz, 1H), 7.57 (d, J=8.0 Hz, 1H), 7.35 (d, J=2.0 Hz, 1H), 7.14 (d, J=9.6, 2.0 Hz, 1H), 6.97 (td, J=8.8, 2.0 Hz, 1H), 3.70 (t, J=7.6 Hz, 2H), 3.54 (t, J=7.6 Hz, 2H), 3.02 (s, 3H).

### Compound 81

### (5-(6-fluoro-1H-indol-3-yl)benzo[d]oxazol-2-yl)methanamine

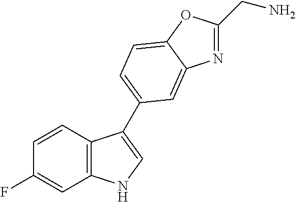 [SMILES: NCc1nc2cc(-c3c[nH]c4cc(F)ccc34)ccc2o1]

To a solution of [5-(6-fluoro-1H-indol-3-yl)-benzooxazol-2-ylmethyl]-carbamic acid tert-butyl ester (Intermediate 103, 63 mg, 0.165 mmol) in MeOH (3 mL) was added HCl/Et₂O (3 mL, 8M) at 0° C. The mixture was stirred at room temperature for 1 hour. The mixture was evaporated to dryness to afford 20 mg (43%) of the title compound as a yellow solid. LC-MS for C₁₆H₁₂FN₃O+H⁺[M+H]⁺: calcd. 282.1. found: 282.1. ¹H NMR (400 MHz, DMSO-d₆) δ [ppm]: 11.53 (br s, 1H), 8.83 (br s, 3H), 8.00 (s, 1H), 7.86-7.82 (m, 2H), 7.77-7.75 (m, 2H), 7.25 (dd, J=10.0, 2.4 Hz, 1H), 6.98 (td, J=8.8, 2.0 Hz, 1H), 4.54-4.52 (m, 2H).

### Compound 82

### 3-(benzofuran-5-yl)-6-fluoro-1H-indole

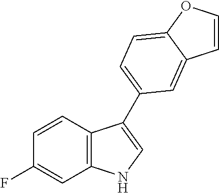 [SMILES: Fc1ccc2c(-c3ccc4occc4c3)c[nH]c2c1]

Following the general method as outlined in Compound 1, starting from 3-(benzofuran-5-yl)-6-fluoro-1-(phenylsulfonyl)-1H-indole (Intermediate 104, 300 mg, 0.77 mmol), 40 mg (21%) of the title compound was obtained as a yellow solid after purification by silica gel chromatography (petroleum ether/EtOAc=5/1, v/v). LC-MS for C₁₆H₁₀FNO—H⁻ [M−H]⁻: calcd. 250.1. found: 250.1. ¹H NMR (400 MHz, DMSO-d₆) δ [ppm]: 11.39 (br s, 1H), 8.00 (d, J=2.0 Hz, 1H), 7.91 (d, J=1.8 Hz, 1H), 7.85 (dd, J=8.8, 5.5 Hz, 1H), 7.66 (s, 1H), 7.60 (dd, J=8.4, 2.0 Hz, 2H), 7.23 (dd, J=9.6, 2.4 Hz, 1H), 6.70-6.93 (m, 2H).

### Compound 83

### 3-(6-(6-fluoro-1H-indol-3-yl)benzo[d]oxazol-2-yl)propanamide

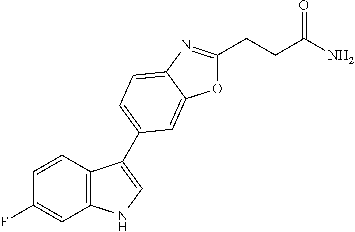 [SMILES: NC(=O)CCc1nc2ccc(-c3c[nH]c4cc(F)ccc34)cc2o1]

To a mixture of 3-[6-(6-fluoro-1H-indol-3-yl)-benzooxazol-2-yl]-propionic acid (Intermediate 108, 52 mg, 0.17 mmol) and NH₄Cl (27 mg, 0.50 mmol) in anhydrous THF was added HATU (127 mg, 0.33 mmol) and TEA (34 mg, 0.33 mmol) at 0° C. The mixture was stirred at room temperature under nitrogen for 1.5 hours before it was quenched with ice water (30 mL) and extracted with EtOAc (30 mL×2). The combined organic layer was washed with 1M NaOH (10 mL×2) and brine (20 mL×2), dried over anhydrous Na₂SO₄ and filtered. The filtrate was evaporated and purified by a preparative TLC (DCM) and a preparative HPLC (NH₃/H₂O as additive) to afford 10 mg (20%) of the title compound as a white solid. LC-MS for C₁₈H₁₄FN₃O₂+H⁺[M+H]⁺: calcd. 324.1. found: 324.1. ¹H NMR (400 MHz, DMSO-d₆) δ [ppm]: 11.48 (brs, 1H), 7.92-7.86 (m, 2H), 7.75 (d, J=2.4 Hz, 1H), 7.69 (d, J=8.0 Hz, 1H), 7.64 (dd, J=8.0, 1.6 Hz, 1H), 7.47 (brs, 1H), 7.24 (dd, J=9.6, 2.0 Hz, 1H), 6.96 (td, J=8.4, 2.0 Hz, 1H), 6.91 (brs, 1H), 3.14 (t J=7.2 Hz, 2H), 2.68 (t J=7.2 Hz, 2H).

### Compound 84

### 2-(6-(6-fluoro-1H-indol-3-yl)-1H-benzo[d]imidazol-1-yl)acetamide

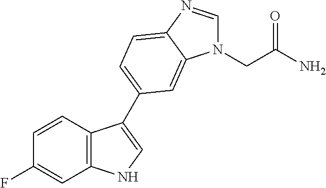 [SMILES: NC(=O)Cn1cnc2ccc(-c3c[nH]c4cc(F)ccc34)cc21]

Following the general method as outlined in Compound 1, starting from 2-(6-(6-fluoro-1-(phenylsulfonyl)-1H-indol-3-yl)-1H-benzo[d]imidazol-1-yl)acetamide (Intermediate 109A, 100 mg, 0.22 mmol), 14 mg (21%) of the title compound was obtained as a white solid after purification by preparative TLC (DCM/MeOH=10/1, v/v). LC-MS for C₁₇H₁₃FN₄O+H [M+H]⁺: calcd. 309.1. found: 309.1. ¹H NMR (400 MHz, DMSO-d₆) δ [ppm]: 11.48 (br s, 1H), 8.14 (s, 1H), 7.88 (dd, J=8.8, 5.5 Hz, 1H), 7.87 (br s, 1H), 7.70 (d, J=1.6 Hz, 1H), 7.68 (d, J=8.4 Hz, 1H), 7.65 (d, J=2.4 Hz, 1H), 7.50 (dd, J=8.4, 1.6 Hz, 1H), 7.37 (br s, 1H), 7.23 (dd, J=9.9, 2.4 Hz, 1H), 6.95 (m, 1H), 4.98 (s, 2H).

### Compound 85

### 2-(5-(6-fluoro-1H-indol-3-yl)-1H-benzo[d]imidazol-1-yl)acetamide

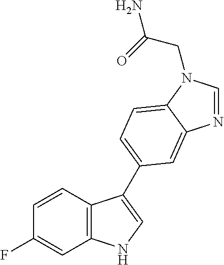 [SMILES: NC(=O)Cn1cnc2cc(-c3c[nH]c4cc(F)ccc34)ccc21]

Following the general method as outlined in Compound 1, starting from 2-(5-(6-fluoro-1-(phenylsulfonyl)-1H-indol-3-yl)-1H-benzo[d]imidazol-1-yl)acetamide (Intermediate 109B, 60 mg, 0.13 mmol), 13 mg (31%) of the title compound was obtained as a white solid after purification by preparative HPLC. LC-MS for C₁₇H₁₃FN₄O+H⁺[M+H]⁺: calcd. 309.1. found: 309.1. ¹H NMR (400 MHz, DMSO-d₆) δ [ppm]: 11.35 (br s, 1H), 8.16 (s, 1H), 7.87 (d, J=1.6 Hz, 1H), 7.84 (dd, J=8.8, 5.5 Hz, 1H), 7.76 (br s, 1H), 7.64 (d, J=2.2 Hz, 1H), 7.56 (dd, J=8.4, 1.6 Hz, 1H), 7.50 (d, J=8.4 Hz, 1H), 7.37 (br s, 1H), 7.22 (dd, J=10.0, 2.4 Hz, 1H), 6.95 (m, 1H), 4.93 (s, 2H).

### Compound 86

### 3-(5-(6-fluoro-1H-indol-3-yl)-1H-indazol-1-yl)-1-morpholinopropan-1-one

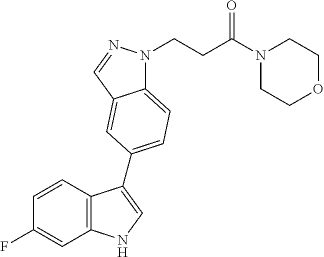 [SMILES: O=C(CCn1ncc2cc(-c3c[nH]c4cc(F)ccc34)ccc21)N1CCOCC1]

A mixture of 3-[5-(6-Fluoro-1H-indol-3-yl)-indazol-1-yl]-propionic acid (Intermediate 111, 180 mg, crude, 0.56 mmol), Morpholine (48 mg, 0.56 mmol), HATU (319 mg, 0.84 mmol) and DIEA (217 mg, 1.68 mmol) in 5 mL of DMF was stirred at room temperature for 2 hr.

Then the mixture was poured into water and extracted with EtOAc (50 mL×3). The combined organic layer was washed with brine, dried over anhydrous Na₂SO₄, filtered and concentrated. The residue was purified by preparative HPLC (CH₃CN/H₂O=30%-75%, NH₄HCO₃) to afford 10 mg (5%) of the title compound as a white solid. LC-MS for C₂₂H₂₁FN₄O₂+H [M+H]⁺: calcd. 393.1. found: 393.1. ¹H NMR (400 MHz, DMSO-d₆) δ [ppm]: 11.37 (br s, 1H), 8.08 (br s, 1H), 7.98 (br s, 1H), 7.86 (dd, J=8.8, 5.6 Hz, 1H), 7.74-7.67 (m, 3H), 7.22 (dd, J=9.6, 2.4 Hz, 1H), 6.96 (td, J=9.4, 2.4 Hz, 1H), 4.65 (t, J=6.8 Hz, 2H), 3.47-3.38 (m, 6H), 3.34-3.30 (m, 2H), 2.96 (t, J=6.8 Hz, 2H).

### Compound 87

### 3-(5-(6-fluoro-1H-indol-3-yl)-1H-indazol-1-yl)-1-(4-methylpiperazin-1-yl)propan-1-one

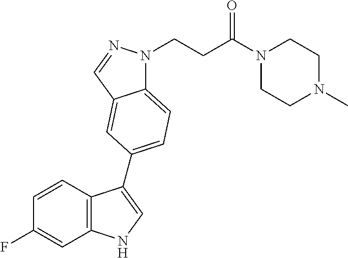 [SMILES: CN1CCN(C(=O)CCn2ncc3cc(-c4c[nH]c5cc(F)ccc45)ccc32)CC1]

A mixture of 3-[5-(6-Fluoro-1H-indol-3-yl)-indazol-1-yl]-propionic acid (Intermediate 111, 300 mg, crude, 0.93 mmol), 1-Methyl-piperazine (93 mg, 0.93 mmol), HATU (530 mg, 1.4 mmol) and DIEA (360 mg, 2.8 mmol) in 5 mL of DMF was stirred at room temperature for 2 hr. Then the mixture was poured into water and extracted with EtOAc (50 mL×3). The combined organic layer was washed with brine, dried over anhydrous Na₂SO₄, filtered and concentrated. The residue was purified by preparative HPLC (CH₃CN/H₂O=30%-75%, NH₄HCO₃) to afford 76 mg (20%) of the title compound as a white solid. LC-MS for C₂₃H₂₄FN₅O+H⁺[M+H]⁺: calcd. 406.2. found: 406.2. ¹H NMR (400 MHz, DMSO-d₆) δ [ppm]: 11.36 (s, 1H), 8.08 (s, 1H), 7.98 (s, 1H), 7.88-7.84 (m, 1H), 7.73-7.66 (m, 3H), 7.22 (dd, J=10.0, 2.4 Hz, 1H), 6.96 (td, J=8.8, 2.4 Hz, 1H), 4.64 (t, J=6.8 Hz, 2H), 3.40-3.38 (m, 2H), 3.32-3.29 (m, 2H), 2.94 (t, J=6.8 Hz, 2H), 2.14-2.09 (m, 7H).

### Compound 88

### N-(2-(dimethylamino)ethyl)-3-(5-(6-fluoro-H-indol-3-yl)-1H-indazol-1-yl)propanamide

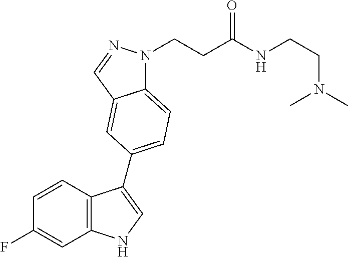 [SMILES: CN(C)CCNC(=O)CCn1ncc2cc(-c3c[nH]c4cc(F)ccc34)ccc21]

A mixture of 3-[5-(6-Fluoro-1H-indol-3-yl)-indazol-1-yl]-propionic acid (Intermediate 111, 300 mg, crude, 0.93 mmol), (2-aminoethyl)dimethylamine (82 mg, 0.93 mmol), HATU (530 mg, 1.4 mmol) and DIEA (360 mg, 2.8 mmol) in 5 mL of DMF was stirred at room temperature for 2 hr. Then the mixture was poured into water and extracted with EtOAc (50 mL×3). The combined organic layer was washed with brine, dried over anhydrous Na₂SO₄, filtered and concentrated. The residue was purified by preparative HPLC (CH₃CN/H₂O=30%-75%, NH₄HCO₃) to afford 60 mg (16%) of the title compound as a white solid. LC-MS for C₂₂H₂₄FN₅O+H⁺[M+H]⁺: calcd. 394.2. found: 394.2. ¹H NMR (400 MHz, DMSO-d₆) δ [ppm]: 11.37 (br s, 1H), 8.07 (s, 1H), 7.97 (s, 1H), 7.88-7.84 (m, 2H), 7.72-7.66 (m, 3H), 7.22 (dd, J=10.0, 1.6 Hz, 1H), 6.96 (t, J=5.2 Hz, 1H), 4.62 (t, J=6.4 Hz, 2H), 3.06 (q, J=6.4 Hz, 2H), 2.69 (t, J=6.4 Hz, 2H), 2.11 (t, J=6.4 Hz, 2H), 2.04 (s, 6H).

### Compound 89

### 3-(5-(6-fluoro-1H-indol-3-yl)-1H-indazol-1-yl)-N-(2-hydroxyethyl)propanamide

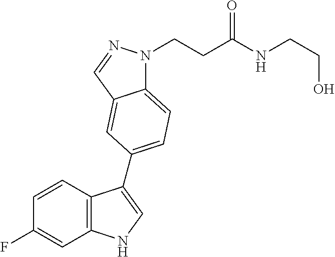 [SMILES: O=C(CCn1ncc2cc(-c3c[nH]c4cc(F)ccc34)ccc21)NCCO]

A mixture of 3-[5-(6-Fluoro-1H-indol-3-yl)-indazol-1-yl]-propionic acid (Intermediate 111, 300 mg, crude, 0.93 mmol), 2-Amino-ethanol (57 mg, 0.93 mmol), HATU (530 mg, 1.4 mmol) and DIEA (360 mg, 2.8 mmol) in 5 mL of DMF was stirred at room temperature for 2 hr. Then the mixture was poured into water and extracted with EtOAc (50 mL×3). The combined organic layer was washed with brine, dried over anhydrous Na₂SO₄, filtered and concentrated. The residue was purified by preparative HPLC (CH₃CN/H₂O=30%-75%, NH₄HCO₃) to afford 53 mg (16%) of the title compound as a white solid. LC-MS for C₂₀H₁₉FN₄O₂+H⁺[M+H]⁺: calcd. 367.2. found: 367.1. ¹H NMR (400 MHz, DMSO-d₆) δ [ppm]: 11.37 (br s, 1H), 8.07 (br s, 1H), 7.98-7.94 (m, 2H), 7.88-7.85 (m, 1H), 7.70-7.67 (m, 3H), 7.22 (d, J=10.0 Hz, 1H), 6.96 (t, J=9.2 Hz, 1H), 4.63-4.61 (m, 3H), 3.36-3.30 (m, 2H), 3.08-3.07 (m, 2H), 2.71 (t, J=6.0 Hz, 2H).

### Compound 90

### 3-(5-(6-fluoro-1H-indol-3-yl)-1H-indazol-1-yl)-N-(2-(methylsulfonyl)ethyl)propanamide

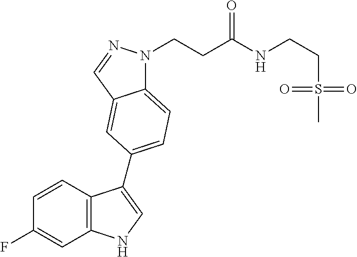 [SMILES: CS(=O)(=O)CCNC(=O)CCn1ncc2cc(-c3c[nH]c4cc(F)ccc34)ccc21]

A mixture of 3-[5-(6-Fluoro-1H-indol-3-yl)-indazol-1-yl]-propionic acid (Intermediate 111, 300 mg, crude, 0.93 mmol), 2-Methanesulfonyl-ethylamine HCl salt (148 mg, 0.93 mmol), HATU (530 mg, 1.4 mmol) and DIEA (360 mg, 2.8 mmol) in 5 mL of DMF was stirred at room temperature for 2 hr. Then the mixture was poured into water and extracted with EtOAc (50 mL×3). The combined organic layer was washed with brine, dried over anhydrous Na₂SO₄, filtered and concentrated. The residue was purified by preparative HPLC (CH₃CN/H₂O=30%-75%, NH₄HCO₃) to afford 64 mg (16%) of the title compound as a white solid. LC-MS for C₂₁H₂₁FN₄O₃S+H⁺[M+H]⁺: calcd. 429.1. found: 429.1. ¹H NMR (400 MHz, DMSO-d₆) δ [ppm]: 11.37 (br s, 1H), 8.25 (t, J=5.6 Hz, 1H), 8.07 (s, 1H), 7.98 (s, 1H), 7.89-7.85 (m, 1H), 7.70 (s, 2H), 7.67 (s, 1H), 7.22 (dd, J=10.0, 2.0 Hz, 1H), 6.96 (td, J=9.2, 2.0 Hz, 1H), 4.63 (t, J=6.8 Hz, 2H), 3.40 (q, J=6.4 Hz, 2H), 3.12 (t, J=6.8 Hz, 2H), 2.89 (s, 3H), 2.72 (t, J=6.8 Hz, 2H).

### Compound 91

### 3-(5-(6-fluoro-1H-indol-3-yl)-1H-indazol-1-yl)-1-(piperazin-1-yl)propan-1-one

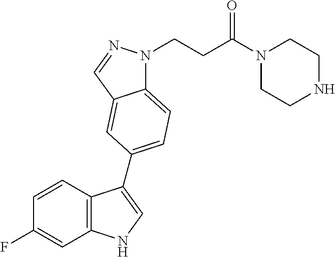 [SMILES: O=C(CCn1ncc2cc(-c3c[nH]c4cc(F)ccc34)ccc21)N1CCNCC1]

A solution of 4-{3-[5-(6-Fluoro-1H-indol-3-yl)-indazol-1-yl]-propionyl}-piperazine-1-carboxylic acid tert-butyl ester (Intermediate 112, 98 mg, 0.2 mmol) in HCl/EtOAc (25%, 10 mL) was stirred at room temperature for 2 hr. Then the mixture was concentrated. The residue was purified by preparative HPLC (CH₃CN/H₂O=30%-75%, NH₄HCO₃) to afford 34 mg (43%) of the title compound as a white solid. LC-MS for C₂₂H₂₂FN₅O+H⁺[M+H]⁺: calcd. 392.2. found: 392.1. ¹H NMR (400 MHz, DMSO-d₆) δ [ppm]: 11.37 (br s, 1H), 8.07 (s, 1H), 7.97 (s, 1H), 7.88-7.85 (m, 1H), 7.74-7.66 (m, 3H), 7.22 (dd, J=10.0, 2.4 Hz, 1H), 6.96 (td, J=9.2, 2.4 Hz, 1H), 4.63 (t, J=6.8 Hz, 2H), 3.32-3.30 (m, 2H), 3.25-3.23 (m, 2H), 2.93 (t, J=6.8 Hz, 2H), 2.55-2.47 (m, 4H).

### Compound 92

### 6-(6-fluoro-1H-indol-3-yl)indolin-2-one

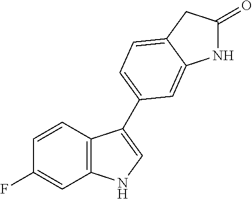 [SMILES: O=C1Cc2ccc(-c3c[nH]c4cc(F)ccc34)cc2N1]

A mixture of 6-(6-fluoro-1-(phenylsulfonyl)-1H-indol-3-yl)indolin-2-one (Intermediate 113, 400 mg, 0.98 mmol), Mg turnings (200 mg, 8.2 mmol) and NH₄Cl (106 mg, 2.0 mmol) in THF (2 mL) and MeOH (8 mL) was stirred for 2 hours at room temperature. The reaction mixture was filtered through Celite, concentrated and purified by preparative TLC (petroleum ether/EtOAc=3/1) to afford 26 mg (10%) of the title compound as a yellow solid. LC-MS for C₁₆H₁₁FN₂O—H⁻ [M−H]⁻: calcd. 265.1. found: 265.1. ¹H NMR (400 MHz, MeOH-d₄) δ [ppm]: 7.68 (dd, J=8.8, 5.2 Hz, 1H), 7.33 (s, 1H), 7.18 (s, 1H), 7.07 (s, 2H), 7.01 (dd, J=9.8, 2.2 Hz, 1H), 6.77 (td, J=9.1, 2.4 Hz, 1H), 3.45 (s, 2H).

### Compound 93

### 3-(6-(6-fluoro-1H-indol-3-yl)-1H-benzo[d]imidazol-1-yl)propanamide

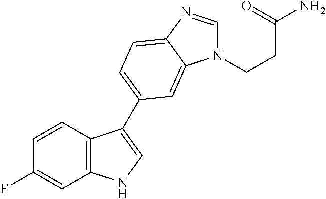 [SMILES: NC(=O)CCn1cnc2ccc(-c3c[nH]c4cc(F)ccc34)cc21]

Following the general method as outlined in Compound 1, starting from 3-(6-(6-fluoro-1-(phenylsulfonyl)-1H-indol-3-yl)-1H-benzo[d]imidazol-1-yl)propanamide (Intermediate 114A, 140 mg, 0.30 mmol), 30 mg (31%) of the title compound was obtained as a white solid after purification by preparative HPLC. LC-MS for C₁₈H₁₅FN₄O+H⁺[M+H]⁺: calcd. 323.1. found: 323.2. ¹H NMR (400 MHz, DMSO-d₆) δ [ppm]: 11.51 (br s, 1H), 8.13 (s, 1H), 7.96 (dd, J=8.8, 5.6 Hz, 1H), 7.85 (s, 1H), 7.69-7.67 (m, 2H). 7.51 (d, J=8.0 Hz, 1H), 7.47 (br s, 1H), 7.24 (dd, J=9.8, 2.2 Hz, 1H), 6.97 (td, J=9.2, 2.0 Hz, 2H), 4.52 (t, J=6.4 Hz, 2H), 2.68 (t, J=6.6 Hz, 2H).

### Compound 94

### 3-(5-(6-fluoro-1H-indol-3-yl)-1H-benzo[d]imidazol-1-yl)propanamide

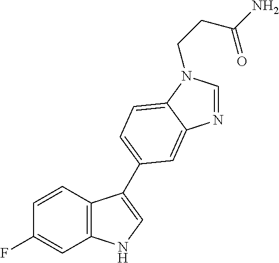 [SMILES: NC(=O)CCn1cnc2cc(-c3c[nH]c4cc(F)ccc34)ccc21]

Following the general method as outlined in Compound 1, starting from 3-(5-(6-fluoro-1-(phenylsulfonyl)-1H-indol-3-yl)-1H-benzo[d]imidazol-1-yl)propanamide (Intermediate 114B, 180 mg, 0.39 mmol), 51 mg (41%) of the title compound was obtained as a white solid after purification by preparative HPLC. LC-MS for C₁₈H₁₅FN₄O+H⁺[M+H]⁺: calcd. 323.1. found: 323.2. ¹H NMR (400 MHz, DMSO-d₆) δ [ppm]: 11.37 (br s, 1H), 8.16 (s, 1H), 7.87 (s, 1H), 7.84 (dd, J=8.4, 5.6 Hz, 1H), 7.68 (d, J=8.0 Hz, 1H), 7.64 (d, J=2.4 Hz, 1H), 7.57 (d, J=8.4 Hz, 1H), 7.42 (s, 1H), 7.22 (dd, J=10.0, 2.4 Hz, 1H), 6.95 (m, 2H), 4.48 (t, J=6.4 Hz, 2H), 2.66 (t, J=6.6 Hz, 2H).

### Compound 95

### 1-(4-(6-(6-fluoro-1H-indol-3-yl)benzo[d]oxazol-2-yl)piperidin-1-yl)ethanone

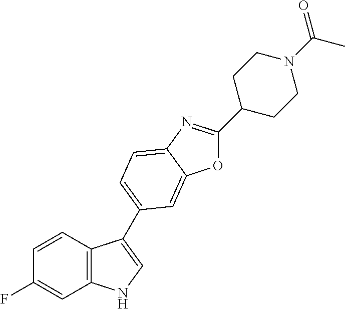 [SMILES: CC(=O)N1CCC(c2nc3ccc(-c4c[nH]c5cc(F)ccc45)cc3o2)CC1]

To a stirred solution of 6-(6-fluoro-1H-indol-3-yl)-2-(piperidin-4-yl)benzo[d]oxazole (Compound 55, 200 mg, 0.60 mmol) and TEA (182 mg, 1.80 mmo) in DCM (10 mL) at room temperature, was added dropwise acetyl chloride (57 mg, 0.72 mmol). The mixture was stirred at room temperature overnight. The mixture was diluted with DCM (10 mL), washed with water (20 mL×2), dried over Na₂SO₄ and filtered. The filtrate was concentrated and purified by preparative HPLC to afford 121 mg (54%) of the title compound as a yellow solid. LC-MS for C₂₂H₂₀FN₃O₂+H+: [M+H]⁺: calcd. 378.2. found: 378.1. ¹H NMR (400 MHz, DMSO-d₆) δ [ppm]: 11.48 (s, 1H), 7.92 (s, 1H), 7.89 (dd, J=5.6, 8.8 Hz, 1H), 7.75 (d, J=2.8 Hz, 1H), 7.73 (d, J=7.2 Hz, 1H), 7.66 (dd, J=1.6, 8.0 Hz, 1H), 7.23 (dd, J=2.4, 10.0 Hz, 1H), 6.97 (td, J=2.4, 9.6 Hz, 1H), 4.32 (d, J=13.6 Hz, 1H), 3.87 (d, J=13.6 Hz, 1H), 3.35-3.27 (m, 2H), 2.88 (t, J=11.2 Hz, 1H), 2.13 (t, J=14.0 Hz, 2H), 2.04 (s, 3H), 1.88-1.77 (m, 1H), 1.72-1.62 (m, 1H).

### Compound 96

### 6-(6-fluoro-1H-indol-3-yl)-2-(1-(methylsulfonyl)piperidin-4-yl)benzo[d]oxazole

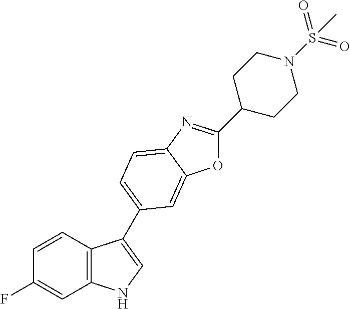 [SMILES: CS(=O)(=O)N1CCC(c2nc3ccc(-c4c[nH]c5cc(F)ccc45)cc3o2)CC1]

A solution of 6-(6-fluoro-1H-indol-3-yl)-2-(piperidin-4-yl)benzo[d]oxazole (Compound 55, 200 mg, 0.60 mmol) and TEA (182 mg, 1.80 mmo) in DCM (10 mL) was stirred at room temperature for 10 min before it was cooled to 0° C. Then Methanesulfonyl chloride (82 mg, 0.72 mmol) was added dropwise. The reaction was stirred at room temperature for 1 hour. The mixture was diluted with DCM (10 mL), washed with water (20 mL×2), dried over Na₂SO₄ and concentrated. The residue was purified by preparative HPLC to afford 159 mg (64%) of the title compound as a pink solid. LC-MS for C₂₁H₂₀FN₃O₃S+H⁺[M+H]⁺: calcd. 414.12. found: 414.10. ¹H NMR (400 MHz, DMSO-d₆) δ [ppm]: 11.48 (s, 1H), 7.93 (s, 1H), 7.88 (dd, J=5.6, 8.8 Hz, 1H), 7.75 (s, 1H), 7.74 (d, J=11.2 Hz, 2H), 7.66 (dd, J=1.6, 8.0 Hz, 1H), 7.24 (dd, J=2.4, 10.0 Hz, 1H), 6.97 (td, J=2.4, 10.0 Hz, 1H), 3.62-3.58 (m, 2H), 3.26-3.19 (m, 1H), 3.00 (td, J=2.4, 12.0 Hz, 2H), 2.91 (s, 3H), 2.24 (dd, J=13.6, 3.6 Hz, 2H), 1.93-1.89 (m, 2H).

### Compound 97

### 5-(6-fluoro-1H-indol-3-yl)indolin-2-one

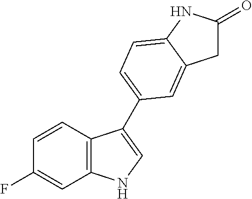 [SMILES: O=C1Cc2cc(-c3c[nH]c4cc(F)ccc34)ccc2N1]

The mixture of 1-benzenesulfonyl-6-fluoro-1′,3′-dihydro-1H-[3,5′]biindolyl-2′-one (Intermediate 116, 300 mg, 0.74 mmol), Mg (178 mg, 7.4 mmol) and NH₄Cl (12 mg, 0.22 mmol) in MeOH was stirred at room temperature under nitrogen for about 5 hrs. After it is done, the mixture was filtered through a pad of celite and concentrated. The residue was purified by pre-TLC (DCM/methanol=20/1) to get the title compound (60 mg, 30%) as a yellow solid. LC-MS for C₁₆H₁₁FN₂O—H⁻ [M−H]⁻: calcd. 265.1. found: 265.1. ¹H NMR (400 MHz, DMSO-d₆) δ [ppm]: 11.31 (s, 1H), 10.38 (s, 1H), 7.78 (dd, J=8.8, 5.6 Hz, 1H), 7.56 (d, J=2.4 Hz, 1H), 7.50 (s, 1H), 7.46 (d, J=8.0 Hz, 1H), 7.19 (dd, J=10.0, 2.3 Hz, 1H), 6.93 (td, J=9.5, 2.4 Hz, 1H), 6.88 (d, J=8.0 Hz, 1H), 3.53 (s, 2H).

### Compound 98

### 6-(6-fluoro-1H-indol-3-yl)benzo[d]oxazol-2-amine

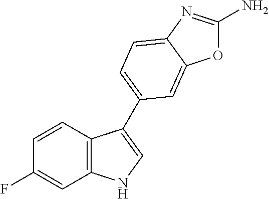 [SMILES: Nc1nc2ccc(-c3c[nH]c4cc(F)ccc34)cc2o1]

The mixture of 6-(1-Benzenesulfonyl-6-fluoro-1H-indol-3-yl)-benzooxazol-2-ylamine (Intermediate 118, 300 mg, 0.74 mmol), Mg (178 mg, 7.4 mmol) and NH₄Cl (12 mg, 0.22 mmol) in MeOH was stirred at room temperature under nitrogen for about 5 hrs. After it is done, the mixture was filtered through a pad of celite and concentrated. The residue was purified by preparative TLC (DCM/methanol=20/1) to get the title compound (40 mg, 25%) as a yellow solid. LC-MS for C₁₅H₁₀FN₃O+H⁺[M+H]⁺: calcd. 268.1. found: 268.0. ¹H NMR (400 MHz, DMSO) δ 11.33 (s, 1H), 7.82 (dd, J=8.8, 5.4 Hz, 1H), 7.61 (d, J=2.4 Hz, 1H), 7.56 (d, J=1.2 Hz, 1H), 7.40 (dd, J=8.0, 1.2 Hz, 1H), 7.36 (s, 2H), 7.24 (d, J=8.1 Hz, 1H), 7.20 (dd, J=10.0, 2.4 Hz, 1H), 6.93 (td, J=9.6, 2.4 Hz, 1H).

### Compound 99

### N-((6-(6-fluoro-1H-indol-3-yl)benzo[d]oxazol-2-yl)methyl)-2-methoxyethanesulfonamide

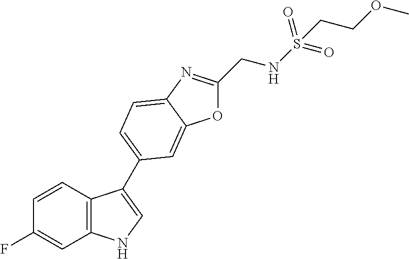 [SMILES: COCCS(=O)(=O)NCc1nc2ccc(-c3c[nH]c4cc(F)ccc34)cc2o1]

To a stirred solution of (6-(6-fluoro-1H-indol-3-yl)benzo[d]oxazol-2-yl)methanamine hydrochloride (Compound 76, 196 mg, 0.62 mmol) and TEA (0.25 mL, 1.86 mmol) in DCM (30 mL) was added 2-methoxyethanesulfonyl chloride (128 mg, 0.8 mmol) at 0° C. under nitrogen. The mixture was stirred at 0° C. for 1 hour before it was diluted with DCM (20 mL). The organic layer was washed with water (50 mL×3), dried over anhydrous Na₂SO₄ and filtered. The filtrate was concentrated and purified by preparative HPLC to afford 15 mg (5%) of the title compound as a yellow solid. LC-MS for C₁₉H₁₈FN₃O₄S+H+[M+H]⁺: Calcd. 404.1. found: 403.8. ¹H NMR (400 MHz, MeOD) δ [ppm]: 7.77-7.71 (m, 2H), 7.63-7.58 (m, 2H), 7.43 (s, 1H), 7.04 (dd, J=10.0, 2.4 Hz, 1H), 6.82 (td, J=9.2, 2.4 Hz, 1H), 4.49 (s, 2H), 3.68 (t, J=6.0 Hz, 2H), 3.34 (t, J=6.0 Hz, 2H), 3.22 (s, 3H).

### Compound 100

### 2-(dimethylamino)-N-((6-(6-fluoro-1H-indol-3-yl)benzo[d]oxazol-2-yl)methyl)ethanesulfonamide

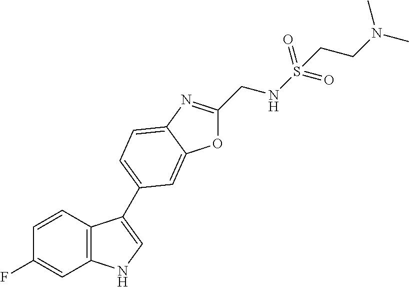 [SMILES: CN(C)CCS(=O)(=O)NCc1nc2ccc(-c3c[nH]c4cc(F)ccc34)cc2o1]

To a stirred solution of N-((6-(6-fluoro-1H-indol-3-yl)benzo[d]oxazol-2-yl)methyl)ethenesulfonamide (Intermediate 119, 147 mg, 0.396 mmol) in MeCN (10 mL) was added dimethylamine in THF (0.99 mL, 2M). The mixture was stirred at room temperature for 0.5 hours before it was neutralized with HCl (12 M). The mixture was diluted with water (60 mL) and extracted with EtOAc (60 mL×3). The combined organic layer was washed with water (60 mL×3), dried over anhydrous Na₂SO₄ and filtered. The filtrate was concentrated and purified by preparative HPLC to afford 20 mg (12%) of the title compound as a yellow solid. LC-MS for C₂₀H₂₁FN₄O₃S+H⁺[M+H]⁺: calcd. 417.1. found: 417.5. ¹H NMR (400 MHz, CDCl₃) δ [ppm]: 8.33 (s, 1H), 7.82 (dd, J=8.8, 5.2 Hz, 1H), 7.76 (d, J=0.9 Hz, 1H), 7.74 (d, J=8.3 Hz, 1H), 7.62 (dd, J=8.2, 1.4 Hz, 1H), 7.38 (d, J=2.3 Hz, 1H), 7.14 (dd, J=9.4, 2.2 Hz, 1H), 6.99 (td, J=9.2, 2.3 Hz, 1H), 4.67 (s, 2H), 3.34 (t, J=6.3 Hz, 2H), 2.86 (t, J=6.3 Hz, 2H), 2.27 (s, 6H).

### Compound 101

### 6-(6-fluoro-1H-indol-3-yl)-2-(2-(methylsulfonyl)ethyl)benzo[d]oxazole

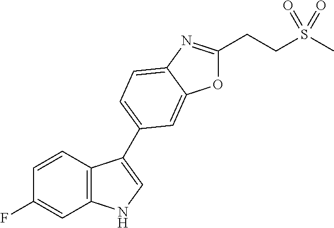 [SMILES: CS(=O)(=O)CCc1nc2ccc(-c3c[nH]c4cc(F)ccc34)cc2o1]

A mixture of 6-fluoro-3-[2-(2-methanesulfonyl-ethyl)-benzooxazol-6-yl]-indole-1-carboxylic acid tert-butyl ester (Intermediate 123, 390 mg, 0.852 mmol) in HCl/EtOAc (5 mL, 2M) was stirred at room temperature for 3 hours. The mixture was diluted with EtOAc (50 mL), washed with aq.NaHCO₃ (20 mL) and brine (20 mL×2), dried over anhydrous Na₂SO₄ and filtered. The filtrate was evaporated and purified by a preparative HPLC to afford 132 mg (43%) of the title compound as a white solid. LC-MS for C₁₈H₁₅FN₂O₃S+H⁺[M+H]⁺: calcd. 359.1. found: 359.1. ¹H NMR (400 MHz, DMSO-d₆) δ [ppm]: 11.49 (br s, 1H), 7.93 (d, J=1.2 Hz, 1H), 7.88 (dd, J=8.8, 1.6 Hz, 1H), 7.76 (d, J=2.4 Hz, 1H), 7.73 (d, J=8.0 Hz, 1H), 7.68 (dd, J=8.4, 1.6 Hz, 1H), 7.24 (dd, J=9.6, 2.4 Hz, 1H), 6.97 (td, J=9.6, 2.4 Hz, 1H), 3.73 (t, J=7.6 Hz, 2H), 3.44 (t, J=7.8 Hz, 2H), 3.11 (s, 3H).

### Compound 102

### 1-((6-(6-fluoro-1H-indol-3-yl)benzo[d]oxazol-2-yl)methyl)urea

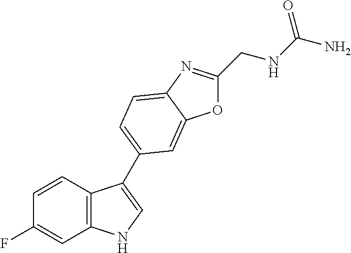 [SMILES: NC(=O)NCc1nc2ccc(-c3c[nH]c4cc(F)ccc34)cc2o1]

To a solution of (6-(6-fluoro-1H-indol-3-yl)benzo[d]oxazol-2-yl)methanamine hydrochloride (Compound 76, 158 mg, 0.50 mmol) and Et₃N (176 mg, 1.74 mmol) in DCM (10 mL) was added trimethylsilyl isocyanate (138 mg, 1.20 mmol). The mixture was stirred at room temperature for 12 h. Then it was concentrated and purified by preparative HPLC to afford 15 mg (9%) of the title compound as a white solid. LC-MS for C₁₇H₁₃FN₄O₂+H⁺ [M+H]⁺: calcd. 325.1. found: 325.1. ¹H NMR (400 MHz, DMSO-d₆) δ [ppm]: 11.48 (br s, 1H), 7.92 (s, 1H), 7.89 (dd, J=8.8, 5.5 Hz, 1H), 7.76 (d, J=1.8 Hz, 1H), 7.72 (d, J=8.4 Hz, 1H), 7.67 (dd, J=8.4, 1.8 Hz, 1H), 7.24 (dd, J=9.9, 2.2 Hz, 1H), 6.97 (t, J=2.0 Hz, 1H), 6.69 (t, J=6.0 Hz, 1H), 5.77 (br s, 2H), 4.50 (d, J=6.0 Hz, 2H).

### Compound 103

### 1-carbamoyl-1-((6-(6-fluoro-1H-indol-3-yl)benzo[d]oxazol-2-yl)methyl)urea

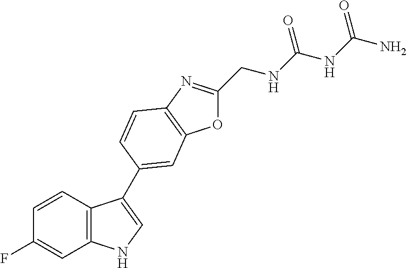 [SMILES: NC(=O)NC(=O)NCc1nc2ccc(-c3c[nH]c4cc(F)ccc34)cc2o1]

The title compound (5 mg, 3%) was obtained as a by-product during the synthesis described for Compound 102. LC-MS for C₁₈H₁₄FN₅O₃+H⁺ [M+H]⁺: calcd. 368.1. found: 368.1. ¹H NMR (400 MHz, DMSO-d₆) δ [ppm]: 11.49 (br s, 1H), 8.92 (s, 1H), 8.30 (br t, 1H), 7.95 (s, 1H), 7.90 (dd, J=8.8, 5.5 Hz, 1H), 7.76 (d, J=2.2 Hz, 1H), 7.74 (d, J=8.4 Hz, 1H), 7.68 (d, J=8.4 Hz, 1H), 7.24 (dd, J=10.0, 2.2 Hz, 1H), 6.96 (m, 1H), 6.7 d (br s, 2H), 4.66 (d, J=5.7 Hz, 2H).

### Compound 104

### 5-(1H-indol-3-yl)-1H-indazole

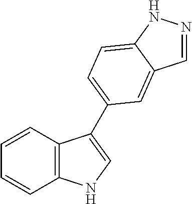 [SMILES: c1ccc2c(-c3ccc4[nH]ncc4c3)c[nH]c2c1]

Following the general method as outlined in Compound 8, the title compound was obtained as a yellow solid after purification by preparative HPLC. LC-MS for C₁₅H₁₁N₃+H⁺ [M+H]⁺: calcd. 234.1. found: 234.1. ¹H NMR (400 MHz, DMSO-d₆) δ [ppm]: 13.01 (br s, 1H), 11.27 (br s, 1H), 8.09 (s, 1H), 8.01 (s, 1H), 7.90 (d, J=7.8 Hz, 1H), 7.69 (d, J=8.6 Hz, 1H), 7.65 (d, J=2.3 Hz, 1H), 7.59 (d, J=8.6 Hz, 1H), 7.45 (d, J=8.0 Hz, 1H), 7.18-7.08 (m, 2H).

### Compound 105

### 6-(6-fluoro-1H-indol-3-yl)-2-(2-(4-methylpiperazin-1-yl)ethyl)benzo[d]oxazole

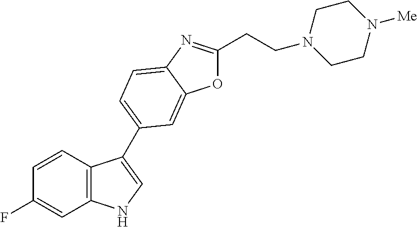 [SMILES: CN1CCN(CCc2nc3ccc(-c4c[nH]c5cc(F)ccc45)cc3o2)CC1]

A solution of 2-(2-chloroethyl)-6-(6-fluoro-1-(phenylsulfonyl)-1H-indol-3-yl)benzo[d]oxazole (Intermediate 125, 100 mg, 0.22 mmol), sodium hydroxide (44 mg, 1.10 mmol) and N-Methylpiperazine (44 mg, 0.44 mmol) in methanol (10 mL) was stirred at 50° C. overnight. The reaction mixture was cooled to room temperature before it was concentrated. The residue was diluted with water (20 mL) and extracted with EtOAc (50 mL×3). The organic layer was concentrated and purified by Preparative HPLC (NH₄HCO₃) to afford 44 mg (53%) of the title compound as a white solid. LC-MS for C₂₂H₂₃FN₄O+H⁺[M+H]⁺: calcd. 379.2. found: 379.2. ¹H NMR (400 MHz, CDCl₃) δ [ppm]: 8.28 (s, 1H), 7.86-7.82 (m 1H), 7.76-7.67 (m, 2H), 7.58 (d, J=8.1 Hz, 1H), 7.37 (d, J=1.9 Hz, 1H), 7.13 (dd, J=9.5, 2.1 Hz, 1H), 7.01-6.96 (m, 1H), 3.19-3.15 (m, 2H), 3.01-2.97 (m, 2H), 2.66-2.47 (m, 8H), 2.32 (s, 3H).

### Compound 106

### N-((6-(6-fluoro-1H-indol-3-yl)benzo[d]oxazol-2-yl)methyl)-2-hydroxyethanesulfonamide

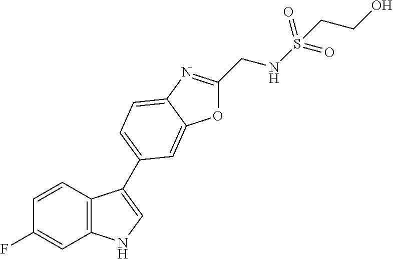 [SMILES: O=S(=O)(CCO)NCc1nc2ccc(-c3c[nH]c4cc(F)ccc34)cc2o1]

To a solution of N-((6-(6-fluoro-1H-indol-3-yl)benzo[d]oxazol-2-yl)methyl)-2-methoxyethanesulfonamide (Compound 99, 120 mg, 0.3 mmol) in DCM (20 mL) was added tribromoborane (0.38 mL, 4.17 mmol) dropwise at 0° C. under nitrogen. The mixture was stirred at room temperature for 1 hour under nitrogen before it was diluted with DCM (30 mL). An aqueous solution of NaHCO₃ (15 mL) was added dropwise into the reaction mixture at 0° C. and stirred for 20 mins. The reaction mixture was extracted with EtOAc (30 mL×2) and water (20 mL×2). The combined organic layer was washed with brine (60 mL×2), dried over anhydrous Na₂SO₄ and filtered. The filtrate was concentrated and purified by preparative HPLC to afford 20 mg (17%) of the title compound as a yellow solid. LC-MS for C₁₈H₁₆FN₃O₄S+H⁺[M+H]⁺: calcd. 390.1. found: 390.5. ¹H NMR (400 MHz, CD₃OH) δ [ppm]: 7.77 (s, 1H), 7.74 (dd, J=8.8, 5.3 Hz, 1H), 7.640-7.58 (m, 2H), 7.43 (s, 1H), 7.04 (dd, J=9.7, 2.3 Hz, 1H), 6.81 (td, J=9.2, 2.3 Hz, 1H), 4.52 (s, 2H), 3.88 (t, J=6.2 Hz, 2H), 3.29 (t, J=6.2 Hz, 2H).

### Compound 107

### N-((6-(6-fluoro-1H-indol-3-yl)benzo[d]oxazol-2-yl)methyl)-2-morpholinoethanesulfonamide

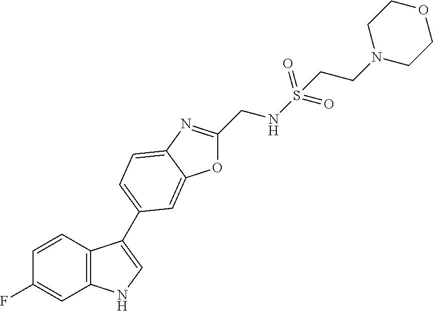 [SMILES: O=S(=O)(CCN1CCOCC1)NCc1nc2ccc(-c3c[nH]c4cc(F)ccc34)cc2o1]

To a solution of N-((6-(6-fluoro-1H-indol-3-yl)benzo[d]oxazol-2-yl)methyl)ethenesulfonamide (Intermediate 119, 433 mg, 1.16 mmol) in MeCN (20 mL) was added morpholine (0.5 mL, 5.83 mmol). The mixture was stirred at room temperature for 0.5 hours. The mixture was diluted with water (60 mL) and extracted with EtOAc (60 mL×3). The combined organic layer was washed with water (60 mL×3), dried over anhydrous Na₂SO₄ and filtered. The filtrate was concentrated and purified by Preparative HPLC to afford 13 mg (2%) of the title compound as a yellow solid. LC-MS for C₂₂H₂₃FN₄O₄S+H⁺ [M+H]⁺: calcd. 459.1. found: 458.8. ¹H NMR (400 MHz, CD₃OH) δ [ppm]: 7.90 (s, 1H), 7.86 (dd, J=8.7, 5.4 Hz, 1H), 7.78-7.71 (m, 2H), 7.56 (s, 1H), 7.17 (dd, J=9.7, 2.3 Hz, 1H), 6.94 (td, J=9.1, 2.2 Hz, 1H), 4.66 (s, 2H), 3.65 (t, J=4.6 Hz, 4H), 3.39 (t, J=7.2 Hz, 2H), 2.84 (t, J=7.6 Hz, 2H), 2.48-2.47 (m, 4H).

### Compound 108

### 6-(1H-indol-3-yl)-1H-indazole

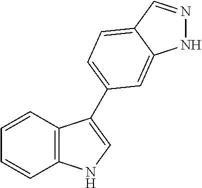 [SMILES: c1ccc2c(-c3ccc4cn[nH]c4c3)c[nH]c2c1]

To a solution of 6-(6-fluoro-1-(phenylsulfonyl)-1H-indol-3-yl)-1H-indazole (Intermediate 4, 148 mg, 0.4 mmol) in MeOH (20 mL) was added a solution of NaOH (158 mg, 4.0 mmol) at room temperature. The reaction was stirred at 80° C. for 1 hour. The mixture was cooled to room temperature and concentrated. The residue was purified by prep-TLC (PE/EtOAc=1/1) to afford 32 mg (36%) of the title compound as a yellow solid. LC-MS for C₁₅H₁₁N₃+H⁺[M+H]⁺: calcd. 234.1. found: 234.0. ¹H NMR (400 MHz, DMSO-d₆) δ [ppm]: 12.93 (s, 1H), 11.40 (s, 1H), 8.04 (s, 1H), 7.91 (d, J=7.6 Hz, 1H), 7.80-7.76 (m, 3H), 7.47 (d, J=8.4 Hz, 2H), 7.20-7.10 (m, 2H).

### Compound 109

### 6-(1H-indol-3-yl)benzo[d]oxazole

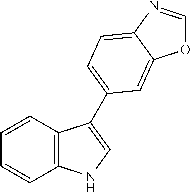 [SMILES: c1ccc2c(-c3ccc4ncoc4c3)c[nH]c2c1]

Following the general method as outlined in Compound 1, starting from 6-(1-Benzenesulfonyl-1H-indol-3-yl)-benzooxazole (Intermediate 127, 330 mg, 0.88 mmol), 6-(1H-Indol-3-yl)-benzooxazole (100 mg, 49%) was obtained as a white solid. LC-MS for C₁₅H₁₀N₂O+H⁺[M+H]⁺: calcd. 235.1. found: 235.5. ¹H NMR (300 MHz, DMSO-d₆) δ [ppm]: 11.45 (s, 1H), 8.71 (s, 1H), 8.01 (d, J=1.1 Hz, 1H), 7.93 (d, J=7.6 Hz, 1H), 7.83 (d, J=8.2 Hz, 1H), 7.79 (d, J=2.6 Hz, 1H), 7.75 (dd, J=8.3, 1.5 Hz, 1H), 7.48-7.45 (m, 1H), 7.20-7.09 (m, 2H).

### Compound 110

### 6-(6-fluoro-1H-indol-3-yl)-2-(2-(piperazin-1-yl)ethyl)benzo[d]oxazole

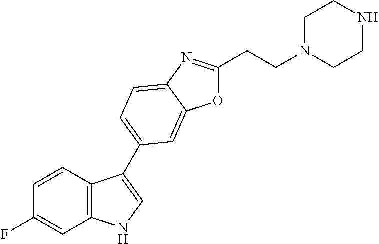 [SMILES: Fc1ccc2c(-c3ccc4nc(CCN5CCNCC5)oc4c3)c[nH]c2c1]

A solution of 6-(6-fluoro-1-(phenylsulfonyl)-1H-indol-3-yl)-2-vinylbenzo[d]oxazole (Intermediate 128, 1.00 g, 2.39 mmol), sodium hydroxide (478 mg, 11.96 mmol) and piperazine (617 mg, 7.17 mmol) in methanol (20 mL) was stirred at 50° C. overnight. The reaction mixture was cooled to room temperature, concentrated, diluted with water and extracted with EtOAc. The organic layers was dried over anhydrous Na₂SO₄ and filtered. The filtrate was concentrated and purified by preparative HPLC (NH₄HCO₃) to afford 609 mg (70%) of the title compound as a white solid. LC-MS for C₂₁H₂₁FN₄O+H⁺[M+H]⁺: calcd. 365.1. found: 365.1. ¹H NMR (400 MHz, DMSO-d₆) δ [ppm]: 11.46 (br s, 1H), 7.90-7.86 (m, 2H), 7.74 (d, J=2.4 Hz, 1H), 7.70 (d, J=8.2 Hz, 1H), 7.64 (dd, J=8.3, 1.5 Hz, 1H), 7.23 (dd, J=9.9, 2.4 Hz, 1H), 6.96 (td, J=9.5, 2.4 Hz, 1H), 3.11 (t, J=7.2 Hz, 2H), 2.80 (t, J=7.4 Hz, 2H), 2.70-2.62 (m, 4H), 2.38 (m, 4H).

### Compound 111

### 5-(6-chloro-1H-indol-3-yl)benzo[d]oxazole

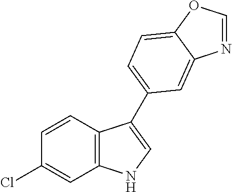 [SMILES: Clc1ccc2c(-c3ccc4ocnc4c3)c[nH]c2c1]

Following the general method as outlined in Compound 1, starting from 5-(6-chloro-1-(phenylsulfonyl)-1H-indol-3-yl)benzo[d]oxazole (Intermediate 129, 330 mg, 0.08 mmol), 115 mg (53%) of the title compound was obtained as a white solid. LC-MS for C₁₅H₉ClN₂O+H⁺[M+H]⁺: calcd. 269.0. found: 269.3. ¹H NMR (400 MHz, DMSO-d₆) δ [ppm]: 11.52 (s, 1H), 8.75 (s, 1H), 8.02 (d, J=1.3 Hz, 1H), 7.88 (d, J=8.6 Hz, 1H), 7.83 (d, J=8.4 Hz, 1H), 7.79 (d, J=2.6 Hz, 1H), 7.74 (dd, J=8.6, 1.7 Hz, 1H), 7.50 (d, J=1.8 Hz, 1H), 7.12 (dd, J=8.6, 2.0 Hz, 1H).

### Compound 112

### 6-(6-fluoro-1H-indol-3-yl)benzo[d]oxazole-2-thiol

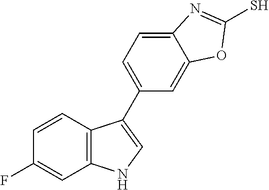 [SMILES: Fc1ccc2c(-c3ccc4nc(S)oc4c3)c[nH]c2c1]

Following the general method as outlined in Compound 1, starting from 6-(1-Benzenesulfonyl-6-fluoro-1H-indol-3-yl)-benzooxazole-2-thiol (Intermediate 130, 450 mg), the title compound (80 mg, 36% for 2 steps) was obtained as a yellow solid. LC-MS for C₁₅H₉FN₂OS—H⁻ [M−H]⁻: calcd. 283.0. found: 283.0. ¹H NMR (400 MHz, DMSO-d₆) δ [ppm]: 13.85 (s, 1H), 11.48 (s, 1H), 7.86 (dd, J=9.2, 5.2 Hz, 1H), 7.77 (d, J=1.1 Hz, 1H), 7.74 (d, J=2.4 Hz, 1H), 7.62 (dd, J=8.4, 1.6 Hz, 1H), 7.29 (d, J=8.4 Hz, 1H), 7.24 (dd, J=9.6, 2.4 Hz, 1H), 6.97 (td, J=9.6, 2.4 Hz, 1H).

### Compound 113

### 6-(6-fluoro-1H-indol-3-yl)-2-(piperidin-4-ylmethyl)benzo[d]oxazole

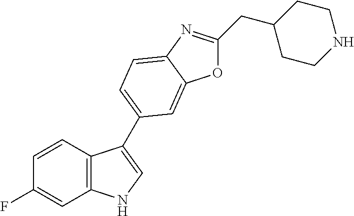 [SMILES: Fc1ccc2c(-c3ccc4nc(CC5CCNCC5)oc4c3)c[nH]c2c1]

Following the general method as outlined in Compound 1, starting from 6-(6-fluoro-1-(phenylsulfonyl)-1H-indol-3-yl)-2-(piperidin-4-ylmethyl)benzo[d]oxazole (Intermediate 132, 290 mg, 0.59 mmol), the title compound (15 mg, 7%) was obtained as a white solid. LC-MS for C₂₁H₂₀FN₃O+H⁺ [M+H]⁺: calcd. 350.2. found: 350.1. ¹H NMR (400 MHz, MeOD) δ [ppm]: 7.73-7.69 (m, 2H), 7.54 (d, J=0.8 Hz, 2H), 7.40 (s, 1H), 7.03 (dd, J=10.0, 2.0 Hz, 1H), 6.80 (td, J=9.2, 2.0 Hz, 1H), 2.99 (d, J=12.4 Hz, 2H), 2.80 (d, J=7.2 Hz, 2H), 2.57 (td, J=12.4, 2.4 Hz, 2H), 2.06-2.00 (m, 1H), 1.70 (d, J=12.8 Hz, 2H), 1.31-1.20 (m, 2H).

### Compound 114

### N-((6-(1H-indol-3-yl)benzo[d]oxazol-2-yl)methyl)methanesulfonamide

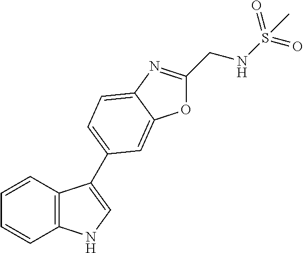 [SMILES: CS(=O)(=O)NCc1nc2ccc(-c3c[nH]c4ccccc34)cc2o1]

To a stirred solution of tert-butyl methylsulfonyl((6-(1-(phenylsulfonyl)-1H-indol-3-yl)benzo[d]oxazol-2-yl)methyl)carbamate (Intermediate 133, 1.25 g, 2.15 mmol) in MeOH (30 mL) was added NaOH (420 mg, 10.75 mmol). The mixture was stirred at 85° C. for 2 hours. The reaction was neutralized with HCl (12 M). The mixture was diluted with water (60 mL) and extracted with EtOAc (60 mL×3). The combined organic layer was washed with brine (30 mL), dried over anhydrous Na₂SO₄ and filtered. The filtrate was concentrated and purified by preparative HPLC to afford 215 mg (30%) of the title compound as a yellow solid. LC-MS for C₁₇H₁₅N₃O₃S+H⁺[M+H]⁺: calcd. 342.1. found: 341.9. ¹H NMR (400 MHz, DMSO-d₆) δ [ppm]: 11.44 (s, 1H), 8.00 (s, 1H), 7.98 (d, J=0.9 Hz, 1H), 7.92 (d, J=7.8 Hz, 1H), 7.78 (dd, J=7.0, 5.5 Hz, 2H), 7.72 (dd, J=8.3, 1.5 Hz, 1H), 7.47 (d, J=8.0 Hz, 1H), 7.20-7.15 (m, 1H), 7.14-7.09 (m, 1H), 4.54 (s, 2H), 3.04 (s, 3H).

### Compound 115

### (S)—N-((6-(6-fluoro-1H-indol-3-yl)benzo[d]oxazol-2-yl)methyl)-1-methylpyrrolidine-2-carboxamide

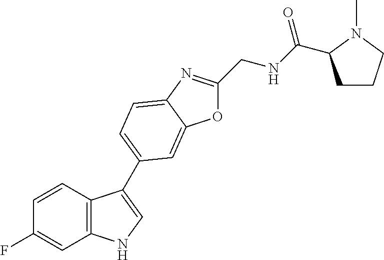 [SMILES: CN1CCC[C@H]1C(=O)NCc1nc2ccc(-c3c[nH]c4cc(F)ccc34)cc2o1]

To a stirred solution of (2S)-1-methylpyrrolidine-2-carboxylic acid (258 mg, 2.0 mmol) in THF/DMF (20 mL/2 mL) at room temperature was added HATU (760 mg, 2.0 mmol), DIPEA (387 mg, 3.0 mmol), and c-[5-(6-fluoro-1H-indol-3-yl)-benzooxazol-2-yl]-methylamine hydrochloride (Compound 81, 317 mg, 1.0 mmol). The mixture was stirred at room temperature for 2 hours, concentrated, diluted with EtOAC (100 mL) and washed with water (10 mL×4). The organic phase was dried over anhydrous Na₂SO₄ and filtered. The filtrate was concentrated and purified by preparative HPLC to afford 29 mg of the title compound. LC-MS for C₂₂H₂₁FN₄O₂+H⁺[M+H]⁺: calcd. 393.2. found: 393.1. ¹H NMR (400 MHz, DMSO-d₆) δ [ppm]: 11.48 (br s, 1H), 8.49 (t, J=5.6 Hz, 1H), 7.91-7.87 (m, 2H), 7.76 (d, J=2.8 Hz, 1H), 7.72 (d, J=8.4 Hz, 1H)), 7.66 (dd, J=8.0, 1.2 Hz, 1H), 7.24 (dd, J=10, 2.4 Hz, 1H), 6.98 (m, 1H), 4.62-4.57 (m, 2H), 3.08-3.07 (m, 1H), 2.83-2.80 (m, 1H), 2.36 (s, 3H), 2.30-2.28 (m, 1H), 2.11-2.07 (m, 1H), 1.78-1.74 (m, 3H).

### Compound 116

### 6-(6-fluoro-1H-indol-3-yl)-2-methoxybenzo[d]oxazole

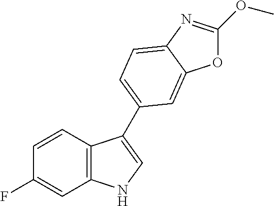 [SMILES: COc1nc2ccc(-c3c[nH]c4cc(F)ccc34)cc2o1]

Following the general method as outlined in Compound 1, starting from 6-(1-benzenesulfonyl-6-fluoro-1H-indol-3-yl)-2-methoxy-benzooxazole (Intermediate 134, 110 mg, 0.26 mmol), 20 mg (27%) of the title compound was obtained as a white solid. LC-MS for C₁₆H₁₁FN₂O₂+H⁺ [M+H]⁺: calcd. 283.1. found: 283.0. ¹H NMR (400 MHz, DMSO-d₆) δ [ppm]: 11.43 (brs, 1H), 7.86 (dd, J=8.8, 5.6 Hz, 1H), 7.79 (s, 1H), 7.69 (s, 1H), 7.59 (dd, J=8.0, 1.6 Hz, 1H), 7.54 (d, J=8.0 Hz, 1H), 7.22 (dd, J=9.6, 2.0 Hz, 1H), 6.98-6.93 (m, 1H), 4.19 (s, 3H).

### Compound 117

### Amino{[6-(6-fluoroindol-3-yl)benzoxazol-2-yl]methyl}sulfonamide

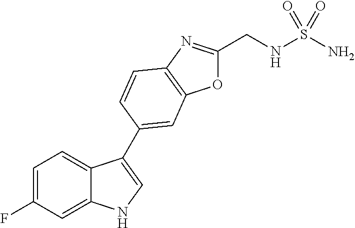 [SMILES: NS(=O)(=O)NCc1nc2ccc(-c3c[nH]c4cc(F)ccc34)cc2o1]

To the solution of tert-Butoxycarbonylamino {[6-(6-fluoroindol-3-yl)benzoxazol-2-yl]methyl}sulfonamide (Intermediate 135, 180 mg, 0.39 mmol) in THF (5 mL) was added EA/HCl (3 mL, 5 N). The mixture was stirred at room temperature overnight. The mixture was neutralized with THF/NH₃ (10 N) and filtered. The solvent was removed and the residue was purified by pre-TLC (DCM/Methanol=10/1) to give the title compound (50 mg, 34%) as a white solid. LC-MS for C₁₆H₁₃FN₄O₃S+H⁺[M+H]⁺: calcd. 361.1. found: 361.0. ¹H NMR (400 MHz, DMSO) δ 11.49 (br s, 1H), 7.94 (s, 1H), 7.89 (dd, J=8.8, 5.2 Hz, 1H), 7.77 (d, J=2.4 Hz, 1H), 7.74 (d, J=8.0 Hz, 1H), 7.68 (dd, J=8.0, 1.2 Hz, 1H), 7.46 (t, J=6.0 Hz, 1H), 7.24 (dd, J=10.0, 2.4 Hz, 1H), 6.97 (td, J=9.6, 2.4 Hz, 1H), 6.77 (s, 2H), 4.40 (d, J=6.4 Hz, 2H).

### Compound 118

### 5-(1H-indol-3-yl)benzo[d]oxazole

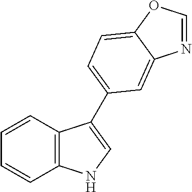 [SMILES: c1ccc2c(-c3ccc4ocnc4c3)c[nH]c2c1]

Following the general method as outlined in Compound 1, starting from 5-(1-(phenylsulfonyl)-1H-indol-3-yl)benzo[d]oxazole (Intermediate 136, 525 mg, 1.4 mmol), 56 mg (36%) of the title compound was obtained as a white solid. LC-MS for C₁₅H₁₀N₂O+H⁺ [M+H]⁺: calcd. 235.1. found: 235.1. ¹H NMR (400 MHz, CDCl₃) δ [ppm]: 8.30 (br s, 1H), 8.13 (s, 1H), 8.06 (d, J=1.1 Hz, 1H), 7.95 (d, J=7.9 Hz, 1H), 7.71 (dd, J=8.5, 1.6 Hz, 1H), 7.65 (dd, J=8.5, 0.5 Hz, 1H), 7.47-7.45 (m, 1H), 7.41 (d, J=2.8 Hz, 1H), 7.30-7.20 (m, 2H).

### Compound 119

### 6-(6-fluoro-1H-indol-3-yl)-2-((1-(methylsulfonyl)piperidin-4-yl)methyl)benzo[d]oxazole

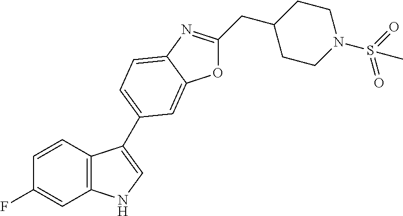 [SMILES: CS(=O)(=O)N1CCC(Cc2nc3ccc(-c4c[nH]c5cc(F)ccc45)cc3o2)CC1]

Following the general method as outlined in Compound 1, starting from 6-(6-fluoro-1-(phenylsulfonyl)-1H-indol-3-yl)-2-((1-(methylsulfonyl)piperidin-4-yl)methyl)benzo[d]oxazole (Intermediate 137, 220 mg, 0.388 mmol), the title compound (40 mg, 24%) was obtained as a white solid. LC-MS for C₂₂H₂₂FN₃O₃S+H⁺ [M+H]⁺: calcd. 428.1. found: 428.1. 1H NMR (400 MHz, DMSO-d₆) δ [ppm]: 11.47 (s, 1H), 7.90-7.86 (m, 2H), 7.75-7.70 (m, 2H), 7.66-7.64 (m, 1H), 7.23 (dd, J=10, 2.4 Hz, 1H), 6.96 (td, J=9.6, 2.4 Hz, 1H), 3.56 (d, J=12.0 Hz, 2H), 2.95 (d, J=6.8 Hz, 2H), 2.85 (s, 3H), 2.74 (td, J=12.4, 2.4 Hz, 2H), 2.07-2.01 (m, 1H), 1.84 (dd, J=12.4, 2.0 Hz, 2H) 1.37 (qd, J=12.4, 4.0 Hz, 2H).

### Compound 120

### 6-(6-fluoro-1H-indol-3-yl)-2-(2-(4-(methylsulfonyl)piperazin-1-yl)ethyl)benzo[d]oxazole

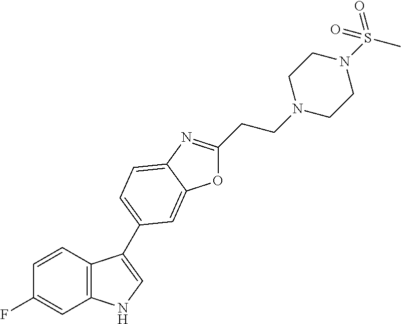 [SMILES: CS(=O)(=O)N1CCN(CCc2nc3ccc(-c4c[nH]c5cc(F)ccc45)cc3o2)CC1]

A solution of 2-(2-chloroethyl)-6-(6-fluoro-1-(phenylsulfonyl)-1H-indol-3-yl)benzo[d]oxazole (Intermediate 125, 110 mg, 0.24 mmol), sodium hydroxide (48 mg, 1.29 mmol) and 1-(methylsulfonyl)piperazine (80 mg, 0.48 mmol) in methanol (10 mL) was stirred at 50° C. overnight. The reaction mixture was cooled to room temperature and concentrated. The residue was diluted with water (20 mL) and extracted with EtOAc (50 mL). The organic layers was dried over anhydrous Na₂SO₄ and filtered. The filtrate was concentrated and purified by preparative HPLC to afford 73 mg (68%) of the title compound as a white solid. LC-MS for C₂₂H₂₃FN₄O₃S+H⁺[M+H]⁺: calcd. 443.2. found: 443.2. ¹H NMR (400 MHz, DMSO-d₆) δ [ppm]: 11.47 (s, 1H), 7.90-7.86 (m, 2H), 7.75 (d, J=2.5 Hz, 1H), 7.70 (d, J=8.3 Hz, 1H), 7.65 (dd, J=8.2, 1.5 Hz, 1H), 7.23 (dd, J=10.1, 2.4 Hz, 1H), 6.97 (td, J=9.6, 2.6 Hz, 1H), 3.15 (t, J=7.2 Hz, 2H), 3.11-3.03 (m, 4H), 2.91 (t, J=7.2 Hz, 2H), 2.85 (s, 3H), 2.63-2.55 (m, 4H).

### Compound 121

### 5-(5-chloro-1H-indol-3-yl)benzo[d]oxazole

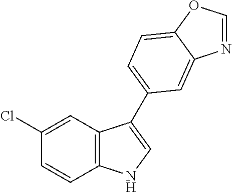 [SMILES: Clc1ccc2[nH]cc(-c3ccc4ocnc4c3)c2c1]

Following the general method as outlined for Compound 47, the title compound was obtained as a yellow solid after purification by preparative HPLC. LC-MS for C₁₅H₉ClN₂O+H [M+H]⁺: calcd. 269.0. found: 268.9. ¹H NMR (400 MHz, DMSO-d₆) δ [ppm]: 11.60 (br s, 1H), 8.76 (s, 1H), 8.01 (d, J=1.8 Hz, 1H), 7.85-7.81 (m, 3H), 7.73 (dd, J=8.4, 1.8 Hz, 1H), 7.49 (d, J=8.6 Hz, 1H), 7.18 (dd, J=8.6, 2.0 Hz, 1H).

### Compound 122

### N-((6-(6-fluoro-1H-indol-3-yl)benzo[d]oxazol-2-yl)methyl)-2-(4-methylpiperazin-1-yl)ethanesulfonamide

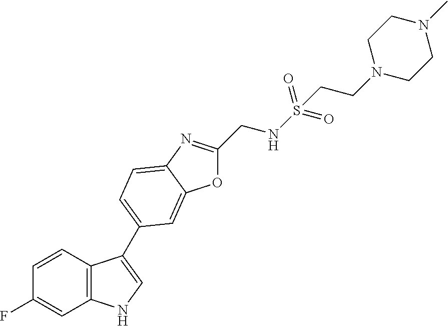 [SMILES: CN1CCN(CCS(=O)(=O)NCc2nc3ccc(-c4c[nH]c5cc(F)ccc45)cc3o2)CC1]

To a solution of N-((6-(6-fluoro-1H-indol-3-yl)benzo[d]oxazol-2-yl)methyl)ethenesulfonamide (Intermediate 119, 86 mg, 0.23 mmol) in MeCN/THF (5 mL/20 mL) was added 1-methylpiperazine (47 mg, 0.46 mmol). The mixture was stirred at room temperature for 72 hours. The mixture was diluted with water (60 mL) and extracted with EtOAc (60 mL×3). The combined organic layer was washed with brine (60 mL), dried over anhydrous Na₂SO₄ and filtered. The filtrate was concentrated and purified by preparative HPLC to afford 50 mg (46%) of the title compound as a white solid. LC-MS for C₂₃H₂₆FN₅O₃S+H⁺[M+H]⁺: calcd. 472.2. found: 472.8. ¹H NMR (400 MHz, DMSO-d₆) δ [ppm]: 11.50 (s, 1H), 8.02 (t, J=6.0 Hz, 1H), 7.96 (d, J=1.0 Hz, 1H), 7.89 (dd, J=8.8, 5.4 Hz, 1H), 7.78-7.75 (m, 2H), 7.70 (dd, J=8.3, 1.5 Hz, 1H), 7.24 (dd, J=9.9, 2.3 Hz, 1H), 6.97 (td, J=9.6, 2.4 Hz, 1H), 4.55 (d, J=5.8 Hz, 2H), 3.29 (d, J=7.8 Hz, 2H), 2.70-2.64 (m, 2H), 2.44-2.23 (m, 7H), 2.13 (s, 3H).

### Compound 123

### N-((6-(6-fluoro-1H-indol-3-yl)benzo[d]oxazol-2-yl)methyl)-2-(piperazin-1-yl)ethanesulfonamide

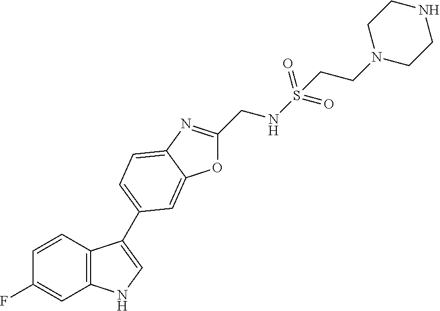 [SMILES: O=S(=O)(CCN1CCNCC1)NCc1nc2ccc(-c3c[nH]c4cc(F)ccc34)cc2o1]

To a solution of piperazine (752 mg, 8.73 mmol) in MeCN/THF (5 mL/30 mL) was added N-((6-(6-fluoro-1H-indol-3-yl)benzo[d]oxazol-2-yl)methyl)ethenesulfonamide (Intermediate 119, 180 mg, 0.485 mmol). The mixture was stirred at room temperature for 5 hours. The aqueous of NH₄Cl (20 mL) was added. The mixture was extracted with EA (60 mL×3). The organic phase was washed with brine (60 mL×3), dried over anhydrous Na₂SO₄ and filtered. The filtrate was concentrated and purified by preparative HPLC to afford 85 mg (35%) of the title compound as a yellow solid. LC-MS for C₂₂H₂₄FN₅O₃S+H [M+H]⁺: calcd. 458.2. found: 457.8. ¹H NMR (400 MHz, DMSO-d₆) δ [ppm]: 11.50 (s, 1H), 7.96 (d, J=1.0 Hz, 1H), 7.89 (dd, J=8.8, 5.4 Hz, 1H), 7.80-7.74 (m, 2H), 7.70 (dd, J=8.3, 1.5 Hz, 1H), 7.24 (dd, J=9.9, 2.3 Hz, 1H), 6.97 (td, J=9.5, 2.4 Hz, 1H), 4.55 (s, 2H), 3.29 (t, J=7.6 Hz, 2H), 2.66-2.61 (m, 6H), 2.33-2.24 (m, 4H).

### Compound 124

### N-((6-(6-fluoro-1H-indol-3-yl)benzo[d]oxazol-2-yl)methyl)-2-(pyrrolidin-1-yl)ethanesulfonamide

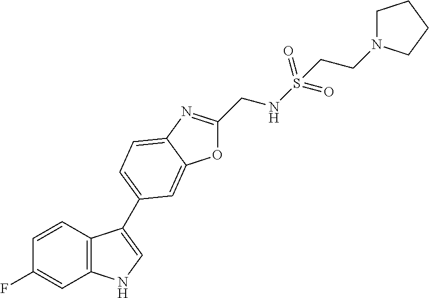 [SMILES: O=S(=O)(CCN1CCCC1)NCc1nc2ccc(-c3c[nH]c4cc(F)ccc34)cc2o1]

To a solution of pyrrolidine (752 mg, 8.73 mmol) in MeCN/THF (5 mL/30 mL) was added N-((6-(6-fluoro-1H-indol-3-yl)benzo[d]oxazol-2-yl)methyl)ethenesulfonamide (Intermediate 119, 180 mg, 0.485 mmol). The mixture was stirred at room temperature for 6 hours. The reaction mixture was concentrated and purified by Preparative HPLC to afford 120 mg (50%) of the title compound as a white solid. LC-MS for C₂₂H₂₃FN₄O₃S+H [M+H]⁺: calcd. 443.2. found: 443.6. ¹H NMR (400 MHz, DMSO-d₆) δ [ppm]: 11.50 (s, 1H), 8.03 (s, 1H), 7.95 (d, J=1.0 Hz, 1H), 7.89 (dd, J=8.8, 5.4 Hz, 1H), 7.79-7.74 (m, 2H), 7.70 (dd, J=8.3, 1.4 Hz, 1H), 7.24 (dd, J=9.9, 2.4 Hz, 1H), 6.97 (td, J=9.4, 2.4 Hz, 1H), 4.54 (s, 2H), 3.31-3.28 (m, 2H), 2.79-2.75 (m, 2H), 2.41 (s, 4H), 1.64 (dt, J=6.3, 3.0 Hz, 4H).

### Compound 125

### 6-(6-fluoro-1H-indol-3-yl)-2-((1-methylpiperidin-4-yl)methyl)benzo[d]oxazole

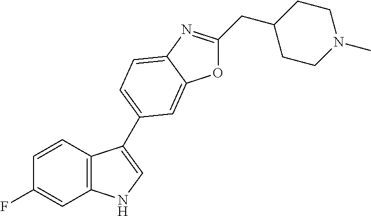 [SMILES: CN1CCC(Cc2nc3ccc(-c4c[nH]c5cc(F)ccc45)cc3o2)CC1]

Following the general method as outlined in Compound 1, starting from 6-(6-fluoro-1-(phenylsulfonyl)-1H-indol-3-yl)-2-((1-methylpiperidin-4-yl)methyl)benzo[d]oxazole (Intermediate 138, 200 mg, 0.4 mmol), the title compound (90 mg, 62%) was obtained as a yellow solid. LC-MS for C₂₂H₂₂FN₃O+H⁺[M+H]⁺: calcd. 364.2. found: 364.1. 1H NMR (400 MHz, DMSO-d₆) δ [ppm]: 11.47 (s, 1H), 7.89-7.86 (m, 2H), 7.74 (d, J=2.4 Hz, 1H), 7.70 (d, J=8.4 Hz, 1H), 7.64 (dd, J=1.6 Hz, J=8.8 Hz, 1H), 7.23 (dd, J=2.4 Hz, J=9.6 Hz, 1H), 6.96 (td, J=2.4 Hz, J=9.6 Hz, 1H), 2.87 (d, J=8.8 Hz, 2H), 2.73 (d, J=11.6 Hz, 2H), 2.13 (s, 3H), 1.88-1.81 (m, 3H), 1.68 (d, J=12.8 Hz, 2H) 1.36-1.28 (m, 2H),

### Compound 126

### N-((6-(6-fluoro-1H-indol-3-yl)benzo[d]oxazol-2-yl)methyl)-2-(4-(methylsulfonamido)piperidin-1-yl)ethanesulfonamide

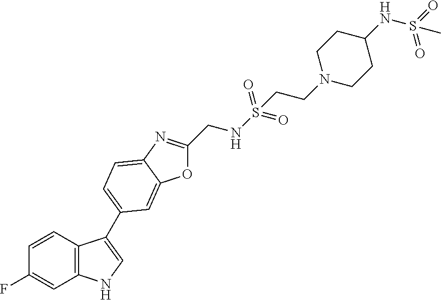 [SMILES: CS(=O)(=O)NC1CCN(CCS(=O)(=O)NCc2nc3ccc(-c4c[nH]c5cc(F)ccc45)cc3o2)CC1]

To a stirred solution of N-(piperidin-4-yl)methanesulfonamide hydrochloride (833 mg, 3.88 mmol) and DBU (591 mg, 3.88 mmol) in MeCN/THF (5 mL/30 mL) was added N-((6-(6-fluoro-1H-indol-3-yl)benzo[d]oxazol-2-yl)methyl)ethenesulfonamide (Intermediate 119, 160 mg, 0.43 mmol). The mixture was stirred at room temperature for 3 hours before it was quenched with aqueous NH₄Cl (30 mL). The mixture was extracted with EA (60 mL×3), washed with brine (60 mL×3), dried over anhydrous Na₂SO₄ and filtered. The filtrate was concentrated and purified by Preparative HPLC to afford 110 mg (47%) of the title compound as a white solid. LC-MS for C₂₄H₂₈FN₅O₅S₂+H⁺[M+H]⁺: calcd. 550.2. found: 550.7. ¹H NMR (400 MHz, DMSO-d₆) δ [ppm]: 11.51 (br s, 1H), 8.06-8.01 (m, 1H), 7.96 (d, J=1.0 Hz, 1H), 7.89 (dd, J=8.8, 5.4 Hz, 1H), 7.80-7.75 (m, 2H), 7.70 (dd, J=8.3, 1.5 Hz, 1H), 7.24 (dd, J=9.9, 2.4 Hz, 1H), 7.03-6.95 (m, 2H), 4.54 (d, J=5.6 Hz, 2H), 3.30 (s, 2H), 3.08 (br s, 1H), 2.86 (s, 3H), 2.76-2.74 (m, 2H), 2.69-2.67 (m, 2H), 2.03-1.98 (m, 2H), 1.77-1.75 (m, 2H), 1.45-1.40 (m, 2H).

### Compound 127

### N-((6-(6-chloro-1H-indol-3-yl)benzo[d]oxazol-2-yl)methyl)methanesulfonamide

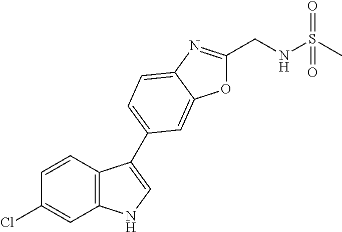 [SMILES: CS(=O)(=O)NCc1nc2ccc(-c3c[nH]c4cc(Cl)ccc34)cc2o1]

Following the general method as outlined for Compound 74, the title compound was obtained as a yellow solid after purification by preparative TLC (DCM/MeOH=10/1, v/v). LC-MS for C₁₇H₁₄ClN₃O₃S+H⁺[M+H]⁺: calcd. 376.0. found: 376.0. ¹H NMR (400 MHz, DMSO-d₆) δ [ppm]: 11.58 (br s, 1H), 7.98-7.95 (m, 2H), 7.92 (d, J=8.6 Hz, 1H), 7.83 (d, J=2.5 Hz, 1H), 7.77 (d, J=8.4 Hz, 1H), 7.70 (dd, J=8.3, 1.6 Hz, 1H), 7.51 (d, J=1.9 Hz, 1H), 7.12 (dd, J=8.6, 2.0 Hz, 1H), 4.54 (s, 2H), 3.03 (s, 3H).

### Compound 128

### N-((6-(5-fluoro-1H-indol-3-yl)benzo[d]oxazol-2-yl)methyl)methanesulfonamide

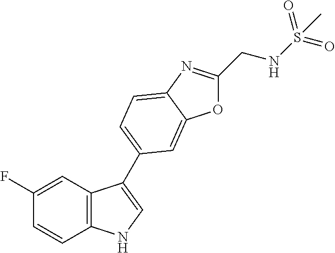 [SMILES: CS(=O)(=O)NCc1nc2ccc(-c3c[nH]c4ccc(F)cc34)cc2o1]

Following the general method as outlined for Compound 74, the title compound was obtained as a yellow solid after purification by preparative TLC (DCM/MeOH=10/1, v/v). LC-MS for C₁₇H₁₄FN₃O₃S+H⁺[M+H]⁺: calcd. 360.1. found: 360.1. ¹H NMR (400 MHz, DMSO-d₆) δ [ppm]: 11.59 (br s, 1H), 8.02 (br t, J=6.1 Hz, 1H), 7.98 (d, J=1.6 Hz, 1H), 7.87 (d, J=2.2 Hz, 1H), 7.76 (d, J=8.4 Hz, 1H), 7.70 (dd, J=8.4, 1.6 Hz, 1H), 7.64 (dd, J=10.6, 2.4 Hz, 1H), 7.47 (dd, J=8.8, 4.8 Hz, 1H), 7.03 (ddd, J=9.5, 8.8, 2.4 Hz, 1H), 4.54 (d, J=6.0 Hz, 2H), 3.04 (s, 3H).

### Compound 129

### 5-((6-(6-fluoro-1H-indol-3-yl)benzo[d]oxazol-2-yl)methyl)imidazolidine-2,4-dione

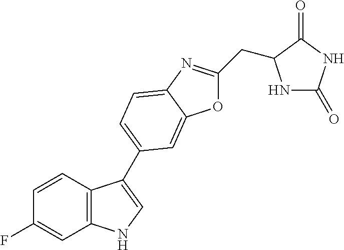 [SMILES: O=C1NC(=O)C(Cc2nc3ccc(-c4c[nH]c5cc(F)ccc45)cc3o2)N1]

To a solution of tert-butyl 3-(2-((2,5-dioxoimidazolidin-4-yl)methyl)benzo[d]oxazol-6-yl)-6-fluoro-1H-indole-1-carboxylate (Intermediate 141, 50 mg, 0.11 mmol) in DCM (1.5 mL) was added TFA (1.0 mL). The reaction mixture was stirred at room temperature for 2 hours and concentrated. The residue was diluted with EtOAc, washed with saturated aqueous NaHCO₃, dried over anhydrous Na₂SO₄ and filtered. The filtrate was concentrated and purified by preparative TLC (DCM/MeOH=5/1, v/v) to afford 9 mg (23%) of the title compound as a yellow solid. LC-MS for C₁₉H₁₃FN₄O₃—H⁻ [M−H]⁻: calcd. 363.1. found: 363.1. ¹H NMR (400 MHz, MeOH-d₄) δ [ppm]: 7.74-7.71 (m, 2H), 7.60-7.58 (m, 2H), 7.42 (s, 1H), 7.09 (dd, J=9.6, 2.0 Hz, 1H), 6.81 (td, J=8.8, 2.4 Hz, 1H), 4.59 (dd, J=7.2, 4.8 Hz, 1H), 3.43 (dd, J=16.4, 4.8 Hz, 1H), 3.27-3.25 (m, 1H).

### Compound 130

### 5-(6-fluoro-1H-indol-3-yl)benzo[d]isothiazol-3(2H)-one 1,1-dioxide

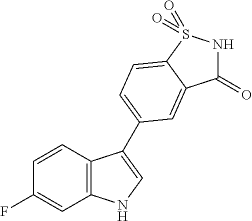 [SMILES: O=C1NS(=O)(=O)c2ccc(-c3c[nH]c4cc(F)ccc34)cc21]

### Step 1

### tert-butyl 3-(1,1-dioxido-3-oxo-2,3-dihydrobenzo[d]isothiazol-5-yl)-6-fluoro-1H-indole-1-carboxylate

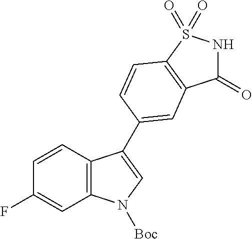 [SMILES: CC(C)(C)OC(=O)n1cc(-c2ccc3c(c2)C(=O)NS3(=O)=O)c2ccc(F)cc21]

To a mixture of 5-bromobenzo[d]isothiazol-3(2H)-one 1,1-dioxide (310 mg, 1.18 mmol, prepared as described in Tetrahedron 2006, 62, 7902.), tert-butyl 6-fluoro-3-(4,4,5,5-tetramethyl-1,3,2-dioxaborolan-2-yl)-1H-indole-1-carboxylate (Intermediate 2, 514 mg, 1.42 mmol) and K₂CO₃ (489 mg, 3.54 mmol) in dioxane (20 mL) and water (2 mL) was added Pd(PPh₃)₄ (69 mg, 0.06 mmol). The mixture was stirred at 110° C. for 2 hours before it was concentrated. The residue was diluted with EtOAc, washed with saturated aqueous NaHCO₃, dried over anhydrous Na₂SO₄ and filtered. The filtrate was concentrated to afford 300 mg (61%) of the title compound as a yellow solid, which was used directly without further purification.

### Step 2

A solution of tert-butyl 3-(1,1-dioxido-3-oxo-2,3-dihydrobenzo[d]isothiazol-5-yl)-6-fluoro-1H-indole-1-carboxylate (Step 1, 300 mg, 0.72 mmol) in HCl/EtOAc (10 mL, 2 M) was stirred at 60° C. for 2 hours. The reaction mixture was quenched with ammonia, concentrated and purified by silica gel chromatography (DCM) to afford 31 mg (14%) of the title compound as a yellow solid. LC-MS for C₁₅H₉FN₂O₃S—H⁻ [M−H]⁻: calcd. 315.0. found: 315.0. ¹H NMR (400 MHz, DMSO-d₆) δ [ppm]: 11.80 (br s, 1H), 8.26 (dd, J=8.4, 1.6 Hz, 1H), 8.17 (d, J=1.6 Hz, 1H), 8.12 (d, J=8.4 Hz, 1H), 8.10 (d, J=2.4 Hz, 1H), 7.93 (dd, J=8.8, 5.3 Hz, 1H), 7.28 (dd, J=9.8, 2.4 Hz, 1H), 7.05 (m, 1H).

### Compound 131

### 2-(6-(5-fluoro-1H-indol-3-yl)-2-oxobenzo[d]oxazol-3(2H)-yl)acetamide

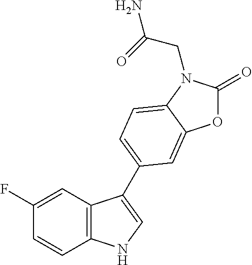 [SMILES: NC(=O)Cn1c(=O)oc2cc(-c3c[nH]c4ccc(F)cc34)ccc21]

To a solution of 2-(6-(5-fluoro-1-(phenylsulfonyl)-1H-indol-3-yl)-2-oxobenzo[d]oxazol-3(2H)-yl)acetamide (Intermediate 143, 281 mg, 0.6 mmol) in MeOH (30 mL) was added NaOH (241 mg, 6.04 mmol). The mixture was stirred at 85° C. for 1 hour. The reaction was neutralized with HCl (12 M). The mixture was diluted with water (60 mL) and extracted with EtOAc (60 mL×3). The combined organic layer was washed with water (60 mL×3), dried over anhydrous Na₂SO₄ and filtered. The filtrate was concentrated and purified by preparative HPLC to afford 100 mg (43%) of the title compound as a white solid. LC-MS for C₁₇H₁₂FN₃O₃—H⁻ [M−H]⁻: calcd. 324.1. found: 324.0. ¹H NMR (400 MHz, DMSO-d₆) δ [ppm]: 11.48 (s, 1H), 11.00 (s, 1H), 9.78 (s, 1H), 7.74 (d, J=2.6 Hz, 1H), 7.54 (dd, J=10.4, 2.4 Hz, 1H), 7.45 (dd, J=8.9, 4.7 Hz, 1H), 7.30 (d, J=8.1 Hz, 1H), 7.24 (d, J=1.9 Hz, 1H), 7.12 (dd, J=8.1, 1.9 Hz, 1H), 7.01 (td, J=9.1, 2.4 Hz, 1H), 4.28 (s, 2H).

### Compound 132

### 5-(6-fluoro-1H-indol-3-yl)isoindoline-1,3-dione

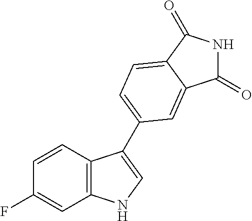 [SMILES: O=C1NC(=O)c2cc(-c3c[nH]c4cc(F)ccc34)ccc21]

A mixture of 4-(6-Fluoro-1H-indol-3-yl)-phthalic acid (Intermediate 145, 136 mg, 0.45 mmol), imidazole (46 mg, 0.68 mmol) and urea (54 mg, 0.90 mmol) were well triturated and placed in a flask followed by the addition of 4 drops of DMF. The mixture was heated at 150° C. in an oil bath for 3 hrs. The mixture was cooled down and poured into a 10% v/v aqueous HCl solution and stirred for 10 min. The solid was filtered off and purified by preparative HPLC to give 90 mg (58%) of the title compound as a yellow solid. LC-MS for C₁₆H₉FN₂O₂—H⁻ [M−H]⁻: calcd. 279.1. found: 279.0. ¹H NMR (400 MHz, DMSO-d₆) δ[ppm]: 11.75 (br s, 1H), 11.25 (br s, 1H), 8.14 (d, J=8.0 Hz, 1H), 8.06 (s, 1H), 8.05 (s, 1H), 7.92 (dd, J=8.8, 5.2 Hz, 1H), 7.85 (d, J=7.6 Hz, 1H), 7.27 (dd, J=9.8, 2.8 Hz, 1H), 7.03 (td, J=9.3, 2.4 Hz, 1H).

### Compound 133

### 6-fluoro-3-(isoindolin-5-yl)-1H-indole

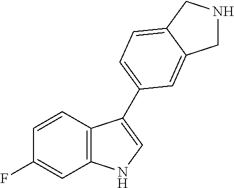 [SMILES: Fc1ccc2c(-c3ccc4c(c3)CNC4)c[nH]c2c1]

A solution of tert-butyl 3-(2-(tert-butoxycarbonyl)isoindolin-5-yl)-6-fluoro-1H-indole-1-carboxylate (Intermediate 146, 525 mg, 1.4 mmol) and CF₃COOH (8 mL) in DCM (20 mL) was stirred at room temperature overnight. The mixture was concentrated. The residue was diluted with water (100 mL) and extracted with EtOAc (300 mL). The organic layers was dried over anhydrous Na₂SO₄ and filtered. The filtrate was concentrated and purified by Preparative HPLC to afford 17 mg (15%) of the title compound as a white solid. LC-MS for C₁₆H₁₃FN₂+H]⁺[M+H]⁺: calcd. 253.1. found: 253.1. ¹H NMR (400 MHz, MeOD) δ [ppm]: 7.81-7.78 (m, 1H), 7.58 (s, 1H), 7.54 (d, J=7.6 Hz, 1H), 7.45 (s, 1H), 7.35 (d, J=7.8 Hz, 1H), 7.13 (dd, J=9.9, 2.4 Hz, 1H), 6.89 (td, J=9.6, 2.4 Hz, 1H), 4.27 (d, J=10.0 Hz, 4H).

### Compound 134

### 6-(6-fluoro-1H-indol-3-yl)benzo[d]isothiazol-3(2H)-one 1,1-dioxide

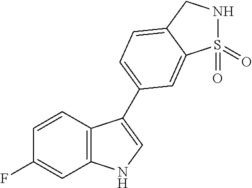 [SMILES: O=S1(=O)NCc2ccc(-c3c[nH]c4cc(F)ccc34)cc21]

To a stirred mixture of 3-(1,1-dioxo-2,3-dihydro-1H-1l6-benzo[d]isothiazol-6-yl)-6-fluoro-indole-1-carboxylic acid tert-butyl ester (Intermediate 147, 50 mg, 0.11 mmol) in DCM (10.0 mL) at room temperature was added TFA (1.0 mL). The mixture reaction was stirred for 2 hours and then it was concentrated under vacuum. The residue was purified by a preparative HPLC to afford 14 mg (31%) of the title compound as a yellow solid. LC-MS for C₁₅H₁₁FN₂O₂S—H⁻ [M−H]⁻: calcd. 301.1. found: 301.0. ¹H NMR (400 MHz, DMSO-d₆) δ [ppm]: 11.60 (s, 1H), 8.03-8.00 (m, 2H), 7.92 (d, J=2.4 Hz, 1H), 7.88-7.86 (m, 2H), 7.86 (s, 1H), 7.62 (d, J=8.0 Hz, 1H), 7.26 (dd, J=9.6, 2.0 Hz, 1H), 7.03-6.99 (m, 1H), 4.44 (s, 2H).

### Compound 135

### 5-(6-fluoro-1H-indol-3-yl)-2,3-dihydrobenzo[d]isothiazole 1,1-dioxide

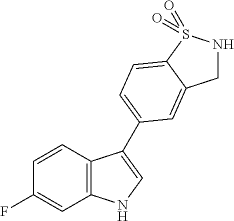 [SMILES: O=S1(=O)NCc2cc(-c3c[nH]c4cc(F)ccc34)ccc21]

### Step 1

### tert-butyl 3-(1,1-dioxido-2,3-dihydrobenzo[d]isothiazol-5-yl)-6-fluoro-1H-indole-1-carboxylate

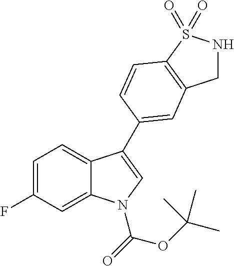 [SMILES: CC(C)(C)OC(=O)n1cc(-c2ccc3c(c2)CNS3(=O)=O)c2ccc(F)cc21]

A solution of 5-bromo-2,3-dihydrobenzo[d]isothiazole 1,1-dioxide (200 mg, 0.8 mmol), tert-butyl 6-fluoro-3-(4,4,5,5-tetramethyl-1,3,2-dioxaborolan-2-yl)-1H-indole-1-carboxylate (Intermediate 2, 292 mg, 0.8 mmol), Pd(PPh₃)₄ (46 mg, 0.08 mmol) and K₂CO₃ (221 mg, 2.4 mmol) in 20 mL of dioxane and 2 mL of water was stirred for 2 hrs at 110° C. The mixture was filtered and the filtrate was evaporated in vacuum. The residue was purified by preparative TLC (EtOAc/Petroleum Ether=1/3), 240 mg (74%) of the title compound as a white solid.

### Step 2

A solution of tert-butyl 3-(1,1-dioxido-2,3-dihydrobenzo[d]isothiazol-5-yl)-6-fluoro-1H-indole-1-carboxylate (240 mg, 0.59 mmol) was dissolved in 10 mL of EA/HCl. The solution was stirred for 2 hrs under 60° C. The mixture was filtered and the filtrate was evaporated in vacuum. The residue was purified by Preparative TLC (EtOAc/Petroleum Ether=1/3), yielding 32 mg (17%) of the final product as a yellow solid. LC-MS for C₁₅H₁₁FN₂O₂S—H⁻ [M−H]⁻: calcd. 301.0. found: 301.0. ¹H NMR (400 MHz, DMSO-d₆) δ [ppm]: 11.64 (br s, 1H), 7.95 (dd, J=8.4, 5.6 Hz, 1H), 7.90-7.80 (m, 4H), 7.74 (s, 1H), 7.27-7.25 (m, 1H), 7.03-6.98 (m, 1H), 4.45 (d, J=4.3 Hz, 2H).

### Compound 136

### 6-(6-fluoro-1H-indol-3-yl)-3-(piperidin-4-yl)benzo[d]oxazol-2(3H)-one

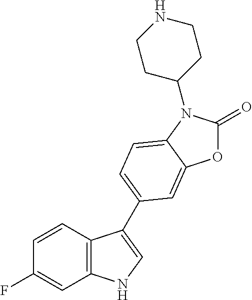 [SMILES: O=c1oc2cc(-c3c[nH]c4cc(F)ccc34)ccc2n1C1CCNCC1]

To a stirred solution of 3-[3-(1-tert-butoxycarbonyl-piperidin-4-yl)-2-oxo-2,3-dihydro-benzooxazol-6-yl]-6-fluoro-indole-1-carboxylic acid tert-butyl ester (Intermediate 150, 125 mg, 0.227 mmol) in DCM (5 mL) was added TFA (1 mL) dropwise at −60° C. The mixture was slowly warmed to room temperature and stirred for a further 16 hours before it was recooled to −78° C. The reaction was quenched with TEA (1.5 mL). The mixture was slowly warmed to room temperature, diluted with water (30 mL) and extracted with EtOAc (50 mL×2). The combined organic layer was washed with brine (20 mL×2), dried over anhydrous Na₂SO₄ and filtered. The filtrate was concentrated and purified by preparative HPLC (NH₄HCO₃ as additive) to afford 50 mg (63%) of the title compound as an off-white solid. LC-MS for C₂₀H₁₈FN₃O₂+H⁺[M+H]⁺: calcd. 352.1. found: 352.1. ¹H NMR (400 MHz, DMSO-d₆) δ [ppm]: 11.42 (br s, 1H), 7.81 (dd, J=8.8, 5.6 Hz, 1H), 7.68 (d, J=2.4 Hz, 1H), 7.60 (s, 1H), 7.53-7.45 (m, 2H), 7.22 (dd, J=10.0, 2.4 Hz, 1H), 6.95 (td, J=9.2, 2.4 Hz, 1H), 4.21-4.15 (m, 1H), 3.08 (d, J=12.0 Hz, 2H), 2.59 (t, J=12.0 Hz, 2H), 2.19-2.07 (m, 3H), 1.76-1.72 (m, 2H).

### Compound 137

### (6-(6-fluoro-1H-indol-3-yl)-2-methylbenzo[d]oxazol-5-yl)methanol

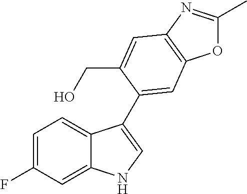 [SMILES: Cc1nc2cc(CO)c(-c3c[nH]c4cc(F)ccc34)cc2o1]

To the solution of 6-(6-Fluoro-1H-indol-3-yl)-2-methyl-benzooxazole-5-carboxylic acid methyl ester (Intermediate 154, 220 mg, 0.68 mmol) in THF (10 mL) was added diisobutylaluminum hydride (1.02 mL, 1.02 mmol 1M in THF). The mixture was stirred at 0° C. for 1 hr. Another batch of diisobutylaluminum hydride (1.02 mL, 1.02 mmol) was added and the mixture was stirred at rt for 4 hrs more before it was quenched with saturated NH₄Cl. The mixture was extracted with EtOAc (30 mL×3). The combined organic layer was washed with brine and dried over Na₂SO₄. The solvent was removed and the residue was purified by pre-TLC (DCM/methanol=20/1) and preparative HPLC to give to afford the title compound (23 mg, 11%) as a white solid. LC-MS for C₁₇H₁₃FN₂O₂+H⁺[M+H]⁺: calcd. 297.1. found: 297.1. ¹H NMR (400 MHz, DMSO) δ 11.40 (s, 1H), 7.81 (s, 1H), 7.59 (s, 1H), 7.55 (d, J=2.0 Hz, 1H), 7.41 (dd, J=8.4, 5.4 Hz, 1H), 7.24 (dd, J=10.0, 2.4 Hz, 1H), 6.89 (td, J=9.6, 2.4 Hz, 1H), 5.19 (t, J=5.6 Hz, 1H), 4.50 (d, J=5.6 Hz, 2H), 2.63 (s, 3H).

### Compound 138

### 5-(6-fluoro-H-indol-3-yl)-2-methylbenzo[d]isothiazol-3(2H)-one 1,1-dioxide

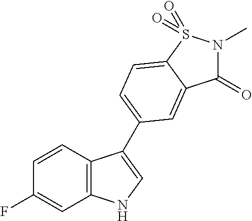 [SMILES: CN1C(=O)c2cc(-c3c[nH]c4cc(F)ccc34)ccc2S1(=O)=O]

A solution of tert-butyl 6-fluoro-3-(2-methyl-1,1-dioxido-3-oxo-2,3-dihydrobenzo[d]isothiazol-5-yl)-1H-indole-1-carboxylate (Intermediate 151, 60 mg, 0.14 mmol) in HCl/EtOAc (10 mL, 2 M) was stirred at 60° C. for 2 hours. The reaction mixture was quenched with ammonia, concentrated and purified by silica gel chromatography (DCM) to afford 35 mg (32%) of the title compound as a yellow solid. LC-MS for C₁₆H₁₁FN₂O₃S—H⁻ [M−H]⁻: calcd. 329.0. found: 329.0.

¹H NMR (400 MHz, DMSO-d₆) δ [ppm]: 11.86 (br s, 1H), 8.35-8.29 (m, 3H), 8.16 (d, J=2.4 Hz, 1H), 7.95 (dd, J=8.6, 5.0 Hz, 1H), 7.29 (dd, J=9.6, 2.0 Hz, 1H), 7.06-7.05 (m, 1H), 3.19 (s, 3H).

### Compound 139

### 2-(2-(cis-3,5-dimethylpiperazin-1-yl)ethyl)-6-(6-fluoro-1H-indol-3-yl)benzo[d]oxazole

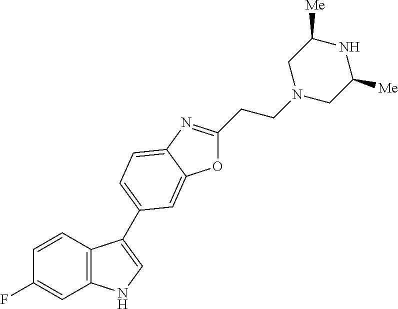 [SMILES: C[C@@H]1CN(CCc2nc3ccc(-c4c[nH]c5cc(F)ccc45)cc3o2)C[C@H](C)N1]

A solution of 6-(6-fluoro-1-(phenylsulfonyl)-1H-indol-3-yl)-2-vinylbenzo[d]oxazole (Intermediate 128, 100 mg, 0.24 mmol), sodium hydroxide (47.8 mg, 1.19 mmol) and cis-2,6-dimethyl-piperazine (133 mg, 1.16 mmol) in methanol (20 mL) was stirred at 50° C. overnight. The reaction mixture was cooled to room temperature and concentrated. The residue was diluted with water (100 mL) and extracted with EtOAc (100 mL*3). The organic layers was dried over anhydrous Na₂SO₄ and filtered. The filtrate was concentrated and purified by preparative HPLC (NH₄HCO₃) to afford 30 mg (32%) of the title compound as a white solid. LC-MS for C₂₃H₂₅FN₄O+H⁺[M+H]⁺: calcd. 393.2. found: 393.2. ¹H NMR (400 MHz, DMSO-d₆) δ [ppm]: 11.46 (br s, 1H), 7.89-7.86 (m, 2H), 7.74 (s, 1H), 7.70 (d, J=8.3 Hz, 1H), 7.64 (dd, J=8.2, 1.5 Hz, 1H), 7.23 (dd, J=9.9, 2.2 Hz, 1H), 6.96 (td, J=9.3, 2.4 Hz, 1H), 3.11 (t, J=7.3 Hz, 2H), 2.85-2.73 (m, 4H), 2.67 (s, 2H), 1.82 (s, 1H), 1.54 (t, J=10.3 Hz, 2H), 0.91 (d, J=6.3 Hz, 6H).

### Compound 140

### 1-(2-(6-(6-fluoro-1H-indol-3-yl)benzo[d]oxazol-2-yl)ethyl)piperidin-4-ol

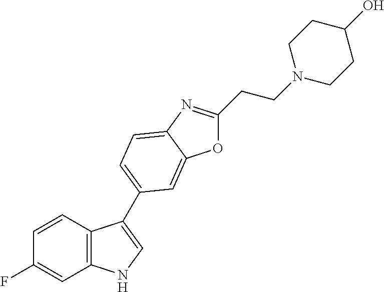 [SMILES: OC1CCN(CCc2nc3ccc(-c4c[nH]c5cc(F)ccc45)cc3o2)CC1]

A solution of 6-(6-fluoro-1-(phenylsulfonyl)-1H-indol-3-yl)-2-vinylbenzo[d]oxazole (Intermediate 128, 100 mg, 0.24 mmol), sodium hydroxide (48 mg, 1.19 mmol) and piperidin-4-ol (125 mg, 1.23 mmol) in methanol (20 mL) was stirred at 50° C. overnight. The reaction mixture was cooled to room temperature and concentrated. The residue was diluted with water (100 mL) and extracted with EtOAc (300 mL). The organic layers was dried over anhydrous Na₂SO₄ and filtered. The filtrate was concentrated and purified by Preparative HPLC (NH₄HCO₃) to give 33 mg (36%) of the title compound as a white solid. LC-MS for C₂₂H₂₂FN₃O₂+H]⁺[M+H]⁺: calcd. 380.2. found: 380.2. ¹H NMR (400 MHz, DMSO-d₆) δ [ppm]: 11.46 (br s 1H), 7.90-7.86 (m, 2H), 7.74 (d, J=2.5 Hz, 1H), 7.70 (d, J=8.4 Hz, 1H), 7.64 (dd, J=8.2, 1.5 Hz, 1H), 7.23 (dd, J=10.0, 2.4 Hz, 1H), 6.96-6.93 (m, 1H), 4.51 (s, 1H), 3.34-3.29 (m, 1H), 3.12-3.08 (m, 2H), 2.83-2.75 (m, 4H), 2.14-2.11 (m, 2H), 1.71-1.62 (m, 2H), 1.39-1.30 (m, 2H).

### Compound 141

### 2-(2-(trans-2,5-dimethylpiperazin-1-yl)ethyl)-6-(6-fluoro-1H-indol-3-yl)benzo[d]oxazole

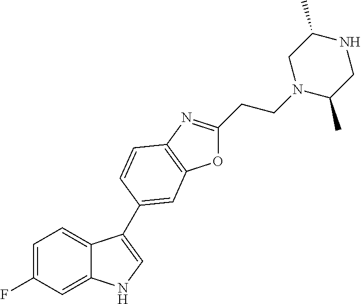 [SMILES: C[C@@H]1CN[C@@H](C)CN1CCc1nc2ccc(-c3c[nH]c4cc(F)ccc34)cc2o1]

A solution of 6-(6-fluoro-1-(phenylsulfonyl)-1H-indol-3-yl)-2-vinylbenzo[d]oxazole (Intermediate 128, 110 mg, 0.26 mmol), sodium hydroxide (53 mg, 1.31 mmol) and trans-2,5-dimethyl-piperazine (150 mg, 1.31 mmol) in methanol (20 mL) was stirred at 50° C. overnight. The reaction mixture was cooled to room temperature and concentrated. The residue was diluted with water (100 mL) and extracted with EtOAc. The organic layers was dried over anhydrous Na₂SO₄ and filtered. The filtrate was concentrated and purified by preparative HPLC (NH₄HCO₃) to afford 6 mg (6%) of the title compound as a white solid. LC-MS for C₂₃H₂₅FN₄O+H⁺[M+H]⁺: calcd: 393.2. found: 393.2. ¹H NMR (400 MHz, MeOD) δ [ppm]: 7.71-7.68 (m, 2H), 7.52 (s, 2H), 7.38 (s, 1H), 6.99 (dd, J=10.0, 2.4 Hz, 1H), 6.77 (td, J=9.2, 2.4 Hz, 1H), 3.25-3.19 (m, 1H), 3.05-3.00 (m, 2H), 2.85-2.79 (m, 2H), 2.78-2.67 (m, 2H), 2.36-2.21 (m, 1H), 2.20 (s, 1H), 2.04 (t, J=10.9 Hz, 1H), 1.10 (m, 6H).

### Compound 142

### (+)-2-(2-((3R,5R)-3,5-dimethylpiperazin-1-yl)ethyl)-6-(6-fluoro-1H-indol-3-yl)benzo[d]oxazole

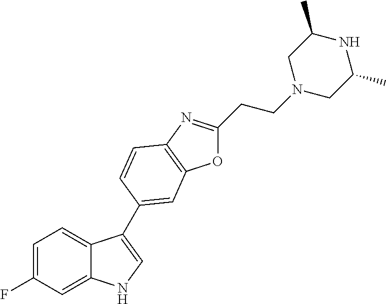 [SMILES: C[C@@H]1CN(CCc2nc3ccc(-c4c[nH]c5cc(F)ccc45)cc3o2)C[C@@H](C)N1]

To a stirred solution of 6-(6-fluoro-1-(phenylsulfonyl)-1H-indol-3-yl)-2-vinylbenzo[d]oxazole (Intermediate 128, 100 mg, 0.24 mmol) in MeOH (10 mL) was added (2R,6R)-2,6-dimethylpiperazine (273 mg, 2.39 mmol; made according to the procedures reported by J. Org. Chem. 1995, 60, 4177) and NaOH (48 mg, 1.20 mmol) at room temperature. The mixture was stirred at 50° C. under N₂ for 16 hrs. The mixture was concentrated and the residue was suspended with EtOAc (50 mL). The suspension was dried over anhydrous Na₂SO₄, filtered, concentrated, and purified by preparative HPLC (NH₄OH as additive) to afford the title compound 30 mg (32%) as a white solid. LC-MS for C₂₃H₂₅FN₄O+H⁺ [M+H]⁺: calcd: 393.2. found: 393.2.

¹H NMR (300 MHz, DMSO-d₆) δ [ppm]: 11.46 (br s, 1H), 7.90-7.85 (m, 2H), 7.74 (d, J=1.8 Hz, 1H), 7.69 (d, J=8.1 Hz, 1H), 7.64 (dd, J=8.1, 1.8 Hz, 1H), 7.23 (dd, J=9.6, 2.4 Hz, 1H), 6.96 (td, J=9.6, 2.4 Hz, 1H), 3.12-3.00 (m, 2H), 2.99-2.95 (m, 2H), 2.81-2.68 (m, 2H), 2.43-2.38 (m, 2H), 2.11-2.08 (m, 2H), 1.68 (brs, 1H), 0.95 (d, J=6.6 Hz, 6H).

[α]²⁰_{D}+10.9° (c=0.2, MeOH).

### Compound 143

### 2-(2-(2,6-diazaspiro[3.3]heptan-2-yl)ethyl)-6-(6-fluoro-1H-indol-3-yl)benzo[d]oxazole

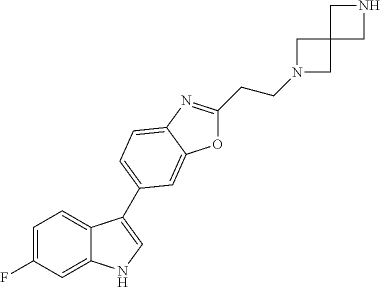 [SMILES: Fc1ccc2c(-c3ccc4nc(CCN5CC6(CNC6)C5)oc4c3)c[nH]c2c1]

To a solution of tert-butyl 6-(2-(6-(6-fluoro-1H-indol-3-yl)benzo[d]oxazol-2-yl)ethyl)-2,6-diazaspiro[3.3]heptane-2-carboxylate (Intermediate 152, 170 mg, 0.35 mmol) in DCM (3 mL) was added TFA (1 mL). The mixture was stirred at room temperature for 2 hs before it was concentrated. The residue was purified by Preparative HPLC to afford the product 66 mg (49%) as a white solid. LC-MS for C₂₂H₂₁FN₄O [M+H]⁺: calcd. 376.2. found: 376.8. ¹H NMR (400 MHz, DMSO-d₆) δ [ppm]: 11.50 (br s, 1H), 7.90-7.86 (m, 2H), 7.75 (s, 1H), 7.71-7.63 (m, 2H), 7.23 (dd, J=10.0, 2.4 Hz, 1H), 6.96 (td, J=18.8, 2.0 Hz, 1H), 3.82 (s, 2H), 3.48 (s, 2H), 3.20 (s, 4H), 2.94-2.90 (m, 2H), 2.83-2.81 (m, 2H).

### Compound 144

### (−)-2-(2-((3S,5S)-3,5-dimethylpiperazin-1-yl)ethyl)-6-(6-fluoro-1H-indol-3-yl)benzo[d]oxazole

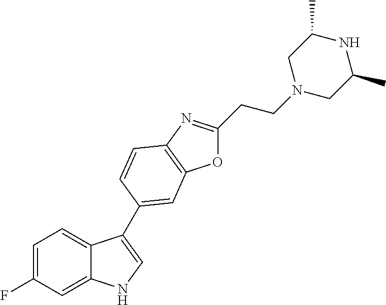 [SMILES: C[C@H]1CN(CCc2nc3ccc(-c4c[nH]c5cc(F)ccc45)cc3o2)C[C@H](C)N1]

To a stirred solution of 6-(6-fluoro-1-(phenylsulfonyl)-1H-indol-3-yl)-2-vinylbenzo[d]oxazole (Intermediate 128, 100 mg, 0.24 mmol) in MeOH (10 mL) was added (2S,6S)-2,6-dimethylpiperazine (136 mg, 1.19 mmol; made according to the procedures reported by J. Org. Chem. 1995, 60, 4177) and NaOH (48 mg, 1.20 mmol) at room temperature. The mixture was stirred at 50° C. under N₂ for 16 hrs. MeOH was removed under vacuum and the residue was suspended with EA (50 mL). The organic layer was dried over anhydrous Na₂SO₄, filtered, concentrated, and purified by preparative HPLC (NH₄OH as additive) to afford 45 mg (49%) of the title compound as white powder.

LC-MS for C₂₃H₂₅FN₄O+H⁺ [M+H]⁺: calcd: 393.2. found: 393.2.

¹H NMR (400 MHz, DMSO-d₆) δ [ppm]: 11.48 (br s, 1H), 7.89-7.86 (m, 2H), 7.75 (d, J=2.4 Hz, 1H), 7.67 (d, J=13.6 Hz, 2H), 7.24 (dd, J=10.0, 2.0 Hz, 1H), 6.96 (td, J=9.6, 2.4 Hz, 1H), 3.12-3.00 (m, 2H), 3.00-2.98 (m, 2H), 2.80-2.77 (m, 2H), 2.43-2.40 (m, 2H), 2.12-2.10 (m, 2H), 1.68 (brs, 1H), 0.97 (d, J=6.4 Hz, 6H).

### Compound 145

### 4-(2-(6-(6-fluoro-1H-indol-3-yl)benzo[d]oxazol-2-yl)ethyl)piperazine-2-carboxylic acid

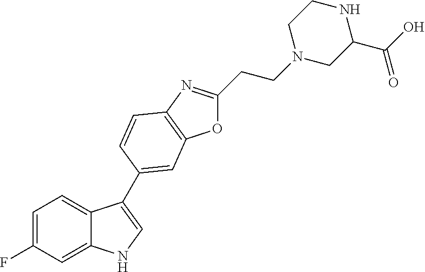 [SMILES: O=C(O)C1CN(CCc2nc3ccc(-c4c[nH]c5cc(F)ccc45)cc3o2)CCN1]

A solution of 6-(6-fluoro-1-(phenylsulfonyl)-1H-indol-3-yl)-2-vinylbenzo[d]oxazole (Intermediate 128, 120 mg, 0.28 mmol), sodium hydroxide (57 mg, 1.43 mmol) and piperazine-2-carboxylic acid (438 mg, 3.4 mmol) in methanol (20 mL) was stirred at 50° C. overnight. The reaction mixture was cooled to room temperature and concentrated. The residue was diluted with water (100 mL) and extracted with EtOAc (200 mL). The organic layers was dried over anhydrous Na₂SO₄ and filtered. The filtrate was concentrated and purified by preparative HPLC (NH₄HCO₃) to afford 12 mg (10%) of the title compound as a white solid. LC-MS for C₂₂H₂₁FN₄O₃+H⁺[M+H]⁺: calcd: 409.2. found: 408.8. ¹H NMR (400 MHz, DMSO-d₆) δ [ppm]: 11.51 (br s, 1H), 7.90-7.87 (m, 2H), 7.75 (s, 1H), 7.71 (d, J=8.4 Hz, 1H), 7.65 (d, J=8.0 Hz, 1H), 7.24 (d, J=10.4 Hz, 1H), 6.96 (t, J=9.6 Hz, 1H), 3.34-3.09 (m, 5H), 3.05 (d, J=11.6 Hz, 1H), 2.92-2.87 (m, 3H), 2.78 (t, J=11.6 Hz, 1H), 2.27-2.18 (m, 2H).

### Compound 146

### 3-(benzofuran-6-yl)-6-fluoro-1H-indole

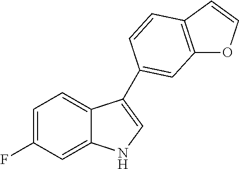 [SMILES: Fc1ccc2c(-c3ccc4ccoc4c3)c[nH]c2c1]

To a stirred solution of tert-butyl 3-(benzofuran-6-yl)-6-fluoro-1H-indole-1-carboxylate (Intermediate 153, 58 mg, 0.32 mmol) in DCM (2.0 mL), anisole (0.2 mL) and TFA (1 mL) were added. The mixture was stirred at room temperature overnight. The reaction mixture was concentrated. The residue was purified with preparative TLC (EtOAc/Petroleum Ether=1/5) and preparative HPLC to afford 16 mg of the title compound. LC-MS for C₁₆H₁₀FNO+H⁺[M+H]⁺: calcd: 252.1. found: 251.9. ¹H NMR (400 MHz, DMSO-d₆) δ [ppm]: 11.47 (br s, 1H), 7.98 (d, J=1.6 Hz, 1H), 7.90 (dd, J=8.8, 5.2 Hz, 1H), 7.84 (s, 1H), 7.55 (d, J=2.4 Hz, 1H), 7.70 (d, J=8.0 Hz, 1H), 7.59 (d, J=8.0 Hz, 1H), 7.24 (dd, J=9.8, 2.4 Hz, 1H), 6.97 (td, J=9.2, 2.4 Hz, 2H).

### II. Biology Examples

### II.1. Assay for TDO2 Enzymatic Activity Determination

The compounds of formula I inhibit the enzymatic activity of human TDO2.

To measure the TDO2 activity, the procedure described in Dolusic et al. J. Med. Chem.; 2011, 54, 5320-533 was adapted: the reaction mixture contained (final concentrations) potassium phosphate buffer (50 mM, pH 7.5), ascorbic acid (0.25 M), methylene blue (0.125 μM), catalase (40 units/mL, from bovine liver, Sigma), and human recombinant TDO2 enzyme (prepared as described in Dolusic et al. J. Med. Chem.; 2011, 54, 5320-5334; 0.9 μg) without or with the compounds of the present invention at the indicated concentrations (total volume 112.5 μL). The reaction was initiated by the addition of 37.5 μL of L-Trp (final concentration 1 mM) at room temperature. The reaction was conducted at room temperature during one hour and stopped by the addition of 30 μL of 30% (w/v) trichloroacetic acid.

To convert N-formylkynurenine into kynurenine, the reaction mixture was incubated at 65° C. for 30 min. Then 150 μL of the reaction mixture was mixed with 120 μL of 2.5% (w/v) 4-(dimethylamino)-benzaldehyde in acetic acid and incubated for 5 min at room temperature. Kynurenine concentrations were determined by measuring the absorbance at 480 nm. A standard curve was made with pure kynurenine. The TDO activity was measured as described above using ten serial concentrations of the compounds of the present invention. Data were fitted using the Prism™ software (GraphPad Software, Inc.) using standard parameters.

The biological activity of representative Compounds is summarized in the following table:

| Compound | IC₅₀ (nM) |
|---|---|
| 1 | 586 |
| 2 | 946 |
| 3 | 2122 |
| 4 | 3179 |
| 5 | 1166 |
| 6 | 7526 |
| 7 | 13770 |
| 8 | 609 |
| 9 | 645 |
| 10 | 427 |
| 11 | 680 |
| 12 | 709 |
| 13 | 407 |
| 14 | 2221 |
| 15 | 1158 |
| 16 | 1990 |
| 17 | 935 |
| 18 | 948 |
| 19 | 2192 |
| 20 | 421 |
| 21 | 2667 |
| 22 | 539 |
| 23 | 322 |
| 24 | 924 |
| 25 | 188 |
| 26 | 388 |
| 46 | 1332 |
| 96 | 2387 |
| 97 | 716 |
| 98 | 489 |
| 100 | 1464 |
| 101 | 1380 |
| 102 | 835 |
| 105 | 913 |
| 106 | 814 |
| 107 | 2796 |
| 110 | 1630 |
| 122 | 2089 |
| 123 | 1359 |
| 124 | 1208 |
| 128 | 6066 |
| 130 | 2423 |

In one embodiment, compounds having an IC50<2000 nm, preferably compound having an IC50<1000 nm are selected.

### II.2. Cellular Assay for TDO2 Activity Determination

### II.2. A h TDO2-Overexpressing P815 Cells

The compounds of Formula I inhibit the activity of human TDO2 in cells.

The assay (adapted from Pilotte L et al., Proc Natl Acad Sci USA, 2012, 109(7), 2497-502) was performed in 96-well flat bottom plates seeded with murine mastocytoma P815 cells overexpressing hTDO2 (prepared as described in Pilotte et al., PNAS, 2012, 109(7), 2497-2502), at a concentration of 5×10⁴ cells/well in a final volume of 200 μL. To determine TDO or IDO activity, the cells were incubated overnight at 37° C. at 5% CO₂ in IMDM (Invitrogen) supplemented with 2% FBS and 2% penicillin/streptomycin in the presence of the compounds of the present invention, at different concentrations. The cells may be obtained from the American Type Cuylture Collecion [ATCC® TIB-64™ or commercially, e.g., from Sigma-Aldrich or Life Technologies.]

The plates were then centrifuged 5 min at 1000 rpm, and 100 μL of the supernatant were collected in a conical plate, 30 uL of TCA 30% were added and a further centrifugated at 3000×g for 10 minutes. 100 μL of the supernatant were collected in a flat bottomed plate and 100 μL of 2% (w/v) 4-(dimethylamino)-benzaldehyde in acetic acid and incubated for 5 min at room temperature. Kynurenine concentrations were determined by measuring the absorbance at 480 nm. A standard curve was made with pure kynurenine. The TDO activity was measured as described above using ten serial concentrations of the compounds of the present invention. Data were fitted using the Prism™ software (GraphPad Software, Inc.) using standard parameters.

The biological activity of representative compounds is summarized in the following table:

| Compound | IC50 (nM) |
|---|---|
| 1 | 154 |
| 26 | 150 |
| 27 | 678 |
| 28 | 1447 |
| 30 | 896 |
| 31 | 2299 |
| 32 | 460 |
| 33 | 635 |
| 34 | 863 |
| 35 | 689 |
| 36 | 1131 |
| 37 | 1534 |
| 38 | 505 |
| 39 | 2864 |
| 40 | 750 |
| 44 | 345 |
| 45 | 595 |
| 47 | 100 |
| 48 | 118 |
| 53 | 79 |
| 54 | 109 |
| 59 | 622 |
| 60 | 638 |
| 62 | 210 |
| 64 | 176 |
| 65 | 109 |
| 66 | 203 |
| 68 | 78 |
| 70 | 251 |
| 74 | 72 |
| 75 | 92 |
| 77 | 246 |
| 78 | 82 |

In one embodiment, compounds having an IC₅₀<2000 nm are selected. In another embodiment, compounds having an IC₅₀<1000 nm are selected.

### II.2.b A172 Cells

The compounds of formula I inhibit the activity of human TDO2 in cells that constitutively express TDO2, such as A172 cells. A172 is a cell line derived from human brain glioblastoma cells. The cells are available from the American Type Culture Collection (ATCC®) as CRL-1620™

The assay (adapted from Pilotte L et al., Proc Natl Acad Sci USA, 2012, 109(7), 2497-502) was performed in 96-well flat bottom plates seeded with human glioblastoma A172 cells, naturally expressing hTDO2 (prepared as described in Tilman et al., Mol Cancer, 2007, 17(6), 80), at a concentration of 1.25×10⁴ cells/well in a final volume of 200 μL. To determine TDO, the cells were incubated overnight at 37° C. at 5% CO₂ in IMDM (Invitrogen) supplemented with 2% FBS and 2% penicillin/streptomycin in the presence of the compounds of the present invention, at different concentrations.

The plates were then centrifuged 5 min at 1000 rpm, and 100 μL of the supernatant were collected in a conical plate, 30 uL of TCA 30% were added and a further centrifugated at 3000×g for 10 minutes. 100 μL of the supernatant were collected in a flat bottomed plate and 100 μL of 2% (w/v) 4-(dimethylamino)-benzaldehyde in acetic acid and incubated for 5 min at room temperature. Kynurenine concentrations were determined by measuring the absorbance at 480 nm. A standard curve was made with pure kynurenine. The TDO activity was measured as described above using ten serial concentrations of the compounds of the present invention. Data were fitted using the Prism™ software (GraphPad Software, Inc.) using standard parameters.

The biological activity of representative compounds is summarized in the following table:

| Compound | IC50 (nM) |
|---|---|
| 1 | 178 |
| 9 | 147 |
| 12 | 517 |
| 20 | 169 |
| 22 | 176 |
| 38 | 512 |
| 41 | 1092 |
| 47 | 479 |
| 55 | 171 |
| 56 | 311 |
| 58 | 3767 |
| 61 | 459 |
| 62 | 335 |
| 63 | 597 |
| 64 | 501 |
| 65 | 335 |
| 66 | 284 |
| 67 | 2520 |
| 68 | 218 |
| 69 | 3284 |
| 70 | 435 |
| 71 | 3532 |
| 72 | 442 |
| 73 | 363 |
| 74 | 152 |
| 75 | 143 |
| 76 | 440 |
| 77 | 265 |
| 78 | 149 |
| 79 | 802 |
| 80 | 842 |
| 81 | 1615 |
| 82 | 602 |
| 83 | 154 |
| 84 | 1020 |
| 85 | 1333 |
| 86 | 646 |
| 87 | 298 |
| 88 | 170 |
| 89 | 205 |
| 90 | 240 |
| 91 | 213 |
| 92 | 508 |
| 93 | 1154 |
| 94 | 4643 |
| 95 | 311 |
| 96 | 589 |
| 97 | 284 |
| 98 | 293 |
| 99 | 351 |
| 100 | 345 |
| 101 | 153 |
| 102 | 261 |
| 103 | 427 |
| 104 | 464 |
| 105 | 223 |
| 106 | 326 |
| 107 | 677 |
| 108 | 599 |
| 109 | 446 |
| 110 | 239 |
| 111 | 18980 |
| 112 | 979 |
| 113 | 284 |
| 114 | 478 |
| 115 | 349 |
| 116 | 372 |
| 117 | 278 |
| 118 | 1529 |
| 119 | 1022 |
| 120 | 443 |
| 121 | 1772 |
| 122 | 344 |
| 123 | 498 |
| 124 | 334 |
| 125 | 148 |
| 126 | 826 |
| 127 | 3783 |
| 128 | 540 |
| 129 | 225 |
| 130 | 83 |
| 131 | 2385 |
| 132 | 197 |
| 133 | 292 |
| 134 | 771 |
| 135 | 74 |
| 136 | 320 |
| 137 | 30680 |
| 138 | 3681 |
| 139 | 383 |
| 140 | 447 |
| 141 | 340 |
| 142 | 480 |
| 143 | 610 |
| 144 | 460 |
| 145 | 26000 |
| 146 | 520 |

In one embodiment, compounds having an IC₅₀<2000 nm are selected. In another embodiment, compounds having an IC₅₀<1000 nm are selected.

### II.3. Pharmacodynamic Assay for TDO2 In Vivo Activity Determination: Increase of Blood Tryptophan Levels in Mice

The compounds of the present invention increase the amount of Tryptophan in mouse blood. Briefly, female BALB/c mice (7-8 weeks old) were treated with either a suspension of one of the compounds of the present invention in 0.5% hydroxypropyl methyl cellulose (HPMC) K4M (4000 mPa·s (cPs), Methocell™, Dow chemical)/0.25% Tween® 20 (Signma Aldrich) at different doses (30, 60 and 100 mg/kg), or with a vehicle control (0.5% HPMC K4M/0.25% Tween 20), by the oral route by gavage (dosing volume 5 mL/kg, 10 mice per group). After two hours, blood was harvested, plasma was prepared and the amount of Tryptophan present was determined by LC-MS-MS (HPLC column Unison UK-Phenyl, 75×4.6, 3 μm, flow rate 0.8 mL/min, 8 minutes gradient from 95% water+0.1% formic acid/5% Acetonitrile+0.1% formic acid to 5% water+0.1% formic acid/95% Acetonitrile+0.1% formic acid, retention time 2.4 min; API4000 MS-MS system from AB Sciex, ESI+ mode, parent ion 205.1, daughter ion 146.1).

Compound 110 increased circulating Tryptophan by 46% at 30 mg/kg (p<0.0001), by 52% at 60 mg/kg (p<0.0001) and by 78% at 100 mg/kg (p<0.0001) compared to vehicle-treated controls, as evidenced in the table below.

Tryptophan concentration in plasma (average±standard error of the mean, nM):

| | Vehicle | 30 mg/kg | 60 mg/kg | 100 mg/kg |
|---|---|---|---|---|
| Compound 110 | 85900 ± 3900 | 125000 ± 7000 | 130300 ± | 153200 ± |
| | | | 7500 | 5700 |

All publications cited in this specification and priority applications including US Provisional Patent Application No. 61/996,975, filed Mar. 18, 2014, are incorporated herein by reference. While the invention has been described with reference to particular embodiments, it will be appreciated that modifications can be made without departing from the spirit of the invention. Such modifications are intended to fall within the scope of the appended claims.

1. A compound of Formula I 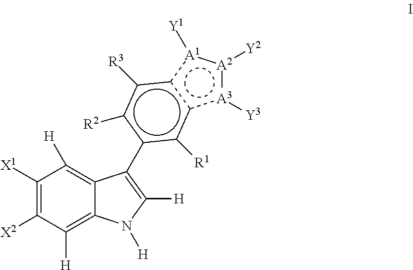
or a pharmaceutically acceptable enantiomer, salt or solvate thereof, wherein:
X¹ and X² represent each independently H, halogen, alkyl, or haloalkyl;
R¹, R² and R³ represent each independently H, halogen, C1-C6 alkyl, alkoxy, or haloalkyl, optionally substituted by one or more substituents selected from halogen, hydroxyl, OR⁴, COOR⁴, CONR⁴R⁵, NR⁴COR⁵, NR⁴R⁵, SO₂R⁴, SO₂NR⁴R⁵, NR⁴SO₂R⁵, SO₂R⁴, aryl, CO-alkyl, or C1-C6 alkyl which is optionally substituted by one or more groups selected from halogen, hydroxyl, amino or COOH; wherein R⁴ and R⁵ represent each independently a hydrogen atom or a group, optionally substituted, selected from C1-C6 alkyl, aryl, arylalkyl, alkylaryl, heteroaryl, heteroarylalkyl, alkylheteroaryl, or amino;
A¹, A² and A³ represent each independently C, N, S or O, wherein when A¹, A² or A³ is S, A¹-Y¹, A²-Y² or A³-Y³ is optionally SO₂;
each of Y¹, Y² and Y³ is either absent or represent independently
e) a hydrogen atom;
f) oxo;
g) SH
h) CR⁶R⁷R⁸, NR⁶R⁷ and OR⁶ wherein R⁶, R⁷ and R⁸ represent each independently:
i) a hydrogen atom;
ii) halogen;
iii) hydroxyl;
iv) OR⁹ or NR⁹R¹⁰ wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, amino, CO-alkyl, or SO₂R¹¹, wherein R¹¹ represents a hydrogen atom or a group, optionally substituted, selected from C1-C6 alkyl, aryl, arylalkyl, alkylaryl, heteroaryl, heteroarylalkyl, alkylheteroaryl, hydroxyl, or amino;
v) C1-C10 alkyl, linear or branched; optionally substituted with up to three substituents selected from halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, or CO-alkyl, wherein R⁹ and R¹⁰ represent each independently a hydrogen atom or a group, optionally substituted, selected from C1-C6 alkyl, heterocyclyl, aryl, arylalkyl, alkylaryl, heteroaryl, heteroarylalkyl, alkylheteroaryl, or amino;
vi) heterocyclyl or C1-C2 alkyl-heterocyclyl. the heterocyclyl being optionally substituted with up to three substituents halogen, hydroxyl, oxo, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, or alkyl, the alkyl group being optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino;
vii) —CO—R¹¹ or —SO₂R¹¹ wherein R¹¹ represents a group selected from hydroxy, amine, alkyl, heterocyclyl; optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SOR⁹, aryl, CO-alkyl, or C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino;
viii) optionally when Y1, Y2 or Y3 is CR⁶R⁷R⁸, R⁶, R⁷ and the carbon atom to which they are attached form together a ring selected from:
cycloalkyl, optionally substituted with up to three substituents selected from halogen, hydroxyl, OR⁹, COOR⁹, CONR⁹R¹⁰ NR⁹COR¹⁰, NR⁹R¹⁰, SO₂R⁹, SO₂NR⁹R¹⁰, NR⁹SO₂R¹⁰, SO₂R⁹, aryl, CO-alkyl, or C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino; or
heterocyclyl, optionally substituted with up to three substituents selected from the group comprising halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹⁰, SO₂R⁹, aryl, CO-alkyl, or a C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino; or
ix) optionally when Y1, Y2 or Y3 is NR⁶R⁷, R⁶, R⁷ and the nitrogen atom to which they are attached form together a ring; optionally substituted with up to three substituents selected from halogen, hydroxyl, OR⁹, COOR⁹, CONR⁹R¹⁰, NR⁹COR¹⁰, NR⁹R¹⁰, SO₂R⁹, SO₂NR⁹R¹⁰, NR⁹SO₂R¹¹, SO₂R⁹, aryl, CO-alkyl, or C1-C6 alkyl which is optionally substituted by one or more groups selected from halogen, hydroxyl, amino or COOH; wherein R⁹ and R¹⁰ represent each independently a hydrogen atom or a group, optionally substituted, selected from C1-C6 alkyl, aryl, arylalkyl, alkylaryl, heteroaryl, heteroarylalkyl, alkylheteroaryl, or amino; and wherein R¹¹ represents a hydrogen atom or an optionally substituted group selected from aryl, arylalkyl, alkylaryl, heteroaryl, heteroarylalkyl, alkylheteroaryl, or amino; or R¹ represents an alkyl group optionally substituted with up to three substituents selected from halogen, hydroxyl, OR¹², COOR¹², CONR¹²R¹³, NR¹²COR¹³, NR¹²R¹³, SO₂R¹², SO₂NR¹²R¹³, NR¹²SO₂R¹³, SO₂R¹², or aryl; wherein R¹² and R¹³ represent each independently a hydrogen atom or a group, optionally substituted, selected from C1-C6 alkyl, aryl, arylalkyl, alkylaryl, heteroaryl, heteroarylalkyl, alkylheteroaryl, or amino;
dotted lines stand for single or double bonds;
provided that A¹, A² and A³ are not all C;
provided that when one of A and A³ is N, the two others are not both C; and provided that when one of A¹, A² and A³ is S, only one S is present, at least one C is present, and the other is C or N;
provided that compound of Formula I is not 3-(benzofuran-5-yl)-6-chloro-1H-indole or 3-(benzo[d][1,3]dioxol-5-yl)-1H-indole.

2. The compound according to claim 1, wherein X¹ and X² are independently H or F.

3. The compound according to claim 2, wherein R¹, R² and R³ represent each independently H, halogen or methyl.

4. The compound according to claim 3, wherein R¹, R² and R³ are each H.

5. The compound according to claim 1, wherein when R⁶, R⁷ or R⁸ represent a halogen, the halogen is F.

6. The compound according to claim 1, wherein when R⁶, R⁷ or R⁸ is heterocyclyl or C1-heterocycyl, the heterocyclyl is piperidine, pyrrolidine, piperazine, morpholine, or 2,6-diazaspiro[3.3]heptane, any of which may be optionally substituted with one or more of C1-C3 alkyl, amino, hydroxyl, halogen, COCH3, COOH, or SO₂CH₃.

7. The compound according to claim 1, wherein when R⁶, R⁷ or R⁸ is C1-C10 alkyl, the alkyl is methyl, ethyl or propyl.

8. The compound according to claim 1, wherein when R¹¹ is heterocycle, the heterocycle is piperidine, pyrrolidine, piperazine or tetrahydrothiopyrandioxide.

9. The compound according to claim 1, wherein when R⁶, R⁷ and the carbon atom to which they are attached form a ring which is a heterocycl, the heterocycle is morpholine, piperazine or piperidine, any of which may be optionally substituted.

10. The compound according to claim 1, wherein when R⁶, R⁷, and the nitrogen atom to which they are attached form a heterocyclyl ring.

11. The compound according to claim 10, wherein the heterocycle is morpholine, piperazine or piperidine, any of which may be optionally substituted.

12. The compound according to claim 1, wherein a first one of A¹, A² and A³ is N, a second one of A, A² and A³ is C and the third one of A, A² and A³ is N or O.

13. The compound according to claim 1, wherein A² is N and one of A and A³ is N or S and the other is C.

14. The compound according to claim 13, wherein when A or A³ is S, A¹-Y¹ or A³-Y³ is SO₂.

15. The compound according to claim 1, wherein A² is C and one of A¹ and A³ is N and the other is N or O.

16. The compound according to claim 15, wherein A¹ or A³ is O, and another of A¹, A² or A³ is C is substituted with CR⁶R⁷R⁸ and R⁶, R⁷ or R⁸ is heterocyclyl or C1-C2 alkyl-heterocycyl, the heterocyclyl is piperidine, pyrrolidine, piperazine, morpholine, or azetidine substituted with azetidine, any of which may be optionally substituted with one or more of C1-C3 alkyl, amino, hydroxyl, halogen, COCH3, COOH, or SO₂CH₃.

17. The compound according to claim 1, wherein one of A¹, A² or A³ is S or A¹-Y¹, A²-Y², or A³-Y³ is SO₂; a second of A¹, A² or A³ is N or C and the third of A¹, A² or A³ is C.

18. The compound according to claim 1, selected from the group consisting of:
6-(6-fluoro-1H-indol-3-yl)-1H-indazole;
2-(6-(6-fluoro-1H-indol-3-yl)-1H-indazol-1-yl)acetamide;
6-(6-fluoro-1H-indol-3-yl)-1-(piperidin-4-ylmethyl)-1H-indazole;
1-(4-((6-(6-fluoro-1H-indol-3-yl)-1H-indazol-1-yl)methyl)piperidin-1-yl)ethanone;
3-(6-(6-fluoro-1H-indol-3-yl)-1H-indazol-1-yl)propanamide;
6-(6-fluoro-1H-indol-3-yl)-1-(piperidin-4-yl)-1H-indazole;
1-(4-(6-(6-fluoro-1H-indol-3-yl)-1H-indazol-1-yl)piperidin-1-yl)ethanone;
5-(6-fluoro-1H-indol-3-yl)-1H-indazole;
2-(5-(6-fluoro-1H-indol-3-yl)-1H-indazol-1-yl)acetamide;
5-(6-fluoro-1H-indol-3-yl)-1-(piperidin-4-ylmethyl)-1H-indazole;
1-(4-((5-(6-fluoro-1H-indol-3-yl)-1H-indazol-1-yl)methyl)piperidin-1-yl)ethanone;
3-(5-(6-fluoro-1H-indol-3-yl)-1H-indazol-1-yl)propanamide;
5-(6-fluoro-1H-indol-3-yl)-1-(piperidin-4-yl)-1H-indazole;
1-(4-(5-(6-fluoro-1H-indol-3-yl)-1H-indazol-1-yl)piperidin-1-yl)ethanone;
2-(6-(6-fluoro-1H-indol-3-yl)-2H-indazol-2-yl)acetamide;
1-(4-((6-(6-fluoro-1H-indol-3-yl)-2H-indazol-2-yl)methyl)piperidin-1-yl)ethanone;
3-(6-(6-fluoro-1H-indol-3-yl)-2H-indazol-2-yl)propanamide;
6-(6-fluoro-1H-indol-3-yl)-2-(piperidin-4-yl)-2H-indazole;
1-(4-(6-(6-fluoro-1H-indol-3-yl)-2H-indazol-2-yl)piperidin-1-yl)ethanone;
2-(5-(6-fluoro-1H-indol-3-yl)-2H-indazol-2-yl)acetamide;
1-(4-((5-(6-fluoro-1H-indol-3-yl)-2H-indazol-2-yl)methyl)piperidin-1-yl)ethanone;
3-(5-(6-fluoro-1H-indol-3-yl)-2H-indazol-2-yl)propanamide;
5-(6-fluoro-1H-indol-3-yl)-2-(piperidin-4-yl)-2H-indazole;
1-(4-(5-(6-fluoro-1H-indol-3-yl)-2H-indazol-2-yl)piperidin-1-yl)ethanone;
5-(6-fluoro-1H-indol-3-yl)-1H-benzo[d]imidazole;
5-(6-fluoro-1H-indol-3-yl)-2-methyl-1H-benzo[d]imidazole;
(5-(6-fluoro-1H-indol-3-yl)-1H-benzo[d]imidazol-2-yl)methanamine;
1-(5-(6-fluoro-1H-indol-3-yl)-1H-benzo[d]imidazol-2-yl)-N,N-dimethylmethanamine;
2-(((5-(6-fluoro-1H-indol-3-yl)-1H-benzo[d]imidazol-2-yl)methyl)amino)ethanol;
N-((5-(6-fluoro-1H-indol-3-yl)-1H-benzo[d]imidazol-2-yl)methyl)acetamide;
2-amino-N-((5-(6-fluoro-1H-indol-3-yl)-1H-benzo[d]imidazol-2-yl)methyl)acetamide;
N-((5-(6-fluoro-1H-indol-3-yl)-1H-benzo[d]imidazol-2-yl)methyl)methanesulfonamide;
5-(6-fluoro-1H-indol-3-yl)-2-((4-methylpiperazin-1-yl)methyl)-1H-benzo[d]imidazole;
5-(6-fluoro-1H-indol-3-yl)-2-((4-(methylsulfonyl)piperazin-1-yl)methyl)-1H-benzo[d]imidazole;
2-(5-(6-fluoro-1H-indol-3-yl)-1H-benzo[d]imidazol-2-yl)ethanamine;
N-(2-(5-(6-fluoro-1H-indol-3-yl)-1H-benzo[d]imidazol-2-yl)ethyl)acetamide;
N-(2-(5-(6-fluoro-1H-indol-3-yl)-1H-benzo[d]imidazol-2-yl)ethyl)methanesulfonamide;
5-(6-fluoro-1H-indol-3-yl)-2-(2-(methylsulfonyl)ethyl)-1H-benzo[d]imidazole;
5-(6-fluoro-1H-indol-3-yl)-2-(piperidin-4-yl)-1H-benzo[d]imidazole;
5-(6-fluoro-1H-indol-3-yl)-2-(1-(methylsulfonyl)piperidin-4-yl)-1H-benzo[d]imidazole;
5-(6-fluoro-1H-indol-3-yl)-1H-benzo[d]imidazol-2-amine;
N-(5-(6-fluoro-1H-indol-3-yl)-1H-benzo[d]imidazol-2-yl)acetamide;
N-(5-(6-fluoro-1H-indol-3-yl)-1H-benzo[d]imidazol-2-yl)methanesulfonamide;
1-(5-(6-fluoro-1H-indol-3-yl)-1H-benzo[d]imidazol-2-yl)-N-methylmethanamine;
4-((5-(6-fluoro-1H-indol-3-yl)-1H-benzo[d]imidazol-2-yl)methyl)morpholine;
3-(5-(6-fluoro-1H-indol-3-yl)-1H-benzo[d]imidazol-2-yl)propanamide;
5-(6-fluoro-1H-indol-3-yl)benzo[d]oxazole;
5-(6-fluoro-1H-indol-3-yl)-2-methylbenzo[d]oxazole;
5-(6-fluoro-1H-indol-3-yl)-2-(piperidin-4-yl)benzo[d]oxazole;
1-(4-(5-(6-fluoro-1H-indol-3-yl)benzo[d]oxazol-2-yl)piperidin-1-yl)ethanone;
N-(5-(6-fluoro-1H-indol-3-yl)benzo[d]oxazol-2-yl)acetamide;
N-(5-(6-fluoro-1H-indol-3-yl)benzo[d]oxazol-2-yl)methanesulfonamide;
6-(6-fluoro-1H-indol-3-yl)benzo[d]oxazole;
6-(6-fluoro-1H-indol-3-yl)-2-methylbenzo[d]oxazole;
6-(6-fluoro-1H-indol-3-yl)-2-(piperidin-4-yl)benzo[d]oxazole;
1-(4-(6-(6-fluoro-1H-indol-3-yl)benzo[d]oxazol-2-yl)piperidin-1-yl)ethanone;
N-(6-(6-fluoro-1H-indol-3-yl)benzo[d]oxazol-2-yl)acetamide;
N-(6-(6-fluoro-1H-indol-3-yl)benzo[d]oxazol-2-yl)methanesulfonamide;
6-(6-fluoro-1H-indol-3-yl)-2-(piperazin-1-ylmethyl)-1H-benzo[d]imidazole;
2-(6-(6-fluoro-1H-indol-3-yl)-1H-benzo[d]imidazol-2-yl)acetamide;
5-(6-fluoro-1H-indol-3-yl)-2-(piperazin-1-ylmethyl)benzo[d]oxazole;
5-(6-fluoro-1H-indol-3-yl)-2-((4-methylpiperazin-1-yl)methyl)benzo[d]oxazole;
5-(6-fluoro-1H-indol-3-yl)-2-(morpholinomethyl)benzo[d]oxazole;
2-(6-(6-fluoro-1H-indol-3-yl)-2-oxobenzo[d]oxazol-3(2H)-yl)acetamide;
5-(6-fluoro-1H-indol-3-yl)-1H-benzo[d]imidazol-2(3H)-one;
6-(6-fluoro-1H-indol-3-yl)-2-(morpholinomethyl)benzo[d]oxazole;
3-(6-(6-fluoro-1H-indol-3-yl)-2-oxobenzo[d]oxazol-3(2H)-yl)propanamide;
6-(6-fluoro-1H-indol-3-yl)-2-((4-methylpiperazin-1-yl)methyl)benzo[d]oxazole;
2-(5-(6-fluoro-1H-indol-3-yl)-2-oxobenzo[d]oxazol-3(2H)-yl)acetamide;
N-((5-(6-fluoro-1H-indol-3-yl)benzo[d]oxazol-2-yl)methyl)methanesulfonamide;
3-(5-(6-fluoro-1H-indol-3-yl)-2-oxobenzo[d]oxazol-3(2H)-yl)propanamide;
3-(benzo[b]thiophen-5-yl)-6-fluoro-1H-indole;
N-((5-(6-fluoro-1H-indol-3-yl)benzo[d]oxazol-2-yl)methyl)acetamide;
N-((6-(6-fluoro-1H-indol-3-yl)benzo[d]oxazol-2-yl)methyl)methanesulfonamide;
N-((6-(6-fluoro-1H-indol-3-yl)benzo[d]oxazol-2-yl)methyl)acetamide;
(6-(6-fluoro-1H-indol-3-yl)benzo[d]oxazol-2-yl)methanamine;
5-(6-fluoro-1H-indol-3-yl)-2-(piperidin-4-ylmethyl)-2H-indazole;
6-(6-fluoro-1H-indol-3-yl)-2-(piperazin-1-ylmethyl)benzo[d]oxazole;
3-(benzo[b]thiophen-6-yl)-6-fluoro-1H-indole;
5-(6-fluoro-1H-indol-3-yl)-2-(2-(methylsulfonyl)ethyl)benzo[d]oxazole;
(5-(6-fluoro-1H-indol-3-yl)benzo[d]oxazol-2-yl)methanamine;
3-(benzofuran-5-yl)-6-fluoro-1H-indole;
3-(6-(6-fluoro-1H-indol-3-yl)benzo[d]oxazol-2-yl)propanamide;
2-(6-(6-fluoro-1H-indol-3-yl)-1H-benzo[d]imidazol-1-yl)acetamide;
2-(5-(6-fluoro-1H-indol-3-yl)-1H-benzo[d]imidazol-1-yl)acetamide;
3-(5-(6-fluoro-1H-indol-3-yl)-1H-indazol-1-yl)-1-morpholinopropan-1-one;
3-(5-(6-fluoro-1H-indol-3-yl)-1H-indazol-1-yl)-1-(4-methylpiperazin-1-yl)propan-1-one;
N-(2-(dimethylamino)ethyl)-3-(5-(6-fluoro-1H-indol-3-yl)-1H-indazol-1-yl)propanamide;
3-(5-(6-fluoro-1H-indol-3-yl)-1H-indazol-1-yl)-N-(2-hydroxyethyl)propanamide;
3-(5-(6-fluoro-1H-indol-3-yl)-1H-indazol-1-yl)-N-(2-(methylsulfonyl)ethyl)propanamide;
3-(5-(6-fluoro-1H-indol-3-yl)-1H-indazol-1-yl)-1-(piperazin-1-yl)propan-1-one;
6-(6-fluoro-1H-indol-3-yl)indolin-2-one;
3-(6-(6-fluoro-1H-indol-3-yl)-1H-benzo[d]imidazol-1-yl)propanamide;
3-(5-(6-fluoro-1H-indol-3-yl)-1H-benzo[d]imidazol-1-yl)propanamide;
1-(4-(6-(6-fluoro-1H-indol-3-yl)benzo[d]oxazol-2-yl)piperidin-1-yl)ethanone;
6-(6-fluoro-1H-indol-3-yl)-2-(1-(methylsulfonyl)piperidin-4-yl)benzo[d]oxazole;
5-(6-fluoro-1H-indol-3-yl)indolin-2-one;
6-(6-fluoro-1H-indol-3-yl)benzo[d]oxazol-2-amine;
N-((6-(6-fluoro-1H-indol-3-yl)benzo[d]oxazol-2-yl)methyl)-2-methoxyethanesulfonamide;
2-(dimethylamino)-N-((6-(6-fluoro-1H-indol-3-yl)benzo[d]oxazol-2-yl)methyl)ethanesulfonamide;
6-(6-fluoro-1H-indol-3-yl)-2-(2-(methylsulfonyl)ethyl)benzo[d]oxazole;
1-((6-(6-fluoro-1H-indol-3-yl)benzo[d]oxazol-2-yl)methyl)urea;
1-carbamoyl-1-((6-(6-fluoro-1H-indol-3-yl)benzo[d]oxazol-2-yl)methyl)urea;
5-(1H-indol-3-yl)-1H-indazole;
6-(6-fluoro-1H-indol-3-yl)-2-(2-(4-methylpiperazin-1-yl)ethyl)benzo[d]oxazole;
N-((6-(6-fluoro-1H-indol-3-yl)benzo[d]oxazol-2-yl)methyl)-2-hydroxyethanesulfonamide;
N-((6-(6-fluoro-1H-indol-3-yl)benzo[d]oxazol-2-yl)methyl)-2-morpholinoethanesulfonamide;
6-(1H-indol-3-yl)-1H-indazole;
6-(1H-indol-3-yl)benzo[d]oxazole;
6-(6-fluoro-1H-indol-3-yl)-2-(2-(piperazin-1-yl)ethyl)benzo[d]oxazole;
5-(6-chloro-1H-indol-3-yl)benzo[d]oxazole;
6-(6-fluoro-1H-indol-3-yl)benzo[d]oxazole-2-thiol;
6-(6-fluoro-1H-indol-3-yl)-2-(piperidin-4-ylmethyl)benzo[d]oxazole;
N-((6-(1H-indol-3-yl)benzo[d]oxazol-2-yl)methyl)methanesulfonamide;
(S)—N-((6-(6-fluoro-1H-indol-3-yl)benzo[d]oxazol-2-yl)methyl)-1-methylpyrrolidine-2-carboxamide;
6-(6-fluoro-1H-indol-3-yl)-2-methoxybenzo[d]oxazole;
Amino {[6-(6-fluoroindol-3-yl)benzoxazol-2-yl]methyl}sulfonamide;
5-(1H-indol-3-yl)benzo[d]oxazole;
6-(6-fluoro-1H-indol-3-yl)-2-((1-(methylsulfonyl)piperidin-4-yl)methyl)benzo[d]oxazole;
6-(6-fluoro-1H-indol-3-yl)-2-(2-(4-(methylsulfonyl)piperazin-1-yl)ethyl)benzo[d]oxazole;
5-(5-chloro-1H-indol-3-yl)benzo[d]oxazole;
N-((6-(6-fluoro-1H-indol-3-yl)benzo[d]oxazol-2-yl)methyl)-2-(4-methylpiperazin-1-yl)ethanesulfonamide;
N-((6-(6-fluoro-1H-indol-3-yl)benzo[d]oxazol-2-yl)methyl)-2-(piperazin-1-yl)ethanesulfonamide;
N-((6-(6-fluoro-1H-indol-3-yl)benzo[d]oxazol-2-yl)methyl)-2-(pyrrolidin-1-yl)ethanesulfonamide;
6-(6-fluoro-1H-indol-3-yl)-2-((1-methylpiperidin-4-yl)methyl)benzo[d]oxazole;
N-((6-(6-fluoro-1H-indol-3-yl)benzo[d]oxazol-2-yl)methyl)-2-(4-(methylsulfonamido)piperidin-1-yl)ethanesulfonamide;
N-((6-(6-chloro-1H-indol-3-yl)benzo[d]oxazol-2-yl)methyl)methanesulfonamide;
N-((6-(5-fluoro-1H-indol-3-yl)benzo[d]oxazol-2-yl)methyl)methanesulfonamide
5-((6-(6-fluoro-1H-indol-3-yl)benzo[d]oxazol-2-yl)methyl)imidazolidine-2,4-dione;
5-(6-fluoro-1H-indol-3-yl)benzo[d]isothiazol-3(2H)-one 1,1-dioxide;
2-(6-(5-fluoro-1H-indol-3-yl)-2-oxobenzo[d]oxazol-3(2H)-yl)acetamide;
5-(6-fluoro-1H-indol-3-yl)isoindoline-1,3-dione;
6-fluoro-3-(isoindolin-5-yl)-1H-indole;
6-(6-fluoro-1H-indol-3-yl)benzo[d]isothiazol-3(2H)-one 1,1-dioxide;
5-(6-fluoro-1H-indol-3-yl)-2,3-dihydrobenzo[d]isothiazole 1,1-dioxide;
6-(6-fluoro-1H-indol-3-yl)-3-(piperidin-4-yl)benzo[d]oxazol-2(3H)-one;
(6-(6-fluoro-1H-indol-3-yl)-2-methylbenzo[d]oxazol-5-yl)methanol;
5-(6-fluoro-1H-indol-3-yl)-2-methylbenzo[d]isothiazol-3(2H)-one 1,1-dioxide;
2-(2-(cis-3,5-dimethylpiperazin-1-yl)ethyl)-6-(6-fluoro-1H-indol-3-yl)benzo[d]oxazole;
1-(2-(6-(6-fluoro-1H-indol-3-yl)benzo[d]oxazol-2-yl)ethyl)piperidin-4-ol;
2-(2-(trans-2,5-dimethylpiperazin-1-yl)ethyl)-6-(6-fluoro-1H-indol-3-yl)benzo[d]oxazole;
(+)-2-(2-((3R,5R)-3,5-dimethylpiperazin-1-yl)ethyl)-6-(6-fluoro-1H-indol-3-yl)benzo[d]oxazole;
2-(2-(2,6-diazaspiro[3.3]heptan-2-yl)ethyl)-6-(6-fluoro-1H-indol-3-yl)benzo[d]oxazole;
(−)-2-(2-((3S,5S)-3,5-dimethylpiperazin-1-yl)ethyl)-6-(6-fluoro-1H-indol-3-yl)benzo[d]oxazole;
4-(2-(6-(6-fluoro-1H-indol-3-yl)benzo[d]oxazol-2-yl)ethyl)piperazine-2-carboxylic acid; or
3-(benzofuran-6-yl)-6-fluoro-1H-indole;
or a pharmaceutically acceptable enantiomer, salt or solvate thereof.

19. A pharmaceutical composition comprising a compound according to claim 1, or a pharmaceutically acceptable enantiomer, salt or solvate thereof, and at least one pharmaceutically acceptable carrier, diluent, excipient and/or adjuvant.

20. A method of treating and/or preventing of cancer, neurodegenerative disorders such as Parkinson's disease, Alzheimer's disease and Huntington's disease, chronic viral infections such as HCV and HIV, depression, and obesity, or inhibiting TD02, said method comprising administering a compound according to claim 1 to a subject in need thereof.

21. The method according to claim 25, wherein said cancer is selected from the group consisting of: bladder carcinoma, hepatocarcinoma, melanoma, mesothelioma, neuroblastoma, sarcoma, breast carcinoma, leukemia, renal cell carcinoma, colorectal carcinoma, head & neck carcinoma, lung carcinoma, brain tumor, glioblastoma, astrocytoma, myeloma and pancreatic carcinoma.

22. Process for manufacturing a compound of Formula I according to claim 1 a pharmaceutically acceptable enantiomer, salt or solvate thereof, comprising:
(a1) reacting a compound of Formula (i) 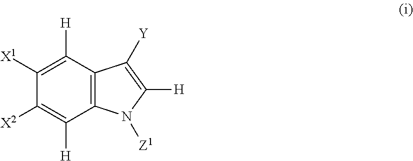
wherein
X¹ and X² represent each independently H, halogen, alkyl, haloalkyl;
Z¹ represents H or an amino protecting group;
Y represents an halogen, an alkylsulfonyloxy having 1-6 carbon atoms or arylsulfonyloxy having 6-10 carbon atoms;
with a compound of Formula (ii) 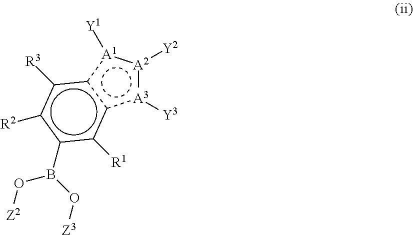
wherein
Z² and Z³ represent H or alkyl groups, with the possibility for Z² and Z³ to form a ring; so as to obtain a compound of Formula (iii), 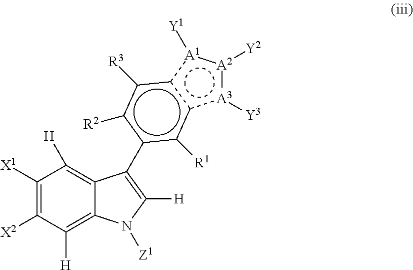
wherein Z¹, X¹, X², R¹, R², R³, A¹, A², A³, Y¹, Y² and Y³ are above; and
(b1) in the case wherein Z¹ is not H, deprotecting the indole amine of compound of Formula (iii), to afford compound of Formula I.

23. The process according to claim 22, wherein the amino protecting group of Z1 is an arylsulphonyl, a tert-butoxy carbonyl, a methoxymethyl, a para-methoxy benzyl, or a benzyl.

24. The process according to claim 22, wherein when Y is the halogen, Y is iodine, bromine or chlorine.

25. The process according to claim 22, wherein when Y is the alkylsulfonyl, it is methylsulfonyloxy or trifluoromethylsulfonyloxy.

26. The process according to claim 22, wherein when Y is the arylsulfonyl, it is phenyl- or p-tolylsulfonyloxy.
